(19) United States
(12) Patent Application Publication
(10) Pub. No.: US 2026/0021069 A1
(43) Pub. Date: Jan. 22, 2026

(54) ADVANTAGEOUS FLUOROBENZOFURANS FOR THE TREATMENT OF MENTAL DISORDERS OR ENHANCEMENT

(71) Applicant: TACTOGEN INC, Palo Alto, CA (US)

(72) Inventor: Matthew J. Baggott, Redwood City, CA (US)

(73) Assignee: TACTOGEN INC, Palo Alto, CA (US)

(21) Appl. No.: 19/345,974

(22) Filed: Sep. 30, 2025

Related U.S. Application Data
(63) Continuation of application No. PCT/US2024/022089, filed on Mar. 28, 2024.
(60) Provisional application No. 63/456,328, filed on Mar. 31, 2023.

(51) Int. Cl. A61K 31/343; C07D 307/79

(52) U.S. Cl. CPC A61K 31/343; C07D 307/79

(57) ABSTRACT

The present invention discloses pharmaceutically active halogenated benzofuran compounds, for example fluorobenzofurans for the treatment of mental disorders or for mental enhancement, including for entactogenic therapy. The present invention also includes fluorobenzofuran compounds, compositions, and methods for generally modulating central nervous system activity and treating central nervous system disorders.

### CROSS-REFERENCE TO RELATED APPLICATIONS

This application is a continuation of International Patent Application No. PCT/US2024/022089, filed in the U.S. Receiving Office on Mar. 28, 2024, which claims the benefit of U.S. Provisional Application No. 63/456,328, filed Mar. 31, 2023. The entirety of this application is hereby incorporated by reference herein for all purposes.

### FIELD OF THE INVENTION

The present invention is in the area of pharmaceutically active halogenated benzofuran compounds, for example fluorobenzofurans, for the treatment of mental disorders or for mental enhancement, including for entactogenic therapy. The present invention also includes fluorobenzofuran compounds, compositions, and methods for generally modulating central nervous system activity and treating central nervous system disorders.

### BACKGROUND

Mental disorders, including Post-Traumatic Stress Disorder (PTSD), are more common in society than most recognize, as they can be silent or hidden. The U.S. National Institute of Mental Health (NIMH) reports that 70% of all adults have experienced at least one traumatic event in their lives, and 20% of these people will get PTSD. NIMH estimates that about 3.6% of U.S. adults have PTSD in a one-year period. PTSD can significantly impair a person's ability to function at work, at home and socially. While many people associate PTSD with veterans and combat, in fact, it is prevalent in all aspects of society.

The World Health Organization reports that depression is a serious medical disorder affecting at least 264 million people globally of all ages. When long lasting and with even moderate intensity or severe intensity, depression can become a serious health condition. It is a leading cause of disability and if not treated can lead to suicidal thoughts and ideation which can progress to suicide as well as addiction. According to WHO, suicide is the second leading cause of death globally in 15-29 year olds.

Other mental disorders that can profoundly affect a person's ability to function normally in society include anxiety disorders such as generalized anxiety disorder, phobia, panic disorder, separation anxiety disorder, stress-related disorders, adjustment disorder, dissociative disorder, eating disorders (e.g., bulimia, anorexia, etc.), attention deficit disorder, sleep disorders, disruptive disorders, neurocognitive disorders, obsessive compulsive disorders, and personality disorders, among others.

While medications are available or in clinical testing for a range of mental disorders, these disorders remain a large burden of disease globally and are insufficiently treated. Further, many of the medications have a long ramp-up time of weeks or more, during which period some patients needing therapy stop the medication out of impatience or belief it doesn't work.

Many mental disorders are caused by, affected by and/or may be treated by altered levels of neurotransmitters, which are chemicals that transmit a signal from a neuron across the synapse to another neuron. Brain neurotransmitter systems include the serotonin system, the noradrenaline (norepinephrine) system, the dopamine system and the cholinergic system. Dopamine, serotonin and noradrenaline (norepinephrine) are classed as phenylethylamines, and noradrenaline is also a catecholamine. Drugs that prevent a neurotransmitter from binding to its receptor are called receptor antagonists. Drugs that bind to a receptor and mimic the normal neurotransmitter are receptor agonists. Other drugs interfere with the deactivation of a neurotransmitter after it has been released, which prolongs its action. This can be accomplished by blocking the re-uptake of the transmitter (reuptake inhibitor) or by inhibiting enzymes that degrade the transmitter. A direct agonist binds directly to its associated receptor site. An indirect agonist increases the binding of a neurotransmitter at the target receptor by stimulating the release or preventing the reuptake of the neurotransmitter.

Dopamine receptors are involved in many neurological processes such as motivation, pleasure, cognition, memory, learning, and fine motor control. It is the primary neurotransmitter involved in the reward pathway. Drugs that increase dopamine may produce euphoria. Some widely used drugs such as methamphetamines alter the functioning of the dopamine transporter (DAT), which is responsible for removing dopamine from the neural synapse.

Norepinephrine, also called noradrenaline, mobilizes the body for activity, and is at a high level during stress or danger. It focuses attention and increases arousal and alertness.

Serotonin (5-hydroxytryptamine or “5-HT”) receptors influence various neurological functions such as aggression, anxiety, appetite, cognition, learning, memory, mood and sleep. 5-HT receptors are the target of FDA approved drugs and unapproved drugs, including antidepressants, antipsychotics, hallucinogens (psychedelics), and entactogens (empathogens). There are seven families of 5-HT receptors and each has subtypes, creating a highly complex signaling system. For example, when 5-HT_{2A} is agonized it often induces hallucinogenic effects, whereas 5-HT_{2B}, which is more predominantly in the periphery than in the brain, when chronically agonized, can cause toxicity such as valvulopathy. In contrast, 5-HT_{1B} when agonized regulates serotonergic neurons and likely contributes to the social effects of entactogens.

Current treatments for a range of mental disorders typically involve the use of selective serotonin reuptake inhibitors (SSRIs), such as citalopram (Celexa), Escitalopram (Lexapro), Fluoxetine (Prozac), Paroxetine (Paxil) and Sertraline (Zoloft). SSRIs block the reabsorption (i.e., reuptake) of serotonin into neurons, thereby increasing levels of serotonin in the brain. However, SSRIs are generally slow to achieve clinically meaningful benefit, requiring weeks to produce therapeutic effects. Moreover, many patients are nonresponders and show no benefit at all (Masand et al., Harv. Rev. Psychiatry, 1999, 4: 69-84; Rosen et al., J. Clin. Psychopharmacol., 1999, 19: 67-85).

Bupropion (Wellbutrin), in contrast, is an anti-depressant that is a norepinephrine-dopamine reuptake inhibitor, which provides more stimulant effects, including weight loss.

Another class of drugs for treatment of CNS mental disorders is monoamine releasers. Monoamine releasers induce the release of one or more monoamine neurotransmitters (e.g., dopamine, serotonin, or epinephrine) from neurons in the brain. Monoamine releasers rapidly modulate the brain systems that are more slowly affected by SSRIs. However, their stimulant and euphoric effects frequently lead them to have high abuse liability. Hence, although the monoamine releasers based on the phenethylamine structure, such as amphetamine (Benzedrine, Dexedrine) and methamphetamine (Obetrol, Pervitin), were widely employed as antidepressants in the mid-20th century, such agents are now used much more cautiously, and primarily treat attention deficit hyperactivity disorder (ADHD).

While the above drugs may be helpful in certain patients or settings, better alternatives are strongly needed. The prevalent use of unapproved drugs for self-medication urges a solution with additional approved drugs that more adequately treat mental disorders or are able to provide mental enhancement.

Entactogens (empathogens) have become the focus of more attention to solve some of these serious health problems. They increase feelings of authenticity and emotional openness while decreasing social anxiety (Baggott et al., Journal of Psychopharmacology 2016, 30.4: 378-87). Entactogens are typically monoamine releasers that appear to produce their effects in part by releasing serotonin which stimulates hypothalamic serotonergic receptors, thus triggering release of the hormone oxytocin, while also stimulating serotonergic 5-HT₁B receptors on cells in the nucleus accumbens area of the brain. They can be distinguished from drugs that are primarily hallucinogenic or psychedelic, and amphetamines, which are primarily stimulants. The most well-known entactogen is MDMA (3,4-methylenedioxymethamphetamine). Other examples of entactogens are MDA, MBDB, MDOH, and MDEA, however, these drugs do have varying and complex effects that result from binding to a range of 5-HT receptors.

The aminoalkylbenzofurans 1-(1-benzofuran-5-yl)-N-methylpropan-2-amine (5-MAPB) and 1-(1-benzofuran-6-yl)-N-methylpropan-2-amine (6-MAPB), among others, are reported to share some effects with entactogens and have undergone preliminary pharmacological profiling (Rickli et al. British Journal of Pharmacology, 2015, 172: 3412-3425; Sahai et al., Progress in Neuropsychopharmacology & Biological Psychiatry, 2017, 75(1-9); Fuwa et al., The Journal of Toxicological Sciences, 2016, 41(3), 329-37).

Before being studied in a laboratory setting, these compounds, and a small number of similar compounds such as 1-(benzofuran-5-yl)-N-methylbutan-2-amine (5-MBPB), were initially sold on the black or gray market and used for self-medication or their euphoric effects (EMCDDA-Europol (2015) Annual Report on the Implementation of Council Decision 2005/387/JHA and European Drug Report, Trends and Developments (2020), European Monitoring Centre for Drugs and Drug Addiction). Additionally, U.S. Pat. No. 7,045,545 discloses certain aminoalkyl benzofurans as agonists of serotonin 5-HT_{2C} receptors.

MDMA is currently in human clinical trials in the United States (clinicaltrials.gov; NCT03537014) and Europe for approval for use in psychotherapy sessions for severe PTSD and has been suggested as useful for aiding social cognition (Preller & Vollenweider, Frontiers in Psychiatry, 2019, 10; Hysek et al., Social cognitive and affective neuroscience, 2015, 9.11, 1645-52). The FDA granted breakthrough therapy designation for the trial and has also agreed to an expanded access program, both indicative of promising results. (Feduccia et al., Frontiers in Psychiatry, 2019, 10: 650; Sessa et al., Frontiers in Psychiatry, 2019, 10: 138). While MDMA has significant therapeutic potential, it has a number of features that potentially make it contraindicated for some patients. This includes its ability to produce acute euphoria, acute hypertensive effects, risk of hyponatremia, and oxidative stress.

Patents and patent applications describing entactogenic compounds include WO 2021/252538, WO 2022/010937, WO 2022/032147, WO 2022/061242, WO 2023/081306, WO 2023/107653, WO 2023/107715, WO 2023/183613, and U.S. Pat. No. 11,767,305, which are assigned to Tactogen Inc. Additional patent applications and publications describing entactogenic compounds and methods of using entactogenic compounds include but are not limited to U.S. Pat. Nos. 7,045,545, 11,603,353, U S. publication no. US2023/233688, PCT publications-WO 2005/058865, WO 2020/169850, WO 2020/169851, WO 2021/257169, WO 2021/225796, WO2022/106947, WO 2022/214889, WO 2022/120181, WO 2022/072808, WO 2022/038171, WO 2023/049480, WO 2023/036473, WO 2023/028022, WO 2023/028092, and WO 2023/028091.

The urgent need for more effective therapies for mental disorders, mental enhancement and other CNS disorders is clear and requires substantial new research and attention.

It is an object of the present invention to provide advantageous compositions and their use and manufacture for the treatment of mental disorders and enhancement. Additional objects are to provide drugs with a more rapid onset to be used in a clinical setting such as counseling, e.g., PTSD and other disorder counseling or a home setting, which open the patient to empathy, sympathy and acceptance. A further objective is to provide effective treatments for a range of CNS disorders.

### SUMMARY OF THE INVENTION

The present invention provides halogenated benzofuran compounds, compositions, and methods to treat mental disorders and more generally central nervous system disorders, as well as for mental enhancement. In certain aspects a halogenated benzofuran compound of the present invention provides advantageous pharmacological properties that are highly desirable as a therapeutic for the treatment of mental disorders. In certain embodiments the halogenated benzofuran compound is a fluorobenzofuran.

The embodiments of the invention are presented to meet the goal of assisting persons with mental disorders, who desire mental enhancement or suffer from other CNS disorders by providing milder therapeutics that are fast acting and that reduce the properties that decrease the patient experience, are counterproductive to the therapy or are undesirably toxic. One goal of the invention is to provide therapeutic compositions that increase empathy, sympathy, openness and acceptance of oneself and others, which can be taken if necessary, as part of therapeutic counseling sessions, or when necessary, episodically, or even consistently, as prescribed by a healthcare provider.

In certain aspects a compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV, or a pharmaceutically acceptable salt or salt mixture thereof is provided.

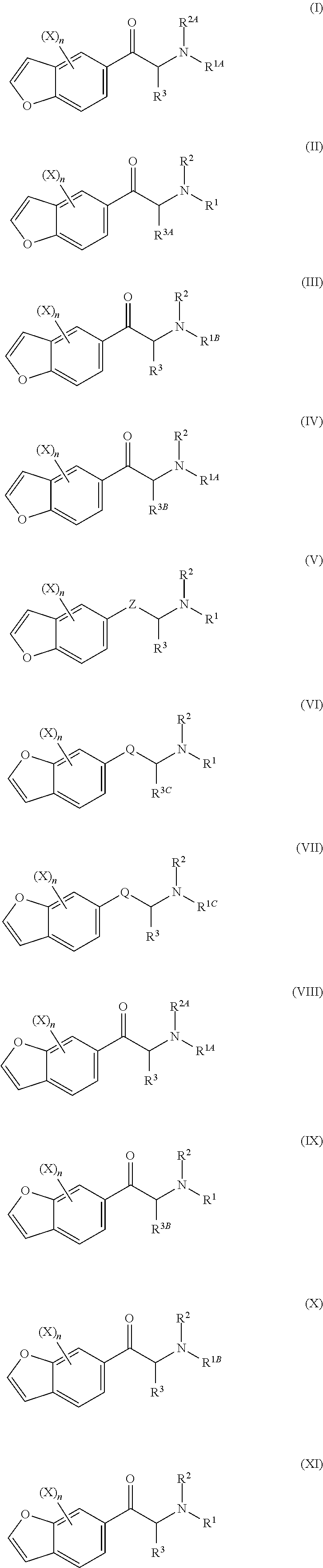

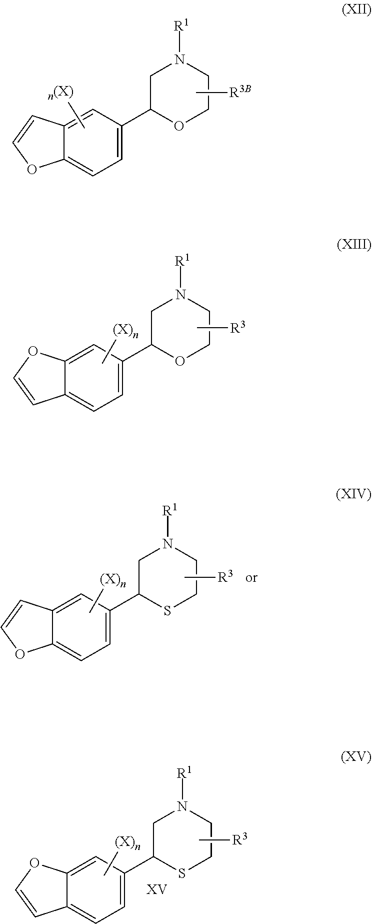

wherein:
R¹ and R² are independently selected from H, C₁-C₄ alkyl, C₁-C₄ haloalkyl, CH₂OH, and —CH₂CH₂OH;
R^{1A} and R^{2A} are independently selected from C₁-C₄ alkyl, C₁-C₄ haloalkyl, CH₂OH, and —CH₂CH₂OH;
R^{1B} is C₂-C₄ alkyl, C₁-C₄ haloalkyl, CH₂OH, or —CH₂CH₂OH;
R^{1C} is C₂-C₄ haloalkyl;
in certain embodiments R^{1C} is CH₂CH₂F, CH₂CHF₂, or CH₂CF₃;
R³ is selected from H, C₁-C₄ alkyl, C₁-C₄ haloalkyl, CH₂OH, and —CH₂CH₂OH; in certain embodiments R³ is C₁-C₂ alkyl;
R⁴ and R⁵ are independently selected from H, C₁-C₄ alkyl, C₁-C₄ haloalkyl, F, Cl, and Br;
R^{3A} is C₄ alkyl, C₁-C₄ haloalkyl, CH₂OH, or —CH₂CH₂OH;
R^{3B} is C₁-C₄ alkyl, C₁-C₄ haloalkyl, CH₂OH, or —CH₂CH₂OH;
R^{3C} is C₁-C₄ haloalkyl;
Q is

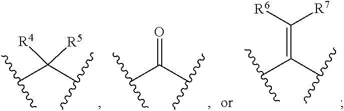

Z is

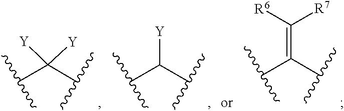

R⁶ and R⁷ are independently selected from H, C₁-C₄ alkyl, C₁-C₄ haloalkyl, F, Cl, and Br;
each X is independently selected from —F, —Cl, and —Br;
each Y is independently selected from —F, —Cl, and —Br; and
n is 1 or 2.

In certain embodiments each X is F.

In certain embodiments each Y is F.

In certain embodiments n is 1.

In certain embodiments the halogenated benzofuran compound is selected from:

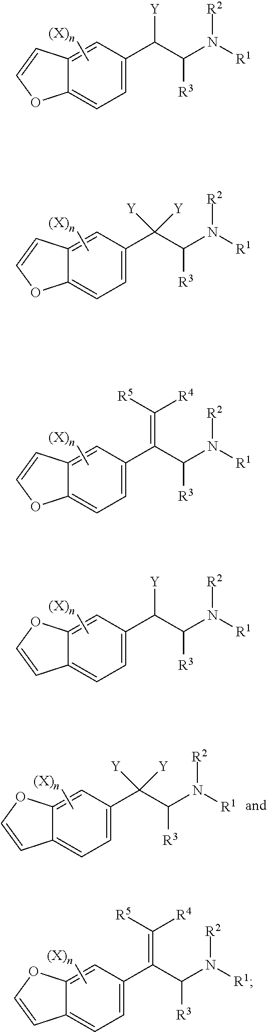

or a pharmaceutically acceptable salt or salt mixture thereof.

When a substituent is depicted with a floating bond on a bicyclic compound described herein, the substituent can be on either cycle unless excluded by context. For example, Formula

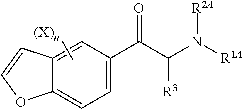

includes the following compounds when n is 1:

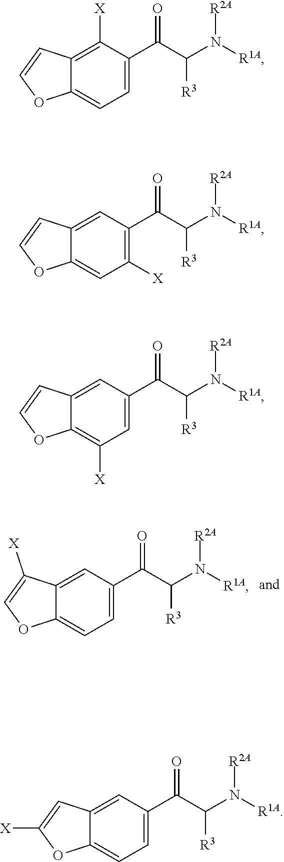

In certain embodiments the halogenated benzofuran compound is of Formula

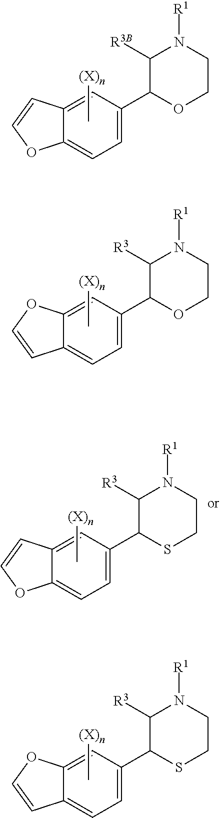

or a pharmaceutically acceptable salt or mixture of salts thereof.

Where multiple chiral centers are present the compound may be a mixture of stereoisomers, a pure stereoisomer, or a chirally enriched mixture of stereoisomers. Stereoisomers of the present invention include

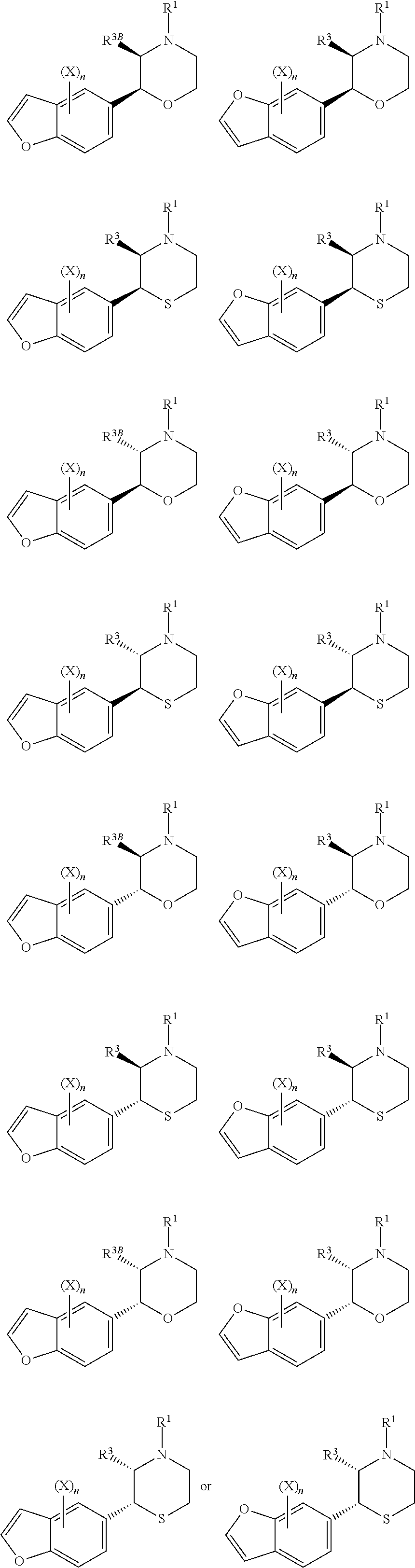

or a pharmaceutically acceptable salt or mixture of salts thereof.

In certain embodiments, the present invention provides an enantiomerically pure or enantiomerically enriched compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV or a pharmaceutically acceptable salt or mixed salt thereof:

In certain other embodiments, the present invention provides an enantiomerically pure or enriched compound of Formula XVI, Formula XVII, Formula XVIII, Formula XIX, or Formula XX or a pharmaceutically acceptable salt or mixed salt thereof.

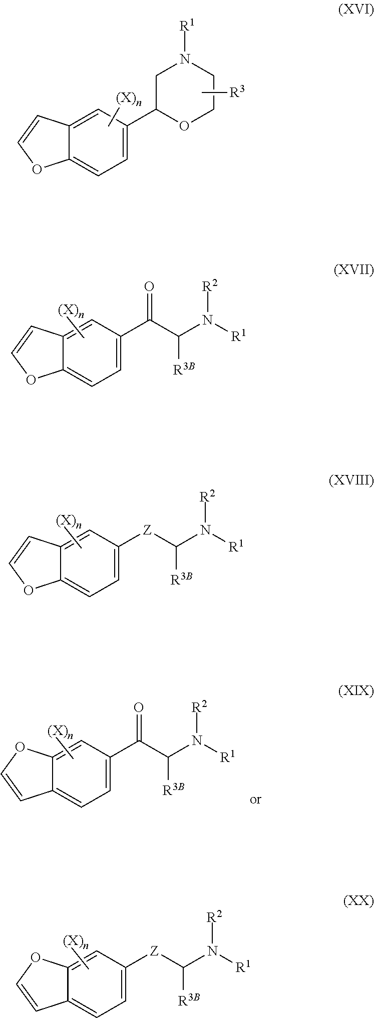

In other aspects, the present invention provides a compound of Formula XVIIIB or Formula XXB or an enantiomerically pure or enriched compound of Formula XVIIIB or Formula XXB or a pharmaceutically acceptable salt or mixed salt thereof.

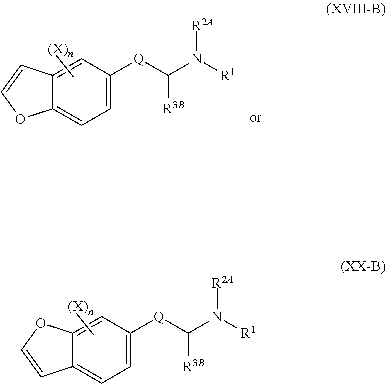

In certain embodiments Q is CH₂.

In certain embodiments Q is C(O).

In certain aspect of the present invention, the use of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, or Formula XX-B or a pharmaceutically acceptable salt or mixed salt thereof, for the treatment of a disorder described herein is provided.

In another aspect of the present invention, the use of a compound of Formula XXI or Formula XXII, or a pharmaceutically acceptable salt or mixed salt thereof, for the treatment of a disorder described herein is provided.

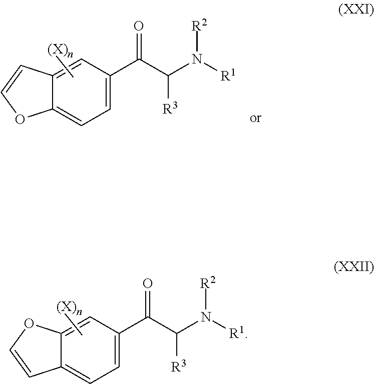

Non-limiting examples of compounds of the present invention include:

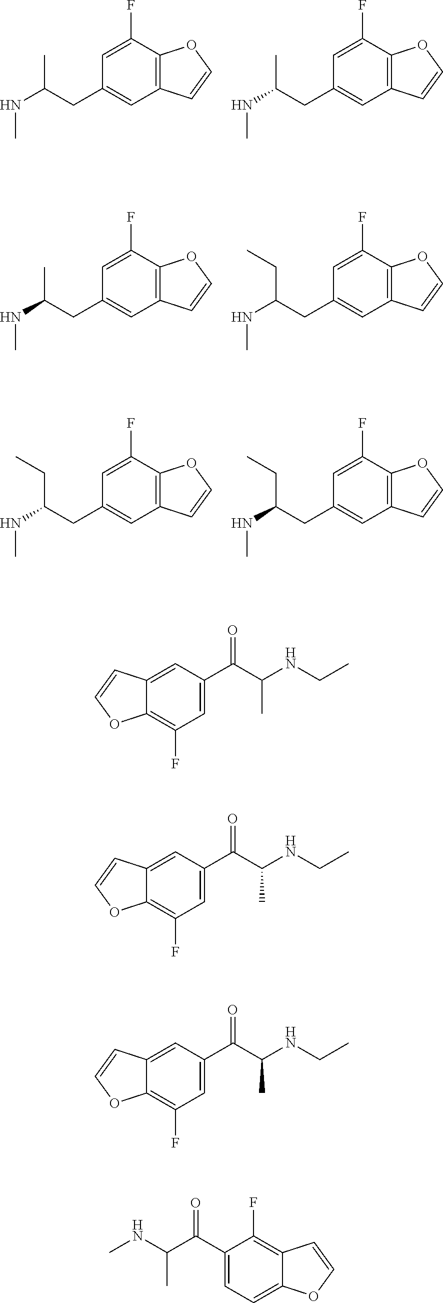

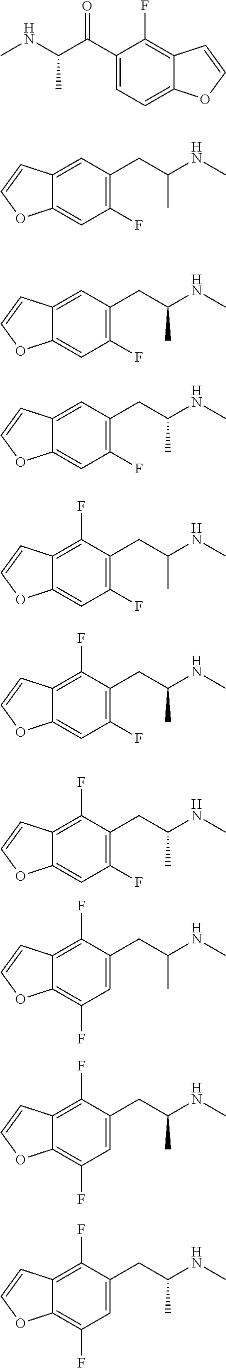

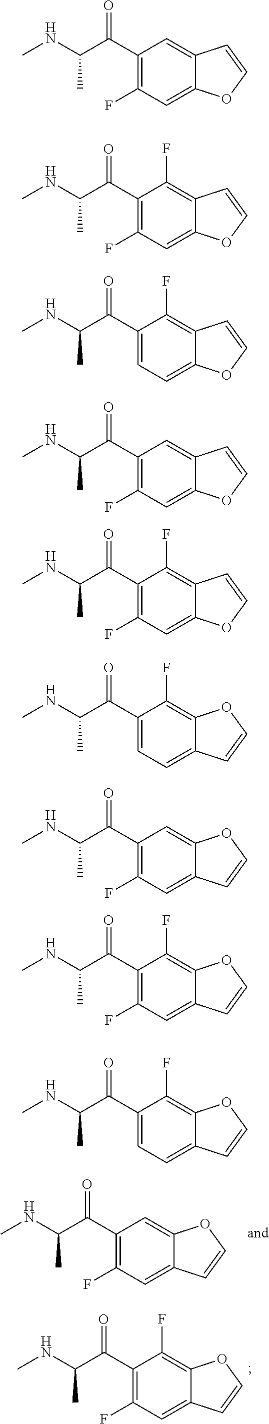

or a pharmaceutically acceptable salt, salt mixture, racemic mixture, or enantiomerically enriched mixture thereof.

In certain embodiments the halogenated benzofuran compound of the present invention is selected from:

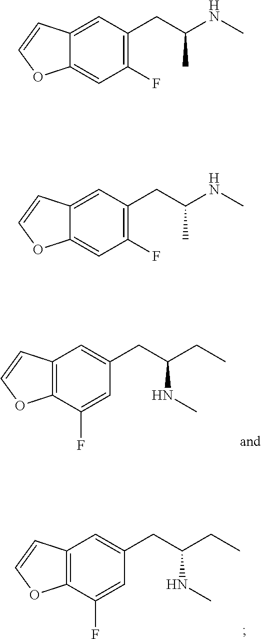 [SMILES: CN[C@@H](C)Cc1cc2ccoc2cc1F ; CN[C@H](C)Cc1cc2ccoc2cc1F ; CC[C@H](Cc1cc(F)c2occc2c1)NC ; CC[C@@H](Cc1cc(F)c2occc2c1)NC]

or a pharmaceutically acceptable salt, salt mixture, racemic mixture, or enantiomerically enriched mixture thereof.

In certain embodiments, isolated enantiomers of the compounds of the present invention show improved binding at the desired receptors and transporters relevant to the goal of treatment for the mental disorder or for mental enhancement. For example, in certain embodiments an S-enantiomer of a halogenated benzofuran compound of the present invention has better binding affinity to 5-HT_{2C} receptor than the R-enantiomer or racemic mixture of the compound. In other embodiments the R-enantiomer of a halogenated benzofuran compound of the present invention has better binding affinity to 5-HT_{2C} receptor than the S-enantiomer or racemic mixture of the compound. In certain aspects enantiomerically enriched mixtures of a halogenated benzofuran compound of the present invention can be used to tune the desired properties of the therapy including the onset, duration, intensity, efficacy, and/or associated side effects.

The compounds described herein may be administered in an effective amount to treat mental disorders described herein or to provide mental enhancement to a human patient in need thereof. In certain embodiments a compound described herein may be used to treat a host such as a human in need thereof as a milder therapeutic than MDMA and which may be acting faster than typical selective serotonin reuptake inhibitors (SSRIs). This enhances the patient experience and encourages the needed medical therapy. In certain embodiments a compound described herein may increase empathy, sympathy, openness and/or acceptance of oneself and others. This compound may be taken, if necessary, as part of one or more therapeutic counseling sessions, or when necessary, episodically, or even consistently, as prescribed by a healthcare provider. In some embodiments, a halogenated benzofuran compound of the present invention may act within a reasonable waiting time in a clinic and lasts for one, two, or several hours or otherwise in a time sufficient to complete the therapy session and then diminishes in effect sufficiently for the patient to leave the clinic and resume normal activities. In other embodiments, the halogenated benzofuran compound may be administered in a periodic or consistent dosage, including a daily dosage in a similar manner to an anti-depressant drug, to enhance self-acceptance, acceptance of others and a general feeling of peace and comfort with surroundings and events.

Thus, a compound described herein may be used to treat mental disorders or provide mental enhancement. For example, in certain embodiments a method for treating a central nervous system disorder or providing mental enhancement comprising administering an effective amount of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, Formula XX-B, Formula XXI, or Formula XXII or a pharmaceutically acceptable salt or salt mixture thereof, optionally in a pharmaceutical composition is provided.

In some embodiments, the halogenated benzofuran compound of the current invention, as a racemic mixture, chirally enriched mixture, or pure chiral compound (for example an enantiomerically pure diastereomer) has a duration of acute therapeutic effects that is less than that of MDMA (reported to be 4.2 hours with a standard deviation of 1.3 hours after 75 or 125 mg MDMA by Vizeli & Liechti. 2017. Journal of Psychopharmacology, 31(5), 576-588). This may be desirable for reducing the costs and resources needed for pharmacotherapy sessions. In other embodiments, the halogenated benzofuran compound of the current invention, as a racemic mixture, chirally enriched mixture, or pure chiral compound has a duration of acute therapeutic effects that is greater than that of MDMA. This avoids the need for re-administration of the entactogen, which produces nonlinear increases in plasma concentrations and greater unwanted effects.

In some embodiments, the halogenated benzofuran compound of the current invention, as a racemic mixture, chirally enriched mixture, or pure chiral compound produces acute cardiovascular effects that are less than those of MDMA. MDMA produces acute tachycardia and hypertension, which requires safety monitoring and may limit its use in those with preexisting cardiovascular disease (Vizeli & Liechti. 2017. Journal of Psychopharmacology, 31(5), 576-588; MDMA Investigator's Brochure, 13th Edition: Mar. 22, 2021).

In some embodiments, a halogenated benzofuran compound of the present invention has favorable pharmacokinetic properties for administration to a mammal, for example a human. These properties may include having more reproducible and less variable pharmacokinetic properties than MDMA. In certain embodiments, a halogenated benzofuran compound has a less variable maximum plasma concentration (Cₘₐₓ) than MDMA. In certain embodiments, a halogenated benzofuran compound has a less variable area-under-the-concentration-versus-time-curve (AUC) than MDMA. An additional potential beneficial property of a halogenated benzofuran compound is reduced inhibition of CYP enzymes compared to MDMA. Inhibition of such enzymes may cause unwanted toxic drug-drug interactions. In certain embodiments, a halogenated benzofuran compound does not inhibit or shows minimal inhibition of cytochrome p450 isozyme 2D6 (CYP2D6). In certain embodiments, a halogenated benzofuran compound shows less potent inhibition of CYP2D6 than MDMA. In other aspects a halogenated benzofuran of the present invention has superior pharmacokinetic properties as compared to the nonhalogenated benzofuran analog thereof. For example, in certain embodiments a halogenated benzofuran of the present invention has higher blood brain penetration than the nonhalogenated benzofuran analog thereof.

In certain embodiments, a halogenated benzofuran compound of the present invention produces fewer toxic metabolites than MDMA, such as dihydroxy and hydroxy-methoxy metabolites and their thioether conjugates. For MDMA, these include 3,4-dihydroxymethamphetamine, 3,4-dihydroxyamphetamine, 5-(N-acetylcystein-S-yl)-alpha-methyldopamine, 5-(glutathion-S-yl)-alpha-methyldopamine, 2,5-bis(glutathion-S-yl)-alpha-methyldopamine, 2,5-bis(N-acetylcystein-S-yl)-alpha-methyldopamine, 5-(N-acetylcystein-S-yl)-3,4-dihydroxymethamphetamine, 5-(glutathion-S-yl)-3,4-dihydroxymethamphetamine, 2,5-bis(glutathion-S-yl)-3,4-dihydroxymethamphetamine, and 2,5-bis(N-acetylcystein-S-yl)-3,4-dihydroxymethamphetamine (e.g., Carvalho et al 2004 doi:10.1007/s00204-003-0510-7; Jones et al 2005 doi:10.1124/jpet.104.077628).

In certain embodiments, a halogenated benzofuran compound of the present invention has favorable pharmacodynamic properties for administration to a mammal, for example a human. These properties may include having greater, more frequent, or less variable therapeutic effects than MDMA. In certain embodiments, these therapeutic effects are decreases in signs or symptoms of a CNS disorder. In certain embodiments, these therapeutic effects are feelings of authenticity, increased self-acceptance and self-compassion, decreased self-criticism, decreased social anxiety, and decreased negative self-beliefs (Baggott et al 2016 doi:10.1177/026988111562634; Falconer et al 2015 doi:10.1111/papt.12056; van der Kolk et al 2023 doi:10.1101/2023.01.03.23284143, Zeifman et al 2022 doi: 10.31234/osf.io/w8j6t). In certain embodiments, a halogenated benzofuran compound of the present invention produces lower, less frequent, or a less variable adverse side effects than MDMA (Vizeli & Liechti 2017, doi:10.1177/0269881117691569). In certain embodiments, these side effects are anxiety, feelings of drunkenness, or feelings of impairment.

In certain embodiments, a halogenated benzofuran compound of the present invention produces less long-term lowering of the presence or activity of serotonin, serotonin transporter, or tryptophan hydroxylase than MDMA, as can be studied in humans with radioligands or in rats or other mammals with radioligands, immunohistochemistry, and other well-known techniques. In certain embodiments, a halogenated benzofuran compound of the present invention produces less hepatotoxicity than MDMA as can be measured in vivo or in vitro. In certain embodiments, a halogenated benzofuran compound of the present invention produces less oxidative stress, mitochondrial impairment, or neuroinflammation than MDMA (Barbosa et al 2011 doi:10.1111/j.1476-5381.2011.01453.x., Bisagno & Cadet 2021 doi:10.1007/978-3-030-71519-9_80-1; Capela & Carvalho 2022 doi:10.1016/j.crtox.2022.100075). In certain embodiments, a halogenated benzofuran compound of the present invention results in less long-term tolerance to the therapeutic effects of entactogens than MDMA, which can have diminishing therapeutic effects with repeated use.

Effects can be compared as differences in maximum effects (Emax) or differences in total effects (area-under-the-effect-versus-time-curve, AUC) or in other ways known to those skilled in the arts. Comparisons may be made on the basis on concentration (i.e., equimolar exposures), but more preferably are made on the basis of relevant pharmacological or therapeutic activity (i.e., equi-active exposures or multiples therefore). For example, equivalent doses between drugs may be calculated based on the dose (ED50 or ED80 or similar) that produces reliable MDMA-like interoceptive stimulus in a rat drug discrimination assay. Both equivalent doses calculated in this manner and multiples of the equivalent doses may be compared.

In further embodiments, a halogenated benzofuran compound of the present invention is a direct 5-HT_{2A} agonist. Most substances that are 5-HT_{2A} agonists have significant side effects that are often undesirable in a therapeutic context. For example, psilocybin often produces labile mood with frequent anxiety, derealization, and depersonalization, which are signs and symptoms that limit clinical use. In some aspects of the present invention, a halogenated benzofuran compound of the present invention releases 5-HT and is a 5-HT_{2A} agonist while displaying greatly decreased side effects compared to psilocybin, LSD, DMT, 5-MeO-DMT, and other clinically used 5-HT_{2A} agonists.

In certain embodiments, pharmaceutical compositions are disclosed which comprise a compound of any of Formulas I-XXII, either racemic, as pure enantiomers, or in an enantiomerically enriched mixture, and which may be in association with another active agent, as well as with a pharmaceutically acceptable carrier, diluent, or excipient.

In further embodiments, pharmaceutical compositions are disclosed which comprise a compound, diastereomerically enriched mixture, enantiomerically enriched mixture, or chirally pure compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, Formula XX-B, Formula XXI, or Formula XXII, and which may be in association with another active agent, in a pharmaceutically acceptable composition that has a carrier, diluent, or excipient. The pharmaceutical compositions of the present invention may in certain embodiments include a salt mixture, wherein a salt mixture may comprise 1, 2 or more different pharmaceutically acceptable salts together to form a single composition. In some embodiments, enantiomers are mixed that each has a different salt or wherein there is a ratio of salts, as in Adderall, for example, which is a mixture of a racemate of amphetamine as an aspartate salt, racemate of amphetamine as a sulfate salt, and D-amphetamine as a saccharate salt and D-amphetamine as a sulfate salt. These kinds of mixtures of racemic, enantiomerically enriched and pure compounds of the present invention may provide advantageous results.

The invention includes methods for modulating the activity of the CNS of a host in need thereof, such as a human, by administering an effective amount of a compound or composition of the invention. Examples are methods for treating a variety of CNS disorders, as generally listed herein, that have been linked to inadequate functioning of serotonergic neurotransmission in mammals, using a compound or composition of the invention. The invention also includes methods of improving CNS functioning such as reducing neuroticism or psychological defensiveness or increasing creativity, decision-making ability, or openness to experience in a human by administering an effective amount of a compound or composition of the invention.

Specifically, the invention includes methods to treat a neurological or psychiatric central nervous system disorder as further described herein, including a mental disorder, or to provide a mental enhancement, a compound of Formula I-XXII described herein or a pharmaceutically acceptable salt or salt mixture thereof.

Additionally, the invention includes a method of treating a patient with primary or secondary headaches is provided, comprising administering an effective amount of a compound, pure enantiomer, or enantiomerically enriched mixture of a Formula described herein.

These and other objects, features, and advantages of the present invention may be more clearly understood and appreciated from a review of the following detailed description of the disclosed embodiments and examples, and by reference to the appended claims.

The present invention thus includes at least the following aspects:
(i) A compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV, or a pharmaceutically acceptable salt or salt mixture, isotopic derivative, or prodrug thereof;
(ii) A diastereomerically or enantiomerically enriched or pure diastereomer or enantiomer of a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, or Formula XX-B, or a pharmaceutically acceptable salt or salt mixture, isotopic derivative, or prodrug thereof,
(iii) A pharmaceutical composition comprising an effective patient-treating amount of a compound of (i), (ii), Formula XXI, or Formula XXII, in a pharmaceutically acceptable carrier or diluent for any of the uses described herein;
(iv) The pharmaceutically acceptable composition of (iii) in a solid or liquid, systemic, oral, topical or parenteral dosage form;
(v) A method for treating a patient with any neurological or psychological CNS disorder as described herein that includes administering an effective amount of a compound of (i), (ii), Formula XXI, or Formula XXII to a patient such as a human in need thereof,
(vi) A method for treating a patient with Post-Traumatic Stress Disorder (PTSD) as described herein that includes administering an effective amount of a compound of (i), (ii), Formula XXI, or Formula XXII to a patient such as a human in need thereof,
(vii) A method for treating any neurological or psychological CNS disorder comprising administering an effective amount of a compound of (i), (ii), Formula XXI, Formula XXII, or a pharmaceutically acceptable salt, isotopic derivative, or prodrug thereof, as described herein, to a patient, typically a human, in need thereof;
(viii) A compound of (i), (ii), Formula XXI, Formula XXII, or a pharmaceutically acceptable salt, salt mixture, isotopic derivative, or prodrug thereof, for use to treat any disorder as described herein in an effective amount as further described herein;
(ix) A compound of (i), (ii), Formula XXI, or Formula XXII for use in the manufacture of a medicament for the treatment of any of the disorders described herein;
(x) Use of a compound of (i), (ii), Formula XXI, Formula XXII, or a pharmaceutically acceptable salt, salt mixture, isotopic derivative, or prodrug thereof, to treat any disorder as described herein in an effective amount as further described herein;
(xi) Processes for the preparation of therapeutic products that contain an effective amount of a compound of (i), (ii), Formula XXI, or Formula XXII, or a pharmaceutically acceptable salt or salt mixtures, isotopic derivatives, or prodrugs or compositions thereof, as described herein.

### DETAILED DESCRIPTION

Among the various aspects of the present invention are compounds, compositions, methods for modulation of CNS activity, and methods for treatment of CNS disorders, such as posttraumatic stress and adjustment disorders, comprising the halogenated benzofurans disclosed herein. Methods to treat headaches and migraines are also provided.

The present invention includes fluorinated benzofuran compound of Formula I, Formula II, Formula III, Formula IV, Formula V, Formula VI, Formula VII, Formula VIII, Formula IX, Formula X, Formula XI, Formula XII, Formula XIII, Formula XIV, or Formula XV, or a pharmaceutically acceptable salt or salt mixture, isotopic derivative, or prodrug or pharmaceutically acceptable composition thereof, as well as methods for modulation of CNS activity, and for treatment of CNS disorders, including but not limited to post-traumatic stress, depression, adjustment disorders, addiction, anxiety and other mental disorders as described herein to a host such as a human in need thereof.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

### Definitions

When introducing elements of the present invention or the typical embodiments thereof, the articles “a,” “an,” “the,” and “said” are intended to mean that there are one or more of the elements. The terms “comprising,” “including,” and “having” are intended to be inclusive and not exclusive (i.e., there may be other elements in addition to the recited elements). Thus, the terms “including,” “may include,” and “include,” as used herein mean, and are used interchangeably with, the phrase “including but not limited to.”

Where a range of values is provided, it is understood that the upper and lower limit, and each intervening value between the upper and lower limit of the range is encompassed within the embodiments.

Unless defined otherwise, all technical and scientific terms herein have the meaning as commonly understood by one of ordinary skill in the art to which this invention belongs. In the event there is a plurality of definitions for a term herein, those in this section prevail unless stated otherwise. Further definitions that may assist the reader to understand the disclosed embodiments are as follows, and such definitions may be used to interpret the defined terms, when those terms are used herein. However, the examples given in the definitions are generally non-exhaustive and must not be construed as limiting the invention. It also will be understood that a substituent should comply with chemical bonding rules and steric compatibility constraints in relation to the particular molecule to which it is attached.

A compound of the invention may contain one or more chiral centers and/or double bonds and therefore, may exist as stereoisomers, such as double-bond isomers (i.e., geometric isomers), enantiomers, or diastereomers. Accordingly, the chemical structures and Formulas depicted herein independently encompass all possible enantiomers and stereoisomers of the illustrated compounds including the stereoisomerically pure form (for example, geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer and not more than 98%. An enantiomerically enriched mixture of an R-enantiomer contains at least 55% of the R-enantiomer, and typically at least about 60%, 65%, 70%, 75%, 80%, 85%, 90% or 95% of the R-enantiomer and not more than 98%. The specific ratio of S or R enantiomer can be selected for the need of the patient according to the health care specialist to balance the desired effect. The term enantiomerically enriched mixture as used herein does not include either a racemic mixture or a substantially pure or pure enantiomer (greater than 98% or 99% or even essentially 100%).

The term enantiomerically enriched mixture as used in this application does not include a racemic mixture and does not include a pure isomer. Notwithstanding, it should be understood that any compound described herein in enantiomerically enriched form can be used as a pure isomer if it achieves the goal of any of the specifically itemized methods of treatment described herein, including but not limited to a compound, pure diastereomer, pure enantiomer, or enantiomerically enriched mixture of a compound of Formula I-XXII described herein.

The term “CNS disorder” as used herein refers to either a neurological condition (one that is typically treated by a neurologist) or a psychiatric condition (one that is typically treated by a psychiatrist). Neurological disorders are typically those affecting the structure, biochemistry or normal electrical functioning of the brain, spinal cord or other nerves. Psychiatric conditions are more typically thought of as mental disorders, which are primarily abnormalities of thought, feeling or behavior that cause significant distress or impairment of personal functioning. Thus, a disclosed compound may be used in an effective amount to improve neurological or psychiatric functioning in a patient in need thereof. Neurological indications include, but are not limited to improved neuroplasticity, including treatment of stroke, brain trauma, dementia, and neurodegenerative diseases. In certain embodiments a halogenated benzofuran compound of the present invention is a psychoplastogen. A psychoplastogen is a small molecule that induces rapid neuroplasticity and has a rapid and sustained effects on neuronal structure and function. For example, in certain embodiments, the disclosed compound or composition may be used to improve stuttering and other dyspraxias or to treat Parkinson's disease or schizophrenia.

The term “neurological disease or disorder” includes for example, Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.

The term “improving psychiatric function” is intended to include mental health and life conditions that are not traditionally treated by neurologists but sometimes treated by psychiatrists and can also be treated by psychotherapists, life coaches, personal fitness trainers, meditation teachers, counselors, and the like. For example, it is contemplated that a disclosed compound will allow individuals to effectively contemplate actual or possible experiences that would normally be upsetting or even overwhelming. This includes individuals with fatal illness planning their last days and the disposition of their estate. This also includes couples discussing difficulties in their relationship and how to address them. This also includes individuals who wish to more effectively plan their career.

The term “inadequate functioning of neurotransmission” is used synonymously with a CNS disorder that adversely affects normal healthy neurotransmission.

Examples of isotopes that can be incorporated into a compound of the invention include isotopes of hydrogen, carbon, nitrogen, oxygen, fluorine and chlorine such as ²H, ³H, ¹¹C, ¹³C, ¹⁴C, ¹³N ¹⁵N, ¹⁷O, ¹⁸O, ¹⁸F, ³⁶Cl, and respectively. In some non-limiting embodiments, an isotopically labelled compound can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound may be particularly desirable for PET or SPECT studies. An isotopically labeled compound of this invention and a prodrug thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, for example, ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is at least 60, 70, 80, 90, 95 or 99% or more enriched in an isotope at any location of interest. In some non-limiting embodiments, deuterium is at least 80, 90, 95 or 99% enriched at a desired location. Unless indicated to the contrary, the deuteration is at least 80% at the selected location. Deuteration can occur at any replaceable hydrogen that provides the desired results.

In some non-limiting embodiments, the substitution of a hydrogen atom for a deuterium atom can be provided in a compound or composition described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). A compound of the invention also includes an isotopically labeled compound where one or more atoms have an atomic mass different from the atomic mass conventionally found in nature. Examples of isotopes that may be incorporated into a compound of the invention include ²H, ³H, ¹³C, ¹⁴C, ¹³N, ¹⁵N, ¹⁸O, ¹⁷O, ³¹P, ³²p ³⁵S, ¹⁸F, and ³⁶Cl.

An alkyl group on the nitrogen of a compound of the invention is subject to enzymatic removal. The N-alkyl may be prepared with a deuterated reagent that replaces one, two, any, or all of the hydrogens on the N-alkyl group, which creates a higher activation energy for bond cleavage and a slower formation of the desalkyl metabolite. In general, when deuterium is substituted for a hydrogen at a location of metabolism in the compound, a more stable compound will result.

Any one of a compound of Formulas I-XXII or a compound, pure diastereomer, pure enantiomer, diastereomerically enriched mixture, or enantiomerically enriched mixture of the invention have chiral centers and thus exist as enantiomers or diastereomers that may be more appropriate for some applications. Accordingly, the present disclosure also includes stereoisomers of a compound described herein, where applicable, either individually or admixed in any proportions. Stereoisomers may include enantiomers, diastereomers, racemic mixtures, and combinations thereof.

In certain embodiments a compound with entactogenic properties as described herein refers to a compound with DAT (dopamine transporter)/SERT (serotonin transporter) ratio of less than about 10, wherein the DAT/SERT ratio is expressed as 1/DAT IC₅₀:1/SERT IC₅₀. In certain aspects, the DAT/SERT ratio of the entactogenic compound is typically less than about 5, and preferably less than about 2. For example, the DAT/SERT ratio can be measured in a cell line which is engineered to express human monoamine transporters, dopamine (hDAT) and serotonin (hSERT) transporter (e.g., the assay of Example 5). In some embodiments the engineered cell is a Chinese hamster ovary (CHO) cell.

“Alkyl” is a branched, straight chain, or cyclic saturated aliphatic hydrocarbon group including from 1 to about 8 carbon atoms, from 1 to about 6 carbon atoms, from 1 to about 4 carbon atoms, from 1 to 3 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having 1, 2, 3, or 4 carbon atoms and is intended to mean that each of these is described as an independent species. Examples of alkyl include, but are not limited to, methyl, ethyl, n-propyl, isopropyl, cyclopropyl, n-butyl, isobutyl, sec-butyl, cyclobutyl, t-butyl, n-pentyl, isopentyl, tert-pentyl, neopentyl, cyclopentyl, n-hexyl, cyclopentyl, 2-methylpentane, 3-methylpentane, 2,2-dimethylbutane, 2,3-dimethylbutane, and hexyl.

“Haloalkyl” indicates both branched and straight-chain alkyl groups substituted with one or more halogen atoms, up to the maximum allowable number of halogen atoms. Examples of haloalkyl include, but are not limited to, trifluoromethyl, monofluoromethyl, difluoromethyl, 2-fluoroethyl, 2,2,2-trifluoroethyl, and penta-fluoroethyl.

“Halogen” or “halo” means fluoro (F), chloro (Cl), bromo (Br), or iodo (I). For groups containing two or more halogens, such as —CHX₂ or —CX₃, and for example “where X is halogen,” it will be understood that each X independently will be selected from the group of halogens.

“Hydroxy” means the radical OH.

“Oxo” means the divalent radical ═O.

“Stereoisomers” includes enantiomers, diastereomers, the components of racemic mixtures, and combinations thereof. Stereoisomers can be prepared or separated as described herein or by using other methods.

Such stereoisomers can be prepared and separated using conventional techniques, either by reacting chiral starting materials, or by separating isomers of a compound disclosed herein. Enantiomers can be separated using multiple techniques such as but not limited to selective and/or fractional crystallization or chromatography (nonlimiting examples of chromatographic techniques for the purification of enantiomers include high performance liquid chromatography (HPLC), ultra-high performance liquid chromatography (UPLC), or supercritical fluid chromatography (SFC), utilizing a chiral stationary phase). Enantiomers can also be separated through crystallization or chromatographically utilizing achiral stationary phases (including but not limited to silica gel, octadecyl functionalized silica (C18) or octyl functionalized silica (C8)). Alternatively, or in conjunction with separation of stereoisomers, individual stereoisomers can be synthesized using asymmetric synthesis techniques. A chiral compound of the invention may be prepared from the racemic or stereoisomerically enriched compound. Pharmaceutically acceptable salts of a chiral compound may be prepared from fractional crystallization of salts from a racemic, diastereomerically- or enantiomerically enriched free amine and a chiral acid. Alternatively, the free amine may be reacted with a chiral auxiliary and the enantiomers separated by chromatography followed by removal of the chiral auxiliary to regenerate the free amine. Furthermore, separation of enantiomers may be performed at any convenient point in the synthesis of a compound of the invention.

“Agonist” refers to a modulator that binds to a receptor or enzyme and activates the receptor to produce a biological response. In some embodiments, “agonist” includes full agonists or partial agonists.

“Stereoisomers” includes enantiomers, diastereomers, the components of racemic mixtures, and combinations thereof. Stereoisomers can be prepared or separated as described herein or by using other methods.

Such stereoisomers can be prepared and separated using conventional techniques, either by reacting enantiomeric starting materials, or by separating isomers of a compound disclosed herein.

An isolated enantiomerically enriched or enantiopure halogenated benzofuran compound may be used as a pure enantiomer or combined with any other enantiomer in any ratio that produces the desired effects. Alternatively, a halogenated benzofuran compound of the present invention is used as a pure diastereomer, or mixture of diastereomers that produces the desired effects. An enantiomerically enriched or enantiomerically pure compound of the invention may be enantiomerically enriched or enantiomerically pure at one chiral center or at both chiral centers if two chiral centers are present.

In certain embodiments the halogenated benzofuran compound is a racemate.

Unless otherwise indicated, when the term “R” or “S” is noted, it refers to the IUPAC stereochemical configuration of the stereocenter based on the Cahn-Ingold-Prelog priority assignment.

In certain embodiments the halogenated benzofuran compound is an enantiomerically enriched mixture or R- and S-enantiomers whenever one of the enantiomers is present in excess of 50% to achieve the desired properties.

Furthermore, the individual enantiomers of the present invention may exist in isolated form or mixed in such a way that one enantiomer is present in a greater amount than the other, referred to herein as an enantiomerically enriched mixture. An enantiomerically enriched mixture is a mixture that contains one enantiomer in a greater amount than the other. The term enantiomerically enriched mixture includes either the mixture enriched with the R-enantiomer or enriched with the S-enantiomer. Unless context clearly indicates otherwise, the term “enantiomerically enriched mixture” can be understood to mean “enantiomerically enriched mixture of the R- or S-enantiomer.” An enantiomerically enriched mixture is a mixture that contains one enantiomer in a greater amount than the other. An enantiomerically enriched mixture of an S-enantiomer contains at least 55% of the S-enantiomer, and, typically at least about 60%, 65%, 70%, 75%, 80%, 85%, 90%, or 95% or more of the S-enantiomer and not more than 98%. An enantiomerically enriched mixture of an R-enantiomer contains at least 55% of the R-enantiomer, and typically at least about 60%, 65%, 70%, 75%, 80%, 85%, 90% or 95% of the R-enantiomer and not more than 98%. The specific ratio of S or R enantiomer can be selected for the need of the patient according to the health care specialist to balance the desired effect. The term enantiomerically enriched mixture as used herein does not include either a racemic mixture or a substantially pure or pure enantiomer (greater than 98% or 99% or even essentially 100%).

The four enantiomers of the compounds of the invention also exist as two different diastereomers. The cis- or trans-diastereomer of the present invention may exist in isolated form or mixed in such a way that one diastereomer is present in a greater amount than the other, referred to herein as a diastereomerically enriched mixture. A diastereomerically enriched mixture is a mixture that contains one diastereomer in a greater amount than the other. The term diastereomerically enriched mixture includes either the mixture enriched with the trans-diastereomer or enriched with the cis-diastereomer. Unless context clearly indicates otherwise, the term “diastereomerically enriched mixture” can be understood to mean “diastereomerically enriched mixture of the cis- or trans-diastereomer.” A diastereomerically enriched mixture is a mixture that contains one diastereomer in a greater amount than the other. A diastereomerically enriched mixture of a cis-diastereomer contains at least 55% of the cis-diastereomer, and, typically at least about 60%, 65%, 70%, 75%, 80%, 85%, 90%, or 95% or more of the cis-diastereomer and not more than 98%. An enantiomerically enriched mixture of a trans-diastereomer contains at least 55% of the trans-diastereomer, and typically at least about 60%, 65%, 70%, 75%, 80%, 85%, 90% or 95% of the trans-diastereomer and not more than 98%. The specific ratio of cis- or trans-diastereomer can be selected for the need of the patient according to the health care specialist to balance the desired effect. The term diastereomerically enriched mixture as used herein does not include either a racemic mixture or a substantially pure or pure diastereomer (greater than 98% or 99% or even essentially 100%).

In some embodiments, a diastereomerically enriched mixture of the cis-diastereomer or pure trans-diastereomer of a halogenated benzofuran compound of the present invention increases the serotonin-receptor-dependent actions that contribute to therapeutic effects and minimizes adverse dopaminergic effects that may contribute to unwanted properties like addictive liability when administered to a host in need thereof, for example a mammal, including a human, relative to the racemic form.

In some embodiments, a diastereomerically enriched mixture of the trans-diastereomer or pure trans-diastereomer of a halogenated benzofuran compound of the present invention increases the serotonin-receptor-dependent actions that contribute to therapeutic effects and minimizes adverse dopaminergic effects that may contribute to unwanted properties like addictive liability when administered to a host in need thereof, for example a mammal, including a human, relative to the racemic form.

In certain embodiments a pure diastereomer of the present invention is enantiomerically enriched. In other embodiments a pure diastereomer of the present invention is enantiomerically pure or racemic.

“Agonist” refers to a modulator that binds to a receptor or enzyme and activates the receptor to produce a biological response. In some embodiments, “agonist” includes full agonists or partial agonists.

“Antagonism” refers to the inactivation of a receptor or enzyme by a modulator, or antagonist. Antagonism of a receptor, for example, is when a molecule binds to the receptor and does not allow activity to occur.

“IC₅₀” refers to the concentration of a substance (for example, a compound or a drug) that is required for 50% inhibition of a biological process. For example, IC₅₀ refers to the half maximal (50%) inhibitory concentration (IC) of a substance as determined in a suitable assay. Similarly, EC₅₀ refers to the concentration of a substance that provokes a response halfway between the baseline activity and maximum response. In some instances, an IC₅₀ or EC₅₀ is determined in an in vitro assay system. In some embodiments as used herein, IC₅₀ (or EC₅₀) refers to the concentration of a modulator that is required for 50% inhibition (or excitation) of a receptor, for example, 5HT_{1B}.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (for example, positive allosteric modulator) of a G protein-coupled receptor (for example, 5-HT_{1B}) are modulators of the receptor.

“Neuroplasticity” refers to the ability of the brain to change its structure and/or function throughout a subject's life. Examples of the changes to the brain include, but are not limited to, the ability to adapt or respond to internal and/or external stimuli, such as due to an injury, and the ability to produce new neurites, dendritic spines, and synapses.

“Treating” or “treatment” of a disease, as used in context, includes (i) inhibiting the disease, i.e., arresting or reducing the development or progression of the disease or its clinical symptoms; or (ii) relieving the disease, i.e., causing regression of the disease or its clinical symptoms. Inhibiting the disease, for example, would include prophylaxis. Hence, one of skill in the art will understand that a therapeutic amount necessary to effect treatment for purposes of this invention will, for example, be an amount that provides for objective indicia of improvement in patients having clinically diagnosable symptoms. Other such measurements, benefits, and surrogate or clinical endpoints, whether alone or in combination, would be understood to those of ordinary skill.

“Therapeutic effect” means the responses(s) in a host after treatment that is judged to be desirable or beneficial. Hence, depending on the CNS disorder to be treated, or improvement in CNS functioning sought, those responses shall differ, but would be readily understood by those of ordinary skill.

### Embodiments of the Present Invention

In certain embodiments the halogenated benzofuran compound is of Formula:

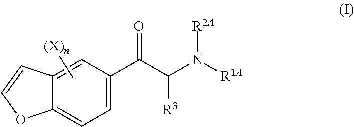

or pharmaceutically acceptable salt or salt mixture thereof.

In certain embodiments the halogenated benzofuran compound is of Formula:

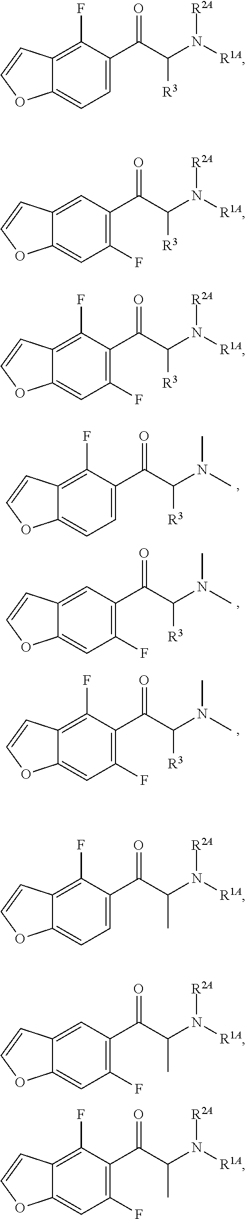

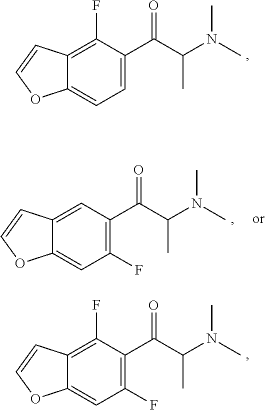

or pharmaceutically acceptable salt or salt mixture thereof.

In certain embodiments the halogenated benzofuran compound is of Formula:

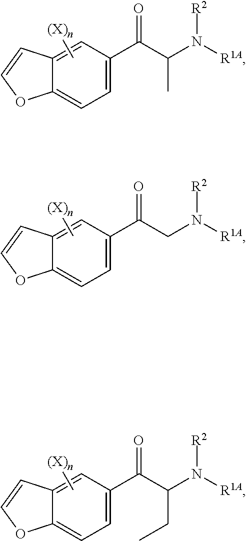

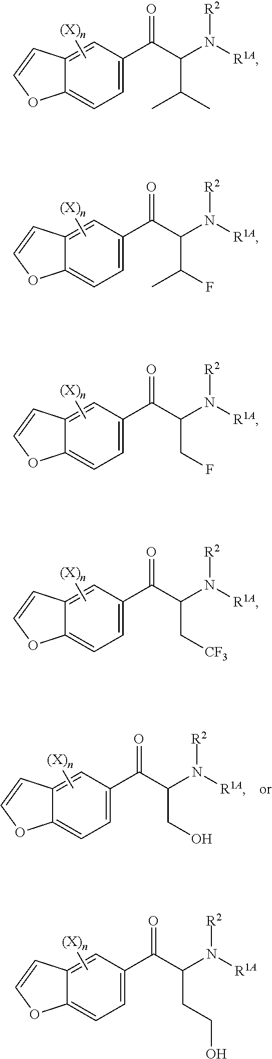

or pharmaceutically acceptable salt or salt mixture thereof.

In certain embodiments the halogenated benzofuran compound is of Formula:

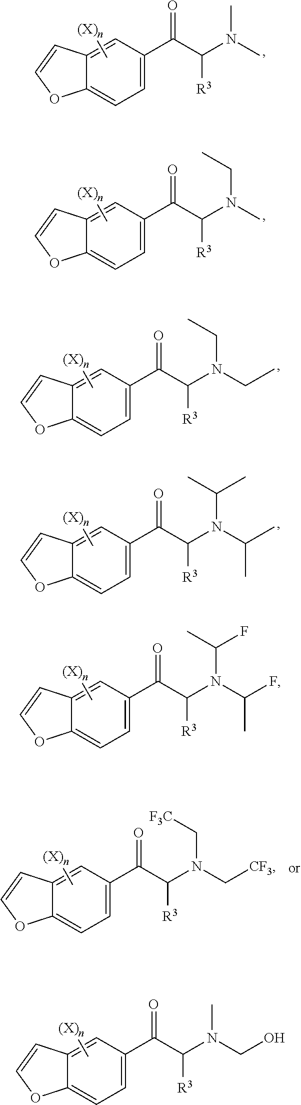

or pharmaceutically acceptable salt or salt mixture thereof.

In certain embodiments the halogenated benzofuran compound is of Formula:

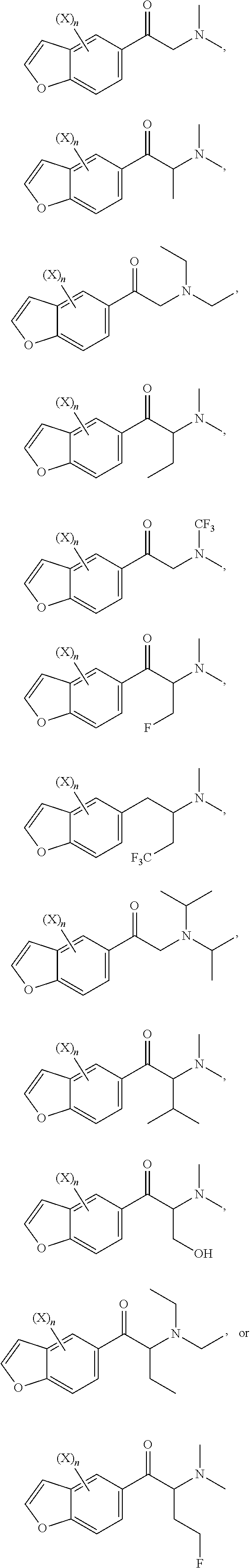

or pharmaceutically acceptable salt or salt mixture thereof.

In certain embodiments the halogenated benzofuran compound is of Formula:

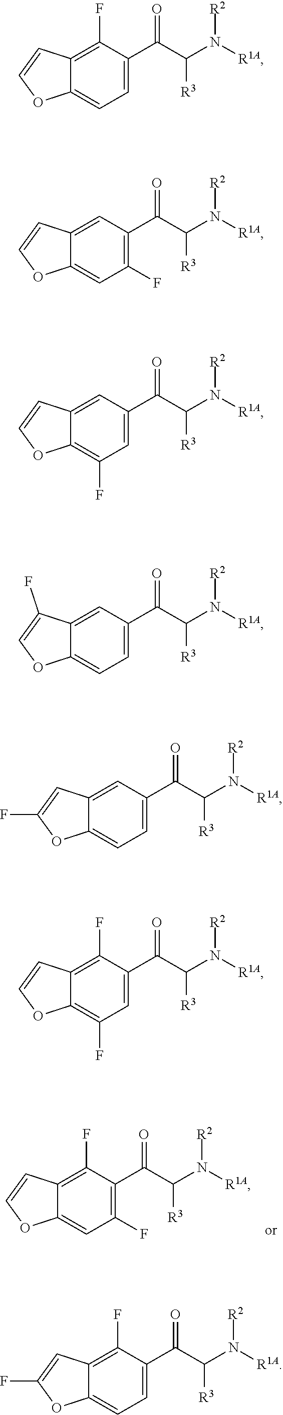

or pharmaceutically acceptable salt or salt mixture thereof.

In certain embodiments the halogenated benzofuran compound is of Formula:

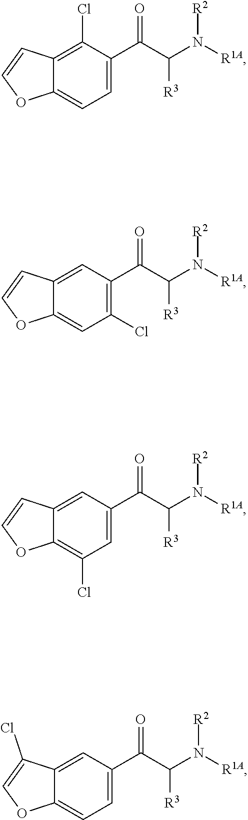

or pharmaceutically acceptable salt or salt mixture thereof.

In certain embodiments the halogenated benzofuran compound is of Formula:

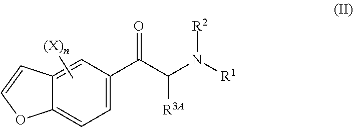

or pharmaceutically acceptable salt or salt mixture thereof.

In certain embodiments the halogenated benzofuran compound is of Formula:

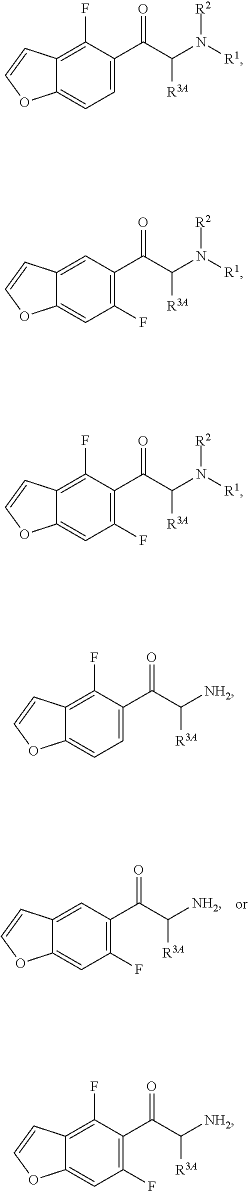

or pharmaceutically acceptable salt or salt mixture thereof.

In certain embodiments the halogenated benzofuran compound is of Formula:

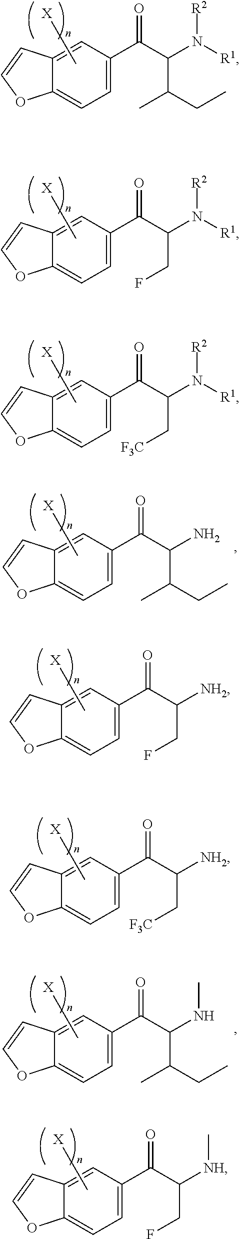

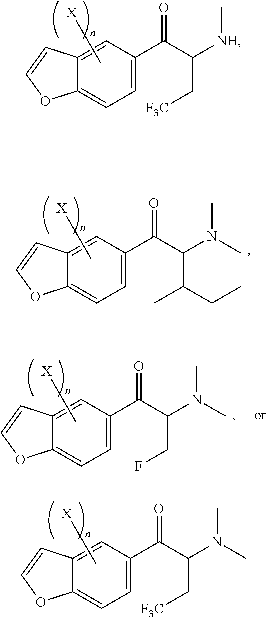

or pharmaceutically acceptable salt or salt mixture thereof.

In certain embodiments the halogenated benzofuran compound is of Formula:

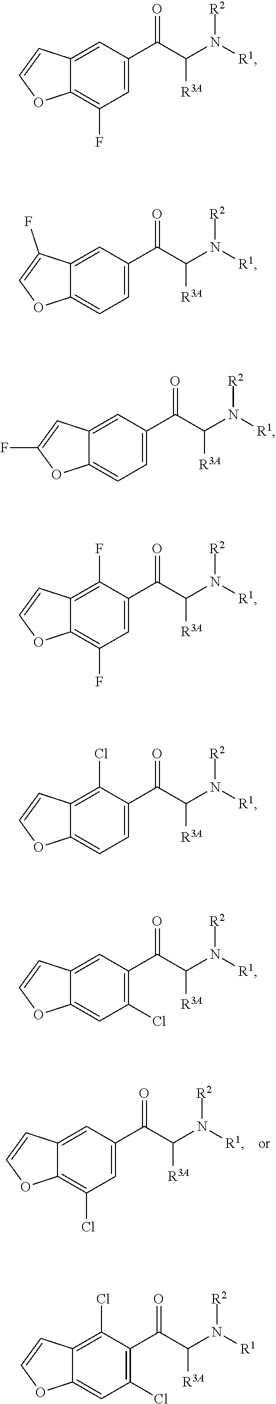

or pharmaceutically acceptable salt or salt mixture thereof.

In certain embodiments the halogenated benzofuran compound is of Formula:

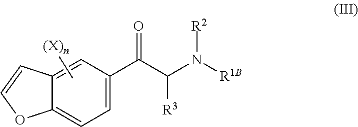

or pharmaceutically acceptable salt or salt mixture thereof.

In certain embodiments the halogenated benzofuran compound is of Formula:

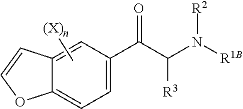

or pharmaceutically acceptable salt or salt mixture thereof.

In certain embodiments the halogenated benzofuran compound is of Formula:

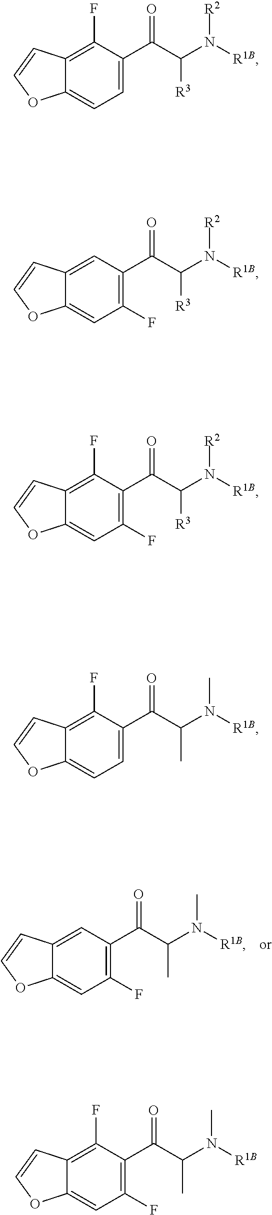

or pharmaceutically acceptable salt or salt mixture thereof.

In certain embodiments the halogenated benzofuran compound is of Formula:

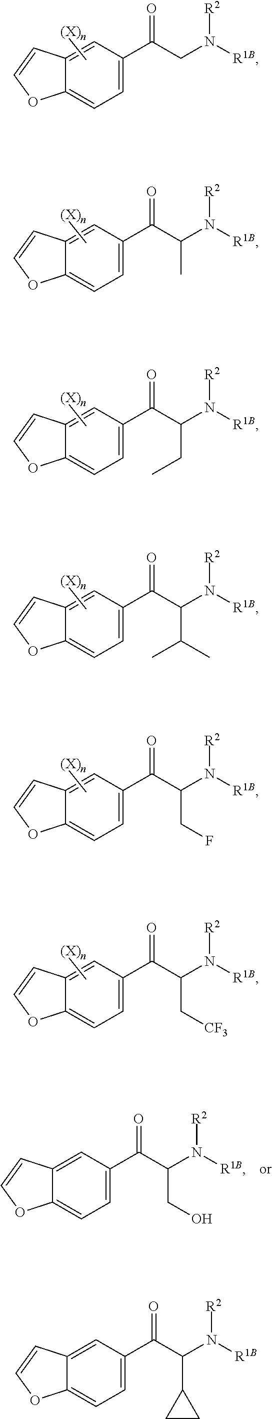

or pharmaceutically acceptable salt or salt mixture thereof.

In certain embodiments the halogenated benzofuran compound is of Formula:

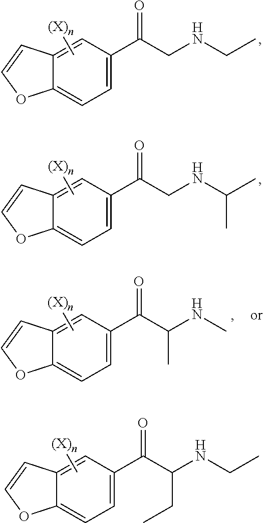

or pharmaceutically acceptable salt or salt mixture thereof.

In certain embodiments the halogenated benzofuran compound is of Formula:

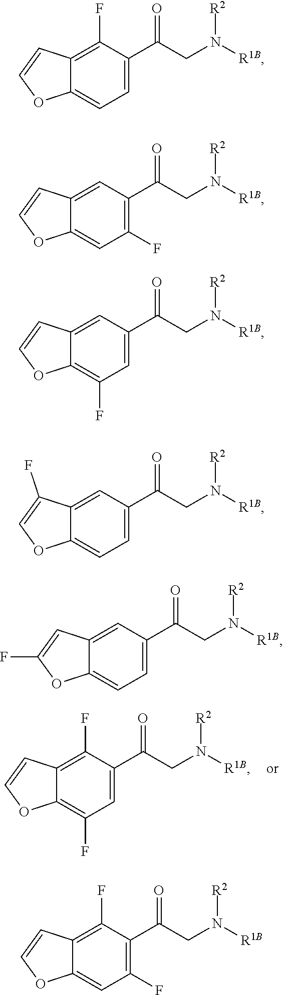

or pharmaceutically acceptable salt or salt mixture thereof.

In certain embodiments the halogenated benzofuran compound is of Formula:

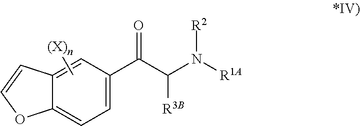

or pharmaceutically acceptable salt or salt mixture thereof.

In certain embodiments the halogenated benzofuran compound is of Formula:

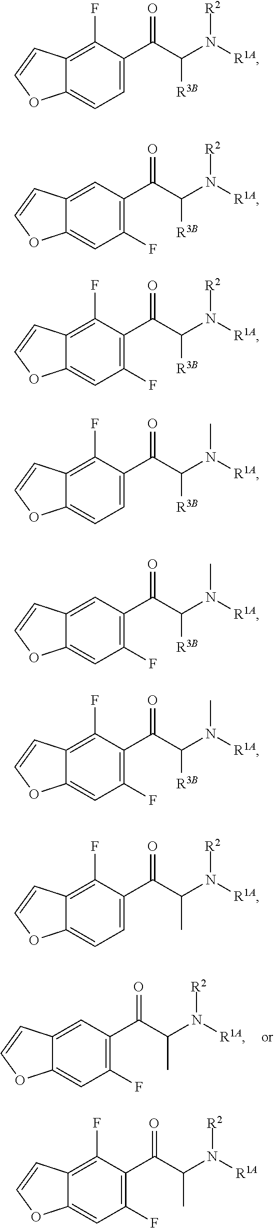

or pharmaceutically acceptable salt or salt mixture thereof.

In certain embodiments the halogenated benzofuran compound is of Formula:

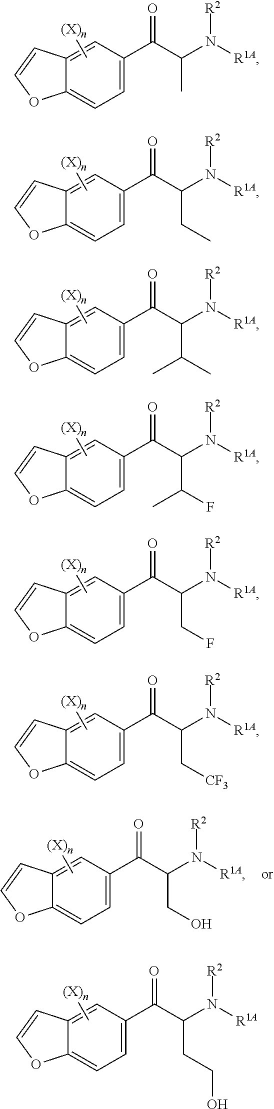

or pharmaceutically acceptable salt or salt mixture thereof.

In certain embodiments the halogenated benzofuran compound is of Formula:

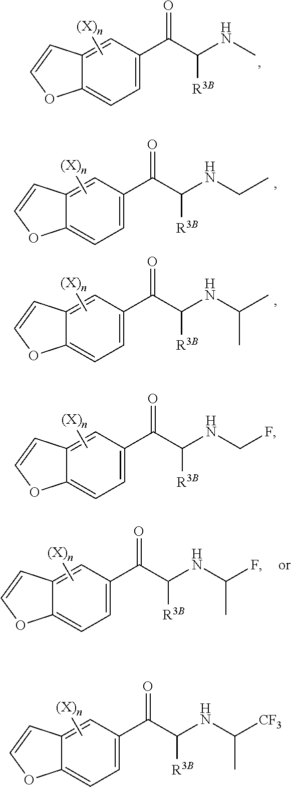

or pharmaceutically acceptable salt or salt mixture thereof.

In certain embodiments the halogenated benzofuran compound is of Formula:

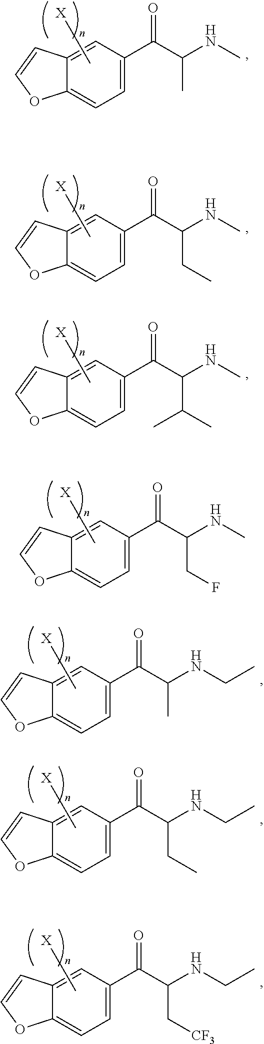

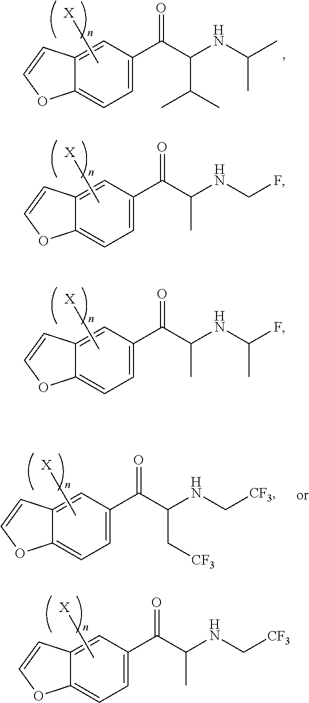

or pharmaceutically acceptable salt or salt mixture thereof.

In certain embodiments the halogenated benzofuran compound is of Formula:

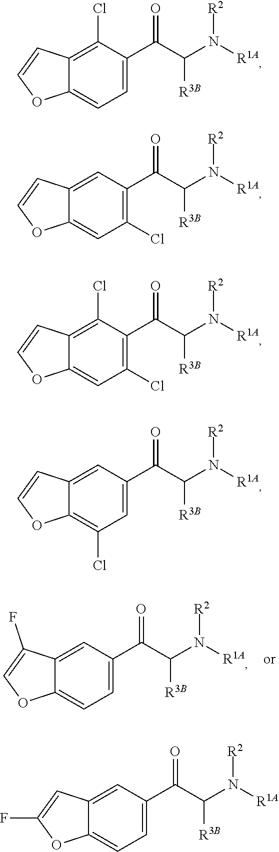

or pharmaceutically acceptable salt or salt mixture thereof.

In certain embodiments the halogenated benzofuran compound is of Formula:

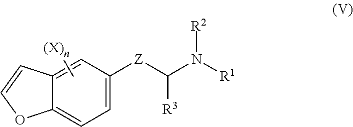

or pharmaceutically acceptable salt or salt mixture thereof.

In certain embodiments the halogenated benzofuran compound is of Formula:

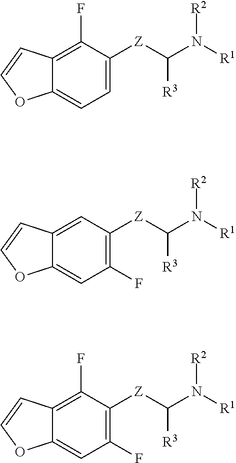

or pharmaceutically acceptable salt or salt mixture thereof.

In certain embodiments the halogenated benzofuran compound is of Formula:

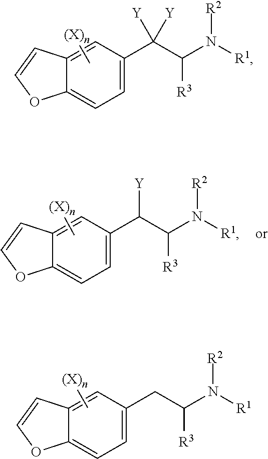

or pharmaceutically acceptable salt or salt mixture thereof.

In certain embodiments the halogenated benzofuran compound is of Formula:

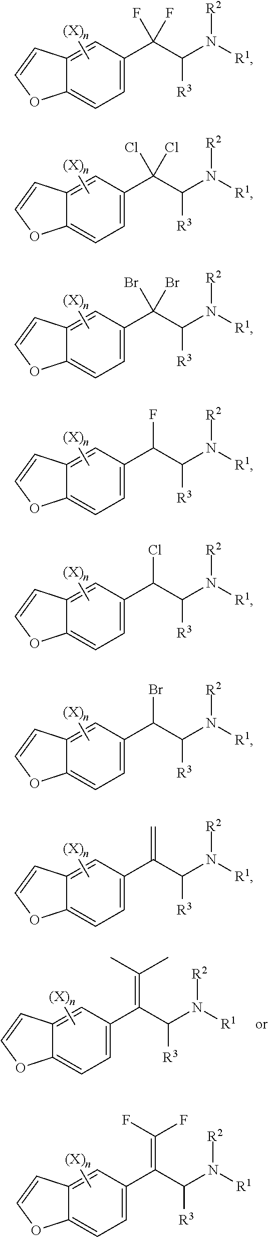

or pharmaceutically acceptable salt or salt mixture thereof.

In certain embodiments the halogenated benzofuran compound is of Formula:

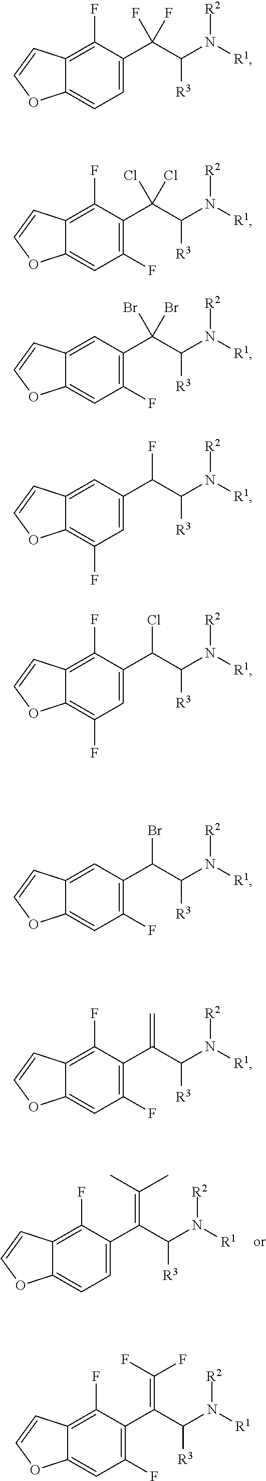

or pharmaceutically acceptable salt or salt mixture thereof.

In certain embodiments the halogenated benzofuran compound is of Formula:

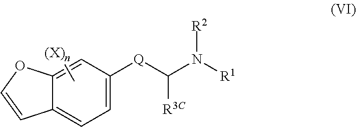

or pharmaceutically acceptable salt or salt mixture thereof.

In certain embodiments the halogenated benzofuran compound is of Formula:

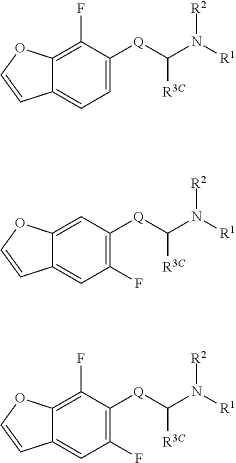

or pharmaceutically acceptable salt or salt mixture thereof.

In certain embodiments the halogenated benzofuran compound is of Formula:

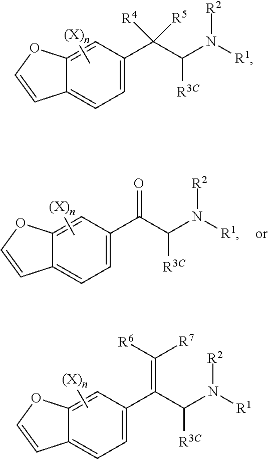

or pharmaceutically acceptable salt or salt mixture thereof.

In certain embodiments the halogenated benzofuran compound is of Formula:

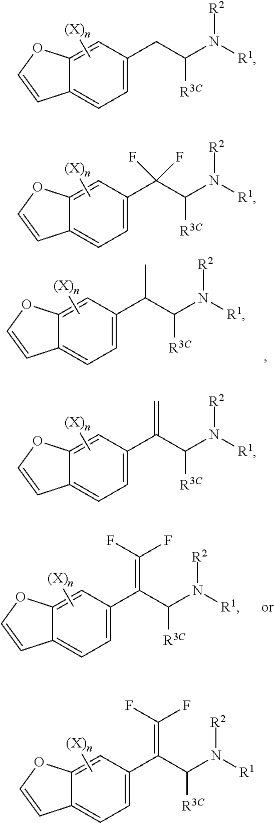

or pharmaceutically acceptable salt or salt mixture thereof.

In certain embodiments the halogenated benzofuran compound is of Formula:

or pharmaceutically acceptable salt or salt mixture thereof.

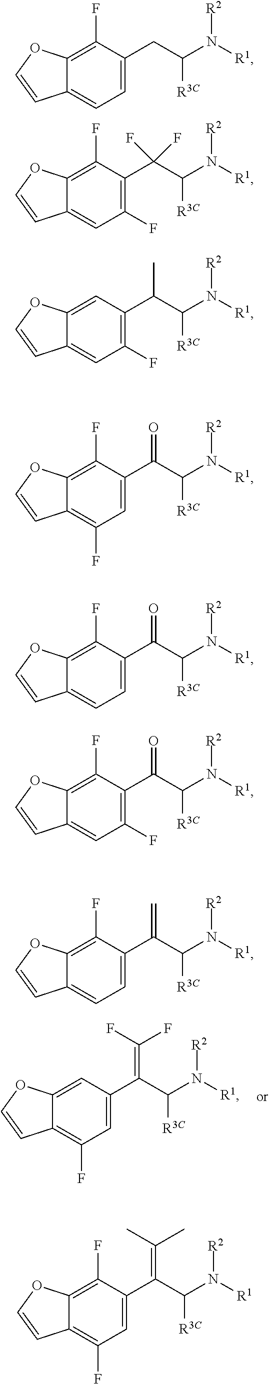

In certain embodiments the halogenated benzofuran compound is of Formula:

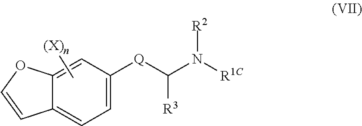

or pharmaceutically acceptable salt or salt mixture thereof.

In certain embodiments the halogenated benzofuran compound is of Formula:

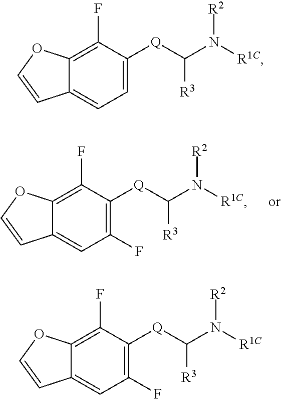

or pharmaceutically acceptable salt or salt mixture thereof.

In certain embodiments the halogenated benzofuran compound is of Formula:

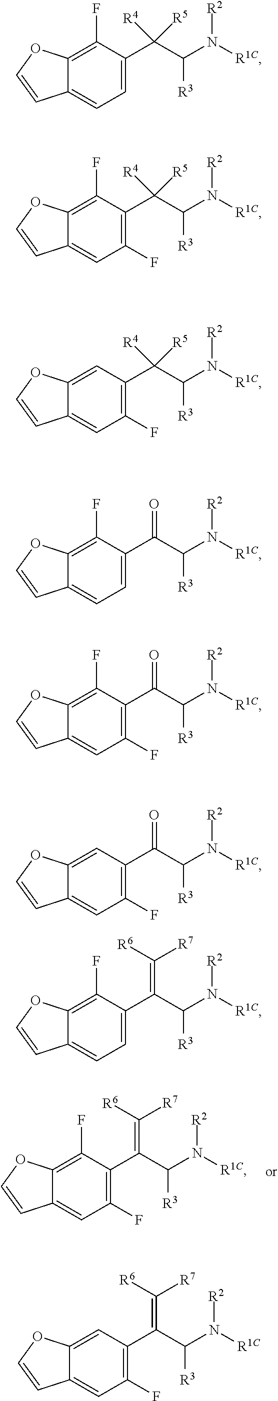

or pharmaceutically acceptable salt or salt mixture thereof.

In certain embodiments the halogenated benzofuran compound is of Formula:

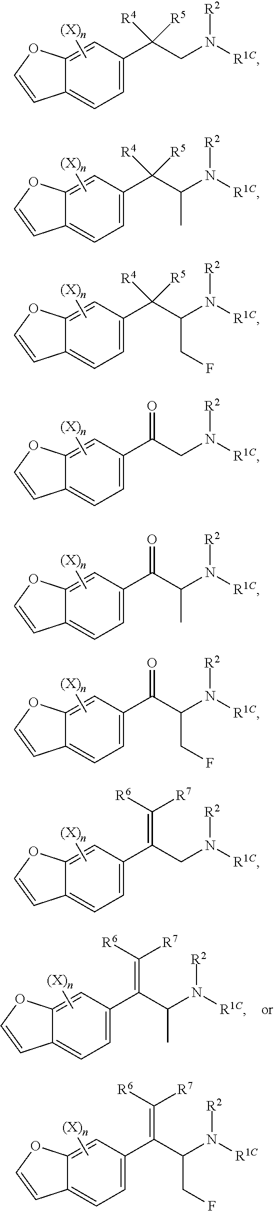

or pharmaceutically acceptable salt or salt mixture thereof.

In certain embodiments the halogenated benzofuran compound is of Formula:

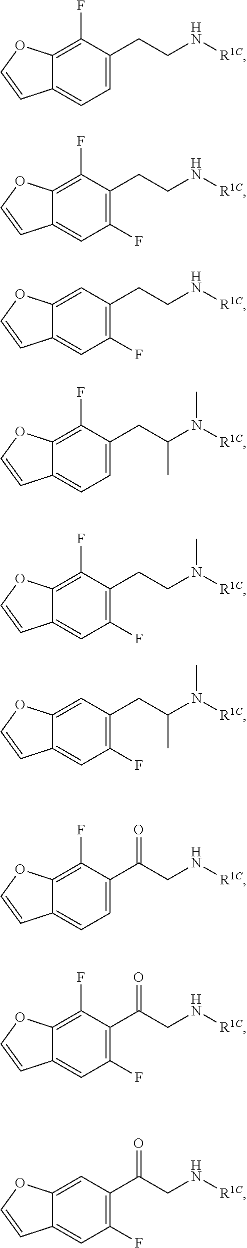

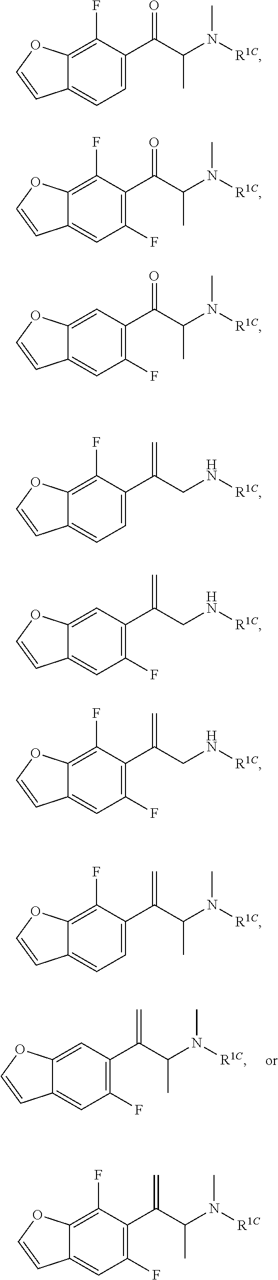

or pharmaceutically acceptable salt or salt mixture thereof.

In certain embodiments the halogenated benzofuran compound is of Formula:

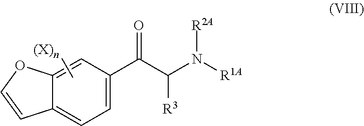

or pharmaceutically acceptable salt or salt mixture thereof.

In certain embodiments the halogenated benzofuran compound is of Formula:

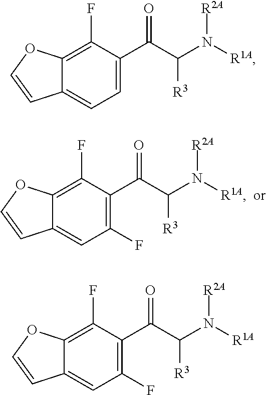

or pharmaceutically acceptable salt or salt mixture thereof.

In certain embodiments the halogenated benzofuran compound is of Formula:

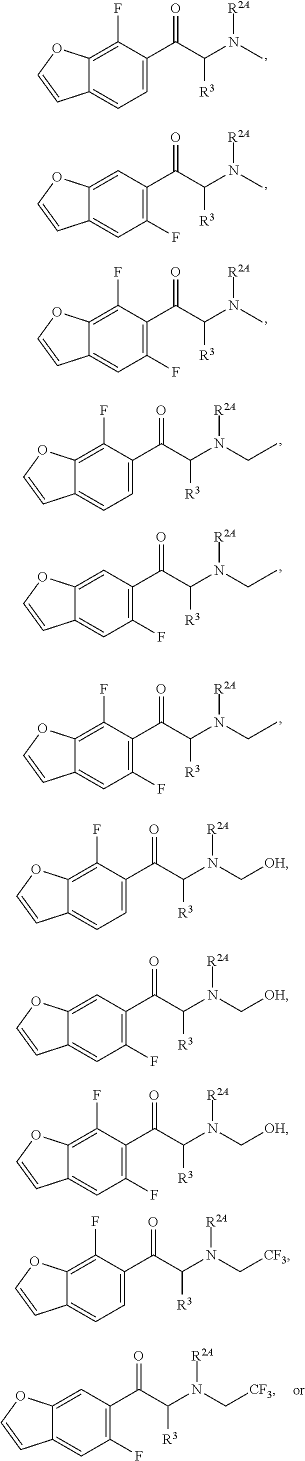

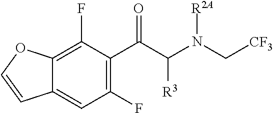

or pharmaceutically acceptable salt or salt mixture thereof.

In certain embodiments the halogenated benzofuran compound is of Formula:

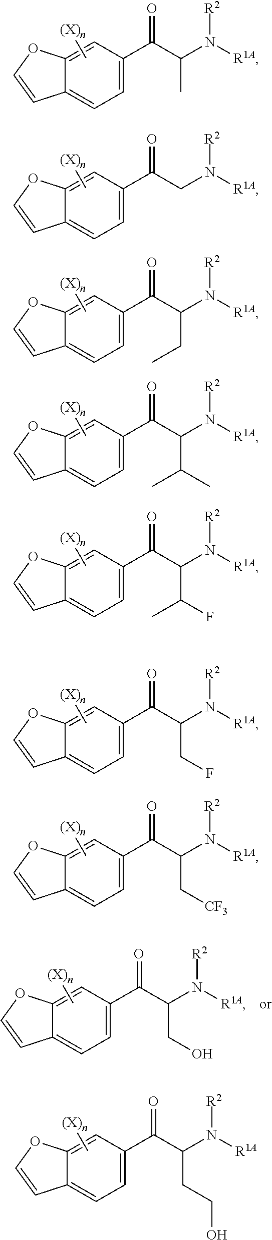

or pharmaceutically acceptable salt or salt mixture thereof.

In certain embodiments the halogenated benzofuran compound is of Formula:

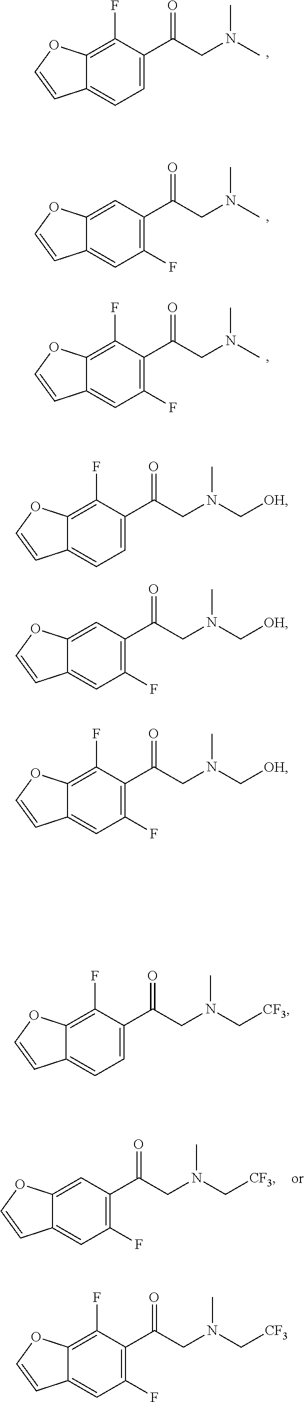

or pharmaceutically acceptable salt or salt mixture thereof.

In certain embodiments the halogenated benzofuran compound is of Formula:

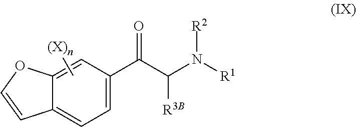

or pharmaceutically acceptable salt or salt mixture thereof.

In certain embodiments the halogenated benzofuran compound is of Formula:

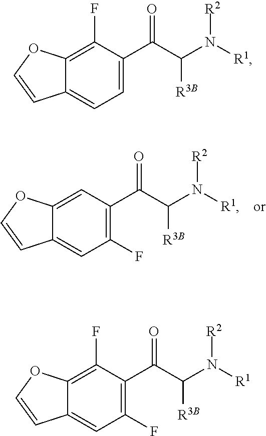

or pharmaceutically acceptable salt or salt mixture thereof.

In certain embodiments the halogenated benzofuran compound is of Formula:

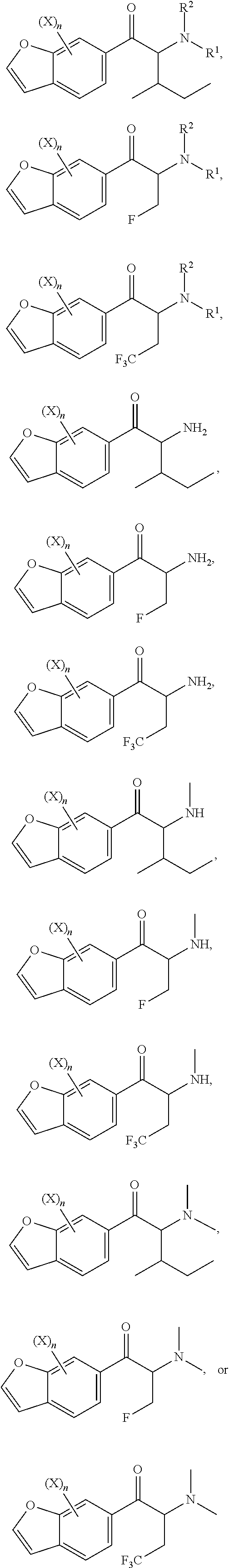

or pharmaceutically acceptable salt or salt mixture thereof.

In certain embodiments the halogenated benzofuran compound is of Formula:

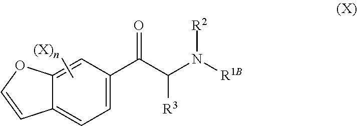

or pharmaceutically acceptable salt or salt mixture thereof.

In certain embodiments the halogenated benzofuran compound is of Formula:

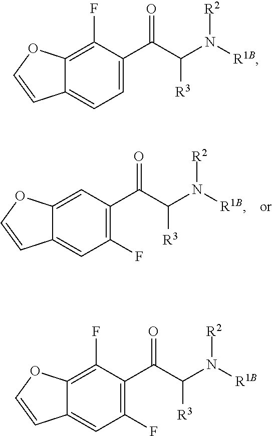

or pharmaceutically acceptable salt or salt mixture thereof.

In certain embodiments the halogenated benzofuran compound is of Formula:

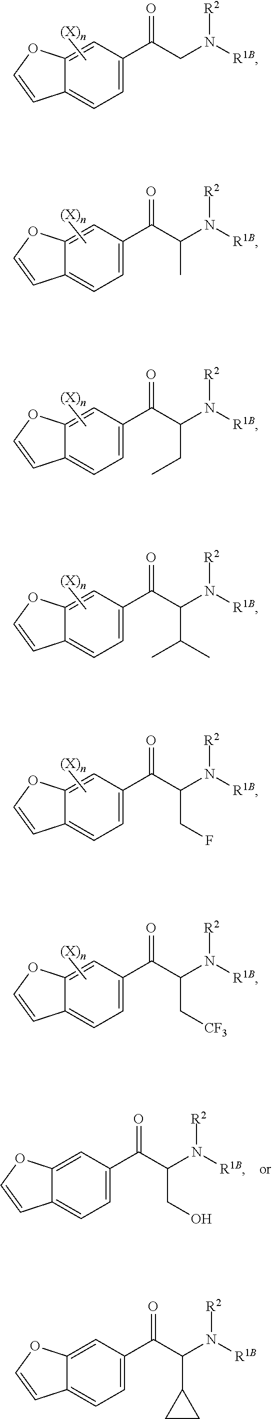

or pharmaceutically acceptable salt or salt mixture thereof.

In certain embodiments the halogenated benzofuran compound is of Formula:

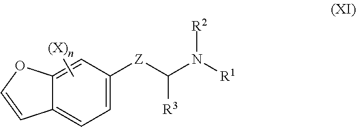

or pharmaceutically acceptable salt or salt mixture thereof.

In certain embodiments the halogenated benzofuran compound is of Formula:

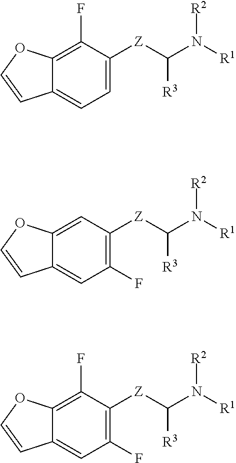

or pharmaceutically acceptable salt or salt mixture thereof.

In certain embodiments the halogenated benzofuran compound is of Formula:

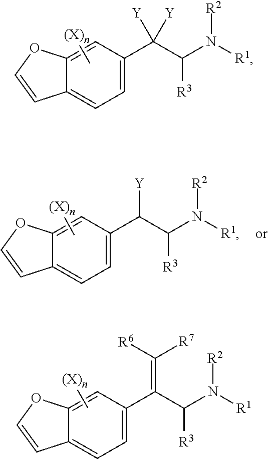

or pharmaceutically acceptable salt or salt mixture thereof.

In certain embodiments the halogenated benzofuran compound is of Formula:

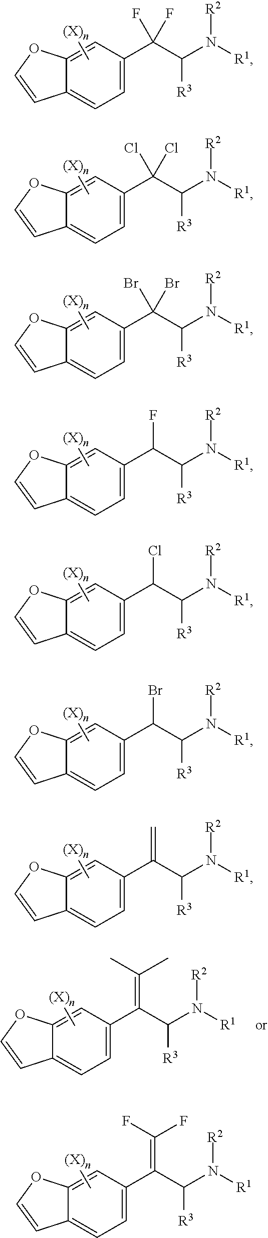

or pharmaceutically acceptable salt or salt mixture thereof.

In certain embodiments the halogenated benzofuran compound is of Formula:

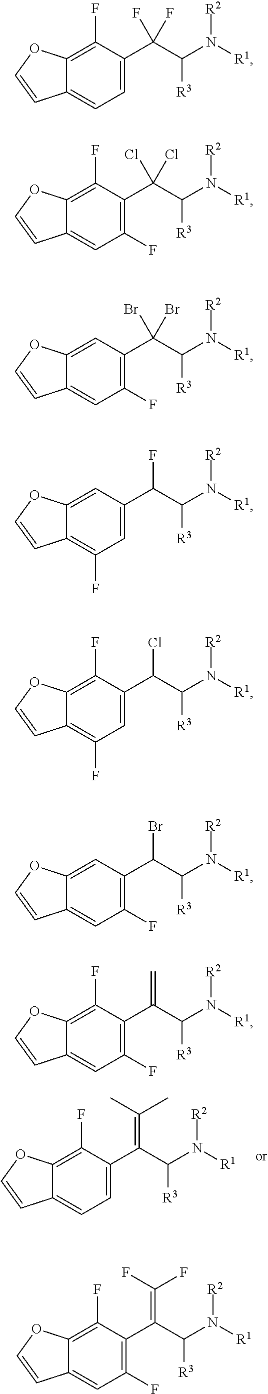

or pharmaceutically acceptable salt or salt mixture thereof.

In certain embodiments the halogenated benzofuran compound is of Formula:

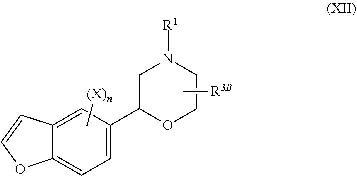

or pharmaceutically acceptable salt or salt mixture thereof.

In certain embodiments the halogenated benzofuran compound is of Formula:

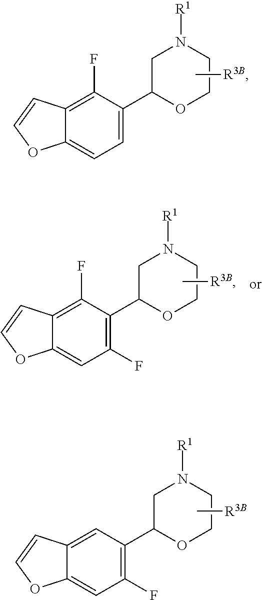

or pharmaceutically acceptable salt or salt mixture thereof.

In certain embodiments the halogenated benzofuran compound is of Formula:

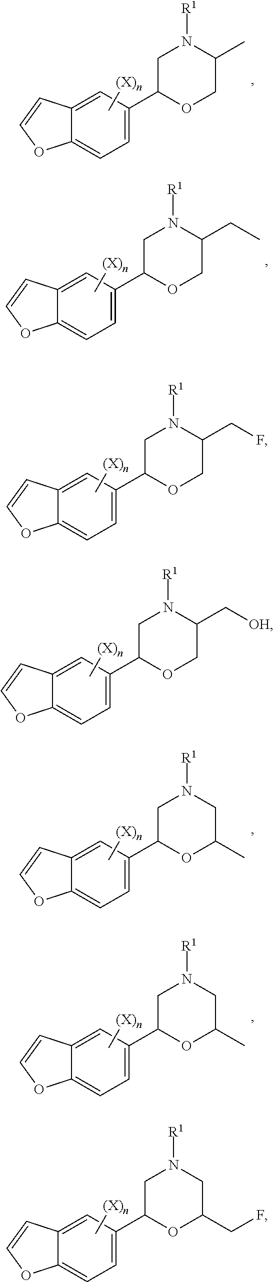

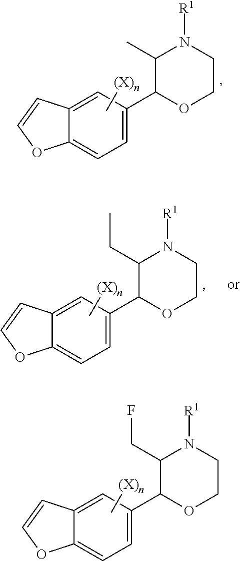

or pharmaceutically acceptable salt or salt mixture thereof.

In certain embodiments the halogenated benzofuran compound is of Formula:

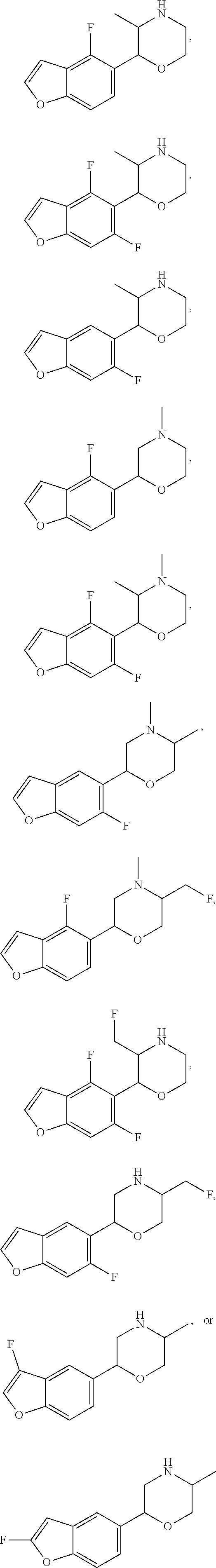

or pharmaceutically acceptable salt or salt mixture thereof.

In certain embodiments the halogenated benzofuran compound is of Formula:

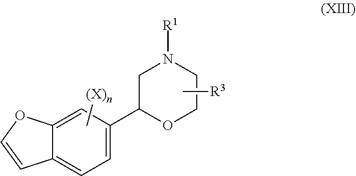

or pharmaceutically acceptable salt or salt mixture thereof.

In certain embodiments the halogenated benzofuran compound is of Formula:

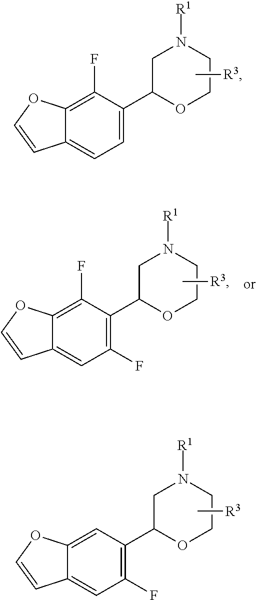

or pharmaceutically acceptable salt or salt mixture thereof.

In certain embodiments the halogenated benzofuran compound is of Formula:

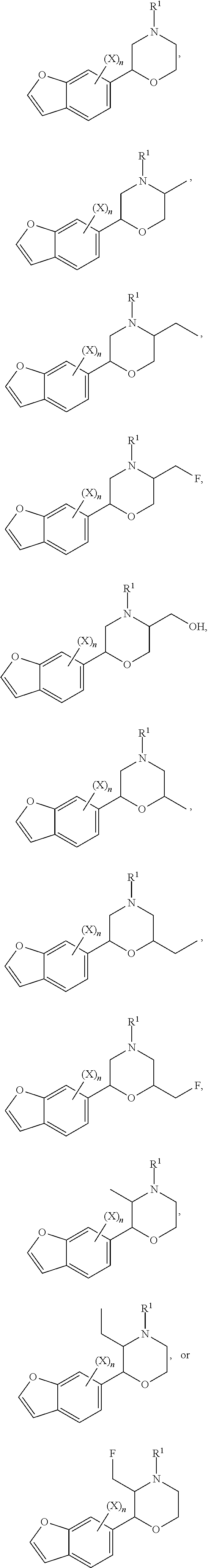

or pharmaceutically acceptable salt or salt mixture thereof.

In certain embodiments the halogenated benzofuran compound is of Formula:

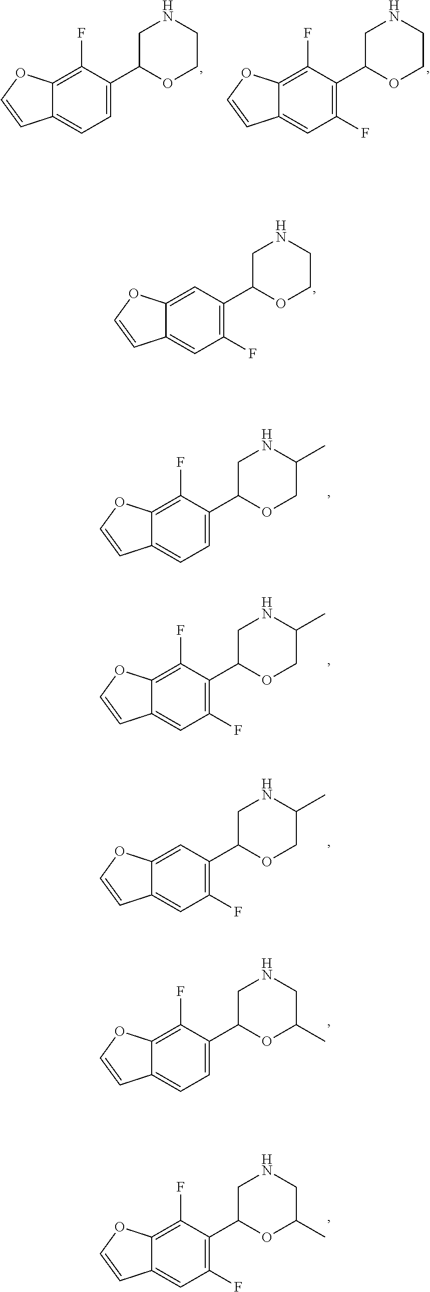

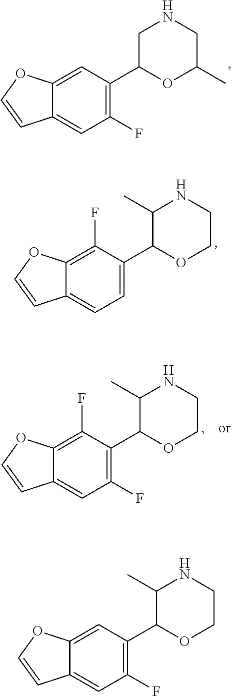 [SMILES: CC1CNCC(c2cc3occc3cc2F)O1 ; CC1NCCOC1c1ccc2ccoc2c1F ; CC1NCCOC1c1c(F)cc2ccoc2c1F ; CC1NCCOC1c1cc2occc2cc1F]

or pharmaceutically acceptable salt or salt mixture thereof.

In certain embodiments the halogenated benzofuran compound is of Formula:

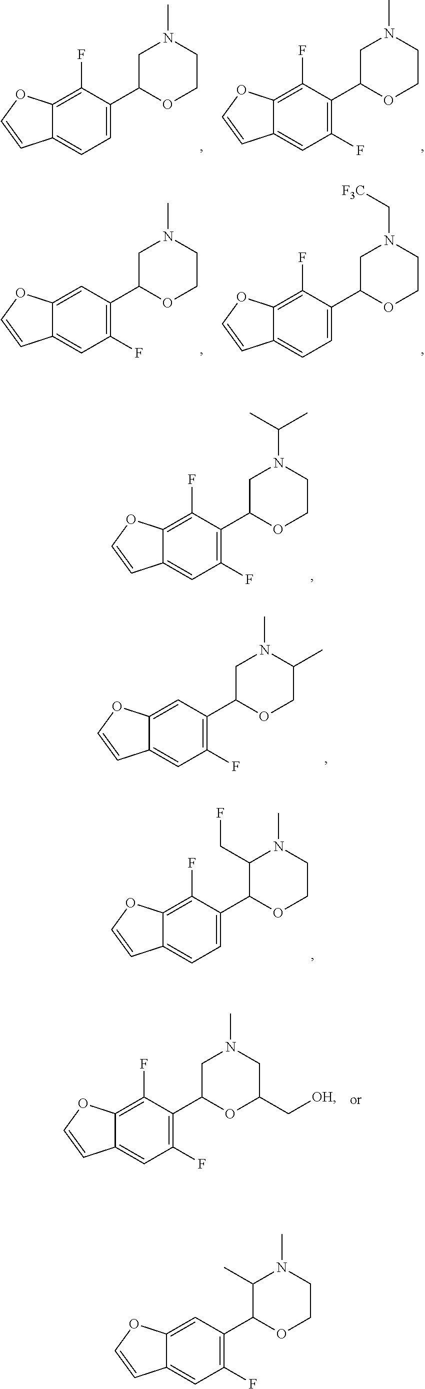

or pharmaceutically acceptable salt or salt mixture thereof.

In certain embodiments the halogenated benzofuran compound is of Formula:

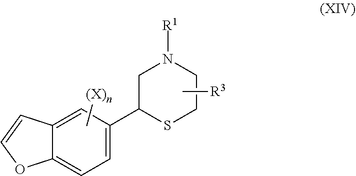

or pharmaceutically acceptable salt or salt mixture thereof.

In certain embodiments the halogenated benzofuran compound is of Formula:

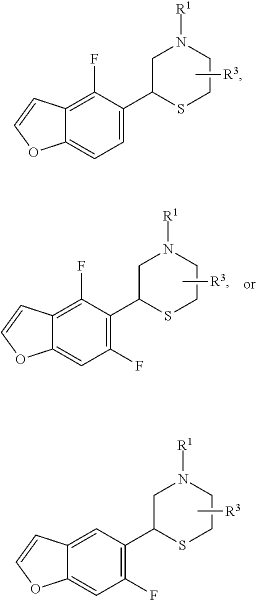

or pharmaceutically acceptable salt or salt mixture thereof.

In certain embodiments the halogenated benzofuran compound is of Formula:

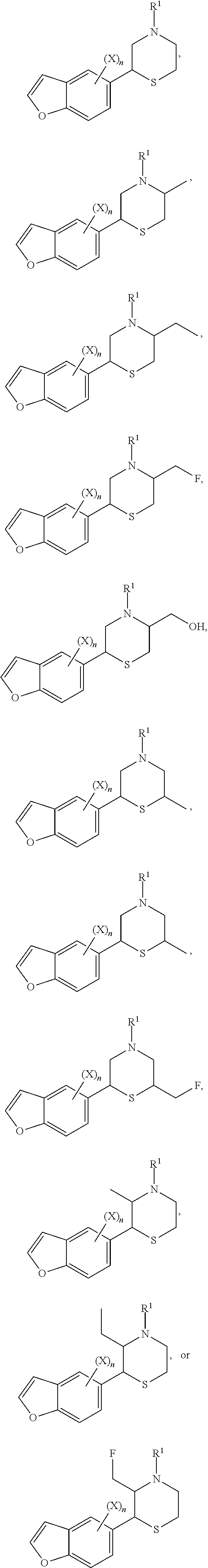

or pharmaceutically acceptable salt or salt mixture thereof.

In certain embodiments the halogenated benzofuran compound is of Formula:

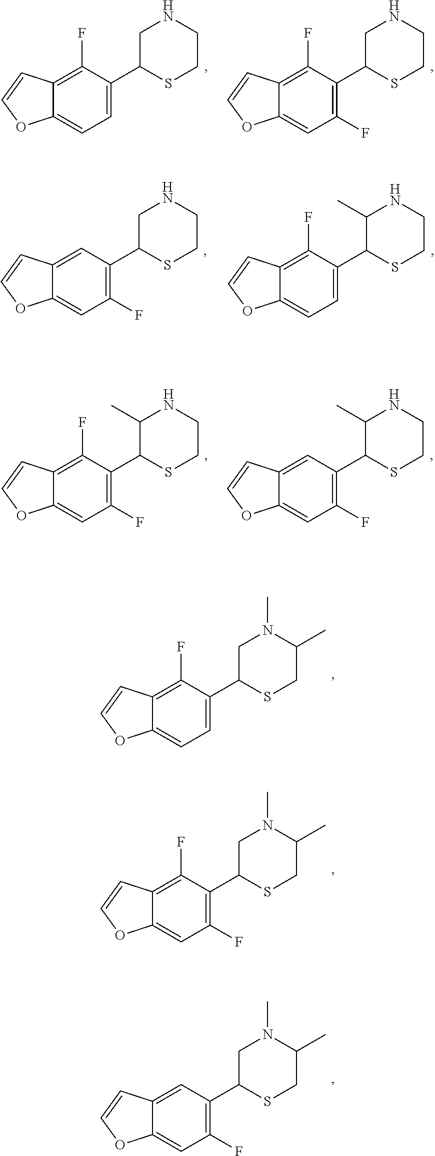

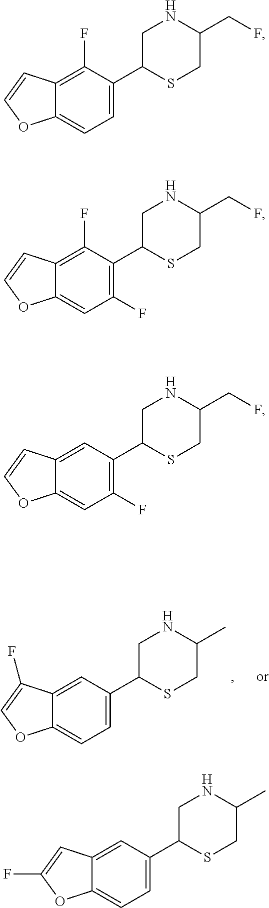 [SMILES: FCC1CSC(c2ccc3occc3c2F)CN1 ; FCC1CSC(c2c(F)cc3occc3c2F)CN1 ; FCC1CSC(c2cc3ccoc3cc2F)CN1 ; CC1CSC(c2ccc3occ(F)c3c2)CN1 ; CC1CSC(c2ccc3oc(F)cc3c2)CN1]

or pharmaceutically acceptable salt or salt mixture thereof.

In certain embodiments the halogenated benzofuran compound is of Formula:

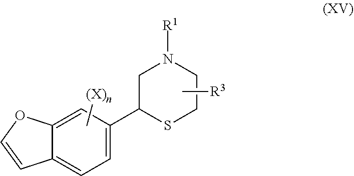

or pharmaceutically acceptable salt or salt mixture thereof.

In certain embodiments the halogenated benzofuran compound is of Formula:

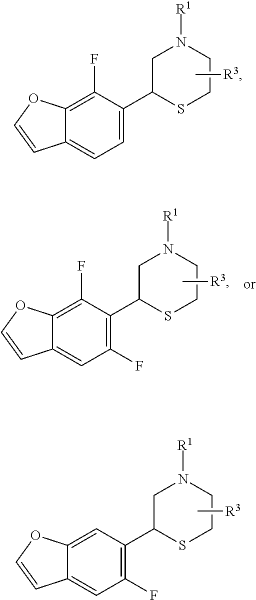

or pharmaceutically acceptable salt or salt mixture thereof.

In certain embodiments the halogenated benzofuran compound is of Formula:

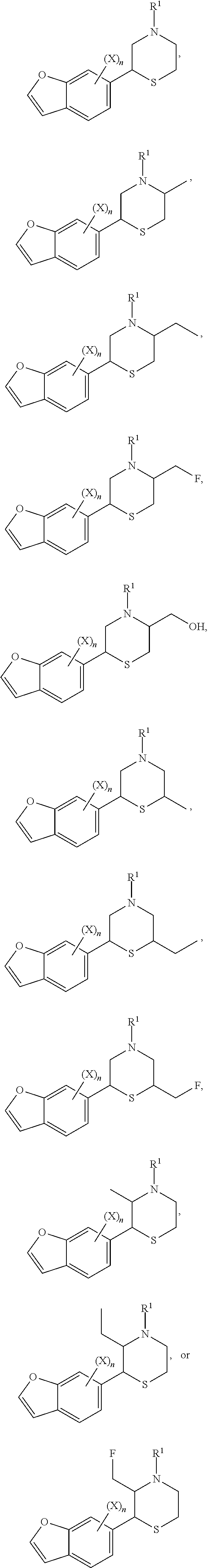

or pharmaceutically acceptable salt or salt mixture thereof.

In certain embodiments the halogenated benzofuran compound is of Formula:

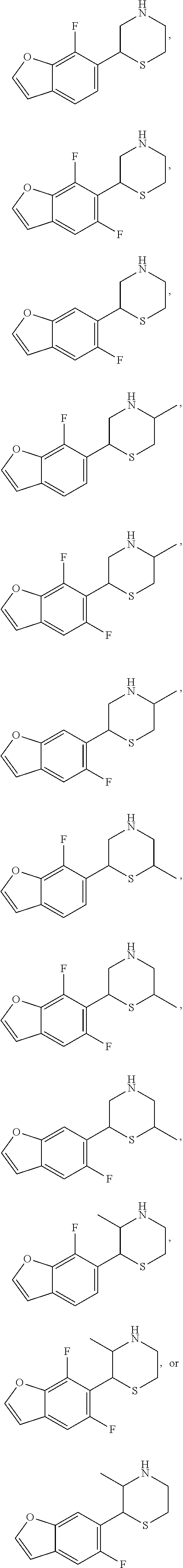

or pharmaceutically acceptable salt or salt mixture thereof.

In certain embodiments the halogenated benzofuran compound is of Formula:

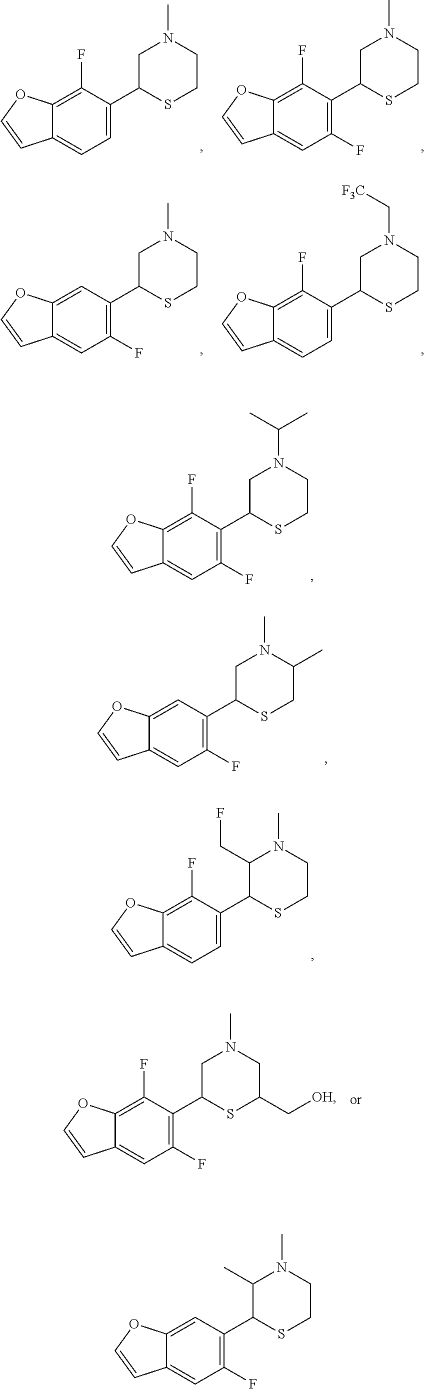

or pharmaceutically acceptable salt or salt mixture thereof.

In certain embodiments an enantiomerically pure or enriched compound of Formula:

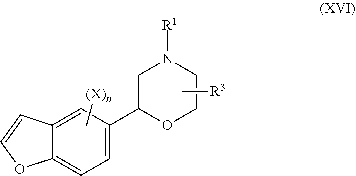

or pharmaceutically acceptable salt or salt mixture thereof.

In certain embodiments an enantiomerically pure or enriched compound of Formula:

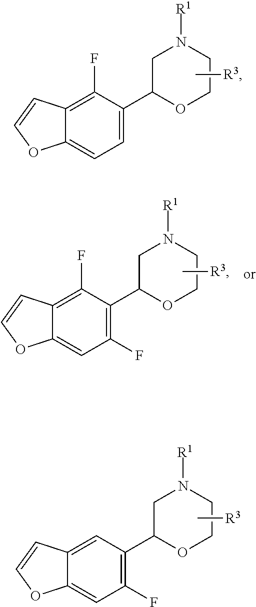

or pharmaceutically acceptable salt or salt mixture thereof.

In certain embodiments an enantiomerically pure or enriched compound of Formula:

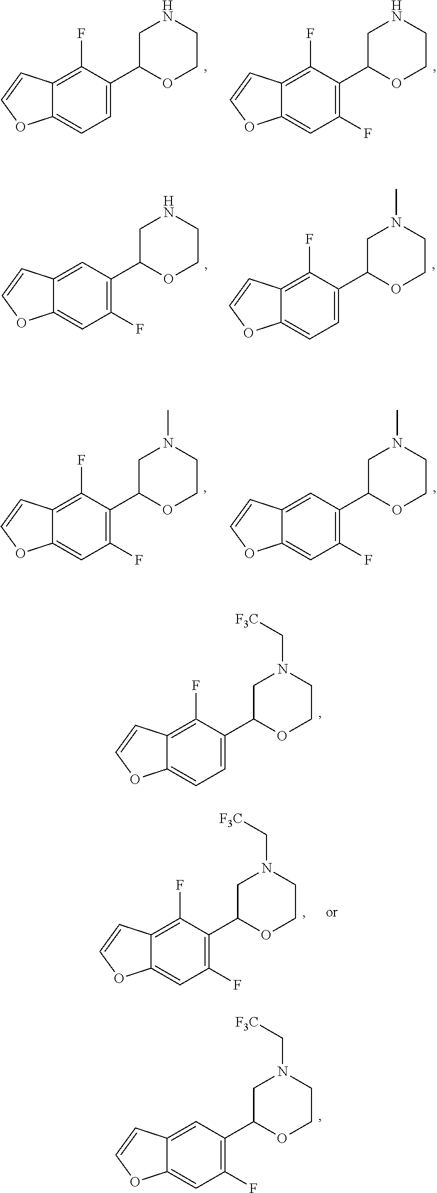

or pharmaceutically acceptable salt or salt mixture thereof.

In certain embodiments an enantiomerically pure or enriched compound of Formula:

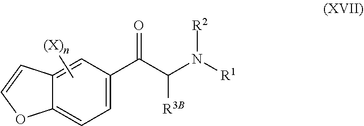

or pharmaceutically acceptable salt or salt mixture thereof.

In certain embodiments an enantiomerically pure or enriched compound of Formula:

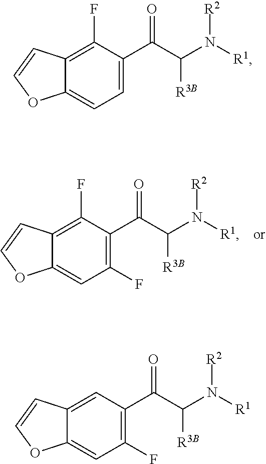

or pharmaceutically acceptable salt or salt mixture thereof.

In certain embodiments an enantiomerically pure or enriched compound of Formula:

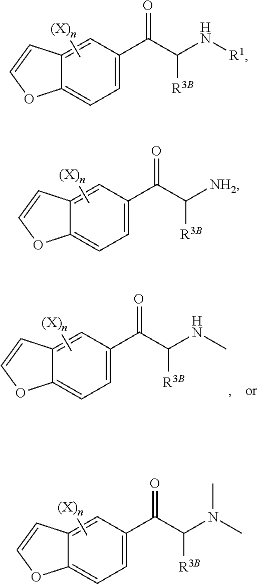

or pharmaceutically acceptable salt or salt mixture thereof.

In certain embodiments an enantiomerically pure or enriched compound of Formula:

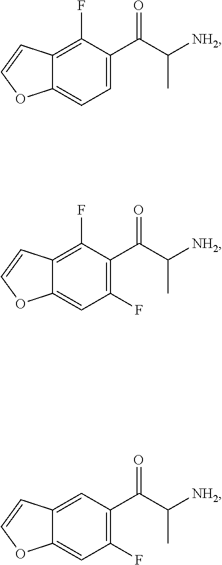 [SMILES: CC(N)C(=O)c1ccc2occc2c1F ; CC(N)C(=O)c1c(F)cc2occc2c1F ; CC(N)C(=O)c1cc2ccoc2cc1F]

or pharmaceutically acceptable salt or salt mixture thereof.

In certain embodiments an enantiomerically pure or enriched compound of Formula:

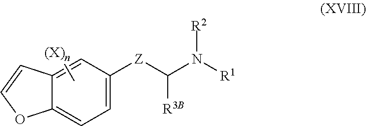

or pharmaceutically acceptable salt or salt mixture thereof.

In certain embodiments an enantiomerically pure or enriched compound of Formula:

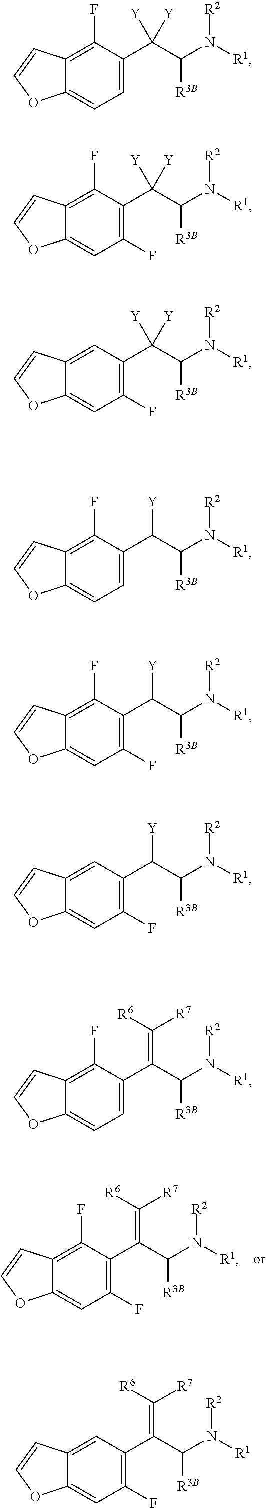

or pharmaceutically acceptable salt or salt mixture thereof.

In certain embodiments an enantiomerically pure or enriched compound of Formula:

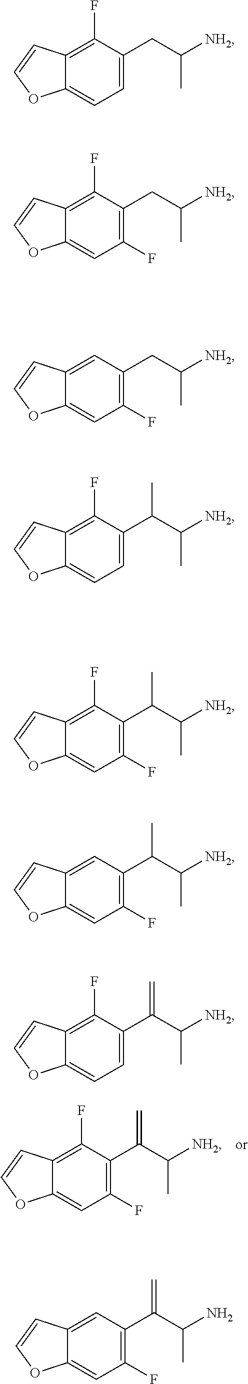

or pharmaceutically acceptable salt or salt mixture thereof.

In certain embodiments an enantiomerically pure or enriched compound of Formula:

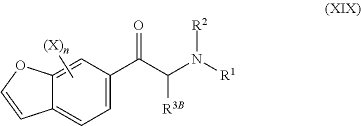

or pharmaceutically acceptable salt or salt mixture thereof.

In certain embodiments an enantiomerically pure or enriched compound of Formula:

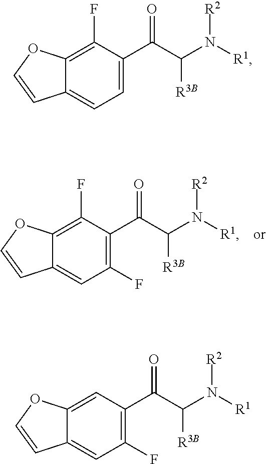

or pharmaceutically acceptable salt or salt mixture thereof.

In certain embodiments an enantiomerically pure or enriched compound of Formula:

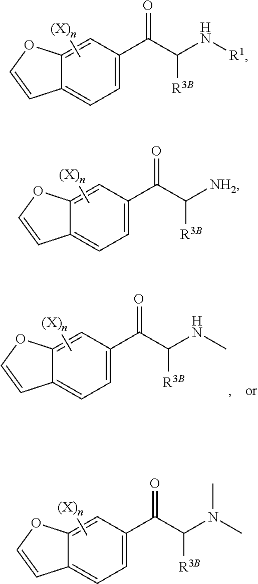

or pharmaceutically acceptable salt or salt mixture thereof.

In certain embodiments an enantiomerically pure or enriched compound of Formula:

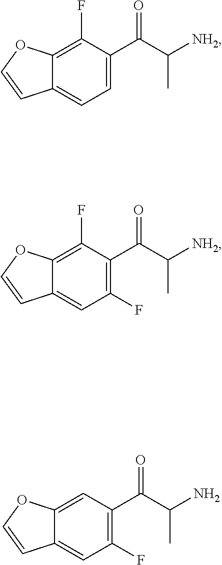 [SMILES: CC(N)C(=O)c1ccc2ccoc2c1F ; CC(N)C(=O)c1c(F)cc2ccoc2c1F ; CC(N)C(=O)c1cc2occc2cc1F]

or pharmaceutically acceptable salt or salt mixture thereof.

In certain embodiments an enantiomerically pure or enriched compound of Formula:

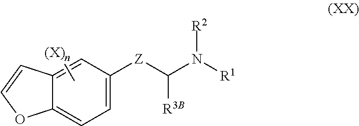

or pharmaceutically acceptable salt or salt mixture thereof.

In certain embodiments an enantiomerically pure or enriched compound of Formula:

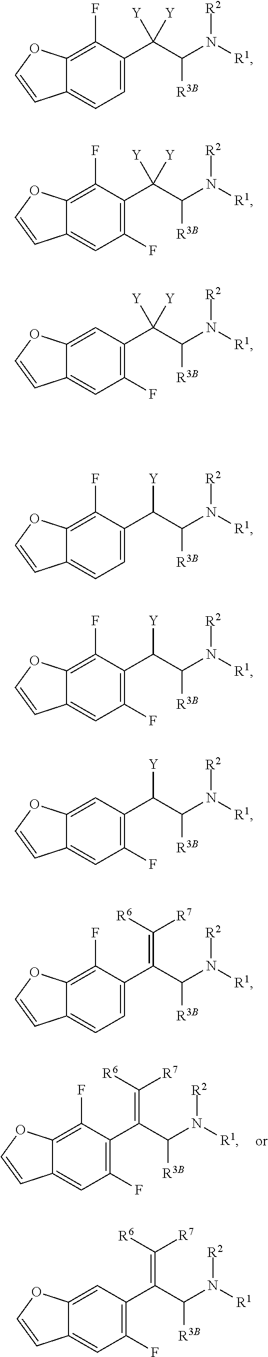

or pharmaceutically acceptable salt or salt mixture thereof.

In certain embodiments an enantiomerically pure or enriched compound of Formula:

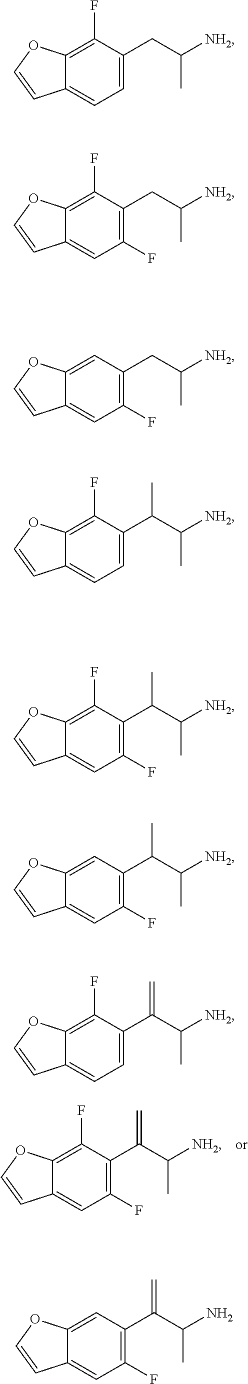

or pharmaceutically acceptable salt or salt mixture thereof.

In certain embodiments the invention provides a use of a compound of Formula:

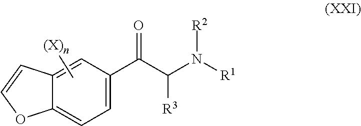

or pharmaceutically acceptable salt or salt mixture thereof.

In certain embodiments the invention provides a use of a compound of Formula:

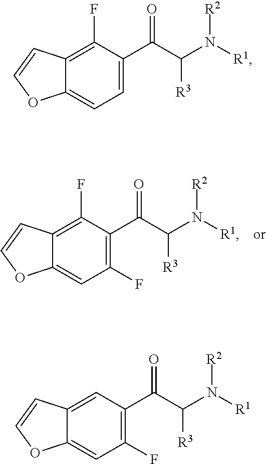

or pharmaceutically acceptable salt or salt mixture thereof.

In certain embodiments the invention provides a use of a compound of Formula:

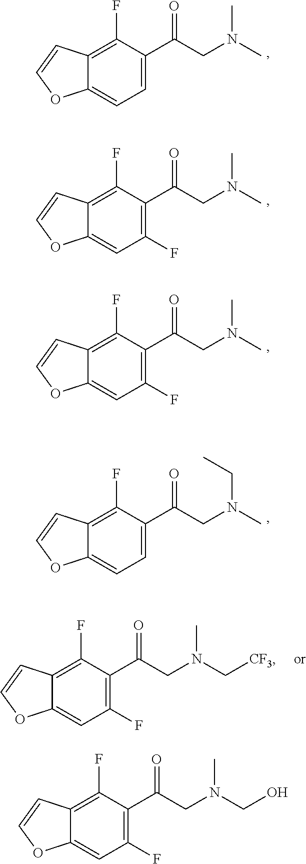

or pharmaceutically acceptable salt or salt mixture thereof.

In certain embodiments the invention provides a use of a compound of Formula:

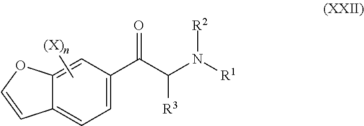

or pharmaceutically acceptable salt or salt mixture thereof.

In certain embodiments the invention provides a use of a compound of Formula:

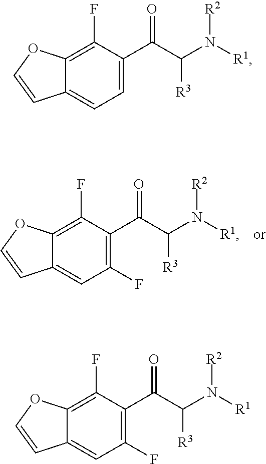

or pharmaceutically acceptable salt or salt mixture thereof.

In certain embodiments the invention provides a use of a compound of Formula:

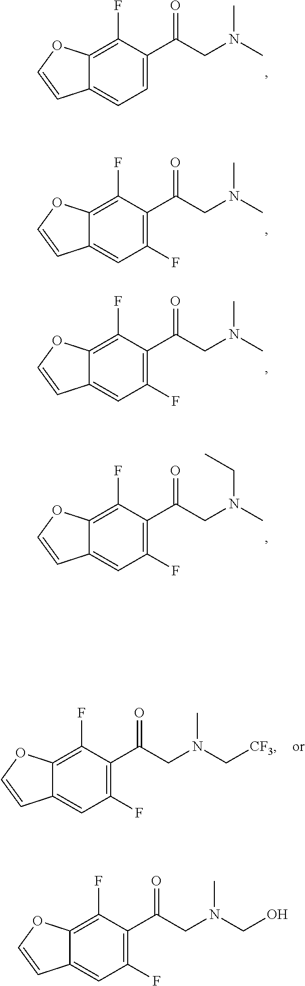

or pharmaceutically acceptable salt or salt mixture thereof.

Non-limiting examples of compounds of the present invention include:

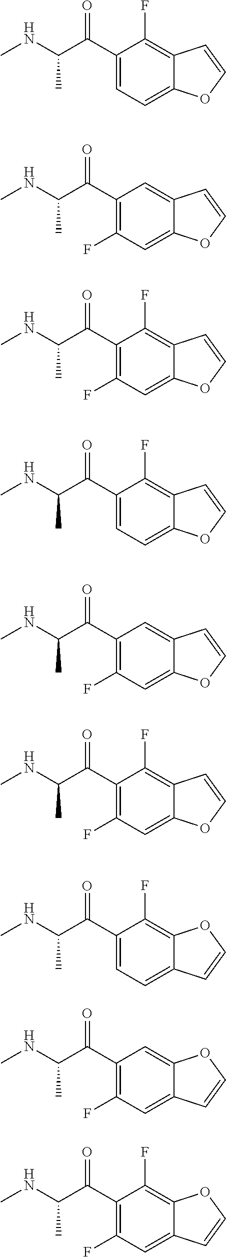

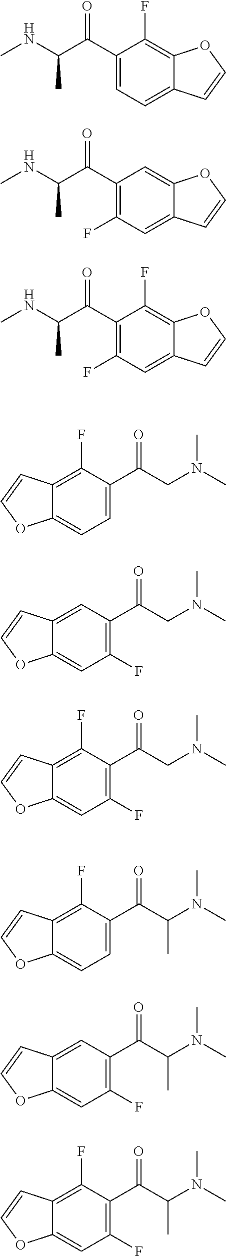

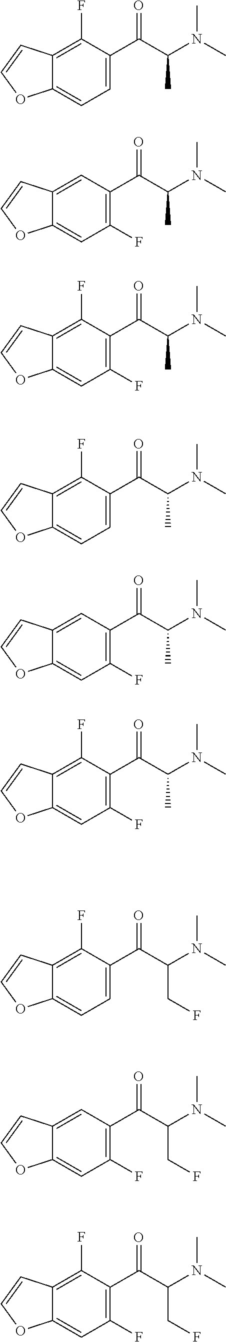

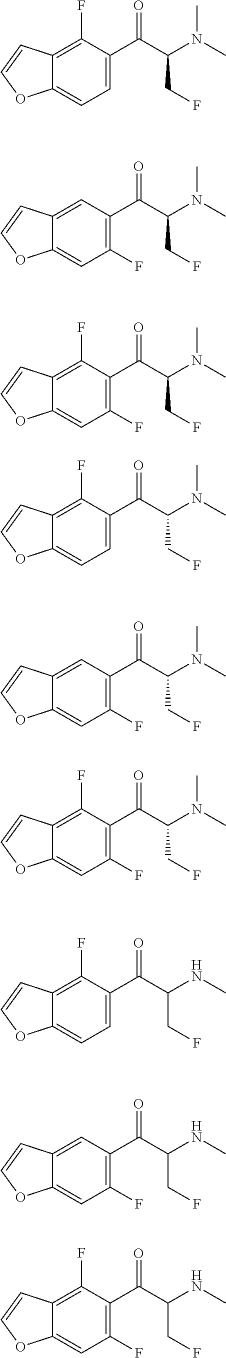

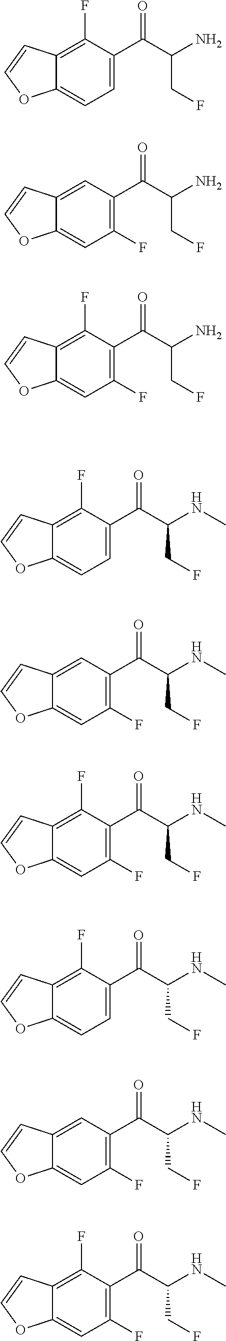

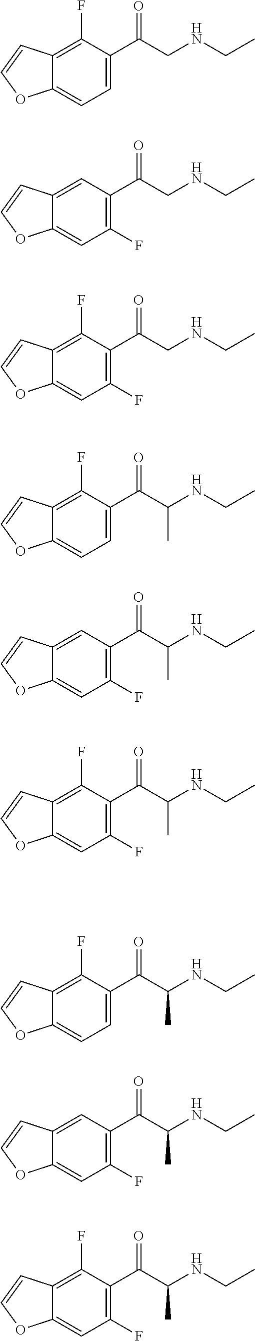

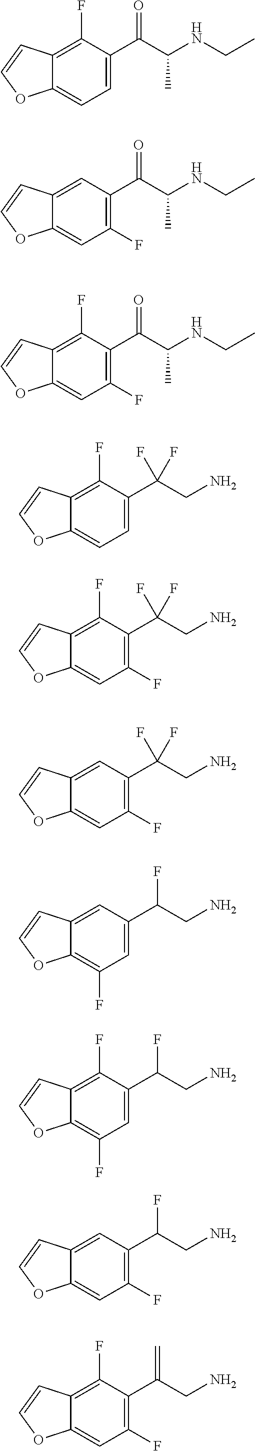

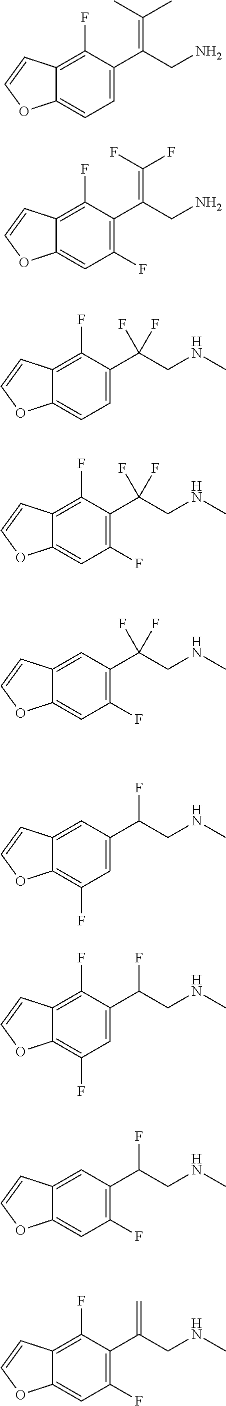

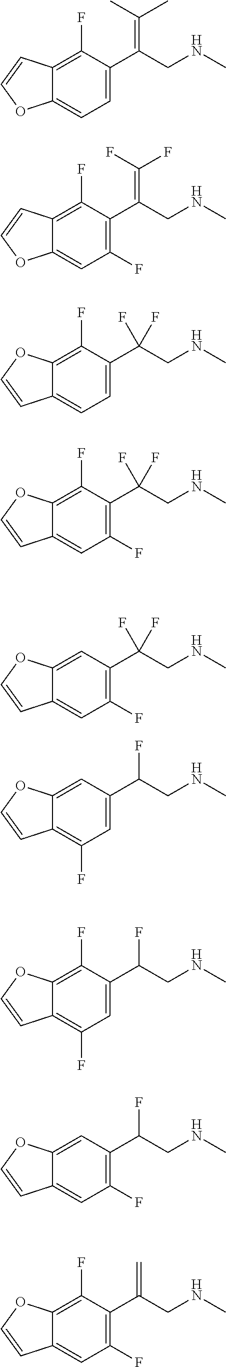

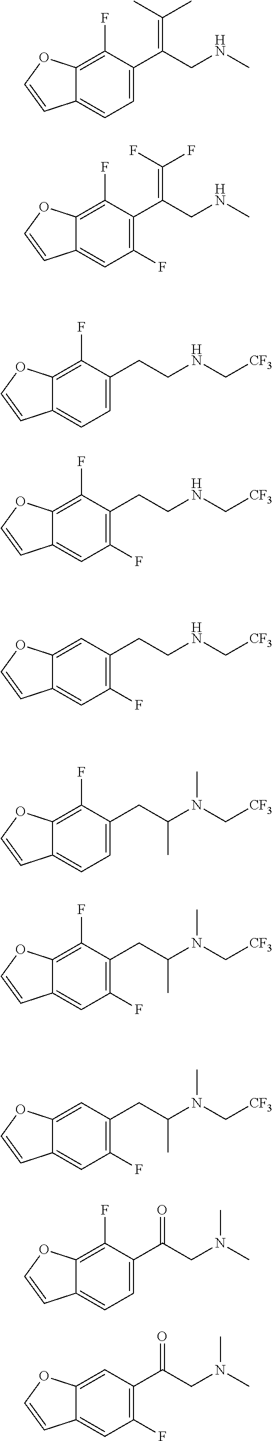

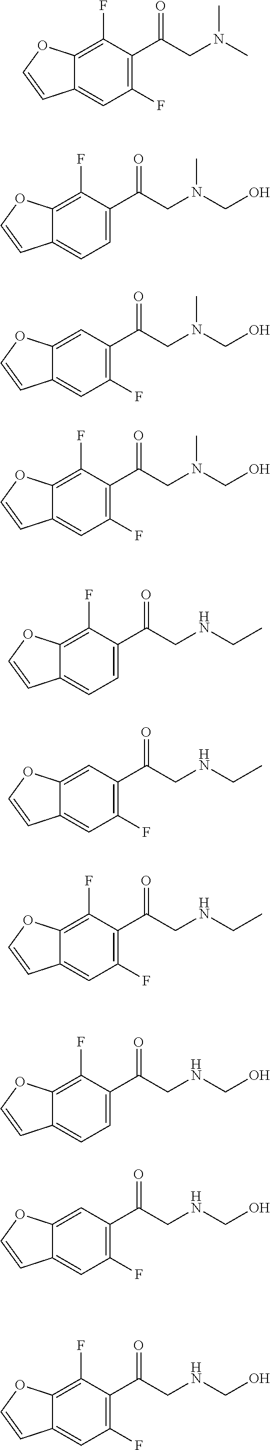

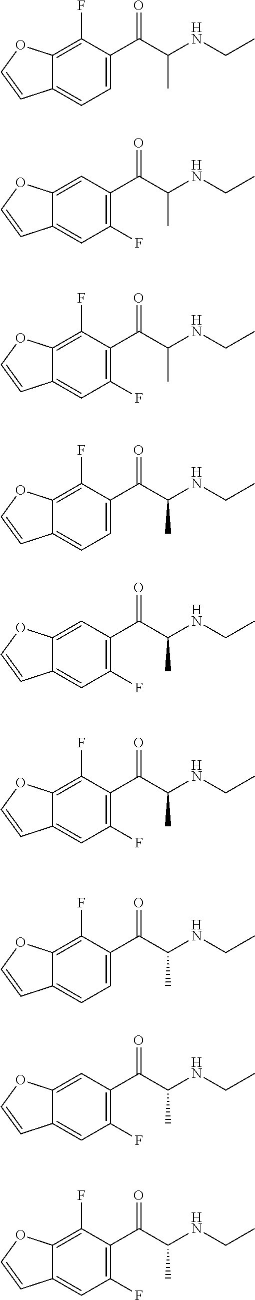

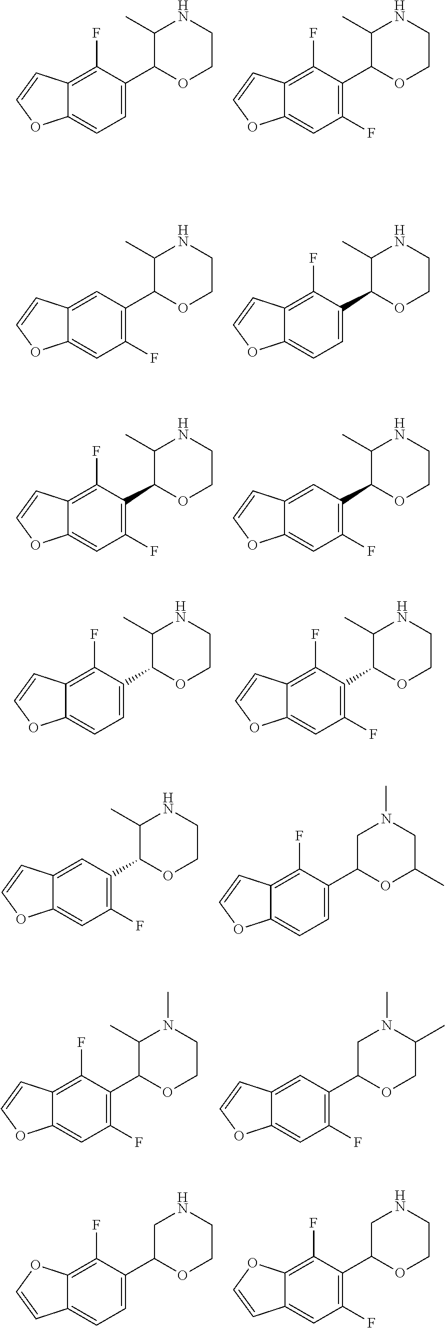

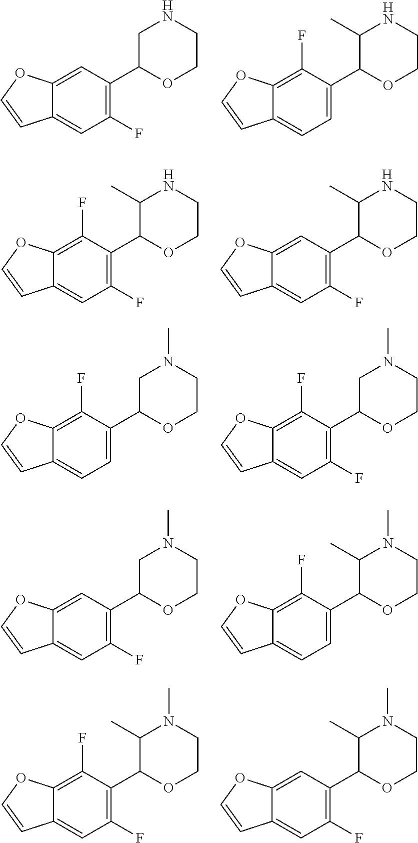

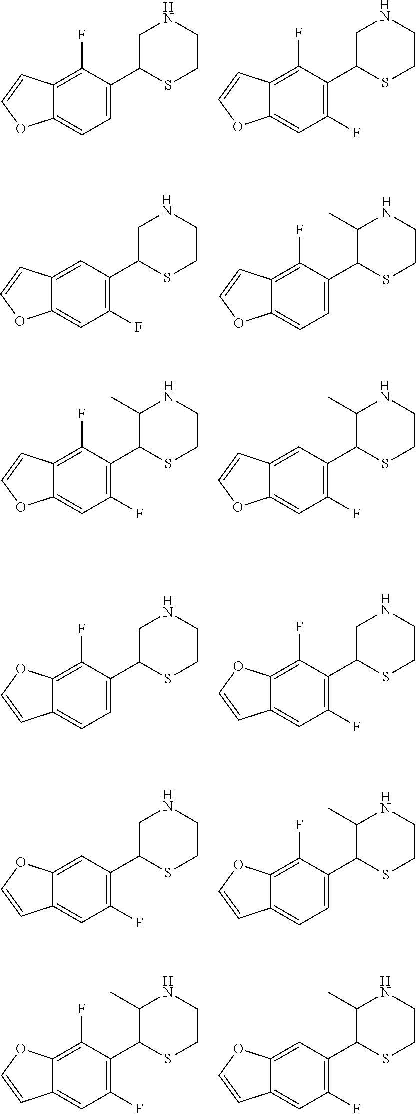

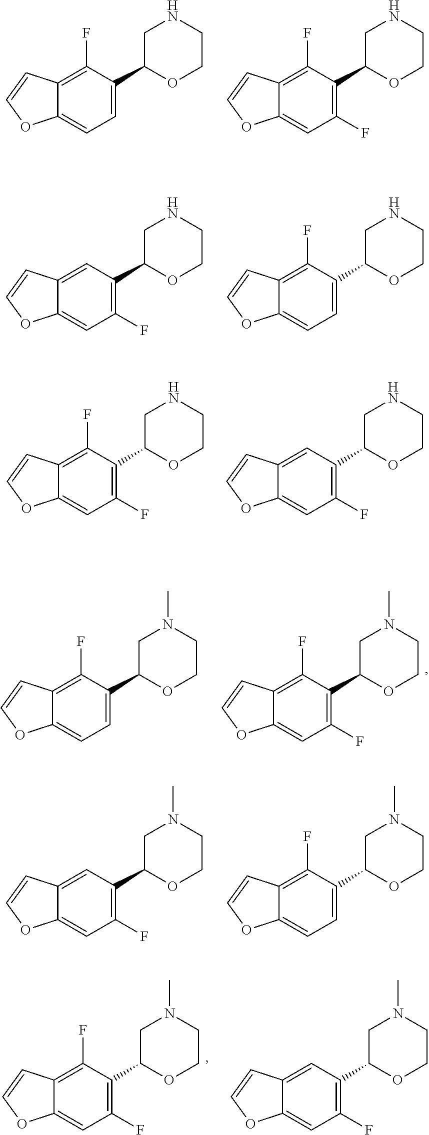

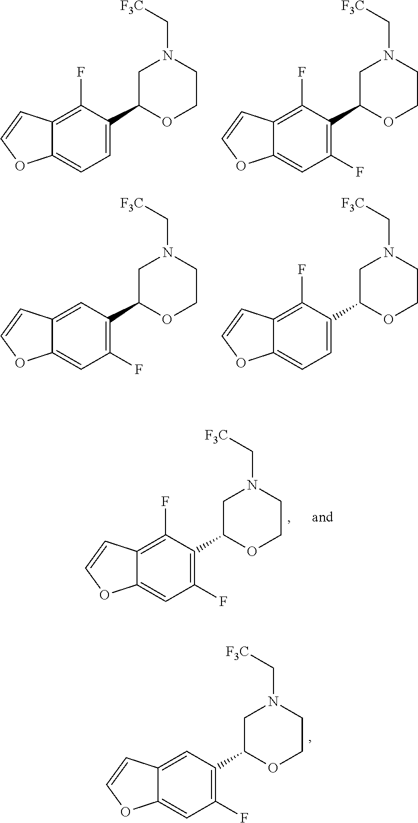

or a pharmaceutically acceptable salt, salt mixture, racemic mixture, or stereoisomer enriched mixture thereof.

Additional non-limiting examples of compounds of the present invention include

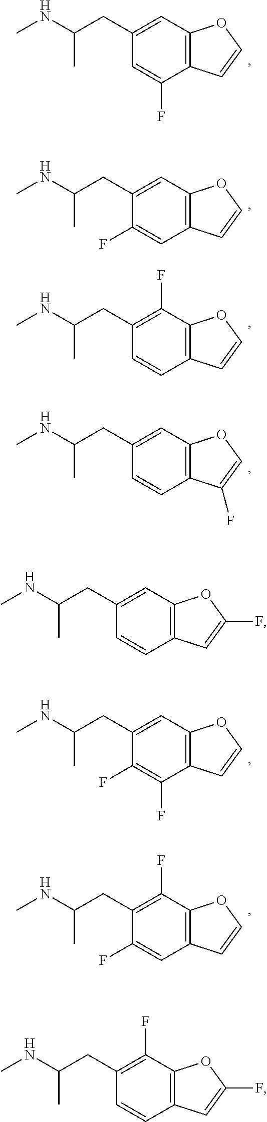

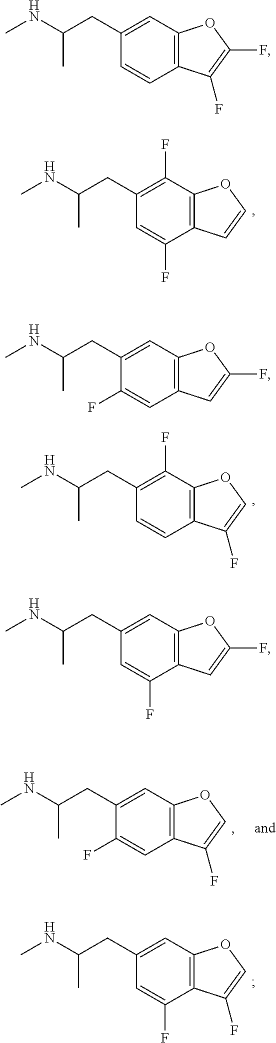

or a pharmaceutically acceptable salt, salt mixture, or enantiomerically enriched mixture thereof.

In certain embodiments the compound of the present invention is selected from:

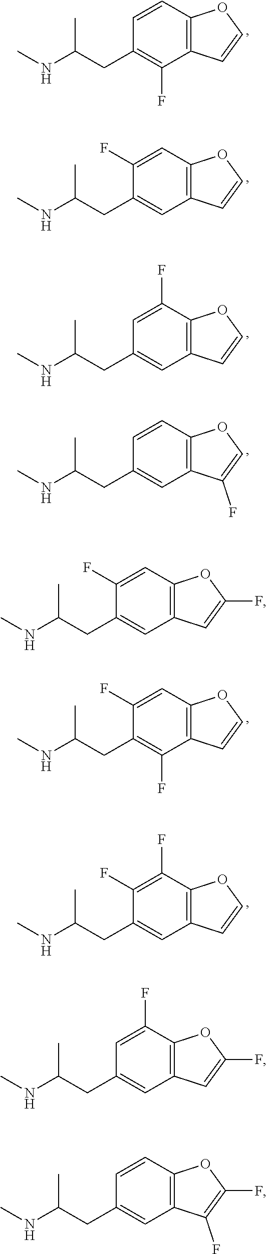

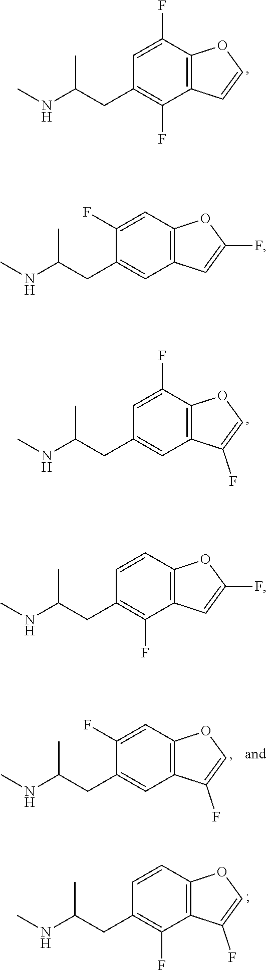 [SMILES: CNC(C)Cc1cc(F)c2occc2c1F ; CNC(C)Cc1cc2cc(F)oc2cc1F ; CNC(C)Cc1cc(F)c2occ(F)c2c1 ; CNC(C)Cc1ccc2oc(F)cc2c1F ; CNC(C)Cc1cc2c(F)coc2cc1F ; CNC(C)Cc1ccc2occ(F)c2c1F]

or a pharmaceutically acceptable salt, salt mixture, or enantiomerically enriched mixture thereof.

In certain embodiments the compound of the present invention is selected from:

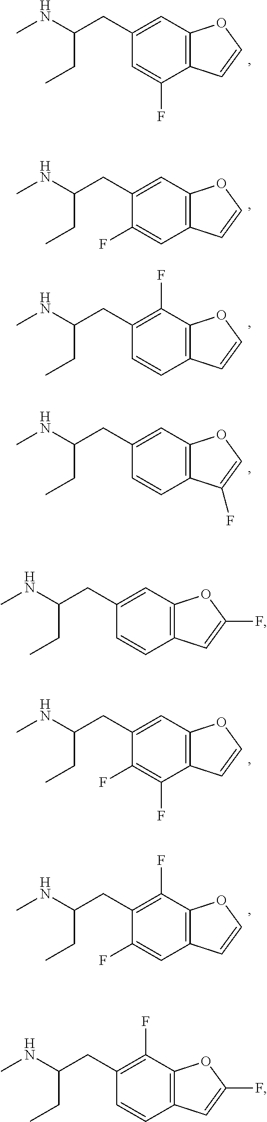

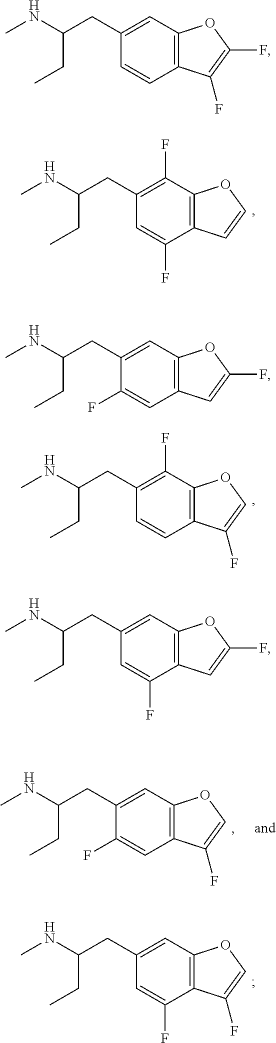

or a pharmaceutically acceptable salt, salt mixture, or enantiomerically enriched mixture thereof.

In certain embodiments the compound of the present invention is selected from:

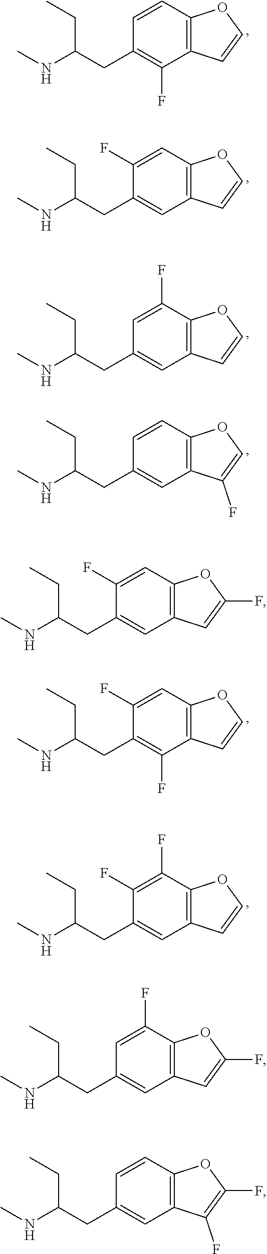

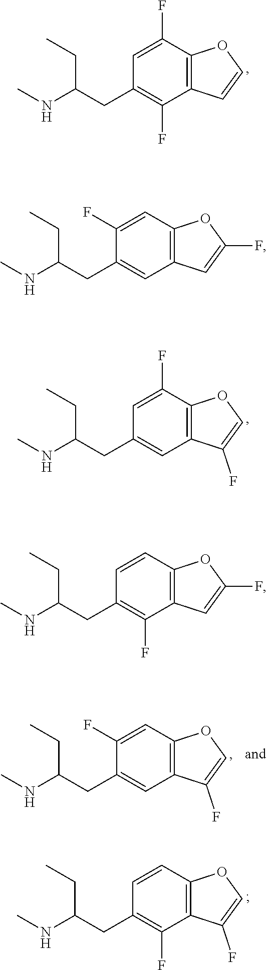 [SMILES: CCC(Cc1cc(F)c2occc2c1F)NC ; CCC(Cc1cc2cc(F)oc2cc1F)NC ; CCC(Cc1cc(F)c2occ(F)c2c1)NC ; CCC(Cc1ccc2oc(F)cc2c1F)NC ; CCC(Cc1cc2c(F)coc2cc1F)NC ; CCC(Cc1ccc2occ(F)c2c1F)NC]

or a pharmaceutically acceptable salt, salt mixture, or enantiomerically enriched mixture thereof.

In certain embodiments the compound of the present invention is selected from:

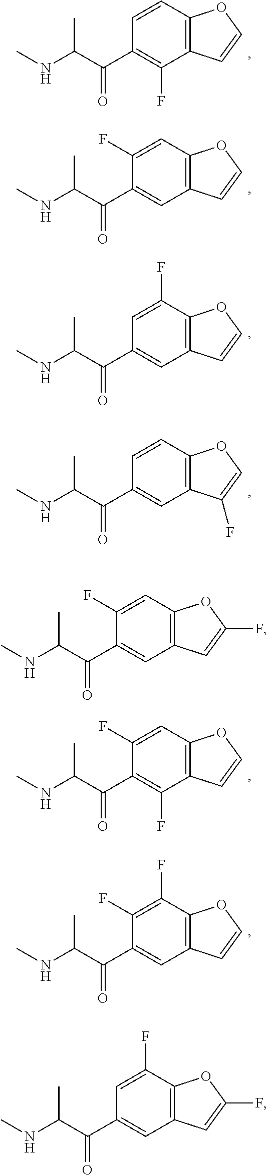

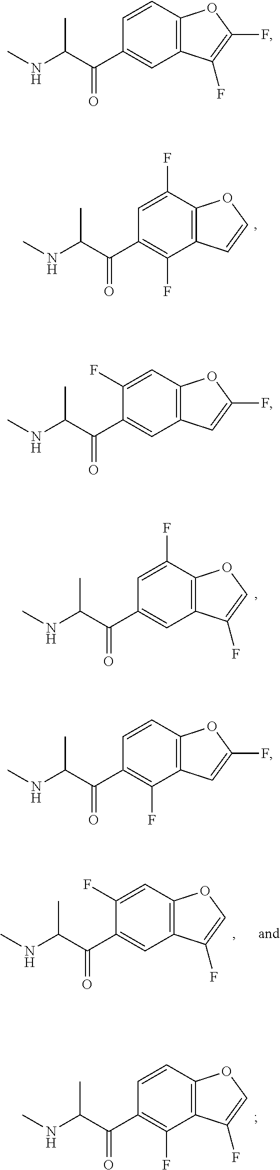

or a pharmaceutically acceptable salt, salt mixture, or enantiomerically enriched mixture thereof.

In certain embodiments the compound of the present invention is selected from:

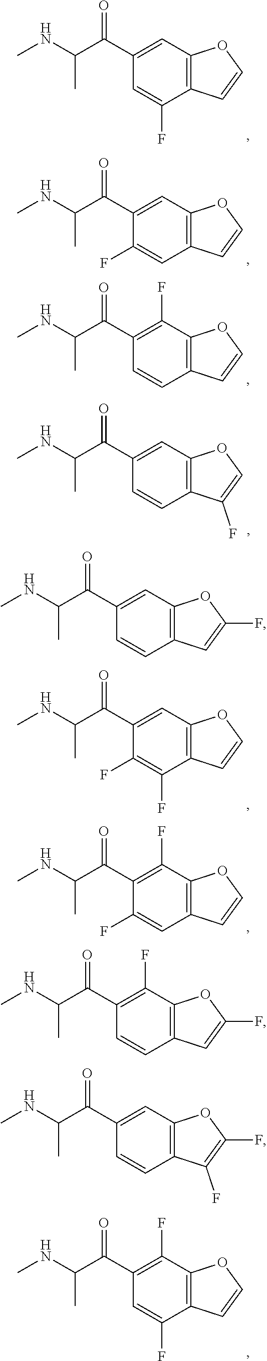

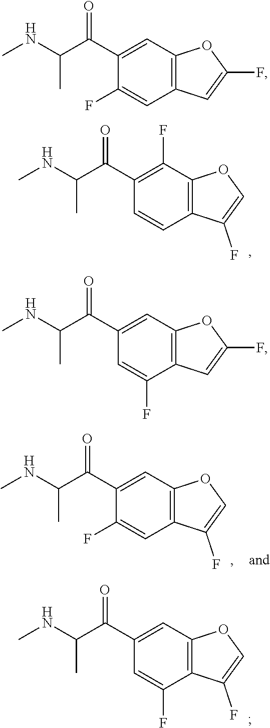

or a pharmaceutically acceptable salt, salt mixture, or enantiomerically enriched mixture thereof.

In certain embodiments the compound of the present invention is selected from:

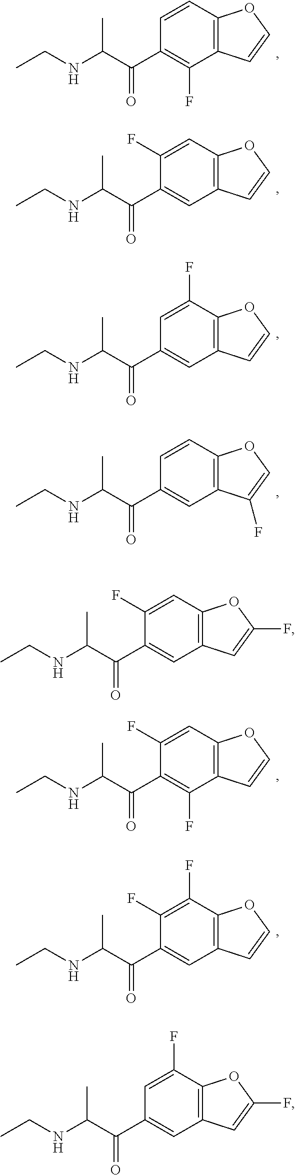

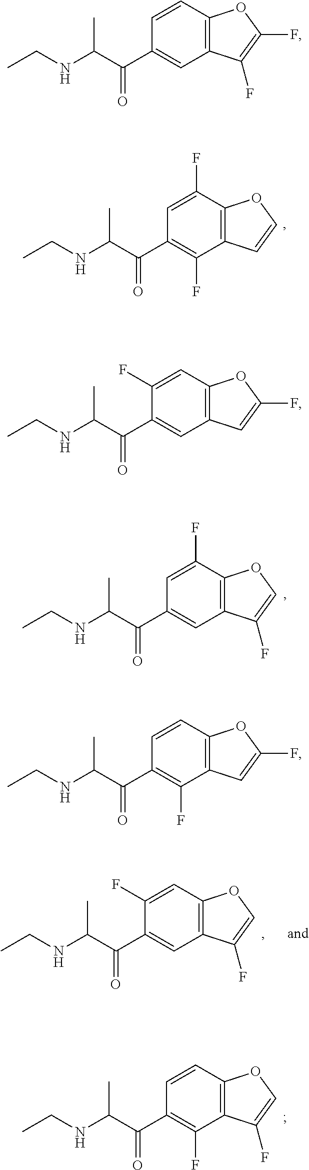

or a pharmaceutically acceptable salt, salt mixture, or enantiomerically enriched mixture thereof.

In certain embodiments the compound of the present invention is selected from:

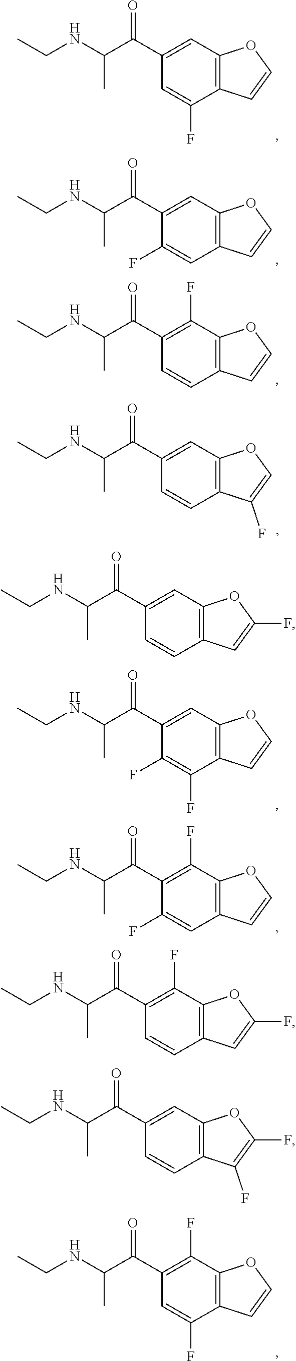

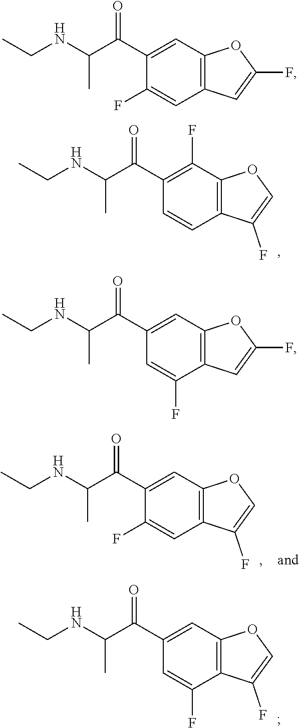

or a pharmaceutically acceptable salt, salt mixture, or enantiomerically enriched mixture thereof.

Additional non-limiting examples of compounds of the present invention include

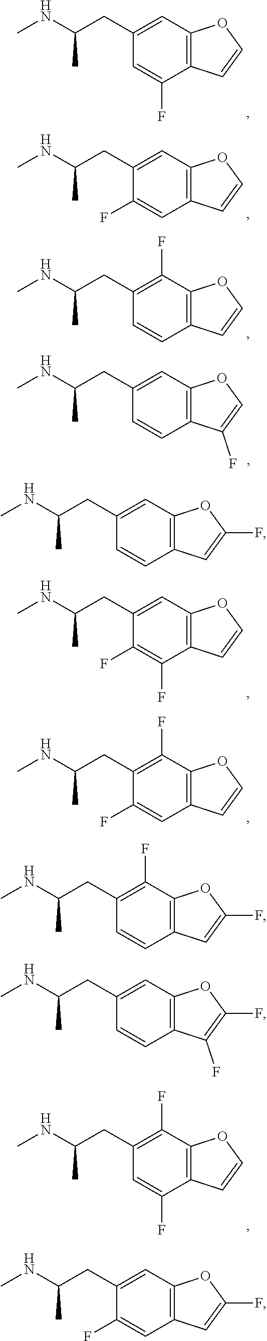

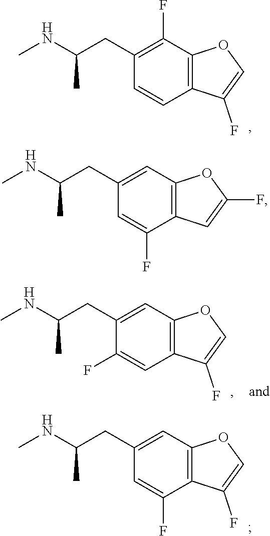

or a pharmaceutically acceptable salt, salt mixture, or enantiomerically enriched mixture thereof.

In certain embodiments the compound of the present invention is selected from:

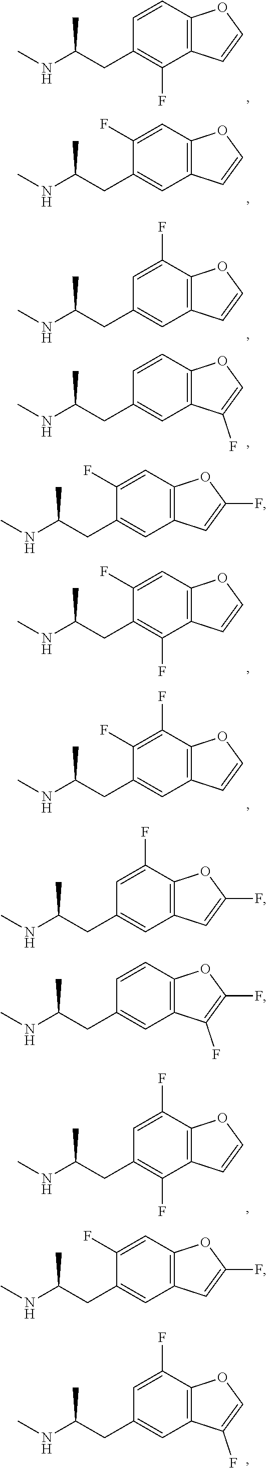

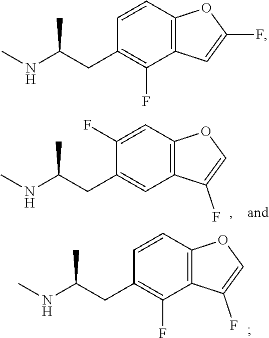

In certain embodiments the compound of the present invention is selected from:

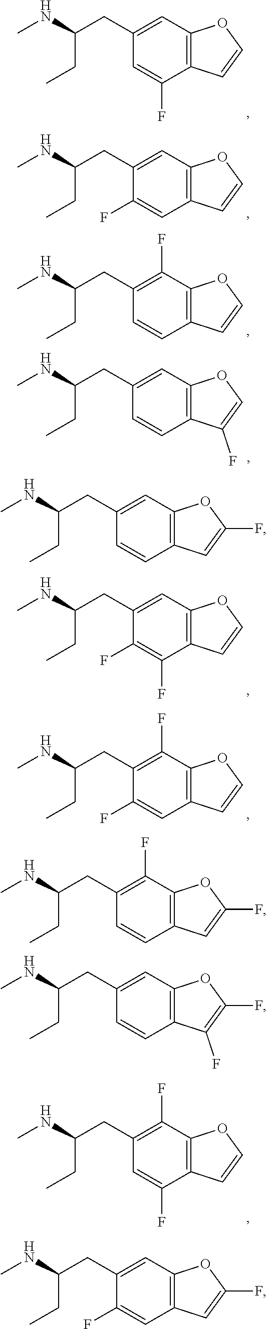

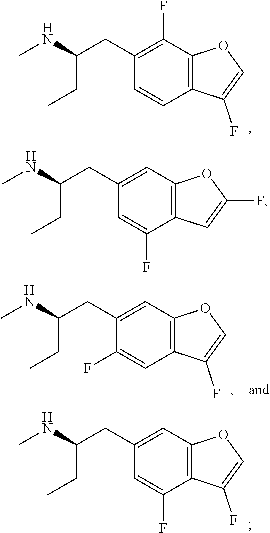

or a pharmaceutically acceptable salt, salt mixture, or enantiomerically enriched mixture thereof.

In certain embodiments the compound of the present invention is selected from:

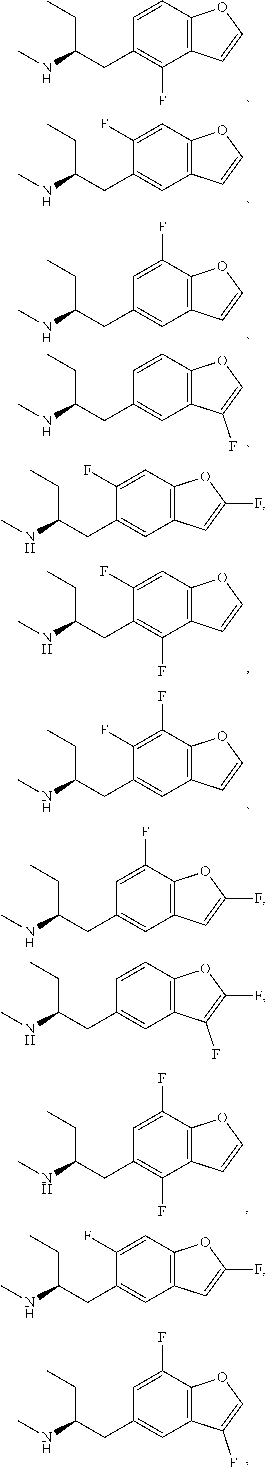

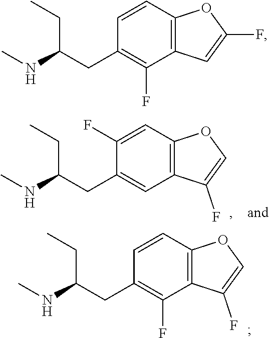

or a pharmaceutically acceptable salt, salt mixture, or enantiomerically enriched mixture thereof.

In certain embodiments the compound of the present invention is selected from:

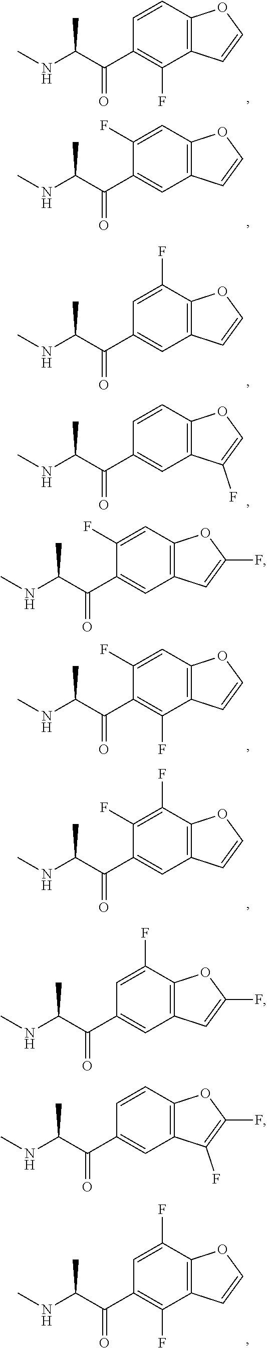

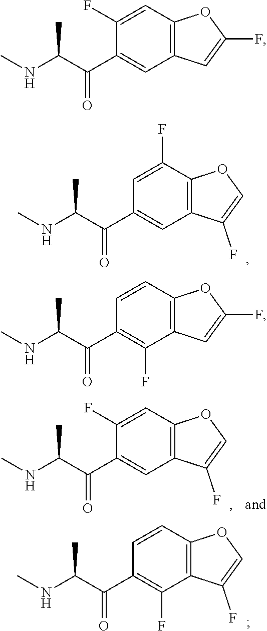

or a pharmaceutically acceptable salt, salt mixture, or enantiomerically enriched mixture thereof.

In certain embodiments the compound of the present invention is selected from:

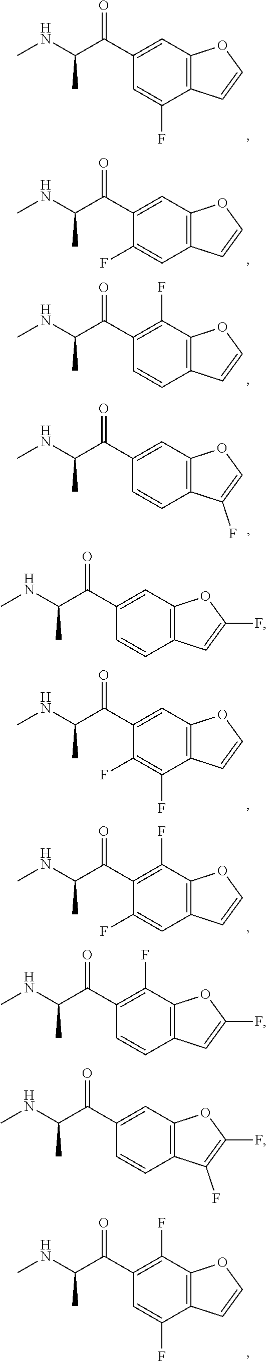

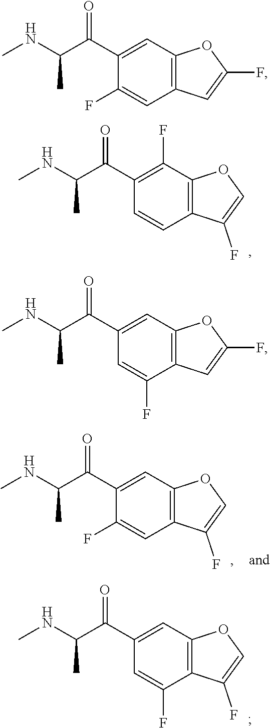

or a pharmaceutically acceptable salt, salt mixture, or enantiomerically enriched mixture thereof.

In certain embodiments the compound of the present invention is selected from:

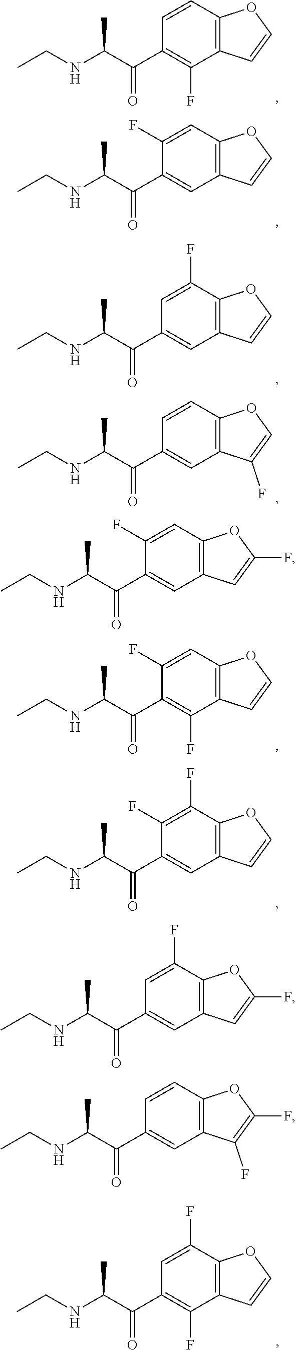

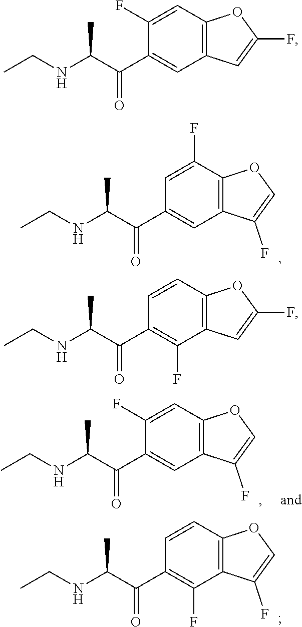

or a pharmaceutically acceptable salt, salt mixture, or enantiomerically enriched mixture thereof.

In certain embodiments the compound of the present invention is selected from:

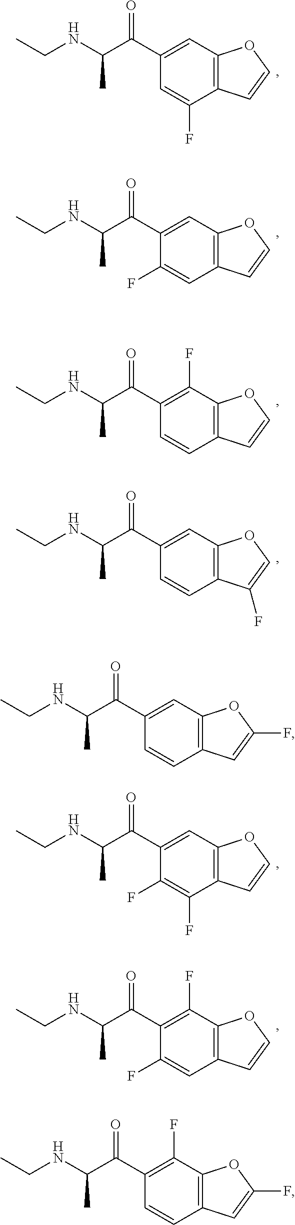

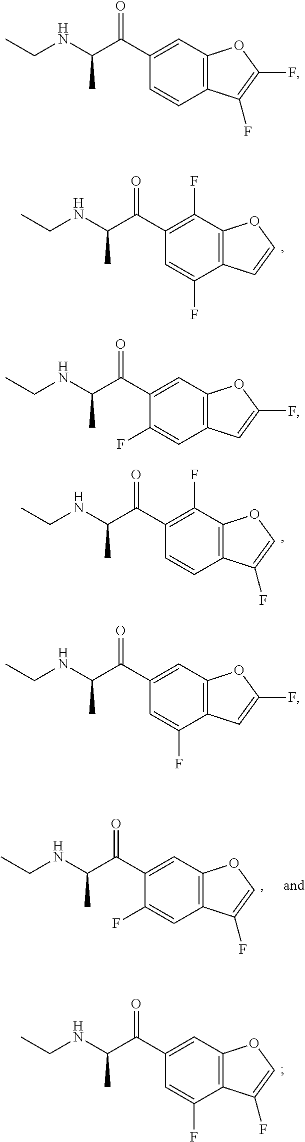

or a pharmaceutically acceptable salt, salt mixture, or enantiomerically enriched mixture thereof.

Additional non-limiting examples of compounds of the present invention include

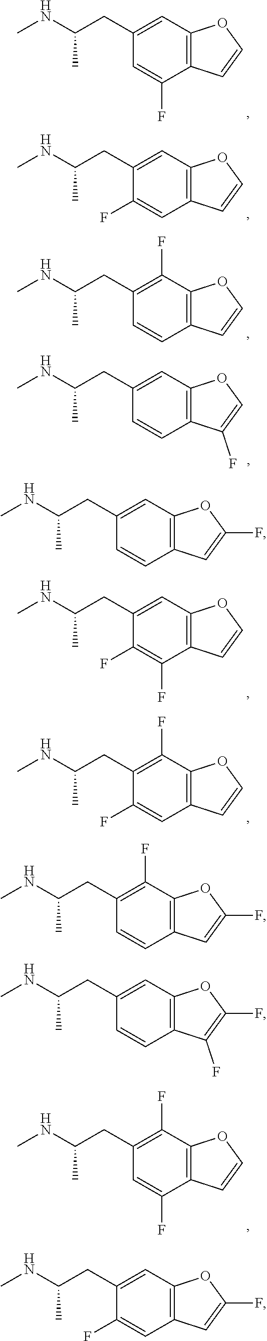

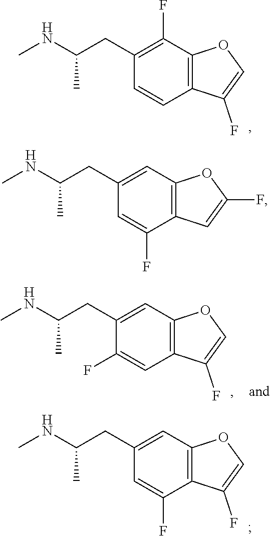

or a pharmaceutically acceptable salt, salt mixture, or enantiomerically enriched mixture thereof.

In certain embodiments the compound of the present invention is selected from:

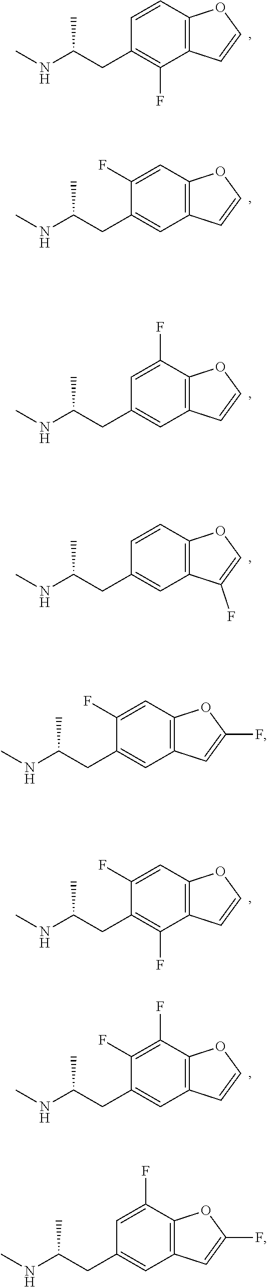

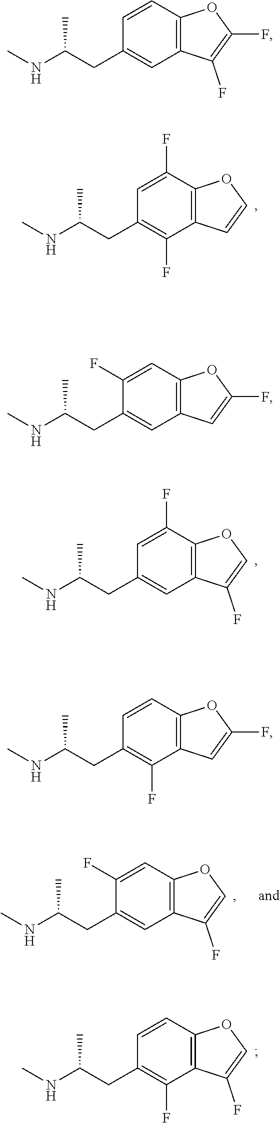

or a pharmaceutically acceptable salt, salt mixture, or enantiomerically enriched mixture thereof.

In certain embodiments the compound of the present invention is selected from:

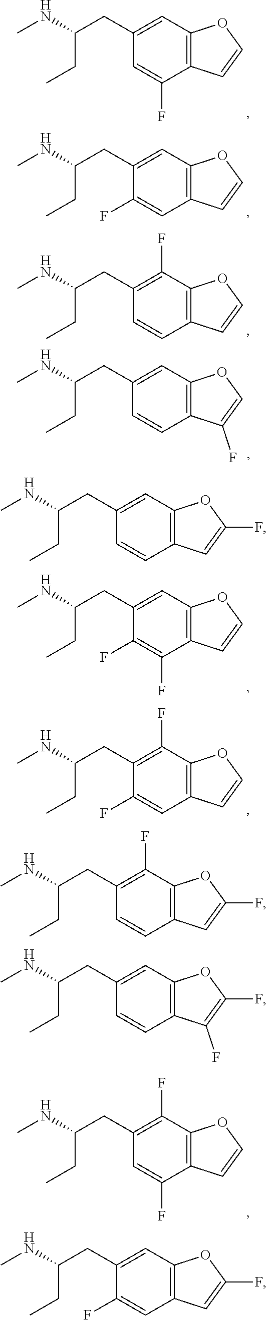

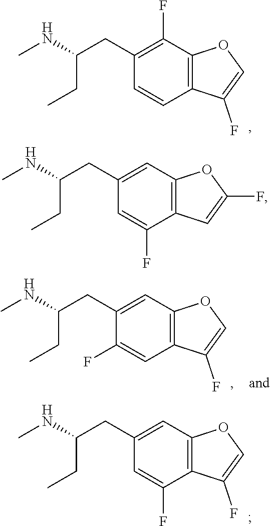

or a pharmaceutically acceptable salt, salt mixture, or enantiomerically enriched mixture thereof.

In certain embodiments the compound of the present invention is selected from:

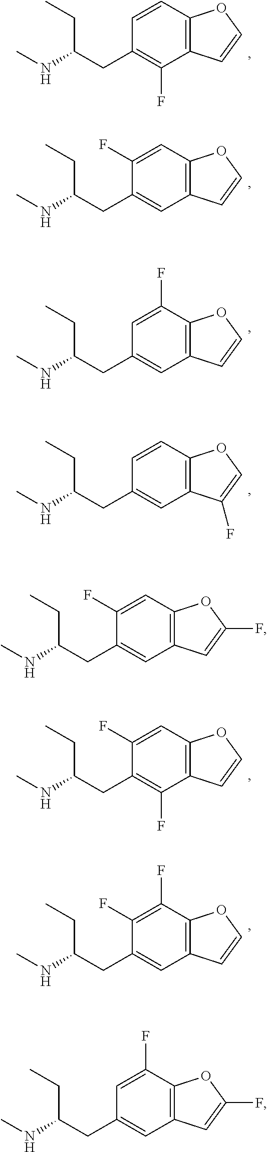

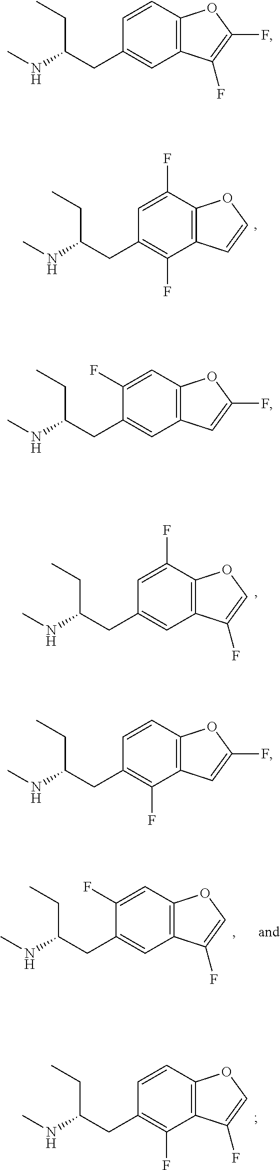

or a pharmaceutically acceptable salt, salt mixture, or enantiomerically enriched mixture thereof.

In certain embodiments the compound of the present invention is selected from:

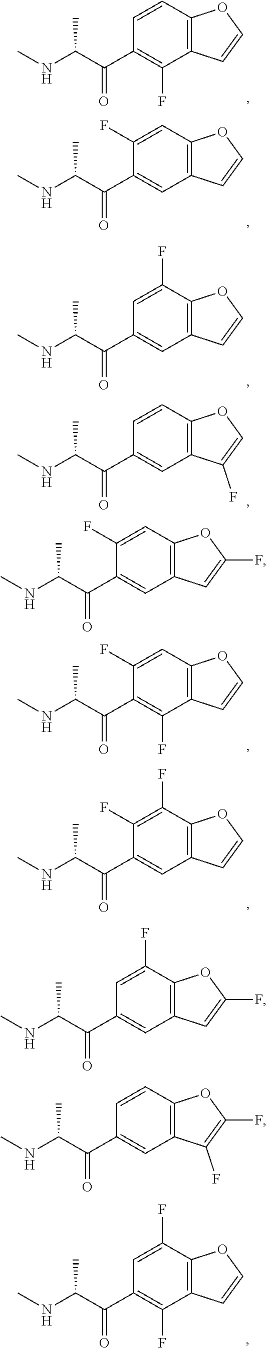

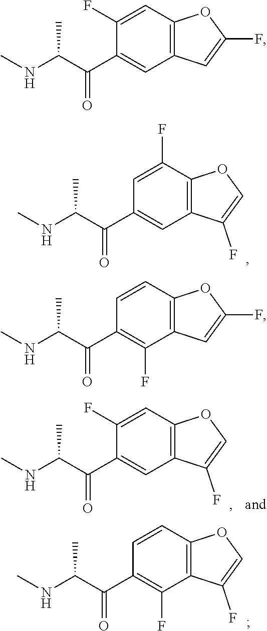

or a pharmaceutically acceptable salt, salt mixture, or enantiomerically enriched mixture thereof.

In certain embodiments the compound of the present invention is selected from:

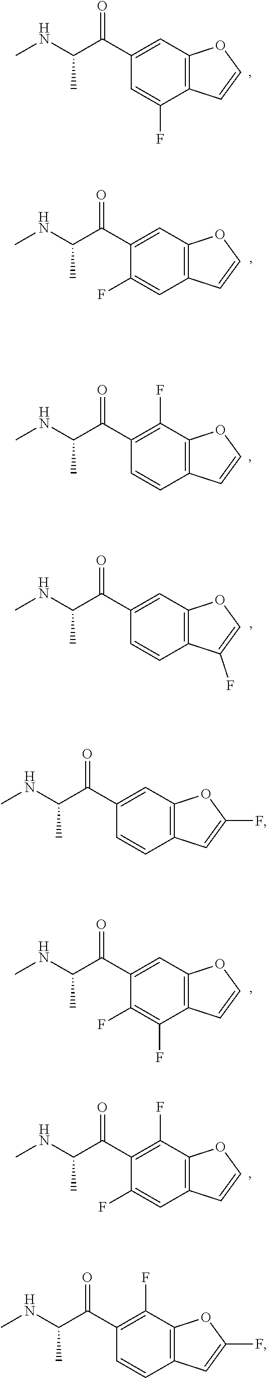

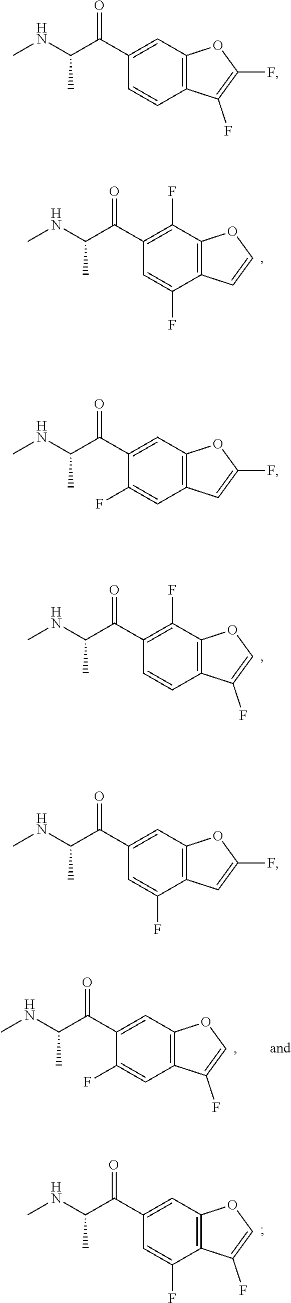

or a pharmaceutically acceptable salt, salt mixture, or enantiomerically enriched mixture thereof.

In certain embodiments the compound of the present invention is selected from:

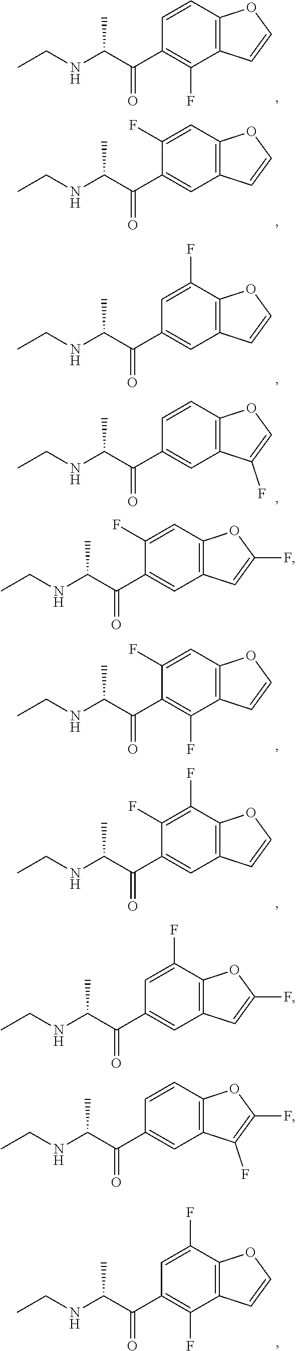

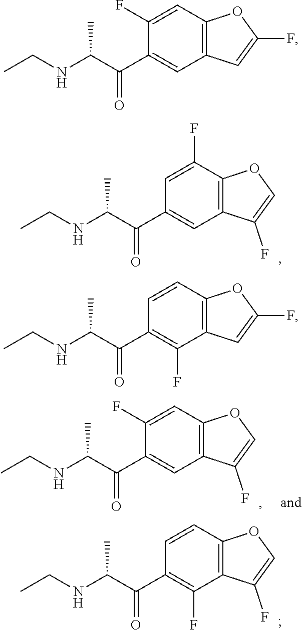

or a pharmaceutically acceptable salt, salt mixture, or enantiomerically enriched mixture thereof.

In certain embodiments the compound of the present invention is selected from:

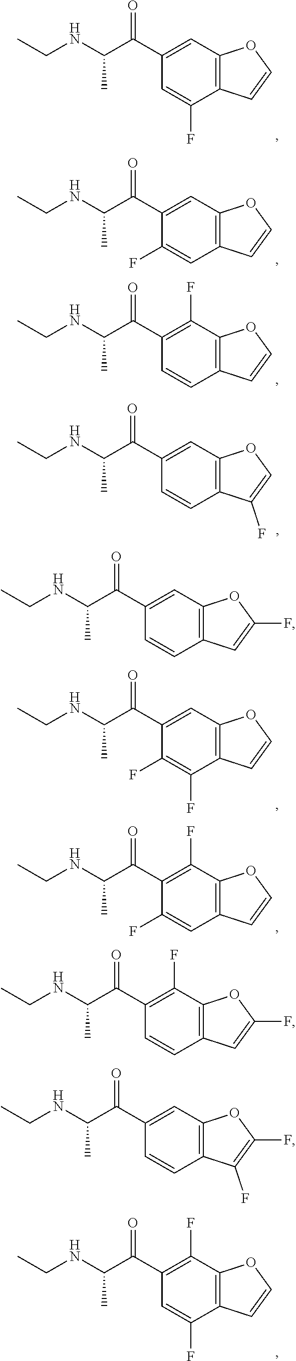

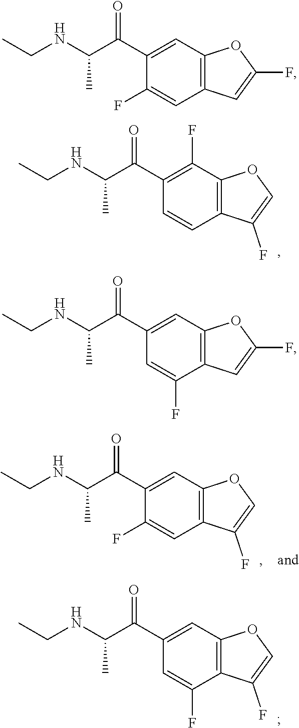

or a pharmaceutically acceptable salt, salt mixture, or enantiomerically enriched mixture thereof.

Additional non-limiting examples of compounds of the present invention include

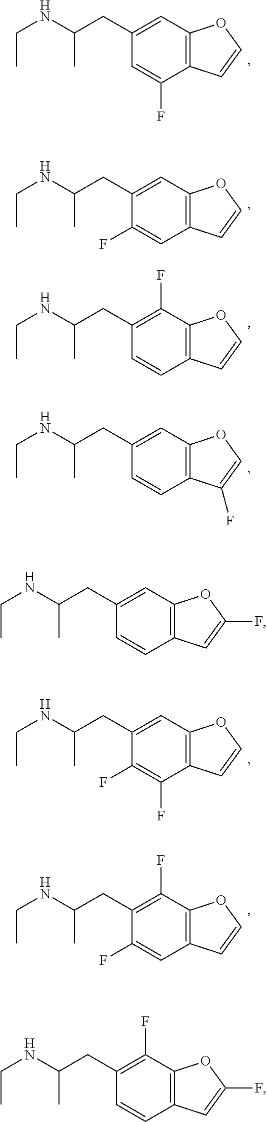

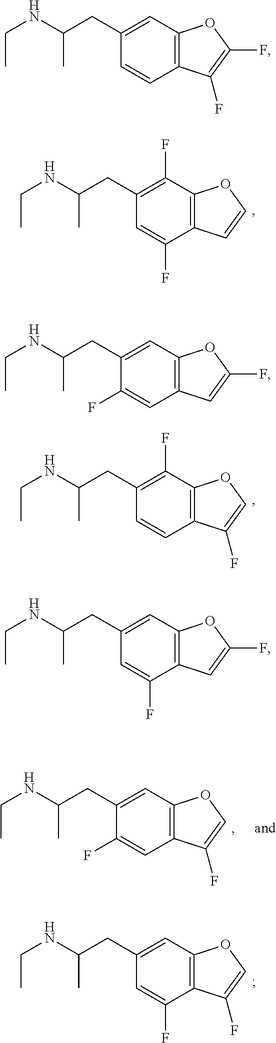

or a pharmaceutically acceptable salt, salt mixture, or enantiomerically enriched mixture thereof.

In certain embodiments the compound of the present invention is selected from:

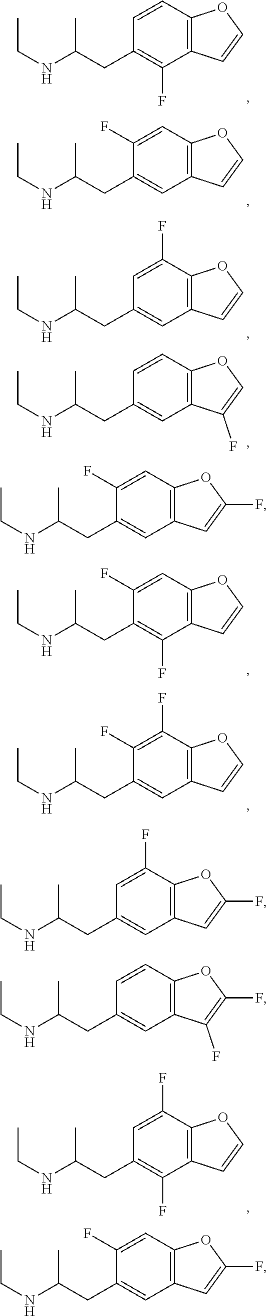

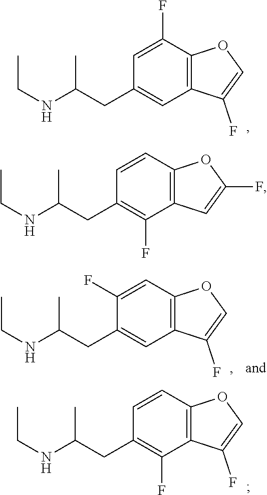

or a pharmaceutically acceptable salt, salt mixture, or enantiomerically enriched mixture thereof.

In certain embodiments the compound of the present invention is selected from:

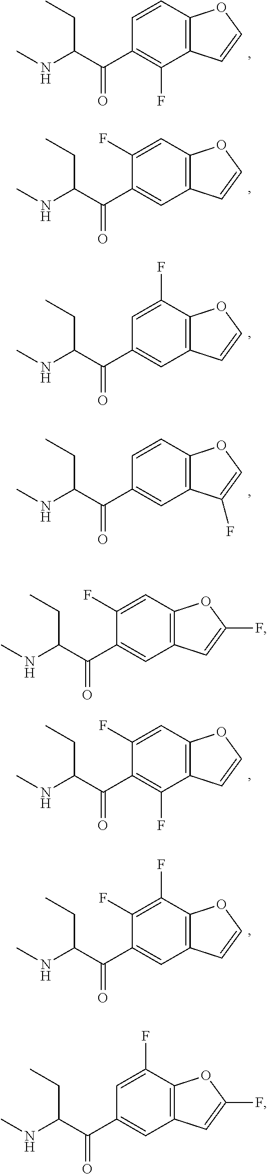

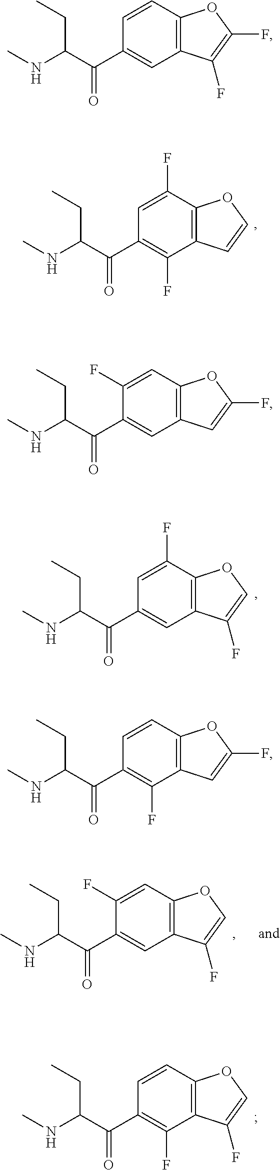

or a pharmaceutically acceptable salt, salt mixture, or enantiomercally enrich ed mixture thereof.

In certain embodiments the compound of the present invention is selected from:

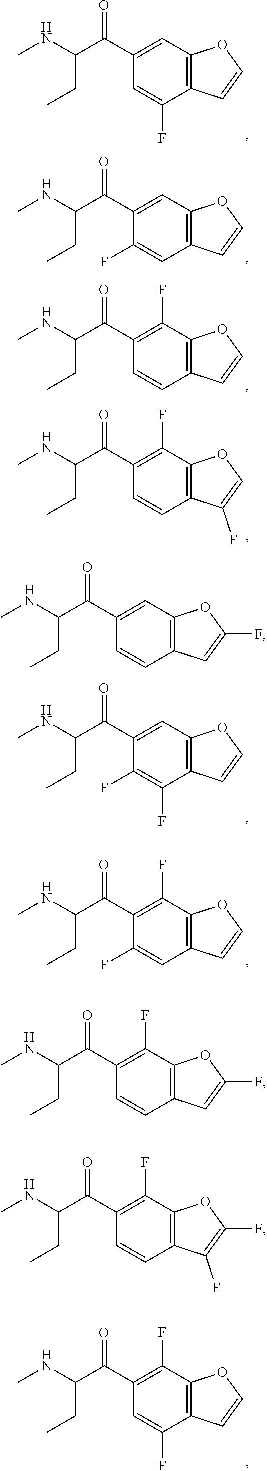

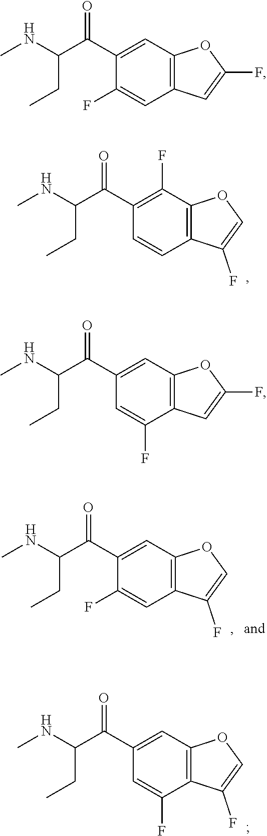 [SMILES: CCC(NC)C(=O)c1cc2oc(F)cc2cc1F ; CCC(NC)C(=O)c1ccc2c(F)coc2c1F ; CCC(NC)C(=O)c1cc(F)c2cc(F)oc2c1 ; CCC(NC)C(=O)c1cc2occ(F)c2cc1F ; CCC(NC)C(=O)c1cc(F)c2c(F)coc2c1]

or a pharmaceutically acceptable salt, salt mixture, or enantiomerically enriched mixture thereof.

Additional non-limiting examples of compounds of the present invention include

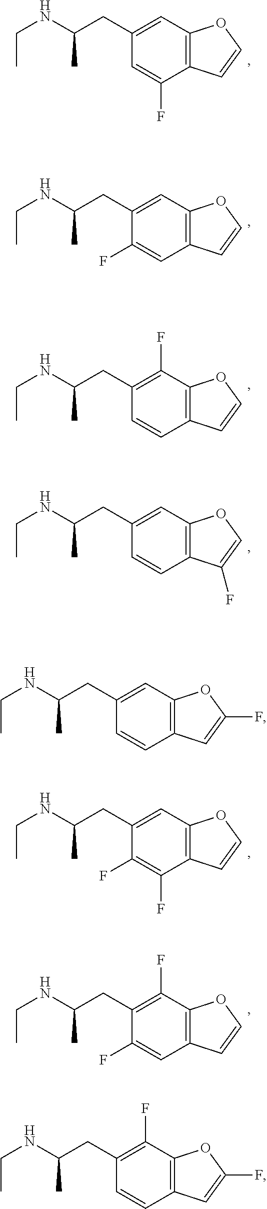

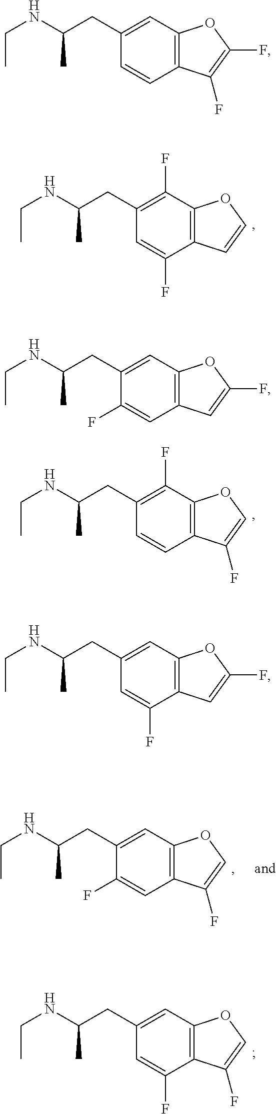

or a pharmaceutically acceptable salt, salt mixture, or enantiomerically enriched mixture thereof.

In certain embodiments the compound of the present invention is selected from:

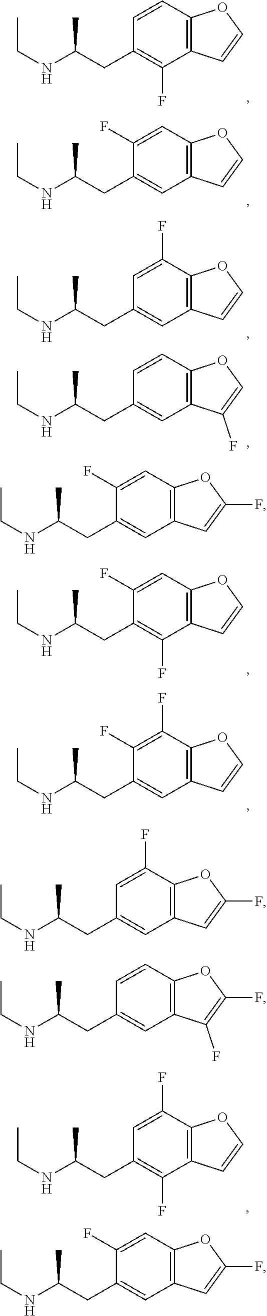

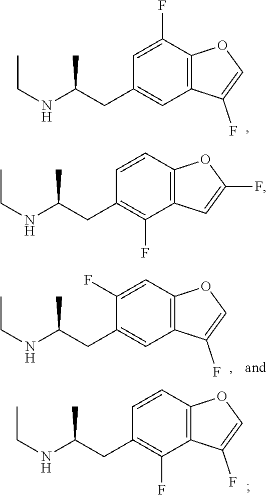

or a pharmaceutically acceptable salt, salt mixture, or enantiomerically enriched mixture thereof.

In certain embodiments the compound of the present invention is selected from:

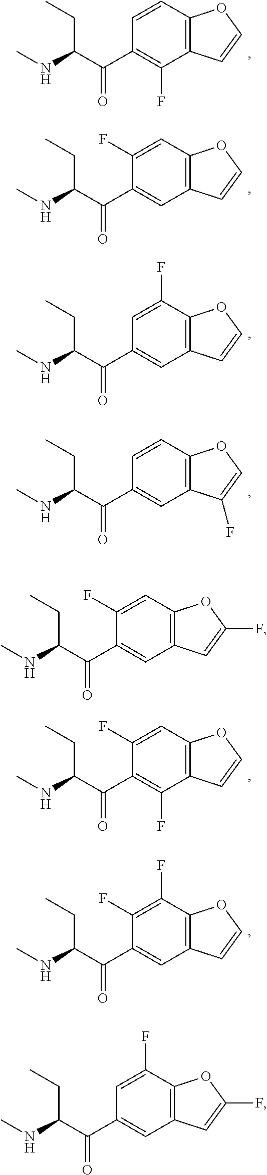

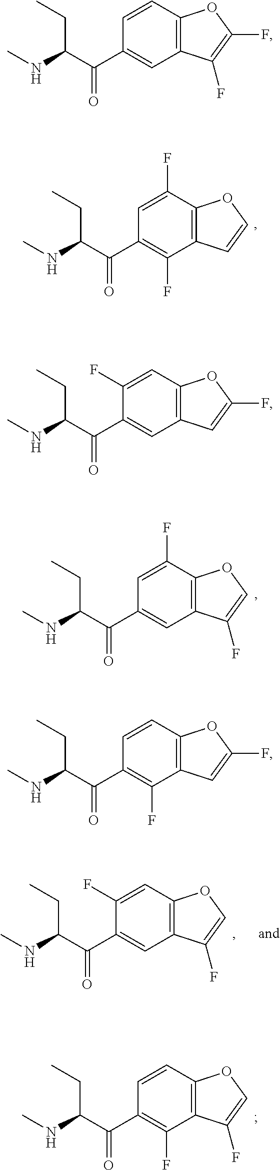

or a pharmaceutically acceptable salt, salt mixture, or enantiomerically enriched mixture thereof.

In certain embodiments the compound of the present invention is selected from:

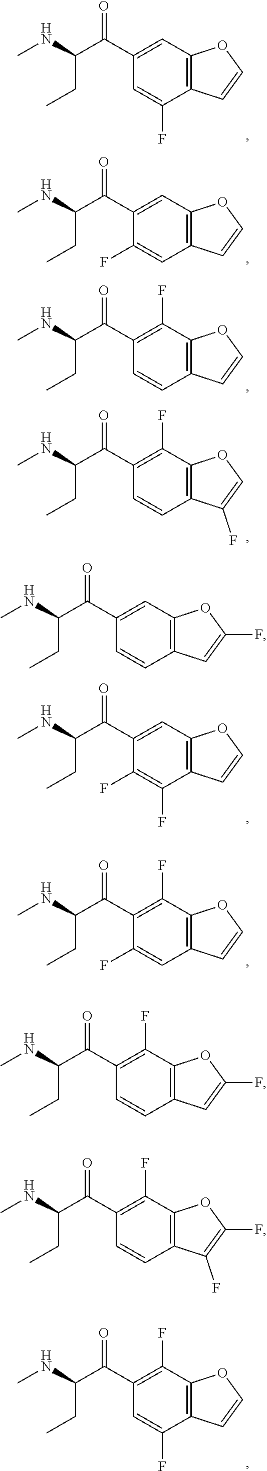

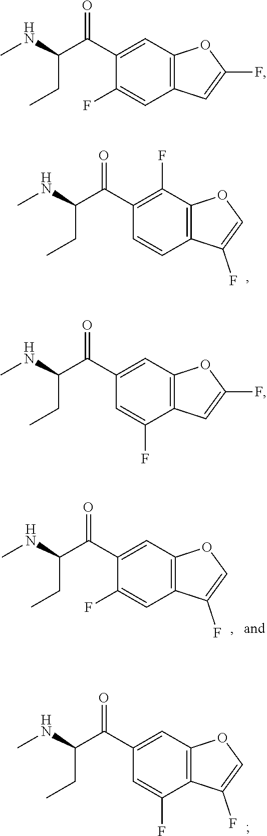 [SMILES: CC[C@@H](NC)C(=O)c1cc2oc(F)cc2cc1F ; CC[C@@H](NC)C(=O)c1ccc2c(F)coc2c1F ; CC[C@@H](NC)C(=O)c1cc(F)c2cc(F)oc2c1 ; CC[C@@H](NC)C(=O)c1cc2occ(F)c2cc1F ; CC[C@@H](NC)C(=O)c1cc(F)c2c(F)coc2c1]

or a pharmaceutically acceptable salt, salt mixture, or enantiomerically enriched mixture thereof.

Additional non-limiting examples of compounds of the present invention include

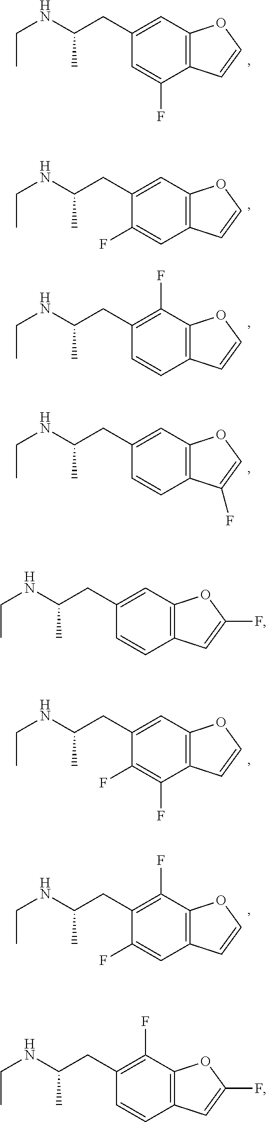

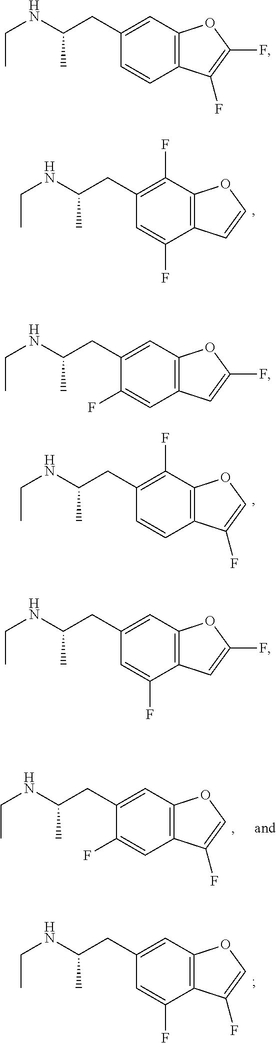

or a pharmaceutically acceptable sat-, salt mixture, or enantiomerically enriched mixture thereof.

In certain embodiments the compound of the present invention is selected from:

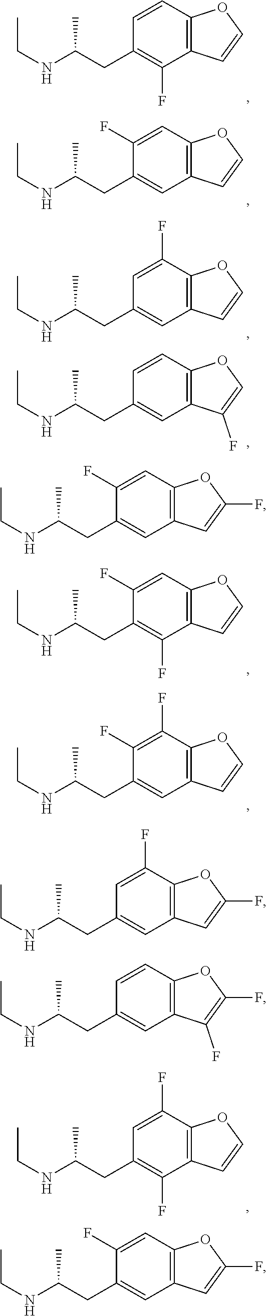

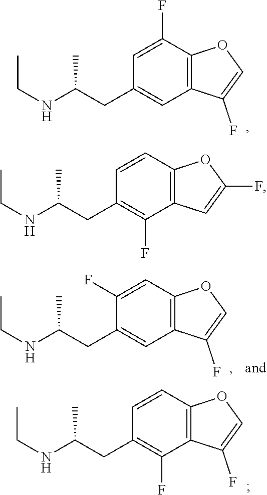

or a pharmaceutically acceptable sat-, salt mixture, or enantiomerically enriched mixture thereof.

In certain embodiments the compound of the present invention is selected from:

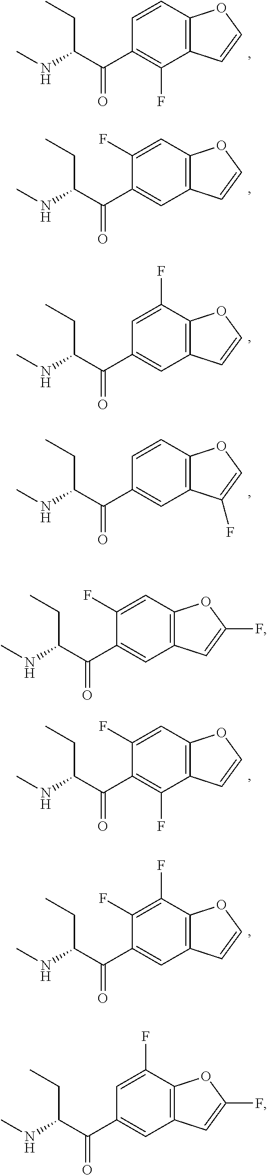

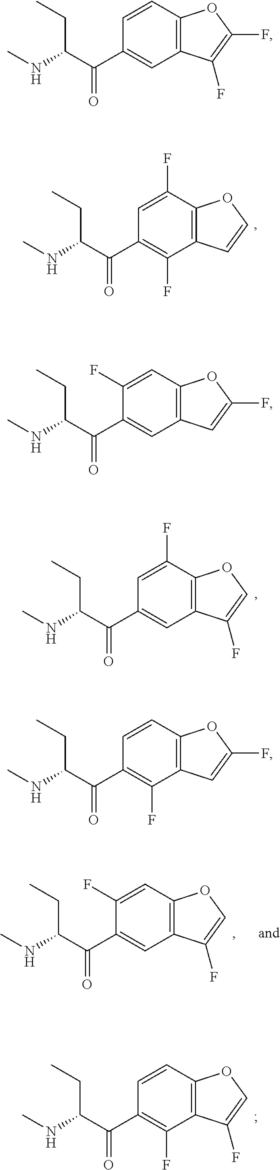

or a pharmaceutically acceptable salt, salt mixture, or enantiomerically enriched mixture thereof.

In certain embodiments the compound of the present invention is selected from:

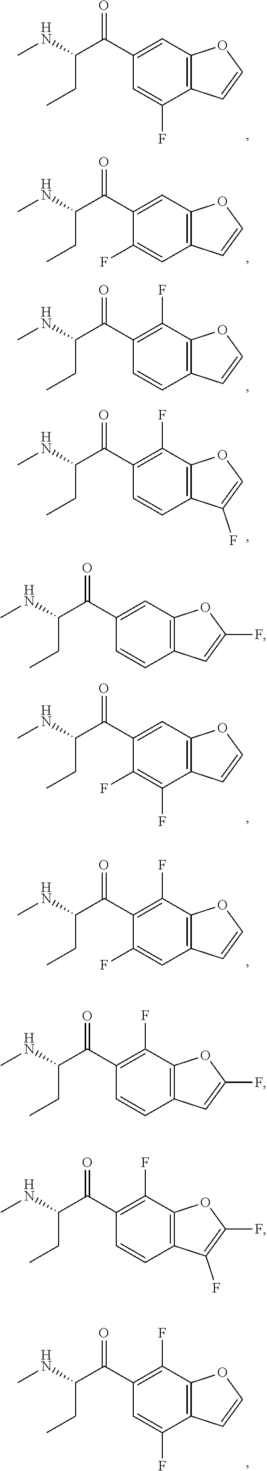

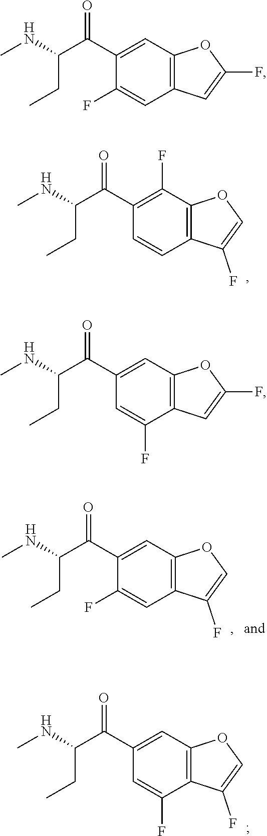 [SMILES: CC[C@H](NC)C(=O)c1cc2oc(F)cc2cc1F ; CC[C@H](NC)C(=O)c1ccc2c(F)coc2c1F ; CC[C@H](NC)C(=O)c1cc(F)c2cc(F)oc2c1 ; CC[C@H](NC)C(=O)c1cc2occ(F)c2cc1F ; CC[C@H](NC)C(=O)c1cc(F)c2c(F)coc2c1]

or a pharmaceutically acceptable salt, salt mixture, or enantiomerically enriched mixture thereof.

### Additional Embodiments of the Present Invention

1. A compound of Formula:

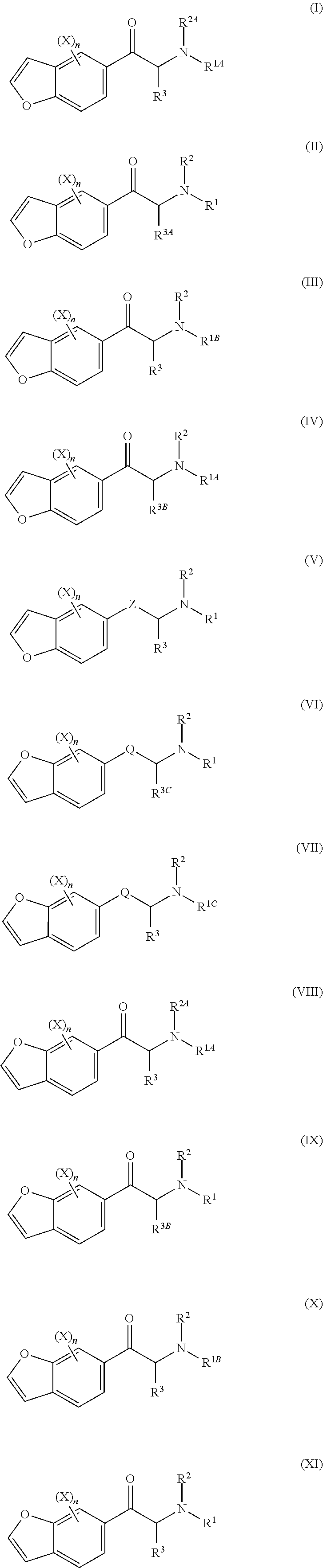

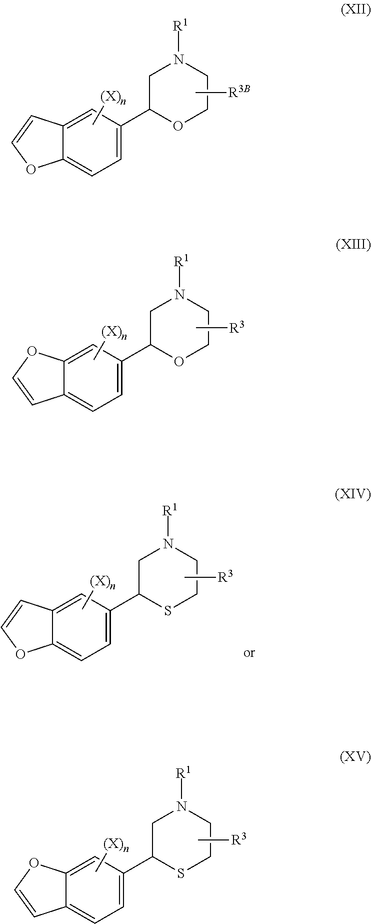

or a pharmaceutically acceptable salt or salt mixture thereof.
wherein:
R¹ and R² are independently selected from H, C₁-C₄ alkyl, C₁-C₄ haloalkyl, CH₂OH, and —CH₂CH₂OH;
R^{1A} and R^{2A} are independently selected from C₁-C₄ alkyl, C₁-C₄ haloalkyl, CH₂OH, and —CH₂CH₂OH;
R^{1B} is C₂-C₄ alkyl, C₁-C₄ haloalkyl, CH₂OH, or —CH₂CH₂OH;
R^{1C} is C₂-C₄ haloalkyl;
R³ is selected from H, C₁-C₄ alkyl, C₁-C₄ haloalkyl, CH₂OH, and —CH₂CH₂OH;
R⁴ and R⁵ are independently selected from H, C₁-C₄ alkyl, C₁-C₄ haloalkyl, F, Cl, and Br;
R^{3A} is C₄ alkyl, C₁-C₄ haloalkyl, CH₂OH, or —CH₂CH₂OH;
R^{3B} is C₁-C₄ alkyl, C₁-C₄ haloalkyl, CH₂OH, or —CH₂CH₂OH;
R^{3C} is C₁-C₄ haloalkyl;
Q is

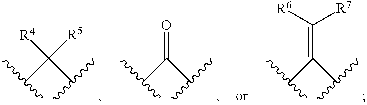

Z is

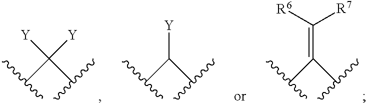

R⁶ and R⁷ are independently selected from H, C₁-C₄ alkyl, C₁-C₄ haloalkyl, F, Cl, and Br;
each X is independently selected from —F, —Cl, and —Br;
each Y is independently selected from —F, —Cl, and —Br; and
n is 1 or 2.
2. The compound of embodiment 1, wherein the compound is of Formula:

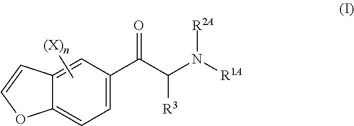

or a pharmaceutically acceptable salt or salt mixture thereof.
3. The compound of embodiment 1, wherein the compound is of Formula:

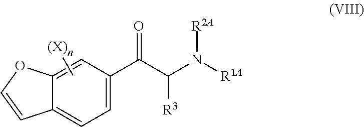

or a pharmaceutically acceptable salt or salt mixture thereof
4. The compound of any one of embodiments 1-3, wherein R^{2A} is C₁-C₄ alkyl.
5. The compound of any one of embodiments 1-3, wherein R^{2A} is methyl, ethyl, or propyl.
6. The compound of any one of embodiments 1-3, wherein R^{2A} is isopropyl or cyclopropyl.
7. The compound of any one of embodiments 1-3, wherein R^{2A} is C₁-C₄ haloalkyl.
8. The compound of any one of embodiments 1-3, wherein R^{2A} is CH₂CH₂F.
9. The compound of any one of embodiments 1-3, wherein R^{2A} is CH₂CH₂OH.
10. The compound of embodiment 1, wherein the compound is of Formula:

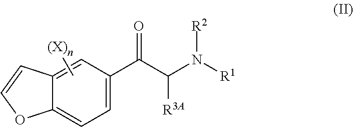

or a pharmaceutically acceptable salt or salt mixture thereof.
11. The compound of embodiment 10, wherein R^{3A} is C₄ alkyl.
12. The compound of embodiment 10, wherein R^{3A} is C₁-C₄ haloalkyl.
13. The compound of embodiment 10, wherein R^{3A} is CH₂F.
14. The compound of embodiment 10, wherein R^{3A} is —CH₂CH₂OH.
15. The compound of embodiment 1, wherein the compound is of Formula:

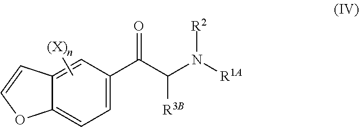

or a pharmaceutically acceptable salt or salt mixture thereof.
16. The compound of embodiment 1, wherein the compound is of Formula:

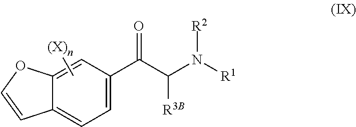

or a pharmaceutically acceptable salt or salt mixture thereof.
17. The compound of embodiment 1, wherein the compound is of Formula:

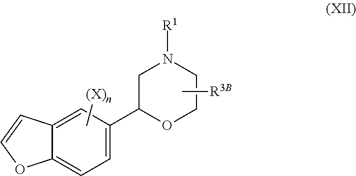

or a pharmaceutically acceptable salt or salt mixture thereof.
18. The compound of any one of embodiments 15-17, wherein R^{3B} is C₁-C₄ alkyl.
19. The compound of any one of embodiments 15-17, wherein R^{3B} is methyl, ethyl, or propyl.
20. The compound of any one of embodiments 15-17, wherein R^{3B} is isopropyl or cyclopropyl.
21. The compound of any one of embodiments 15-17, wherein R^{3B} is C₁-C₄ haloalkyl.
22. The compound of any one of embodiments 15-17, wherein R^{3B} is —CH₂F.
23. The compound of any one of embodiments 15-17, wherein R^{3B} is —CH₂CH₂OH.
24. The compound of embodiment 1, wherein the compound is of Formula:

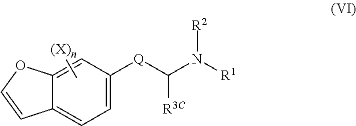

or a pharmaceutically acceptable salt or salt mixture thereof.

25. The compound of embodiment 24, wherein R^{3C} is C₁-C₃ haloalkyl.
26. The compound of embodiment 24, wherein R^{3C} is CH₂F.
27. The compound of embodiment 24, wherein R^{3C} is CHF₂.
28. The compound of embodiment 24, wherein R^{3C} is CF₃.
29. The compound of embodiment 24, wherein R^{3C} is C₂ haloalkyl.
30. The compound of any one of embodiments 24-29, wherein Q is

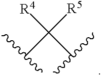

31. The compound of embodiment 30, wherein R⁴ and R⁵ are both hydrogen.
32. The compound of embodiment 30, wherein R⁴ is H.
33. The compound of embodiment 30, wherein R⁴ is C₁-C₄ alkyl.
34. The compound of embodiment 30, wherein R⁴ is C₁-C₄ haloalkyl.
35. The compound of embodiment 30, wherein R⁴ is F.
36. The compound of any one of embodiments 32-35, wherein R⁵ is H.
37. The compound of any one of embodiments 32-35, wherein R⁵ is C₁-C₄ alkyl.
38. The compound of any one of embodiments 32-35, wherein R⁵ is C₁-C₄ haloalkyl.
39. The compound of any one of embodiments 32-35, wherein R⁵ is F.

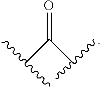 [SMILES: *C(*)=O]

40. The compound of any one of embodiments 24-29, wherein Q is.

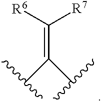

41. The compound of any one of embodiments 24-29, wherein Q is.
42. The compound of embodiment 41, wherein R⁶ and R⁷ are H.
43. The compound of embodiment 41, wherein R⁶ and R⁷ are F.
44. The compound of embodiment 41, wherein R⁶ is F and R⁷ are H.
45. The compound of embodiment 1, wherein the compound is of Formula:

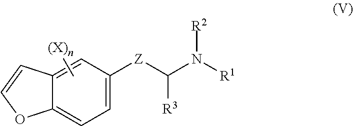

or a pharmaceutically acceptable salt or salt mixture thereof.
46. The compound of embodiment 1, wherein the compound is of Formula:

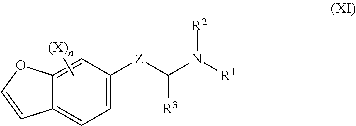

or a pharmaceutically acceptable salt or salt mixture thereof.
47. The compound of embodiment 45 or embodiment 46, wherein Z is

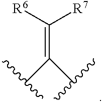

48. The compound of embodiment 47, wherein R⁶ and R⁷ are H.
49. The compound of embodiment 47, wherein R⁶ and R⁷ are F.
50. The compound of embodiment 47, wherein R⁶ is F and R⁷ are H.
51. The compound of embodiment 45 or embodiment 46, wherein Z is

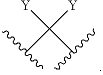

52. The compound of embodiment 45 or embodiment 46, wherein Z is.

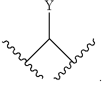

53. The compound of embodiment 51 or embodiment 52, wherein Y is F.
54. The compound of embodiment 1, wherein the compound is of Formula:

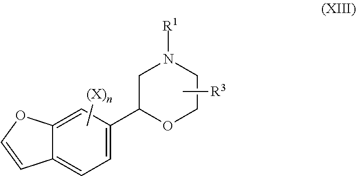

or a pharmaceutically acceptable salt or salt mixture thereof.
55. The compound of embodiment 1, wherein the compound is of Formula:

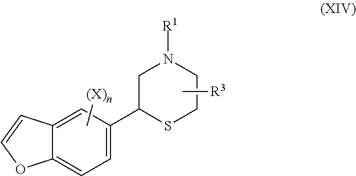

or a pharmaceutically acceptable salt or salt mixture thereof.
56. The compound of embodiment 1, wherein the compound is of Formula:

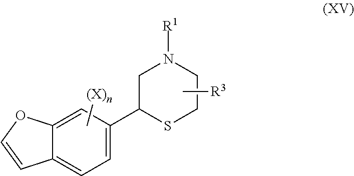

or a pharmaceutically acceptable salt or salt mixture thereof.
57. The compound of any one of embodiments 1-56, wherein R¹ is H.
58. The compound of any one of embodiments 1-56, wherein R¹ is methyl.
59. The compound of any one of embodiments 1-56, wherein R¹ is ethyl.
60. The compound of any one of embodiments 1-56, wherein R¹ is propyl.
61. The compound of any one of embodiments 1-56, wherein R¹ is isopropyl or cyclopropyl.
62. The compound of any one of embodiments 1-56, wherein R¹ is C₁-C₄ haloalkyl.
63. The compound of any one of embodiments 1-56, wherein R¹ is CH₂CH₂F.
64. The compound of embodiment 1, wherein the compound is of Formula:

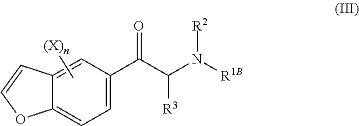

or a pharmaceutically acceptable salt or salt mixture thereof.
65. The compound of embodiment 1, wherein the compound is of Formula:

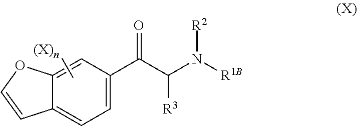

or a pharmaceutically acceptable salt or salt mixture thereof
66. The compound of embodiment 64 or embodiment 65 wherein R^{1B} is C₂-C₄ alkyl.
67. The compound of embodiment 64 or embodiment 65 wherein R^{1B} is ethyl.
68. The compound of embodiment 64 or embodiment 65 wherein R^{1B} is C₁-C₄ haloalkyl.
69. The compound of embodiment 1, wherein the compound is of Formula:

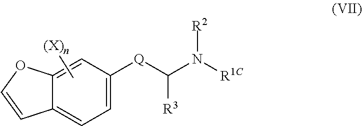

or a pharmaceutically acceptable salt or salt mixture thereof.
70. The compound of embodiment 69, wherein R^{1C} is C₂-C₃ haloalkyl.
71. The compound of embodiment 69, wherein R^{1C} is C₂ haloalkyl.
72. The compound of any one of embodiments 69-71, wherein Q is

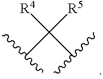

73. The compound of any one of embodiments 69-71, wherein Q is

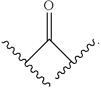 [SMILES: *C(*)=O]

74. The compound of any one of embodiments 69-71, wherein Q is

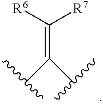

75. The compound of any one of embodiments 45-74, wherein R³ is H.
76. The compound of any one of embodiments 45-74, wherein R³ is C₁-C₄ alkyl.
77. The compound of any one of embodiments 45-74, wherein R³ is methyl, ethyl, or propyl.
78. The compound of any one of embodiments 45-74, wherein R³ is isopropyl or cyclopropyl.
79. The compound of any one of embodiments 45-74, wherein R³ is C₁-C₄ haloalkyl.
80. The compound of any one of embodiments 45-74, wherein R³ is CH₂CH₂F.
81. The compound of any one of embodiments 1-80, wherein n is 1.
82. The compound of embodiment 81, wherein X is F.
83. The compound of embodiment 81, wherein X is Cl.
84. The compound of embodiment 81, wherein X is Br.
85. The compound of any one of embodiments 1-80, wherein n is 2.
86. The compound of embodiment 85, wherein each X is F.
87. The compound of embodiment 85, wherein one X is F and the other is Cl.
88. The compound of any of embodiments 1-87, wherein the compound is an enantiomerically enriched mixture or pure enantiomer.
89. An enantiomerically pure or enriched compound of Formula:

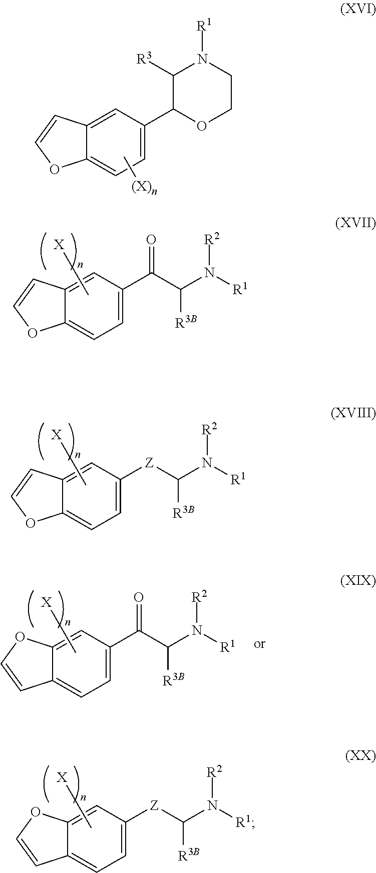

or a pharmaceutically acceptable salt or salt mixture thereof wherein the variables are as defined in embodiments 1-87.
90. A compound selected from

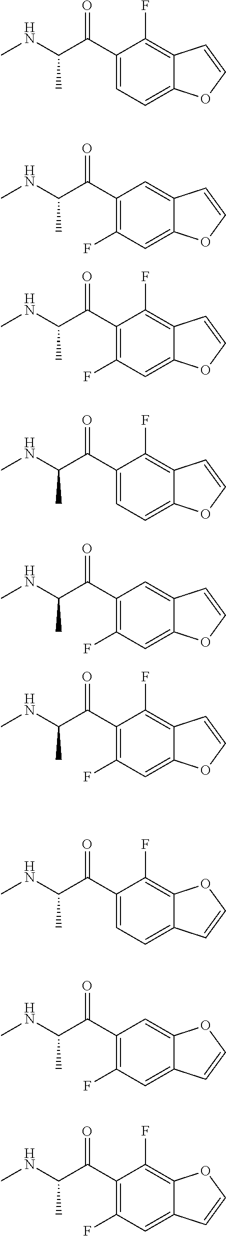

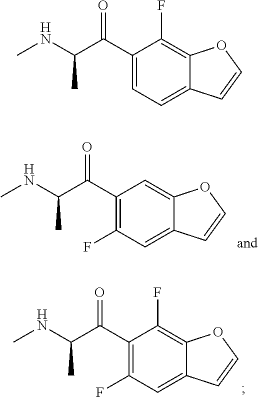 [SMILES: CN[C@H](C)C(=O)c1ccc2ccoc2c1F ; CN[C@H](C)C(=O)c1cc2occc2cc1F ; CN[C@H](C)C(=O)c1c(F)cc2ccoc2c1F]

or a pharmaceutically acceptable salt, salt mixture, or enantiomerically enriched mixture thereof.
91. A compound selected from

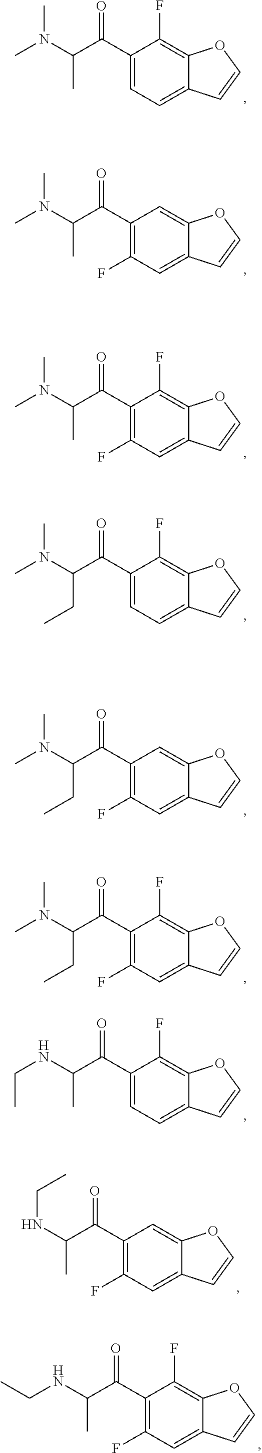

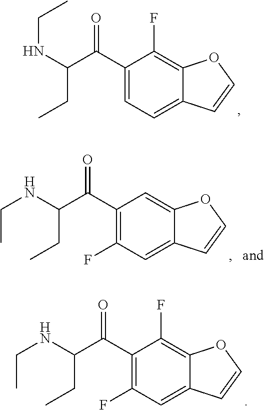 [SMILES: CCNC(CC)C(=O)c1ccc2ccoc2c1F ; CCNC(CC)C(=O)c1cc2occc2cc1F ; CCNC(CC)C(=O)c1c(F)cc2ccoc2c1F]

92. A compound selected from

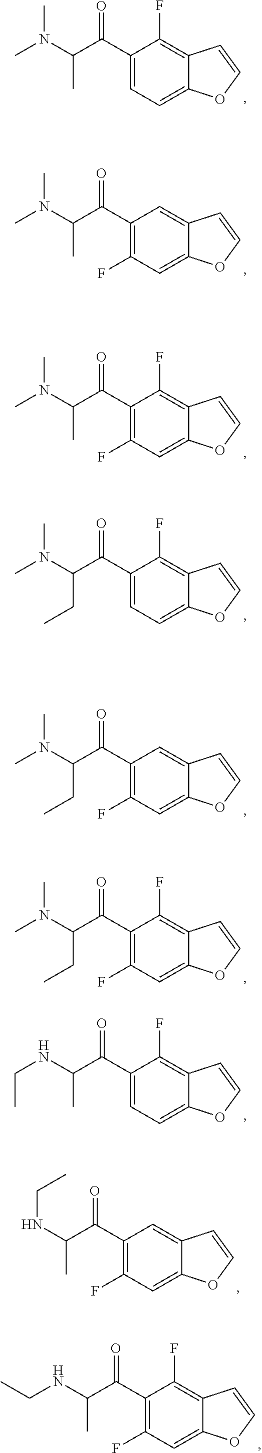

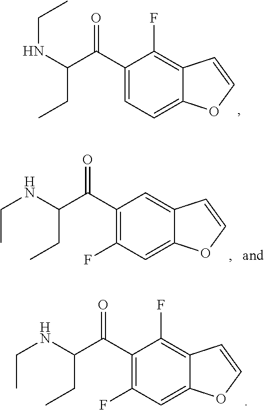 [SMILES: CCNC(CC)C(=O)c1ccc2occc2c1F ; CCNC(CC)C(=O)c1cc2ccoc2cc1F ; CCNC(CC)C(=O)c1c(F)cc2occc2c1F]

93. A pharmaceutical composition comprising an effective patient-treating amount of a compound, pure enantiomer, or enantiomerically enriched mixture of any one of embodiments 1-92 and a pharmaceutically acceptable carrier or excipient.
94. The pharmaceutical composition of embodiment 93 wherein the composition is administered systemically.
95. The pharmaceutical composition of embodiment 93 wherein the composition is administered orally.
96. The pharmaceutical composition of embodiment 93 wherein the composition is administered to mucosal tissue.
97. The pharmaceutical composition of embodiment 93 wherein the composition is administered rectally.
98. The pharmaceutical composition of embodiment 93 wherein the composition is administered topically.
99. The pharmaceutical composition of embodiment 93 wherein the composition is administered subcutaneously.
100. The pharmaceutical composition of embodiment 93 wherein the composition is administered intravenously.
101. The pharmaceutical composition of embodiment 93 wherein the composition is administered intramuscularly.
102. The pharmaceutical composition of embodiment 93 wherein the composition is administered via inhalation.
103. The pharmaceutical composition of embodiment 93 wherein the composition is administered as a tablet.
104. The pharmaceutical composition of embodiment 93 wherein the composition is administered as a gelcap.
105. The pharmaceutical composition of embodiment 93 wherein the composition is administered as a capsule.
106. The pharmaceutical composition of embodiment 93 wherein the composition is administered as an aqueous emulsion.
107. The pharmaceutical composition of embodiment 93 wherein the composition is administered as an aqueous solution.
108. The pharmaceutical composition of embodiment 93 wherein the composition is administered as a pill.
109. The pharmaceutical composition of embodiment 93 wherein the composition is administered as a buccal tablet.
110. The pharmaceutical composition of embodiment 93 wherein the composition is administered as a sublingual tablet.
111. The pharmaceutical composition of embodiment 93 wherein the composition is administered as a sublingual strip.
112. The pharmaceutical composition of embodiment 93 wherein the composition is administered as a sublingual liquid.
113. The pharmaceutical composition of embodiment 93 wherein the composition is administered as a sublingual spray.
114. The pharmaceutical composition of embodiment 93 wherein the composition is administered as a sublingual gel.
115. The pharmaceutical composition of embodiment 93 wherein the composition is administered as a cream.
116. The pharmaceutical composition of embodiment 93 wherein the composition is administered as a topical solution.
117. The pharmaceutical composition of embodiment 93 wherein the composition is administered as an aqueous solution.
118. The pharmaceutical composition of embodiment 93 wherein the composition is administered as a powder.
119. The pharmaceutical composition of embodiment 93 wherein the composition is administered as an aerosol.
120. A method for treating a central nervous system disorder comprising administering an effective amount of a compound, pure enantiomer, or enantiomerically enriched mixture of any one of embodiments 1-92 or a pharmaceutical composition of any one of embodiments 93-119 to a host in need thereof.
121. A method for treating a central nervous system disorder comprising administering an effective amount of a compound, pure enantiomer, or enantiomerically enriched mixture of a compound of Formula XXI or XXII to a host in need thereof.

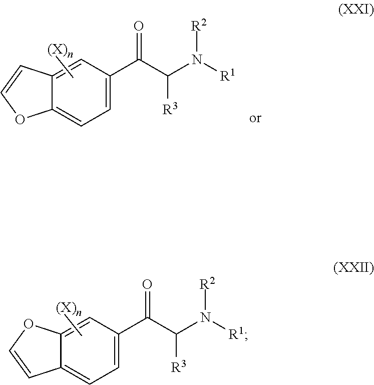

or a pharmaceutically acceptable salt or salt mixture thereof, wherein the variables are as defined in embodiments 1-87.
122. The method of embodiment 120 or 121 wherein the host is a human.
123. The method of any one of embodiments 120-122 wherein the central nervous system disorder is selected from: post-traumatic stress disorder, depression, dysthymia, anxiety, generalized anxiety, social anxiety, panic, adjustment disorder, feeding and eating disorders, binge behaviors, body dysmorphic syndromes, addiction, drug abuse or dependence disorders, substance use disorders, disruptive behavior disorders, impulse control disorders, gaming disorders, gambling disorders, memory loss, dementia of aging, attention deficit hyperactivity disorder, personality disorders, attachment disorders, autism, dissociative disorders and headache disorders.
124. The method of any one of embodiments 120-122 wherein the central nervous system disorder is post-traumatic stress disorder.
125. The method of any one of embodiments 120-122 wherein the central nervous system disorder is adjustment disorder.
126. The method of any one of embodiments 120-122 wherein the central nervous system disorder is generalized anxiety.
127. The method of any one of embodiments 120-122 wherein the central nervous system disorder is social anxiety.
128. The method of any one of embodiments 120-122 wherein the central nervous system disorder is depression.
129. The method of any one of embodiments 120-122 wherein the central nervous system disorder is a substance use disorder.
130. The method of any one of embodiments 120-122 wherein the central nervous system disorder is an attachment disorder.
131. The method of any one of embodiments 120-122 wherein the central nervous system disorder is schizophrenia.
132. The method of any one of embodiments 120-122 wherein the central nervous system disorder is a headache disorder.
133. The method of any one of embodiments 120-122 wherein the central nervous system disorder is a migraine disorder.
134. The method of any one of embodiments 120-122 wherein the central nervous system disorder is a seizure disorder.
135. The method of any one of embodiments 120-122 wherein the central nervous system disorder is an eating disorder.
136. The method of embodiment 135 wherein the eating disorder is bulimia.
137. The method of embodiment 135 wherein the eating disorder is binge eating.
138. The method of embodiment 135 wherein the eating disorder is anorexia.
139. The method of any one of embodiments 120-122 wherein the central nervous system disorder is a neurological disorder.
140. The method of embodiment 139 wherein the neurological disorder is stroke.
141. The method of embodiment 139 wherein the neurological disorder is brain trauma.
142. The method of embodiment 139 wherein the neurological disorder is dementia.
143. The method of embodiment 139 wherein the neurological disorder is a neurodegenerative disease or disorder.
144. The method of embodiment 143 wherein the neurodegenerative disease or disorder is selected from: Alzheimer's disease, mild cognitive impairment (MCI), Parkinson's disease, Parkinson's disease dementia, multiple sclerosis, adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, X-linked spinal muscular atrophy, synucleinopathy, progranulinopathy, tauopathy, amyloid disease, prion disease, protein aggregation disease, and movement disorder.
145. The method of any one of embodiments 120-144 wherein the compound, pure enantiomer, or enantiomerically enriched mixture is administered in a clinical setting.
146. The method of any one of embodiments 120-144 wherein the compound, pure enantiomer, or enantiomerically enriched mixture is administered in an at-home setting.
147. The method of any one of embodiments 120-144 wherein the compound, pure enantiomer, or enantiomerically enriched mixture is administered during a psychotherapy session.
148. The method of any one of embodiments 120-144 wherein the compound, pure enantiomer, or enantiomerically enriched mixture is administered during a counseling session.

### Embodiments of “Alkyl”

In certain embodiments “alkyl” is a C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

Non-limiting examples of “alkyl” include: methyl, ethyl, propyl, and isopropyl.

Additional non-limiting examples of “alkyl” include: butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “Haloalkyl”

In certain embodiments “haloalkyl” is C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has two carbons and one halogen.

In certain embodiments “haloalkyl” has two carbons and two halogens.

In certain embodiments “haloalkyl” has two carbons and three halogens.

In certain embodiments “haloalkyl” has two carbons and four halogens.

In certain embodiments “haloalkyl” has two carbons and five halogens.

In certain embodiments “haloalkyl” has three carbons.

In certain embodiments “haloalkyl” has three carbons and one halogen.

In certain embodiments “haloalkyl” has three carbons and two halogens.

In certain embodiments “haloalkyl” has three carbons and three halogens.

In certain embodiments “haloalkyl” has three carbons and four halogens.

In certain embodiments “haloalkyl” has three carbons and five halogens.

In certain embodiments “haloalkyl” has three carbons and six halogens.

In certain embodiments “haloalkyl” has three carbons and seven halogens.

In certain embodiments “haloalkyl” has four carbons.

In certain embodiments “haloalkyl” has five carbons.

Non-limiting examples of “haloalkyl” include:

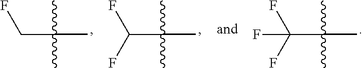

Additional non-limiting examples of “haloalkyl” include:

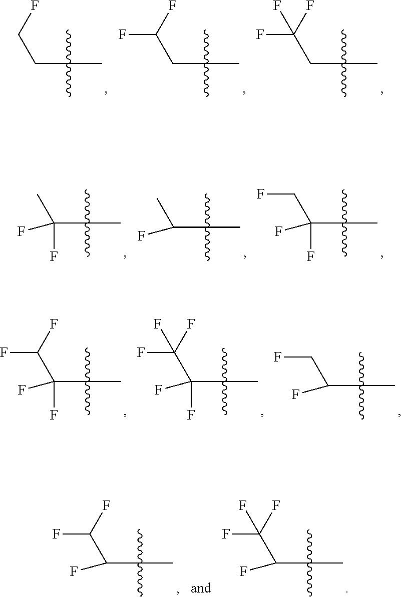

Additional non-limiting examples of “haloalkyl” include:

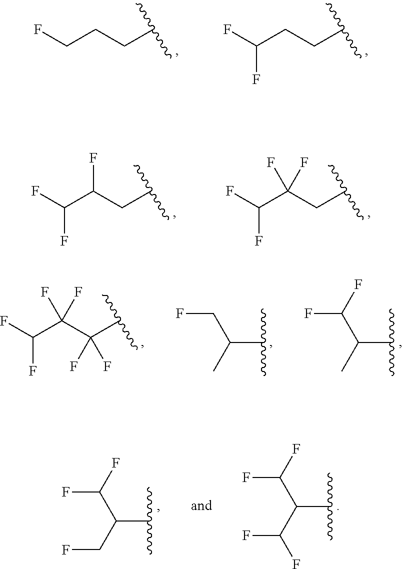

Additional non-limiting examples of “haloalkyl” include:

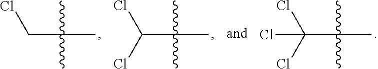

Additional non-limiting examples of “haloalkyl” include:

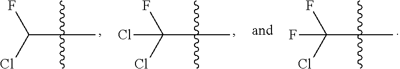

### Embodiments of “Cycloalkyl”

In certain embodiments “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₆-C₈cycloalkyl, or C₆-C₈cycloalkyl.

In certain embodiments “cycloalkyl” has three carbons.

In certain embodiments “cycloalkyl” has four carbons.

In certain embodiments “cycloalkyl” has five carbons.

In certain embodiments “cycloalkyl” has six carbons.

In certain embodiments “cycloalkyl” has seven carbons.

In certain embodiments “cycloalkyl” has eight carbons.

In certain embodiments “cycloalkyl” has nine carbons.

In certain embodiments “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

### Embodiments of R¹

In certain embodiments R¹ is hydrogen.

In certain embodiments R¹ is C₁-C₄ alkyl.

In certain embodiments R¹ is methyl.

In certain embodiments R¹ is ethyl.

In certain embodiments R¹ is n-propyl.

In certain embodiments R¹ is isopropyl.

In certain embodiments R¹ is C₁-C₄ haloalkyl.

In certain embodiments R¹ is —CF₃.

In certain embodiments R¹ is —CH₂OH.

In certain embodiments R¹ is —CH₂CH₂OH.

### Embodiments of R²

In certain embodiments R² is hydrogen.

In certain embodiments R² is C₁-C₄ alkyl.

In certain embodiments R² is methyl.

In certain embodiments R² is ethyl.

In certain embodiments R² is n-propyl.

In certain embodiments R² is isopropyl.

In certain embodiments R² is C₁-C₄ haloalkyl.

In certain embodiments R² is —CF₃.

In certain embodiments R² is —CH₂OH.

In certain embodiments R² is —CH₂CH₂OH.

### Embodiments of R^{1A}

In certain embodiments R^{1A} is C₁-C₄ alkyl.

In certain embodiments R^{1A} is methyl.

In certain embodiments R^{1A} is ethyl.

In certain embodiments R^{1A} is n-propyl.

In certain embodiments R^{1A} is isopropyl.

In certain embodiments R^{1A} is C₁-C₄ haloalkyl.

In certain embodiments R^{1A} is —CF₃.

In certain embodiments R^{1A} is —CH₂OH.

In certain embodiments R^{1A} is —CH₂CH₂OH.

### Embodiments of R^{1B}

In certain embodiments R^{1B} is C₂-C₄ alkyl.

In certain embodiments R^{1B} is ethyl.

In certain embodiments R^{1B} is n-propyl.

In certain embodiments R^{1B} is isopropyl.

In certain embodiments R^{1B} is C₁-C₄ haloalkyl.

In certain embodiments R^{1B} is —CF₃.

In certain embodiments R^{1B} is —CH₂OH.

In certain embodiments R^{1B} is —CH₂CH₂OH.

### Embodiments of R^{1C}

In certain embodiments R^{1C} is C₂-C₄ haloalkyl.

In certain embodiments R^{1C} is —CH₂CF₃.

In certain embodiments R^{1C} is —CH₂CHF₂.

In certain embodiments R^{1C} is —CH₂CH₂F.

### Embodiments of R^{2A}

In certain embodiments R^{2A} is C₁-C₄ alkyl.

In certain embodiments R^{2A} is methyl.

In certain embodiments R^{2A} is ethyl.

In certain embodiments R^{2A} is n-propyl.

In certain embodiments R^{2A} is isopropyl.

In certain embodiments R^{2A} is C₁-C₄ haloalkyl.

In certain embodiments R^{2A} is —CF₃.

In certain embodiments R^{2A} is —CH₂OH.

In certain embodiments R^{2A} is —CH₂CH₂OH.

### Embodiments of R³

In certain embodiments R³ is hydrogen.

In certain embodiments R³ is C₁-C₄ alkyl.

In certain embodiments R³ is methyl.

In certain embodiments R³ is ethyl.

In certain embodiments R³ is n-propyl.

In certain embodiments R³ is isopropyl.

In certain embodiments R³ is C₁-C₄ haloalkyl.

In certain embodiments R³ is —CF₃.

In certain embodiments R³ is —CH₂OH.

In certain embodiments R³ is —CH₂CH₂OH.

### Embodiments of R^{3A}

In certain embodiments R^{3A} is C₄ alkyl.

In certain embodiments R^{3A} is C₁-C₄ haloalkyl.

In certain embodiments R^{3A} is —CF₃.

In certain embodiments R^{3A} is —CH₂OH.

In certain embodiments R^{3A} is —CH₂CH₂OH.

### Embodiments of R^{3B}

In certain embodiments R^{3B} is C₁-C₄ alkyl.

In certain embodiments R^{3B} is methyl.

In certain embodiments R^{3B} is ethyl.

In certain embodiments R^{3B} is n-propyl.

In certain embodiments R^{3B} is isopropyl.

In certain embodiments R^{3B} is C₁-C₄ haloalkyl.

In certain embodiments R^{3B} is —CF₃.

In certain embodiments R^{3B} is —CH₂OH.

In certain embodiments R^{3B} is —CH₂CH₂OH.

### Embodiments of R^{3C}

In certain embodiments R^{3C} is C₁-C₄ haloalkyl.

In certain embodiments R^{3C} is —CF₃.

### Embodiments of R⁴

In certain embodiments R⁴ is hydrogen.

In certain embodiments R⁴ is —F.

In certain embodiments R⁴ is —Cl.

In certain embodiments R⁴ is —Br.

In certain embodiments R⁴ is C₁-C₄ alkyl.

In certain embodiments R⁴ is methyl.

In certain embodiments R⁴ is ethyl.

In certain embodiments R⁴ is n-propyl.

In certain embodiments R⁴ is isopropyl.

In certain embodiments R⁴ is C₁-C₄ haloalkyl.

In certain embodiments R⁴ is —CF₃.

### Embodiments of R⁵

In certain embodiments R⁵ is hydrogen.

In certain embodiments R⁵ is —F.

In certain embodiments R⁵ is —Cl.

In certain embodiments R⁵ is —Br.

In certain embodiments R⁵ is C₁-C₄ alkyl.

In certain embodiments R⁵ is methyl.

In certain embodiments R⁵ is ethyl.

In certain embodiments R⁵ is n-propyl.

In certain embodiments R⁵ is isopropyl.

In certain embodiments R⁵ is C₁-C₄ haloalkyl.

In certain embodiments R⁵ is —CF₃.

### Embodiments of R⁶

In certain embodiments R⁶ is hydrogen.

In certain embodiments R⁶ is —F.

In certain embodiments R⁶ is —Cl.

In certain embodiments R⁶ is —Br.

In certain embodiments R⁶ is C₁-C₄ alkyl.

In certain embodiments R⁶ is methyl.

In certain embodiments R⁶ is ethyl.

In certain embodiments R⁶ is n-propyl.

In certain embodiments R⁶ is isopropyl.

In certain embodiments R⁶ is C₁-C₄ haloalkyl.

In certain embodiments R⁶ is —CF₃.

### Embodiments of X

In certain embodiments X is —F.

In certain embodiments X is —Cl.

In certain embodiments X is —Br.

### Embodiments of Y

In certain embodiments Y is —F.

In certain embodiments Y is —Cl.

In certain embodiments Y is —Br.

### Embodiments of Q

In certain embodiments Q is

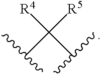

In certain embodiments Q is

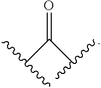 [SMILES: *C(*)=O]

In certain embodiments Q is

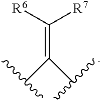

### Embodiments of Q

In certain embodiments Q is

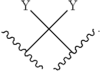

In certain embodiments Q is

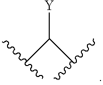

In certain embodiments Q is

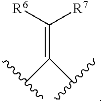

### Embodiments of Chirality

In certain embodiments the compound of the present invention is an R-enantiomer of a structure drawn herein without regard to stereochemistry or an R-enantiomer enriched mixture of enantiomers.

In certain embodiments the compound of the present invention is an S-enantiomer of a structure drawn herein without regard to stereochemistry or an S-enantiomer enriched mixture of enantiomers.

### Methods of Treatment

The present invention also provides methods for modulating the CNS in mammals by administering a pharmaceutically effective amount of a halogenated benzofuran compound of the present invention or a pharmaceutically acceptable salt, salt mixture, or pharmaceutical composition thereof to a host in need thereof, for example a human.

A halogenated benzofuran compound of the present invention can be used in methods for treating a variety of diseases or disorders linked to inadequate functioning of neurotransmission in the CNS of mammals. Included among such disorders are depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders. Also included among such disorders are primary or secondary headaches. Also included among such disorders are seizure disorders, such as epilepsy disorders.

In addition to treating various diseases and disorders, the employed methods of modulating activity of the serotonergic system in particular can be used to improve CNS functioning in non-disease states, such as reducing neuroticism and psychological defensiveness, increasing openness to experience, increasing creativity, and aiding decision-making. Any of these methods can employ a halogenated benzofuran compound of the present invention, either as a racemate, an individual enantiomer, an enantiomerically enriched mixture, or with deuterium-substitution, or more than one of these in combination. When referring to compounds herein, the terms accordingly should be understood to refer not only to the racemates of those structures, but also to single enantiomers, enantiomerically enriched mixtures, and structures with deuterium-substitution(s) or other modifications, as the context indicates and supports.

In certain embodiments, the compositions and compounds of the present invention demonstrate permeability properties that indicate the compounds are fast-acting in humans. This represents a significant improvement over SSRIs, the current standard of care for many CNS and psychological disorders. The slow onset of effects is one of the most pronounced shortcomings of SSRI therapeutics. In contrast, in one embodiment, the compounds of the present invention act as fast-acting treatments, which represents a significant advance for clinical use. It is advantageous to use a fast-acting therapeutic in a clinical therapeutic setting that typically lasts for one, two, or several hours.

In certain aspects, the entactogenic properties of certain compounds can be improved by administering an effective amount to a host such as a human, in need thereof, in a composition of an enantiomerically enriched composition that has an abundance of one enantiomer over the other, or for some of the compounds described herein, a substantially pure enantiomer (or diastereomer, where relevant). It has been discovered that certain entactogens in enantiomerically enriched form act differently from the racemate on various 5-HT receptors, dopamine receptors, nicotinic acetylcholine receptors, and norepinephrine receptors, producing variable effects, and that those effects can be selected for based on desired outcome for the patient. This could not be predicted in advance given the complexity of the neurotransmitter system.

This invention also provides the use of a compound, pure enantiomer or enantiomerically enriched mixture of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, Formula XX-B, Formula XXI, or Formula XXII for the manufacture of a medicament for the treatment of maladaptive response to perceived psychological threats. Additionally, this invention provides a pharmaceutical formulation adapted for the treatment of maladaptive response to perceived psychological threats containing a compound, pure enantiomer or enantiomerically enriched mixture of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, Formula XX-B, Formula XXI, or Formula XXII. Furthermore, this invention includes a method for the treatment of maladaptive response to perceived psychological threats that comprises administering an effective amount of a compound, pure enantiomer or enantiomerically enriched mixture of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, Formula XVII, Formula XVIII, Formula XVIII-B, Formula XIX, Formula XX, Formula XX-B, Formula XXI, or Formula XXII, given either in the context of psychotherapy or as a stand-alone treatment.

### Methods to Treat Headache Disorders

In certain embodiments, a method of treating a patient with primary or secondary headaches is provided, comprising administering an effective amount of a compound, pure enantiomer, or enantiomerically enriched mixture of a halogenated benzofuran compound of the present invention, or a pharmaceutically acceptable salt thereof. While administration of such a compound or preparation typically occurs as needed (i.e., at the onset of headache or prodromal syndrome), in some cases, administration may be monthly, weekly, daily, twice daily, or a similar interval to achieve adequate symptom relief. Because some headache disorders have cyclical or other patterns to their occurrence, it is contemplated that medication could be taken using a personalized schedule that is based on use of an algorithm to predict the onset of headache. Administration may be oral, but other routes including buccal, sublingual, inhaled, and other parenteral routes are contemplated. In some embodiments, routes with fast onset of therapeutic effects are considered advantageous.

As used herein, primary headaches include, but are not limited to migraine, migraine signs and symptoms without cephalgia, tension-type headaches, cluster headaches and other trigeminal autonomic cephalalgias, new daily persistent headache, hypnic headaches, stabbing headaches, and other primary headache disorders. Secondary headaches referred to herein can refer to those due to trauma or injury, cranial or cervical vascular disorder, non-vascular intracranial disorder, headaches due to substance use or substance withdrawal, and other secondary headaches.

In certain embodiments a halogenated benzofuran compound of the present invention is used to treat a migraine, headache, or cluster headache. Non-limiting examples of migraines include migraine without aura, migraine with aura, chronic migraine, abdominal migraine, acephalgic migraine, silent migraine, migraine with brainstem aura, hemiplegic migraine, retinal migraine, and status migrainosus.

Improvement in headache disorders is typically indicated by improvements (lessening of severity or frequency) in pain, nausea, photophobia, and phonophobia and the accompanying disruption of normal activities (Loder and Burch 2012. Cephalalgia, 32(3), pp. 1179-182; Vingen et al. 1998. Cephalalgia, 18(5), pp. 250-256; Sauro et al. 2010. Headache: The Journal of Head and Face Pain, 50(3), pp. 383-395). As such, it is anticipated that the methods of treatment disclosed within will result in improvements of one or more of these signs and symptoms.

### Methods to Treat Seizure Disorders

In certain embodiments, a method of treating a patient with a seizure disorder is provided, comprising administering an effective amount of a compound, pure enantiomer, or enantiomerically enriched mixture of a halogenated benzofuran compound of the present invention, or a pharmaceutically acceptable salt thereof. While administration of such a compound or preparation typically occurs daily or twice daily, in some cases, administration may be as needed (i.e., at the onset of a prodromal syndrome). Because some seizure disorders have cyclical or other patterns to their occurrence, it is contemplated that medication could be taken using a personalized schedule that is based on use of an algorithm to predict the onset of seizures. Administration may be oral, but other routes including buccal, sublingual, inhaled, and other parenteral routes are contemplated. In some embodiments, routes with fast onset of therapeutic effects are considered advantageous.

As used herein, seizure disorders include, but are not limited to focal aware seizures, focal impaired awareness seizures, bilateral tonic-clonic seizures, absence seizures, atyptical absence seizures, tonic-clonic seizures, atonic seizures, clonic seizures, tonic seizures, myoclonic seizures, gelastic seizures, and dacrystic seizures. In certain embodiments a halogenated benzofuran compound is used to treat epilepsy. In certain embodiments, the form of epilepsy is severe myoclonic epilepsy of infancy.

Improvement in seizure disorders is understood to mean a decrease in frequency or severity (or both frequency and severity), which can be assessed with both self-report and objective measures (e.g., EEG, including wearable devices) (Cramer and French 2001. Epilepsia, 42(1), pp. 119-129; Karoly, Goldenholz, and Cook 2018. Current opinion in neurology, 31(2), pp. 162-168). As such, it is anticipated that the methods of treatment disclosed within will result in improvements of one or more of these signs and symptoms.

### Non-Limiting Examples of Pharmacotherapeutic Counseling Use

Psychotherapy, cognitive enhancement, or life coaching conducted with a compound or pharmaceutically acceptable salt as described herein employed as an adjunct (hereafter, “pharmacotherapy” or “pharmacotherapy counseling”) is typically conducted in widely spaced sessions with one, two, or rarely three or more administrations of an entactogen per session. These sessions can be as frequent as weekly but are more often approximately monthly or even less frequently. In most cases, a small number of pharmacotherapy counseling sessions, on the order of one to three, is needed for the patient to experience significant clinical progress, as indicated, for example, by a reduction in signs and symptoms of mental distress, by improvement in functioning in some domain of life, by arrival at a satisfactory solution to some problem, or by increased feelings of closeness to and understanding of some other person. In some embodiments, the psychotherapy, cognitive enhancement, or life coaching is conducted with an effective amount of a halogenated benzofuran compound or an effective amount of enantiomerically enriched halogenated benzofuran compound or a pharmaceutically acceptable salt thereof.

The following sections provide detailed examples of pharmacotherapy. While common procedures are described, these are intended as illustrative, non-restrictive examples. It is anticipated that the prescribing physician and therapy team may wish to specify different procedures than those described here based on their clinical judgment concerning the needs of the patient.

The example methods of treatment can also be modified with very minor changes to treat multiple patients at once, including couples or families. Hence, “patient” should be understood to mean one or more individuals.

Use of a Compound or Composition of the Present Invention in Conjunction with Conventional Psychotherapy or Coaching

In certain embodiments, the use of a described halogenated benzofuran compound or composition of the present invention as pharmacotherapy is integrated into the patient's ongoing psychotherapy or coaching (hereafter abbreviated as “psychotherapy”). If a patient in need of the pharmacotherapy is not in ongoing psychotherapy, then psychotherapy may be initiated and the pharmacotherapy counseling added later, after the prescribing physician and treating psychotherapist, physician, coach, member of the clergy, or other similar professional or someone acting under the supervision of such a professional (hereafter, “therapist”) agree that the pharmacotherapy counseling is indicated and that there have been sufficient meetings between the patient and therapist to establish an effective therapeutic alliance.

If the patient is not experienced with the pharmacotherapy, a conversation typically occurs in which the therapist or other members of the therapy team addresses the patient's questions and concerns about the medicine and familiarizes the patient with the logistics of pharmacotherapy-assisted session. The therapist describes the kinds of experience that can be expected during the pharmacotherapy session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contra-indicated medicines or drugs for an adequate period before and after the pharmacotherapy).

A compound or composition of the invention (or alternately herein for convenience, the “medicine”) is administered shortly before or during a scheduled psychotherapy session, with timing optionally selected so that therapeutic effects begin by the time the psychotherapy session begins. It is to be understood that references to administering the medicine “during” a psychotherapeutic or other session are intended to refer to timing the administration of the medicine such that the therapeutic effects of the medicine at least partly temporally overlap with the therapeutic effects of the session. Either shortly before or after administration of the medicine, it is common for the therapist to provide some reminder of their mutual commitments and expected events during the session.

The psychotherapy session is carried out by the therapist, who, optionally, may be remote and in communication with the patient using a communication means suitable for telehealth or telemedicine, such as a phone, video, or other remote two-way communication method. Optionally, video or other monitoring of the patient's response or behavior is used to document or measure the session. The therapist uses their clinical judgment and available data to adjust the session to the needs of the patient. Many therapists view their responsibility as being to facilitate rather than direct the patient's experience. This may sometimes involve silent empathic listening, while other times it may include more active support to help the patient arrive at new perspectives on their life.

It is anticipated that the therapeutic effects of the medicine will allow the patient to make more rapid therapeutic progress than would normally be possible. These effects include decreased neuroticism and increased feelings of authenticity. Patients are often able to calmly contemplate actual or possible experiences that would normally be upsetting or even overwhelming. This can facilitate decision making and creativity in addition to mental wellness.

Optionally, the prescribing physician may allow a second or even third administration of the medicine or another psychotherapeutic agent in order to extend the therapeutic effects. Optionally, a pharmaceutical preparation with modified release is employed to make this unnecessary.

Because the duration of the scheduled psychotherapy session may be shorter than the therapeutic effects of the medicine, the therapist may suggest to the patient activities to support further psychotherapeutic progress after the psychotherapy session has ended. Alternatively, the therapist may continue to work with the patient until the therapeutic effects of the medicine have become clinically minimal.

In a subsequent non-pharmacological psychotherapy session, the therapist and patient will typically discuss the patient's experiences from the pharmacotherapy session and the therapist will often aid the patient in recalling the therapeutic effects and help them to incorporate the experiences into their everyday lives.

Pharmacotherapy sessions may be repeated as needed, based on the judgment of the treating physician and therapy team regarding the needs of the patient.

### Use of a Compound or Composition of the Present Invention Outside of Conventional Psychotherapy

In certain embodiments, a compound or composition of the present invention is administered outside of a conventional psychotherapy. This method is a broader, more flexible approach to pharmacotherapy that is not centered on supervision by a therapist. These pharmacotherapy sessions can take place in many different quiet and safe settings, including the patient's home. The setting is typically chosen to offer a quiet setting, with minimal disruptions, where the patient feels psychologically safe and emotionally relaxed. The setting may be the patient's home but may alternatively be a clinic, retreat center, or hotel room.

Optionally, a checklist may be followed to prepare the immediate environment to minimize distractions and maximize therapeutic or decision-making benefits. This checklist can include items such as silencing phones and other communications devices, cleaning and tidying the environment, preparing light refreshments, preparing playlists of appropriate music, and pre-arranging end-of-session transportation if the patient is not undergoing pharmacotherapy at home.

Before the pharmacotherapy session, there may be an initial determination of the therapeutic or other life-related goals (for example, decision-making, increasing creativity, or simply appreciation of life) that will be a focus of the session. These goals can optionally be determined in advance with support from a therapist.

Optionally, the therapist may help the patient select stimuli, such as photographs, videos, augmented or virtual reality scenes, or small objects such as personal possessions, that will help focus the patient's attention on the goals of the session or on the patient's broader life journey. As examples that are intended to be illustrative and not restrictive, these stimuli can include photographs of the patient from when they were young, which can increase self-compassion, or can include stimuli relating to traumatic events or phobias experienced by the patient, which can help the patient reevaluate and change their response to such stimuli. Optionally, the patient selects these stimuli without assistance (for example, without the involvement of the therapist) or does not employ any stimuli. Optionally, stimuli are selected in real time by the therapist or an algorithm based on the events of the session with the goal of maximizing benefits to the patient.

If the patient is not experienced with the pharmacotherapy, a conversation occurs in which the therapist addresses the patient's questions and concerns about the medicine and familiarizes the patient with the logistics of a pharmacotherapy-assisted session. The therapist describes the kinds of experience that can be expected during the pharmacotherapy-assisted session. Optionally, parts of this conversation employ written, recorded, or interactive digital explanations, as might be used in the informed consent process in a clinical trial. The therapist may additionally make commitments to support the participant's healthcare and wellness process. In turn, the patient may be asked to make commitments of their own (such as not to hurt themselves or others and to abstain from contraindicated medicines or drugs for an adequate period before and after the pharmacotherapy).

Selected session goals and any commitments or other agreements regarding conduct between the patient and therapy team are reviewed immediately before administration of the medicine. Depending on the pharmaceutical preparation and route of administration, the therapeutic effects of the medicine may begin within one hour. Typical therapeutic effects include decreased neuroticism and increased feelings of authenticity. Patients are often able to calmly contemplate experiences or possible experiences that would normally be upsetting or even overwhelming. This can facilitate decision making and creativity in addition to mental wellness.

Optionally, sleep shades and earphones with music or soothing noise may be used to reduce distractions from the environment. Optionally, a virtual reality or immersive reality system may be used to provide stimuli that support the therapeutic process. Optionally, these stimuli are preselected; optionally, they are selected in real time by a person, or an algorithm based on events in the session with the goal of maximizing benefits to the patient. Optionally, a therapist or other person well-known to the patient is present or available nearby or via phone, video, or other communication method in case the patient wishes to talk, however the patient may optionally undergo a session without the assistance of a therapist. Optionally, the patient may write or create artwork relevant to the selected session goals. Optionally, the patient may practice stretches or other beneficial body movements, such as yoga (“movement activity”).

Optionally, in other embodiments the patient may practice movement activity that includes more vigorous body movements, such as dance or other aerobic activity. Movement activity also may make use of exercise equipment such as a treadmill or bicycle.

In some additional embodiments, the patient may be presented with music, video, auditory messages, or other perceptual stimuli. Optionally, these stimuli may be adjusted based on the movements or other measurable aspects of the patient. Such adjustment may be done by the therapist with or without the aid of a computer, or by a computer alone in response to the patient aspects, including by an algorithm or artificial intelligence, and “computer” broadly meaning any electronic tool suitable for such purposes, whether worn or attached to a patient (for example, watches, fitness trackers, “wearables,” and other personal devices; biosensors or medical sensors; medical devices), whether directly coupled or wired to a patient or wirelessly connected (and including desktop, laptop, and notebook computers; tablets, smartphones, and other mobile devices; and the like), and whether within the therapy room or remote (for example, cloud-based systems).

For example, measurable aspects of a patient (for example, facial expression, eye movements, respiration rate, pulse rate, skin color change, patient voice quality or content, patient responses to questions) from these tools may be individually transformed into scores on standardized scales by subtracting a typical value and then multiplying by a constant and these scores may be further multiplied by constants and added together to create an overall score that can optionally be transformed by multiplication with a link function, such as the logit function, to create an overall score. This score may be used to select or adjust stimuli such as selecting music with higher or lower beats-per-minute or with faster or slower notes, selecting images, audio, or videos with different emotionality or autobiographical meaning, or selecting activities for the patient to engage in (such as specific movements, journaling prompts, or meditation mantras).

It should be readily appreciated that a patient can participate in numerous therapeutically beneficial activities, where such participation follows or is in conjunction with the administration of a compound or composition of the invention, including writing about a preselected topic, engaging in yoga or other movement activity, meditating, creating art, viewing of photographs or videos or emotionally evocative objects, using a virtual reality or augmented reality system, talking with a person, and thinking about a preselected problem or topic, and it should be understood that such participation can occur with or without the participation or guidance of a therapist.

Optionally, the prescribing physician may allow a second or even third administration of the medicine or another psychotherapeutic agent in order to extend the therapeutic effects. Optionally, a pharmaceutical preparation with modified release is employed to make this unnecessary.

The patient typically remains in the immediate environment until the acute therapeutic effects of the medicine are clinically minimal, usually within eight hours. After this point, the session is considered finished.

The treatment plan will often include a follow-up session with a therapist. This follow-up session occurs after the pharmacotherapy counseling session has ended, often the next day but sometimes several days later. In this session, the patient discusses their experiences from the pharmacotherapy counseling session with the therapist, who can aid them in recalling the therapeutic effects and help them to incorporate the experiences into their everyday lives.

Pharmacotherapy counseling sessions may be repeated as needed, based on the judgment of the treating physician and therapy team regarding the needs of the patient.

### Combination Therapy

In certain aspects an effective amount of a halogenated benzofuran compound of the present invention or a salt, salt mixture, or pharmaceutical composition thereof is used in combination with one or more additional active agents to treat a disorder described herein or provide mental enhancement.

The pharmaceutical compositions of the invention are not limited to combinations of a single active compound and a single carrier, diluent, or excipient alone, but also include combinations of multiple such Structures, other active compounds, and/or multiple carriers, diluents, and excipients. Pharmaceutical compositions of this invention thus may comprise one or more Structures (or their derivatives and analogues) in combination, together with one or more pharmaceutically acceptable carriers, diluents, and/or excipients, and additionally with one or more other active compounds.

Different embodiments of the invention include the following examples: Pharmaceutically acceptable complex derivatives of each drug in each group, including solvates, salts, esters, enantiomers, isomers (stereoisomers and/or constitutional, including ones based on substituting deuterium for hydrogen), derivatives or prodrugs of halogenated benzofuran compound or pure enantiomer, pure diastereomer, diastereomerically enriched mixture, or enantiomerically enriched mixture thereof, or a compound of Formulas I-XXII. Other embodiments of the invention include multiple variations in the pharmaceutical dosages of each drug in the combination as further outlined below. Other embodiments of the invention include various forms of preparations including using solids, liquids, immediate or delayed or extended-release forms. Many types of variations are possible as known to those skilled in the art.

In some aspects, a halogenated benzofuran compound is formulated in a pharmaceutical preparation with other active compounds to increase therapeutic efficacy, decrease unwanted effects, increase stability/shelf-life, and/or alter pharmacokinetics. Such other active compounds include, but are not limited to antioxidants (such alpha-lipoate in acid or salt form, ascorbate in acid or salt form, selenium, or N-acetylcysteine); substrates or inhibitors of cytochrome p450 2D6 (such as dextromethorphan, fluoxetine, paroxetine, bupropion, duloxetine, or quinidine), H2-receptor agonists or antagonists (such as famotidine); stimulants (such as dextroamphetamine, amphetamine, lisdexamphetamine, methylphenidate, or methamphetamine); entactogens (such as MDMA, 3,4-methylenedioxy-N-ethylamphetamine, [1-(2H-1,3-benzodioxol-5-yl)butan-2-yl](methyl)amine, 1-(1-benzofuran-6-yl)propan-2-amine, or [1-(1-benzofuran-5-yl)propan-2-yl](methyl)amine); anti-inflammatories (such as ibuprofen or ketoprofen); matrix metalloproteinase inhibitors (such as doxycycline); NOS inhibitors (such as S-methyl-L-thiocitrulline); proton pump inhibitors (such as omeprazole); phosphodiesterase 5 inhibitors (such as sildenafil); drugs with cardiovascular effects (beta antagonists such as propranolol, mixed alpha and beta antagonists such as carvedilol, alpha antagonists such as prazosin, imidazoline receptor agonists such as rilmenidine or moxonidine; serotonin antagonists such as ketanserin or lisuride); norepinephrine transporter blockers (such as reboxetine); acetylcholine nicotinic receptor modulators (such as bupropion, hydroxybupropion, methyllycaconitine, memantine, or mecamylamine); gastrointestinal acidifying agents (such as ascorbic acid or glutamic acid hydrochloride); alkalinizing agents (such as sodium bicarbonate), NMDA receptor antagonists (such as ketamine); TrkB agonists (such as 7,8-dihydroxyflavone, 7,8,3′-trihydroxyflavone, or N-acetylserotonin), or serotonin receptor agonists (such as 5-methoxy-N-methyl-N-isopropyltryptamine, N,N-Dimethyl-2-(2-methyl-1H-indol-1-yl)ethan-1-amine, psilocin, or psilocybin). The ingredients may be in ion, freebase, or salt form and may be isomers or prodrugs.

The pharmacological agents that make up the combination therapy disclosed herein may be a combined dosage form or in separate dosage forms intended for substantially simultaneous administration. The pharmacological agents that make up the combination therapy may also be administered sequentially, with either therapeutic compound being administered by a regimen calling for two-step administration. The two-step administration regimen may call for sequential administration of the active agents or spaced-apart administration of the separate active agents.

The time period between the multiple administration steps may range from, a few minutes to several hours, depending upon the properties of each pharmacological agent, such as potency, solubility, bioavailability, plasma half-life and kinetic profile of the pharmacological agent. Circadian variation of the target molecule concentration may also determine the optimal dose interval. For example, a halogenated benzofuran compound may be administered while the other pharmacological agent is being administered (concurrent administration) or may be administered before or after other pharmacological agent is administered (sequential administration).

In cases where the two (or more) drugs included in the fixed-dose combinations of the present invention are incompatible, cross-contamination can be avoided, for example, by incorporation of the drugs in different drug layers in the oral dosage form with the inclusion of a barrier layer(s) between the different drug layers, wherein the barrier layer(s) comprise one or more inert/non-functional materials.

In certain typical embodiments, the formulations of the present invention are fixed-dose combinations of any one of a compound of Formulas I-XXII and at least one other pharmacological agent. In certain typical embodiments, the formulations of the present invention are fixed-dose combinations of a halogenated benzofuran compound or pure enantiomer, pure diastereomer, diastereomerically enriched mixture, or enantiomerically enriched mixture thereof and at least one other pharmacological agent. Fixed-dose combination formulations may contain therapeutically efficacious fixed-dose combinations of formulations of a halogenated benzofuran compound or pure enantiomer, pure diastereomer, diastereomerically enriched mixture, or enantiomerically enriched mixture thereof, or any one of a compound of Formulas I-XXII and other pharmacological agents in the form of single-layer monolithic tablet or multi-layered monolithic tablet or in the form of a core tablet-in-tablet or multi-layered multi-disk tablet or beads inside a capsule or tablets inside a capsule.

Pharmaceutical Combinations with Dextroamphetamine

In certain typical embodiments, halogenated benzofuran compound or a pure enantiomer or enantiomerically enriched mixture thereof, or any one of a compound of Formulas I-XXII, either racemic, pure enantiomer, pure diastereomer, diastereomerically enriched mixture, or enantiomerically enriched mixture thereof, and with zero to five hydrogens replaced with deuterium, is formulated in a pharmaceutical composition that contains a pharmaceutically acceptable salt of dextroamphetamine, for example, in the amount between about 2 mg to 25 mg, such as, 2 mg, 4 mg, 5 mg, 7 mg, 10 mg, 15 mg, 20 mg, or 25 mg. The required amount of dextroamphetamine will vary depending on the needs of the patient.

In another typical embodiment halogenated benzofuran compound or a pure enantiomer or enantiomerically enriched mixture thereof, or any one of a compound of Formulas I-XXII, either racemic, pure enantiomer, pure diastereomer, diastereomerically enriched mixture, or enantiomerically enriched mixture thereof, and with zero to five hydrogens replaced with deuterium, are formulated in a pharmaceutical composition that contains a pharmaceutically acceptable salt of dextroamphetamine with dextroamphetamine, for example, in a ratio by weight of 1:2, 1:3, 1:4, 1:5, 1:6, 1:7, 1:8, 1:9 or 1:10 to the halogenated benzofuran compound or a pure enantiomer or enantiomerically enriched mixture thereof, or any one of a compound of Formulas I-XXII. The required amount of dextroamphetamine will vary depending on the needs of the patient.

Pharmaceutical Combinations with MDMA

In some typical embodiments, a halogenated benzofuran compound or a pure enantiomer or enantiomerically enriched mixture thereof, or any one of a compound of Formulas I-XXII, either racemic, pure enantiomer, pure diastereomer, diastereomerically enriched mixture, or enantiomerically enriched mixture thereof, and with zero to five hydrogens replaced with deuterium, is formulated in a pharmaceutical composition that contains a pharmaceutically acceptable salt of MDMA, for example, in an amount between 5 and 180 mg, typically 15-60 mg. The required amount of MDMA will vary depending on the needs of the patient.

In some typical embodiments, a halogenated benzofuran compound or a pure enantiomer or enantiomerically enriched mixture thereof, or any one of a compound of Formulas I-XXII, either racemic, pure enantiomer, pure diastereomer, diastereomerically enriched mixture, or enantiomerically enriched mixture thereof, and with zero to five hydrogens replaced with deuterium, is formulated in a pharmaceutical composition that contains a pharmaceutically acceptable salt of MDMA with MDMA, for example, in a ratio by weight of 1:2, 1:3, 1:4, 1:5, 1:6, 1:7, 1:8, 1:9 or 1:10 to the halogenated benzofuran compound or a pure enantiomer or enantiomerically enriched mixture thereof. The required amount of MDMA will vary depending on the needs of the patient.

### Non-Limiting Examples of Combination Formulations

Capsules, each containing 40 mg of the halogenated benzofuran compound, are made as follows:

| Ingredient | Quantity (mg/capsule) |
|---|---|
| A compound of one of Formulas I-XXII | 10 |
| A compound of one of Formulas I-XXII | 30 |
| Amphetamine sulfate | 5 |
| Starch | 109 |
| Magnesium stearate | 1 |

The active ingredients, cellulose, starch, and magnesium stearate are blended, passed through a No. 20 mesh U.S. sieve, and filled into hard gelatin capsules in 155 mg quantities.

Capsules, each containing 40 mg of the halogenated benzofuran compound, are made as follows:

| Ingredient | Quantity (mg/capsule) |
|---|---|
| A compound of one of Formulas I-XXII | 30 |
| A compound of one of Formulas I-XXII | 10 |
| Psilocybin | 20 |
| Alpha lipoic acid | 40 |
| Starch | 72 |
| Magnesium stearate | 1 |

The active ingredients, cellulose, starch, and magnesium stearate are blended, passed through a No. 20 mesh U.S. sieve, and filled into hard gelatin capsules in 155 mg quantities.

It should be readily appreciated that the formulation examples are illustrative only. Accordingly, it should be understood that reference to particular compounds is likewise illustrative, and the compounds in any of the non-limiting examples may be substituted by other compounds of the invention. Likewise, any of the other active compounds (for example, amphetamine sulfate or psilocybin hydrochloride) may be substituted by a different other active compound, as may be the inactive compounds.

Moreover, for any active compound of the invention, for example a compound of Formula I-XXII, substitution of the compound by its prodrug, free base, salt, or hydrochloride salt shall be understood to provide merely an alternative embodiment still within the scope of the invention. Further, compositions within the scope of the invention should be understood to be open-ended and may include additional active or inactive compounds and ingredients.

The type of formulation employed for the administration of a compound employed in the methods of the present invention generally may be dictated by the compound(s) employed, the type of pharmacokinetic profile desired from the route of administration and the compound(s), and the state of the patient.

### Dosage Regimes

A compound or pharmaceutically acceptable formulation of the present invention may be administered to the host in any amount, and with any frequency, that achieves the goals of the invention as used by the healthcare provider, or otherwise by the host in need thereof, typically a human, as necessary or desired.

In certain embodiments, the composition as described herein is provided only in a controlled counseling session, and administered only once, or perhaps 2, 3, 4, or 5 or more times in repeated counseling sessions to address a mental disorder as described herein.

In other embodiments, the composition as described herein is provided outside of a controlled counseling session, and perhaps self-administered, as needed to perhaps 2, 3, 4, or 5 or more times in to address a mental disorder as described herein.

In other embodiments, the composition of the present invention may be administered on a routine basis for mental wellbeing or for entactogenic treatment.

A halogenated benzofuran compound may be administered in a variety of doses, routes of administration, and dosing regimens, based on the indication and needs of the patient. Non-limiting examples of therapeutic use include discrete psychotherapeutic sessions, ad libitum use for treatment of episodic disorders, and ongoing use for treatment of subchronic and chronic disorders.

### Psychotherapeutic Sessions

For some indications, the selected fluorobenzofuran compound medicine of the present invention is taken in discrete psychotherapy or other beneficial sessions. It is anticipated that these sessions will typically be separated by more than 5 half-lives of the medicine and, for most patients, will typically occur only 1 to 5 times each year.

For these sessions, it will typically be desirable to induce clearly perceptible entactogenic effects that will facilitate fast therapeutic progress. Non-exhaustive examples of oral doses of medicine that produce clearly perceptible entactogenic effects for exemplary purposes for any compound described herein includes (using compounds for illustrative purposes only): about 40 to about 120 mg of any one of a compound of Formulas I-XXII, about 40 to about 120 mg of any one of a compound of Formulas I-XXII, about 50 to about 300 mg of any one of a compound of Formulas I-XXII, about 50 to about 300 mg any one of a compound of Formulas I-XXII, about 75 to about 500 mg any one of a compound of Formulas I-XXII, about 75 to about 500 mg of any one of a compound of Formulas I-XXII, about 75 to about 800 mg of any one of a compound of Formulas I-XXII, about 75 to about 800 mg any one of a compound of Formulas I-XXII. Non-exhaustive examples of oral doses of medicine that produce clearly perceptible entactogenic effects for exemplary purposes for any compound described herein includes (using compounds for illustrative purposes only): about 40 to about 120 mg of a halogenated benzofuran compound, about 50 to about 300 mg of a halogenated benzofuran compound, about 75 to about 500 mg of a halogenated benzofuran compound, or about 75 to about 800 mg of a halogenated benzofuran compound.

It is anticipated that the medicine would be taken once or, more rarely, two or three times in a single therapeutic session. In these cases, it is common for each subsequent dose to be half of the previous dose or lower. Multiple doses within a session typically occur because either the patient's sensitivity to the medicine was unknown and too low of an initial dose was employed or because the patient is experiencing a productive session and it is desirable to extend the duration of therapeutic effects. Controlled release preparations may be used to lengthen the duration of therapeutic effects from a single administration of the medicine. In cases where multiple administrations are used in a session, it is anticipated that individual doses will be lower so that plasma concentrations remain within a desired therapeutic range.

Non-limiting, non-exhaustive examples of indications that may benefit from psychotherapeutic sessions include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders. Also included as exemplary situations where an individual would benefit from a psychotherapeutic session are situations from a reduction of neuroticism or psychological defensiveness, an increase in openness to experience, an increase in creativity, or an increase in decision-making ability.

Ad Libitum Use for Treatment of Episodic Disorders For some indications, such as social anxiety, where the patient has need for relief from episodic occurrence of a disorder, it is anticipated that the medicine would be taken as needed but that uses should be separated by more than 5 half-lives of the medicine to avoid bioaccumulation and formation of tolerance.

For treating episodic disorders, clearly perceptible entactogenic effects are often not desirable, as they may impair some aspects of functioning. Non-exhaustive examples of oral doses of medicine for any compound described herein includes (using compounds for illustrative purposes only) that produce subtle, barely perceptible therapeutic effects include: about 10 to about 60 mg of any one of a compound of Formulas I-XXII, about 10 to about 60 mg of any one of a compound of Formulas I-XXII, about 10 to about 100 mg of any one of a compound of Formulas I-XXII, about 10 to about 100 mg any one of a compound of Formulas I-XXII, about 20 to about 150 mg of any one of a compound of Formulas I-XXII, about 20 to about 150 mg of any one of a compound of Formulas I-XXII, about 20 to about 200 mg of any one of a compound of Formulas I-XXII, and about 20 to about 200 mg of a compound of Formulas I-XXII. Non-exhaustive examples of oral doses of medicine for any compound described herein includes (using compounds for illustrative purposes only) that produce subtle, barely perceptible therapeutic effects include: about 10 to about 60 mg of a halogenated benzofuran compound, about 10 to about 100 mg of a halogenated benzofuran compound about 20 to about 150 mg of a halogenated benzofuran compound, and about 20 to about 200 mg of a halogenated benzofuran compound.

Non-limiting, non-exhaustive examples of indications that may benefit from episodic treatment include post-traumatic stress disorder, depression, dysthymia, anxiety and phobia disorders, feeding, eating, and binge disorders, body dysmorphic syndromes, alcoholism, tobacco abuse, drug abuse or dependence disorders, disruptive behavior disorders, impulse control disorders, gaming disorders, gambling disorders, personality disorders, attachment disorders, autism, and dissociative disorders, provided that clinically significant signs and symptoms worsen episodically or in predictable contexts.

### Ongoing Use for Treatment of Subchronic and Chronic Disorders

For some indications, such as substance use disorders, inflammatory conditions, chronic pain, and neurological indications, including treatment of stroke, brain trauma, dementia, and neurodegenerative diseases, where the patient has need for ongoing treatment, it is anticipated that the medicine would be taken daily, twice daily, or three times per day. With some indications (subchronic disorders), such as treatment of stroke or traumatic brain injury, it is anticipated that treatment duration will be time-limited and dosing will be tapered when the patient has recovered. An example dose taper regimen is a reduction in dose of 10% of the original dose per week for nine weeks. With other, chronic disorders, such as dementia, it is anticipated that treatment will be continued as long as the patient continues to receive clinically significant benefits.

For treating subchronic and chronic disorders, clearly perceptible entactogenic effects are often not desirable. Non-exhaustive examples of oral doses of medicine for any compound described herein includes (using compounds for illustrative purposes only) that produce subtle, barely perceptible therapeutic effects with ongoing dosing include: about 5 to about 60 mg of any one of a compound of Formulas I-XXII, about 5 to about 60 mg of any one of a compound of Formulas I-XXII, about 5 to about 100 mg of any one of a compound of Formulas I-XXII, about 5 to about 100 mg of any one of a compound of Formulas I-XXII, about 10 to about 150 mg of any one of a compound of Formulas I-XXII, or about 10 to about 150 mg of any one of a compound of Formulas I-XXII. Non-exhaustive examples of oral doses of medicine for any compound described herein includes (using compounds for illustrative purposes only) that produce subtle, barely perceptible therapeutic effects with ongoing dosing include: about 5 to about 60 mg of a halogenated benzofuran compound, about 5 to about 100 mg of a halogenated benzofuran compound, about 10 to about 150 mg of a halogenated benzofuran compound, and about 10 to about 200 mg of a halogenated benzofuran compound.

Non-limiting, non-exhaustive examples of subchronic and chronic disorders that may benefit from regular treatment include migraine, headaches (for example, cluster headache), neurodegenerative disorders, Alzheimer's disease, Parkinson's disease, schizophrenia, stroke, traumatic brain injury, phantom limb syndrome, chronic pain syndromes, and other conditions where increasing neuronal plasticity is desirable.

### Pharmaceutical Compositions and Salts

While it is possible to administer a compound employed in the methods of this invention directly without any formulation, a compound is typically administered in the form of pharmaceutical compositions comprising a pharmaceutically acceptable carrier, diluent, or excipient. “Pharmaceutically acceptable” as used in connection with an excipient, carrier, or diluent means an excipient, carrier, or diluent that is useful in preparing a pharmaceutical composition that is generally safe, non-toxic, and neither biologically nor otherwise undesirable for veterinary use and/or human pharmaceutical use. These compositions can be administered by a variety of routes including systemic, topical, parenteral, oral, mucosal (for example, buccal, sublingual), rectal, transdermal, subcutaneous, intravenous, intramuscular, inhaled, and intranasal. Such compositions are prepared in a manner well known in the pharmaceutical art and comprise at least one active compound. (See, for example, Remington, 2005, Remington: The science and practice of pharmacy, 21st ed., Lippincott Williams & Wilkins.)

The pharmaceutical composition may be formulated as any pharmaceutically useful form, for example, a solid dosage form, a liquid, an aerosol, a cream, a gel, a pill, an injection or infusion solution, a capsule, a tablet, a syrup, a transdermal patch, a subcutaneous patch, a dry powder, an inhalation formulation, a suppository, a buccal or sublingual formulation, a parenteral formulation, an ophthalmic solution, or in a medical device. Some dosage forms, such as tablets and capsules, are subdivided into suitably sized unit doses containing appropriate quantities of the active components, for example, an effective amount to achieve the desired purpose.

A “pharmaceutically acceptable composition” thus refers to at least one compound (which may be a mixture of enantiomers or diastereomers, as fully described herein) of the invention and a pharmaceutically acceptable vehicle, excipient, diluent, or other carrier in an effective amount to treat a host, typically a human, who may be a patient.

In certain nonlimiting embodiments the pharmaceutical composition is a dosage form that contains from about 0.1 mg to about 1500 mg, from about 10 mg to about 1000 mg, from about 100 mg to about 800 mg, or from about 200 mg to about 600 mg of the active compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 20, 25, 40, 50, 100, 125, 150, 200, 250, 300, 400, 500, 600, 700, or 750 mg of active compound, or its salt or salt mixture.

The pharmaceutical compositions described herein can be formulated into any suitable dosage form, including aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. Generally, one will desire to administer an amount of the active agents of the present invention that is effective to achieve a plasma level commensurate with the concentrations found to be effective in vivo for a period of time effective to elicit a desired therapeutic effect without abuse liability.

In making the compositions employed in the present invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, solutions, slurries, syrups, aerosols (as a solid or in a liquid medium), ointments containing for example up to 10% by weight of the active compound, soft and hard gelatin capsules, suppositories, dry powders for inhalation, liquid preparations for vaporization and inhalation, topical preparations, transdermal patches, sterile injectable solutions, and sterile packaged powders. Compositions may be formulated as immediate release, controlled release, sustained (extended) release or modified release formulations.

Other embodiments of the invention include multiple routes of administration, which may differ in different patients according to their preference, co-morbidities, side effect profile, and other factors (IV, PO, transdermal, etc.). Other embodiments of the invention include the presence of other substances with the active drugs, known to those skilled in the art, such as fillers, carriers, gels, skin patches, lozenges, or other modifications in the preparation to facilitate absorption through various routes (such as gastrointestinal, transdermal, etc.) and/or to extend the effect of the drugs, and/or to attain higher or more stable serum levels or to enhance the therapeutic effect of the active drugs in the combination.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, for example, about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick, sustained or delayed release of the active ingredient after administration to the patient by employing procedures known in the art.

The compositions in certain non-limiting embodiments formulated in a unit dosage form, each dosage containing from about 0.05 to about 350 mg, more typically about 1.0 to about 180 mg, of the active ingredients. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier, diluent, or excipient.

For example, some dosages fall within the range of at least about 0.007 to about 4 mg/kg or less. In the treatment of adult humans, the range of at least about 0.1 to about 3 mg/kg or less, in single dose may be useful.

It will be understood that the amount of the compound actually administered will be determined by a physician, in light of the relevant circumstances, including the condition to be treated, the chosen route of administration, the actual compound or compounds administered, the age, weight, and response of the individual patient, and the severity of the patient's symptoms, and therefore the above dosage ranges are not intended to limit the scope of the invention in any way.

In some instances, dosage levels below the lower limit of the aforesaid range may be more than adequate, while in other cases still larger doses may be employed without causing any harmful side effects, provided for instance that such larger doses may be first divided into several smaller doses for administration.

Generally, the pharmaceutical compositions of the invention may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions, but shall be able to be determined with ordinary skill.

In other embodiments, a powder comprising the active agents of the present invention formulations described herein may be formulated to comprise one or more pharmaceutical excipients and flavors. Such a powder may be prepared, for example, by mixing the active agents of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

### Oral Formulations

In certain embodiments, a compound Formulas I-XXII of the present invention or a pure enantiomer, diastereomer, diastereomerically enriched mixture, or enantiomerically enriched mixture thereof may be formulated in a pharmaceutically acceptable oral dosage form. Oral dosage forms may include but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

In some embodiments, the solid dosage forms of the present invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, for example, capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the active agents of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the active agent of the present invention formulation. In certain embodiments, some or all of the active agent of the present invention particles are coated. In another embodiment, some or all of the active agent of the present invention particles are microencapsulated. In yet another embodiment, some or all of the active agent of the present invention is amorphous material coated and/or microencapsulated with inert excipients. In still another embodiment, the active agent of the present invention particles are not microencapsulated and are uncoated.

Suitable carriers for use in the solid dosage forms described herein include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include lactose, calcium carbonate, calcium phosphate, dibasic calcium phosphate, calcium sulfate, microcrystalline cellulose (for example, Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include natural starch such as corn starch or potato starch, a pregelatinized starch such as National 1551 or Amijel®, or a sodium starch glycolate such as Promogel® or Explotab®, a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft- or hard-shell capsules and in tablet formulation, binders ensure that the tablet remains intact after compression and help assure blend uniformity prior to a compression or fill step. Materials suitable for use as binders in the solid dosage forms described herein include carboxymethylcellulose, methylcellulose (for example, Methocel®), hydroxypropylmethylcellulose (for example, Hypromellose USP Pharmacoat-603, hydroxypropylmethylcellulose acetate stearate (Agoate HS-LF and HS), hydroxyethylcellulose, hydroxypropylcellulose (for example, Klucel®), ethylcellulose (for example, Ethocel®), and microcrystalline cellulose (for example, Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (for example, Dipac®), glucose, dextrose, molasses, mannitol, sorbitol, xylitol (for example, Xylitab®), lactose, a natural or synthetic gum such as acacia, tragacanth, ghatti gum, mucilage of isapol husks, starch, polyvinylpyrrolidone (for example, Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are typically used in powder-filled gelatin capsule formulations. Binder usage level in tablet formulations is a function of whether direct compression, wet granulation, roller compaction, or usage of other excipients such as fillers which themselves can act as moderate binders are used. Formulators skilled in the art can determine the binder level for the formulations, but binder usage level of up to 70% in tablet formulations is common.

Suitable lubricants or glidants for use in the solid dosage forms described herein include stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (for example, having a density of about 0.45 g/cm3, for example Avicel®, powdered cellulose), and talc.

Suitable wetting agents for use in the solid dosage forms described herein include oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (for example, Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, for example, Pluronic® (BASF), and the like.

Suitable suspending agents for use in the solid dosage forms described here include polyvinylpyrrolidone, for example, polyvinylpyrrolidone K12, polyvinylpyrrolidone K17, polyvinylpyrrolidone K25, or polyvinylpyrrolidone K30, polyethylene glycol, for example, the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, for example, gum tragacanth and gum acacia, guar gum, xanthans, including xanthan gum, sugars, cellulosic, such as, for example, sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining superdisintegrants such as Croscarmellose sodium and different grades of microcrystalline cellulose in different ratios. To aid disintegration, sodium starch glycolate will be added.

The above-listed additives should be taken as merely examples and not limiting, of the types of additives that can be included in solid dosage forms of the present invention. The amounts of such additives can be readily determined by one skilled in the art, according to the particular properties desired.

Oral liquid dosage forms include solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol, glycerol, and propylene glycol. Ethers, such as poly(ethylene glycol), petroleum hydrocarbons such as mineral oil and petrolatum, and water may also be used in suspension formulations.

In some embodiments, formulations are provided comprising particles of a compound of Formulas I-XXII of the present invention or a pure enantiomer or enantiomerically enriched mixture thereof and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous particles consisting of multiple effective particle sizes such that the drug is absorbed in a controlled manner over time. In certain embodiments, the aqueous dispersion or suspension is an immediate-release formulation. In another embodiment, an aqueous dispersion comprising amorphous particles is formulated such that a portion of the particles of the present invention are absorbed within, for example, about 0.75 hours after administration and the remaining particles are absorbed 2 to 4 hours after absorption of the earlier particles.

In other embodiments, addition of a complexing agent to the aqueous dispersion results in a larger span of the particles to extend the drug absorption phase of the active agents such that 50-80% of the particles are absorbed in the first hour and about 90% are absorbed by about 4 hours. Dosage forms for oral administration can be aqueous suspensions selected from the group including pharmaceutically acceptable aqueous oral dispersions, emulsions, solutions, and syrups. See, for example, Singh et al., Encyclopedia of Pharm. Tech., 2nd Ed., 754-757 (2002). In addition to the active agents of the present invention particles, the liquid dosage forms may comprise additives, such as (a) disintegrating agents; (b) dispersing agents; (c) wetting agents; (d) at least one preservative; (e) viscosity enhancing agents; (f) at least one sweetening agent; and (g) at least one flavoring agent.

Examples of disintegrating agents for use in the aqueous suspensions and dispersions include a starch, for example, a natural starch such as corn starch or potato starch, a pregelatinized starch such as National 1551 or Amijel®, or sodium starch glycolate such as Promogel® or Explotab®; a cellulose such as a wood product, microcrystalline cellulose, for example, Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (for example, HPC, HPC-SL, and HPC-L), hydroxypropylmethylcellulose and hydroxypropylmethylcellulose ethers (for example, HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, for example, S-630), 4-(1,1,3,3-tetramethylbutyl)-phenol polymer with ethylene oxide and formaldehyde (also known as tyloxapol), poloxamers (for example, Pluronics F68®, F88®, and F108®, which are block copolymers of ethylene oxide and propylene oxide); and poloxamines (for example, Tetronic 908®, also known as Poloxamine 908®, which is a tetrafunctional block copolymer derived from sequential addition of propylene oxide and ethylene oxide to ethylenediamine (BASF Corp., Parsippany, N.J.)).

In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (for example, HPC, HPC-SL, and HPC-L); hydroxypropyl methylcellulose and hydroxypropyl methylcellulose ethers (for example, HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (for example, Pluronics F68®, F88®, and F108®, which are block copolymers of ethylene oxide and propylene oxide); or poloxamines (for example, Tetronic 908® or Poloxamine 908®).

Wetting agents (including surfactants) suitable for the aqueous suspensions and dispersions described herein are known in the art and include acetyl alcohol, glycerol monostearate, polyoxyethylene sorbitan fatty acid esters (for example, the commercially available Tweens® such as for example, Tween 20® and Tween 80® (ICI Specialty Chemicals)), and polyethylene glycols (for example, Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include potassium sorbate, parabens (for example, methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In some embodiments, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1 weight % and propylparaben from about 0.01 to about 0.02 weight % of the aqueous dispersion.

Suitable viscosity enhancing agents for the aqueous suspensions or dispersions described herein include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the additives listed above, the liquid active agents of the present invention formulations can also comprise inert diluents commonly used in the art, such as water or other solvents, solubilizing agents, emulsifiers, and/or sweeteners.

In still other embodiments, effervescent powders containing a compound of Formulas I-XXII of the present invention or a pure enantiomer, pure diastereomer, diastereomerically enriched mixture, or enantiomerically enriched mixture thereof. Effervescent salts have been used to disperse medicines in water for oral administration. Effervescent salts are granules or coarse powders containing a medicinal agent in a dry mixture, for example a granule or coarse powder composed of sodium bicarbonate, citric acid and/or tartaric acid. When salts of the present invention are added to water, the acids and the base react to liberate carbon dioxide gas, thereby causing “effervescence.” Examples of effervescent salts include sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

Tablets of the invention described here can be prepared by methods well known in the art. Various methods for the preparation of the immediate release, modified release, controlled release, and extended-release dosage forms (for example, as matrix tablets, tablets having one or more modified, controlled, or extended-release layers, etc.) and the vehicles therein are well known in the art. Generally recognized compendia of methods include: Remington: The Science and Practice of Pharmacy, Alfonso R. Gennaro, Editor, 20th Edition, Lippincott Williams & Wilkins, Philadelphia, PA; and Sheth et al. (1980), Compressed tablets, in Pharmaceutical dosage forms, Vol. 1, edited by Lieberman and Lachtman, Dekker, NY.

In certain embodiments, solid dosage forms, for example, tablets, effervescent tablets, and capsules, are prepared by mixing the active agents of the present invention particles with one or more pharmaceutical excipients to form a bulk blend composition. When referring to these bulk blend compositions as homogeneous, it is meant that the active agents of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agents of the present invention formulations can be manufactured by conventional pharmaceutical techniques.

Conventional pharmaceutical techniques for preparation of solid dosage forms include, for example, one or a combination of methods: (1) dry mixing, (2) direct compression, (3) milling, (4) dry or non-aqueous granulation, (5) wet granulation, or (6) fusion. See, for example, Lachman et al., Theory and Practice of Industrial Pharmacy (1986). Other methods include, for example, spray drying, pan coating, melt granulation, granulation, fluidized bed spray drying or coating (for example, Wurster coating), tangential coating, top spraying, tableting, extruding and the like.

Compressed tablets are solid dosage forms prepared by compacting the bulk blend the active agents of the present invention formulations described above. In various embodiments, compressed tablets which are designed to dissolve in the mouth will comprise one or more flavoring agents. In other embodiments, the compressed tablets will comprise a film surrounding a final compressed tablet. In some embodiments, the film coating can provide a delayed release of the active agents of the present invention formulation. In other embodiments, the film coating aids in patient compliance (for example, Opadry® coatings or sugar coating). Film coatings comprising Opadry® typically range from about 1% to about 3% of the tablet weight. Film coatings for delayed-release may comprise 2-6% of a tablet weight or 7-15% of a spray-layered bead weight. In other embodiments, the compressed tablets comprise one or more excipients.

A capsule may be prepared, for example, by placing the bulk blend the active agents of the present invention formulation, described above, inside of a capsule. In some embodiments, the active agents of the present invention formulations (non-aqueous suspensions and solutions) are placed in a soft gelatin capsule. In other embodiments, the active agents of the present invention formulations are placed in standard gelatin capsules or non-gelatin capsules such as capsules comprising HPMC. In other embodiments, the active agents of the present invention formulations are placed in a sprinkle capsule, wherein the capsule may be swallowed whole or the capsule may be opened and the contents sprinkled on food prior to eating. In some embodiments of the present invention, the therapeutic dose is split into multiple (for example, two, three, or four) capsules. In some embodiments, the entire dose of the active agents of the present invention formulation is delivered in a capsule form.

In certain embodiments, ingredients (including or not including the active agents) of the invention are wet granulated. The individual steps in the wet granulation process of tablet preparation include milling and sieving of the ingredients, dry powder mixing, wet massing, granulation, drying, and final grinding. In various embodiments, the active agents of the present invention composition are added to the other excipients of the pharmaceutical formulation after they have been wet granulated. Alternatively, the ingredients may be subjected to dry granulation, for example, via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In some embodiments, the active agents of the present invention formulation are dry granulated with other excipients in the pharmaceutical formulation. In other embodiments, the active agents of the present invention formulation are added to other excipients of the pharmaceutical formulation after they have been dry granulated.

In other embodiments, the formulation of the present invention formulations described herein is a solid dispersion. Methods of producing such solid dispersions are known in the art and include U.S. Pat. Nos. 4,343,789, 5,340,591, 5,456,923, 5,700,485, 5,723,269, and U.S. Pub. No. 2004/0013734. In some embodiments, the solid dispersions of the invention comprise both amorphous and non-amorphous active agents of the present invention and can have enhanced bioavailability as compared to conventional active agents of the present invention formulations. In still other embodiments, the active agents of the present invention formulations described herein are solid solutions. Solid solutions incorporate a substance together with the active agents and other excipients such that heating the mixture results in the dissolution of the drug and the resulting composition is then cooled to provide a solid blend that can be further formulated or directly added to a capsule or compressed into a tablet.

### Non-Limiting Examples of Formulations for Oral Delivery

The examples below provide non-limiting embodiments of formulations for oral delivery, which can be used to deliver any of a compound described herein as a pure enantiomer, pure diastereomer, diastereomerically enriched mixture, or enantiomerically enriched mixture thereof. Therefore, while the compounds below are specified, any desired purity form or compound can be used if it achieves the desired goal of treatment.

In one non-limiting embodiment, hard gelatin capsules comprising the following ingredients are prepared by mixing the ingredients and filling into hard gelatin capsules in 340 mg quantities.

Hard gelatin capsules containing the following ingredients are prepared:

| Ingredient | Quantity (mg/capsule) |
|---|---|
| A halogenated benzofuran compound | 30 |
| Starch | 205 |
| Alpha lipoic acid | 100 |
| Magnesium stearate | 5 |

The above ingredients are mixed and filled into hard gelatin capsules in 340 mg quantities.

A tablet formula is prepared using the ingredients below:

| Ingredient | Quantity (mg/tablet) |
|---|---|
| A halogenated benzofuran compound | 25 |
| Cellulose, microcrystalline | 200 |
| Colloidal silicon dioxide | 10 |
| Stearic acid | 5 |

The components are blended and compressed to form tablets, each weighing 240 mg.

Tablets, each containing 30 mg of active ingredient, are prepared as follows:

| Ingredient | Quantity (mg/tablet) |
|---|---|
| A compound of one of Formulas I-XXII | 20 |
| A compound of one of Formulas I-XXII | 10 |
| Starch | 45 |
| Microcrystalline cellulose | 35 |
| Polyvinylpyrrolidone (as 10% solution in | 4 |
| water) | |
| Sodium carboxymethyl starch | 4.5 |
| Magnesium stearate | 0.5 |
| Talc | 1 |

The active ingredients, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

Capsules, each containing 40 mg of active ingredients are made as follows:

| Ingredient | Quantity (mg/capsule) |
|---|---|
| A compound of one of Formulas I-XXII | 10 |
| A compound of one of Formulas I-XXII | 30 |
| Starch | 109 |
| Magnesium stearate | 1 |

The active ingredients, cellulose, starch, an magnesium stearate are bended, passe through a No. 20 mesh U.S. sieve, and filled into hard gelatin capsules in 150 mg quantities.

Capsules, each containing 100 mg of active ingredient, are made as follows:

| Ingredient | Amount (mg/capsule) |
|---|---|
| A compound of one of Formulas I-XXII | 100 |
| Starch | 407 |
| Magnesium stearate | 3 |

The active ingredient, cellulose, starch, and magnesium stearate are blended, passed through a No. 20 mesh U.S. sieve, and filled into hard gelatin capsules in 510 mg quantities.

### Extended-Release Formulations

Depending on the desired release profile, the oral solid dosage forms of the present invention may contain a suitable amount of controlled-release agents, extended-release agents, and/or modified-release agents (for example, delayed-release agents). The pharmaceutical solid oral dosage forms comprising the active agents of the present invention described herein may be further formulated to provide a modified or controlled release of the active agents of the present invention. In some embodiments, the solid dosage forms described herein may be formulated as a delayed release dosage form such as an enteric-coated delayed release oral dosage forms, i.e., as an oral dosage form of a pharmaceutical composition as described herein which uses an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agents of the formulations described herein are delivered using a pulsatile dosage form. Pulsatile dosage forms comprising the active agents of the present invention formulations described herein may be administered using a variety of formulations known in the art. For example, such formulations include those described in U.S. Pat. Nos. 5,011,692, 5,017,381, 5,229,135, and 5,840,329. Other dosage forms suitable for use with the active agents of the present invention formulations are described in, for example, U.S. Pat. Nos. 4,871,549, 5,260,068, 5,260,069, 5,508,040, 5,567,441 and 5,837,284.

In some embodiments, the controlled release dosage form is pulsatile release solid oral dosage form comprising at least two groups of particles, each containing active agents of the present invention as described herein. The first group of particles provides a substantially immediate dose of the active agents of the present invention upon ingestion by a subject. The first group of particles can be either uncoated or comprise a coating and/or sealant. The second group of particles comprises coated particles, which may comprise from about 2% to about 75%, typically from about 2.5% to about 70%, or from about 40% to about 70%, by weight of the total dose of the active agents of the present invention in the formulation, in admixture with one or more binders.

Coatings for providing a controlled, delayed, or extended-release may be applied to a compound of Formulas I-XXII of the present invention or a pure enantiomer, pure diastereomer, diastereomerically enriched mixture, or enantiomerically enriched mixture thereof or to a core containing a halogenated benzofuran compound or pure enantiomer, pure diastereomer, diastereomerically enriched mixture, or enantiomerically enriched mixture thereof. The coating may comprise a pharmaceutically acceptable ingredient in an amount sufficient, for example, to provide an extended release from, for example, about 1 hours to about 7 hours following ingestion before release of a halogenated benzofuran compound or pure enantiomer, pure diastereomer, diastereomerically enriched mixture, or enantiomerically enriched mixture thereof, or any one of a compound of Formulas I-XXII. Suitable coatings include one or more differentially degradable coatings such as, by way of example only, pH-sensitive coatings (enteric coatings) such as acrylic resins (for example, Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, for example, ethylcellulose, or non-enteric coatings having variable thickness to provide differential release of the active agents of the present invention formulation.

Many other types of controlled/delayed/extended-release systems known to those of ordinary skill in the art and are suitable for use with the active agents of the present invention formulations described herein. Examples of such delivery systems include polymer-based systems, such as polylactic and polyglycolic acid, polyanhydrides and polycaprolactone, cellulose derivatives (for example, ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, for example, Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

### Systemic Formulations

The formulations of the present invention suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agents of the present invention can be dissolved at concentrations of >1 mg/ml using water-soluble beta cyclodextrins (for example, beta-sulfobutyl-cyclodextrin and 2-hydroxypropyl-betacyclodextrin. Proper fluidity can be maintained, for example, by the use of a coating such as a lecithin, by the maintenance of the required particle size in the case of dispersions, and by the use of surfactants.

The formulations of the present invention suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The active agents of the present invention in suspension formulations designed for extended-release via subcutaneous or intramuscular injection can avoid first-pass metabolism and lower dosages of the active agents of the present invention will be necessary to maintain plasma levels of about 50 ng/ml. In such formulations, the particle size of the active agents of the present invention particles and the range of the particle sizes of the active agents of the present invention particles can be used to control the release of the drug by controlling the rate of dissolution in fat or muscle.

In certain embodiments of the present invention, pharmaceutical compositions containing a compound of Formulas I-XXII of the present invention or pure enantiomer, pure diastereomer, diastereomerically enriched mixture, or enantiomerically enriched mixture thereof may be formulated into a dosage form suitable for parenteral use. For example, the dosage form may be a lyophilized powder, a solution, suspension (for example, depot suspension).

In other embodiments, pharmaceutical compositions containing a compound of Formulas I-XXII of the present invention or pure enantiomer, pure diastereomer, diastereomerically enriched mixture, or enantiomerically enriched mixture thereof may be formulated into a topical dosage form such as, but not limited to, a patch, a gel, a paste, a cream, an emulsion, liniment, balm, lotion, and ointment.

Another typical formulation employed in the methods of the present invention employs transdermal delivery devices (“patches”). Such transdermal patches may be used to provide continuous or discontinuous infusion of a halogenated benzofuran compound in controlled amounts. The construction and use of transdermal patches for the delivery of pharmaceutical agents is well known in the art. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. Indirect techniques may involve formulating the compositions to provide for drug latentiation by the conversion of hydrophilic drugs into lipid-soluble drugs or prodrugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

### Non-Limiting Examples of Formulations for Systemic Delivery

The examples below provide non-limiting embodiments of formulations, which may be used to deliver any of a compound described herein in enantiomerically enriched form, pure form or even a racemic mixture. Therefore, while the compounds below are specified, any desired purity form or compound may be used if it achieves the desired goal of treatment.

A dry powder inhaler formulation is prepared containing the following components:

| Ingredient | Weight % |
|---|---|
| A compound of one of Formulas I-XXII | 5 |
| Lactose | 95 |

The active mixture is mixed with the lactose and the mixture is added to a dry powder inhaling appliance.

Suppositories, each containing 25 mg of active ingredient are made as follows:

| Ingredient | Quantity (mg) |
|---|---|
| A compound of one of Formulas I-XXII | 25 |
| Saturated fatty acid glycerides | 2000 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Suspensions, each containing 50 mg of active ingredient per 5.0 ml dose are made as follows:

| Ingredient | Amount | |
|---|---|---|
| A compound of one of Formulas I-XXII | 50 | mg |
| Xanthan gum | 4 | mg |
| Sodium carboxymethyl cellulose (11%) | 50 | mg |
| Microcrystalline cellulose (89%) | 50 | mg |
| Sucrose | 1.75 | g |
| Sodium benzoate | 10 | mg |
| Flavor and Color | q.v. | |
| Purified water | To 5 | ml |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

An intravenous formulation may be prepared as follows:

| Ingredient | Amount | |
|---|---|---|
| A compound of one of Formulas I-XXII | 250 | mg |
| Isotonic saline | 1000 | ml |

A topical formulation may be prepared as follows:

| Ingredient | Amount (g) |
|---|---|
| A compound of one of Formulas I-XXII | 10 |
| Emulsifying Wax | 30 |
| Liquid Paraffin | 20 |
| White Soft Paraffin | To 100 |

The white soft paraffin is heated until molten. The liquid paraffin and emulsifying wax are incorporated and stirred until dissolved. The active ingredient is added and stirring is continued until dispersed. The mixture is then cooled until solid.

Sublingual or buccal tablets, each containing 20 mg of active ingredient, may be prepared as follows:

| Ingredient | Amount (mg/tablet) |
|---|---|
| A compound of one of Formulas I-XXII | 20 |
| Glycerol | 210.5 |
| Water | 143 |
| Sodium Citrate | 4.5 |
| Polyvinyl Alcohol | 26.5 |
| Polyvinylpyrrolidone | 15.5 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

A liquid formulation is prepared containing the following components:

| Ingredient | Quantity (units) | |
|---|---|---|
| A compound of one of Formulas I-XXII | 500 | mg |
| Propylene Glycol | 2 | ml |
| Glycerin | 2 | ml |

The active mixture is mixed and added to a liquid vaporization appliance.

### Pharmaceutically Acceptable Salts

A halogenated benzofuran compound is an amine and thus basic, and therefore, reacts with inorganic and organic acids to form pharmaceutically acceptable acid addition salts. In some embodiments, a halogenated benzofuran compound as free amines is oily and has decreased stability at room temperature. In this case it may be beneficial to convert the free amine to a pharmaceutically acceptable acid addition salt for ease of handling and administration because in some embodiments, the pharmaceutically acceptable salt is solid at room temperature.

A compound described herein, including an enantiomerically enriched mixture, may be administered if desired as a pharmaceutically acceptable salt or a salt mixture. A salt mixture may be useful to increase solubility of the active substances, to alter pharmacokinetics, or for controlled release or other objective. A salt mixture may comprise 2, 3, 4, 5, 6, or more pharmaceutically acceptable salts together to form a single composition.

Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like.

Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, finnarate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge et al. (1977) “Pharmaceutical Salts,” J. Pharm. Sci. 66:1-19.) Most typical pharmaceutically acceptable salts are those employing a hydrochloride anion.

### Prodrugs

One of ordinary skill would understand that a compound, pure enantiomer or enantiomerically enriched mixture of the invention shall also include the prodrugs thereof. Prodrugs are compounds that are metabolized or otherwise transformed inside the body to the active pharmacologic agent(s) of interest. Thus, prodrug will contain the “active” component (for example, a compound, pure enantiomer, pure diastereomer, diastereomerically enriched mixture, or enantiomerically enriched mixture thereof. Examples include N-alpha-acyloxyalkoxycarbonyl derivatives or addition of amino acids to the amine, which can be removed within the body by esterases or similar enzymes, but other prodrugs and precursors should be understood to be within the scope of the invention.

### Synthetic Approaches for Compounds of the Present Invention

Methods for synthesis of the compounds described herein and/or starting materials are either described in the art or will be readily apparent to the skilled artisan in view of general references well-known in the art (see, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the compounds of the invention.

Additional references include: Taniguchi et al. 2010. Journal of mass spectrometry, 45(12), 1473-1476; Shulgin & Shulgin. 1992. PiHKAL. A chemical love story, Transform Press, Berkeley CA; Glennon et al. 1986. J. Med. Chem., 29(2), 194-199; Nichols et al. 1991. J. Med. Chem., 34(1), 276-281; Kedrowski et al. 2007. Organic Letters, 9(17), 3205-3207; Heravi & Zadsirjan. 2016. Current Organic Synthesis, 13(6), 780-833; Keri et al. 2017. European J. Med. Chem., 138, 1002-1033; Perez-Silanes et al. 2001. J. Heterocyclic Chem, 38(5), 1025-1030; and references therein.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched (having either more (R)-enantiomer than (S)-enantiomer, or more (S)-enantiomer than (R)-enantiomer), racemic mixture, diastereomerically pure, diastereomerically enriched, or a stereoisomeric mixture. Enantiomerically enriched compounds may have an enantiomeric excess of one enantiomer of at least 60%, at least 65%, at least 70%, at least 75%, at least 80%, at least 85%, at least 90%, at least 95%, or at least 98%.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

Stereoisomers may include enantiomers, diastereomers, racemic mixtures, and combinations thereof. Such stereoisomers can be prepared and separated using conventional techniques, either by reacting enantiomeric starting materials, or by separating isomers of compounds disclosed herein.

Isomers may include geometric isomers. Examples of geometric isomers include cis isomers or trans isomers across a double bond. Other isomers are contemplated among the compounds of the present disclosure. The isomers may be used either in pure form or in admixture with other isomers of the structures of Formulas described herein.

Various methods are known in the art for preparing optically active forms and determining activity. Such methods include standard tests described herein and other similar tests which are well known in the art. Examples of methods that can be used to obtain optical isomers of the compounds according to the present disclosure include the following:
i) physical separation of crystals whereby macroscopic crystals of the individual enantiomers are manually separated. This technique may particularly be used if crystals of the separate enantiomers exist (i.e., the material is a conglomerate), and the crystals are visually distinct;
ii) simultaneous crystallization whereby the individual enantiomers are separately crystallized from a solution of the racemate, possible only if the latter is a conglomerate in the solid state;
iii) enzymatic resolutions whereby partial or complete separation of a racemate by virtue of differing rates of reaction for the enantiomers with an enzyme;
iv) enzymatic asymmetric synthesis, a synthetic technique whereby at least one step of the synthesis uses an enzymatic reaction to obtain an enantiomerically pure or enriched synthetic precursor of the desired enantiomer;
v) chemical asymmetric synthesis whereby the desired enantiomer is synthesized from an achiral precursor under conditions that produce asymmetry (i.e., chirality) in the product, which may be achieved using chiral catalysts or chiral auxiliaries;
vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors whereby the desired enantiomer is obtained from non-chiral starting materials and where the stereochemical integrity is not or is only minimally compromised over the course of the synthesis;
x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography whereby the racemate is volatilized and enantiomers are separated by virtue of their differing interactions in the gaseous mobile phase with a column containing a fixed non-racemic chiral adsorbent phase;
xii) extraction with chiral solvents whereby the enantiomers are separated by virtue of preferential dissolution of one enantiomer into a particular chiral solvent; and
xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane, which allows only one enantiomer of the racemate to pass through.

### Example 1: Synthesis Schemes

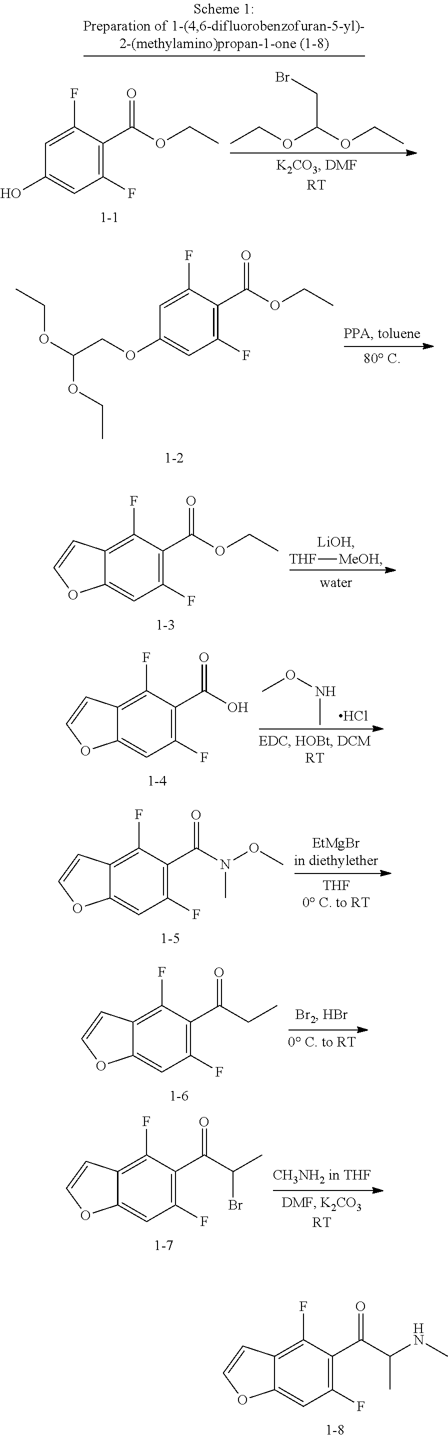

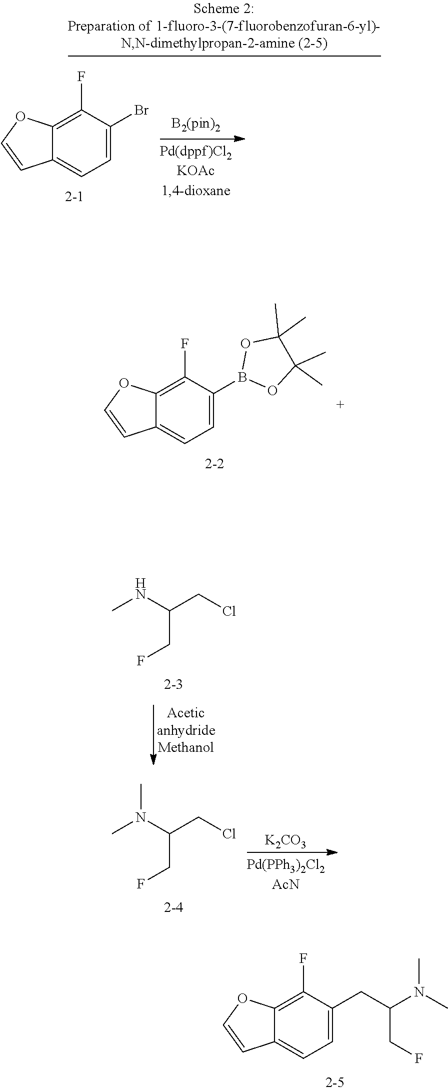

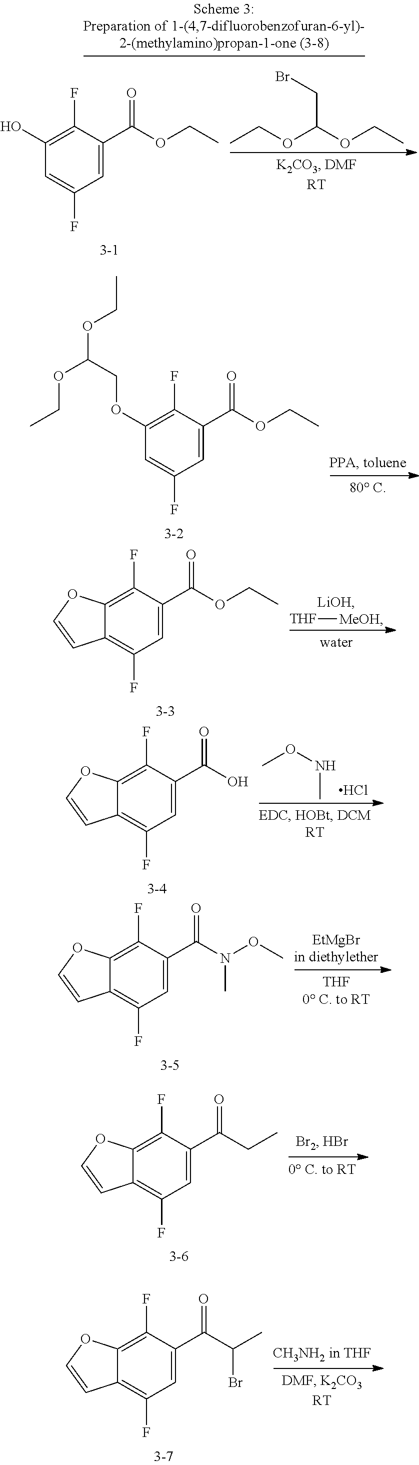

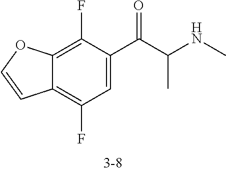 [SMILES: CNC(C)C(=O)c1cc(F)c2ccoc2c1F]

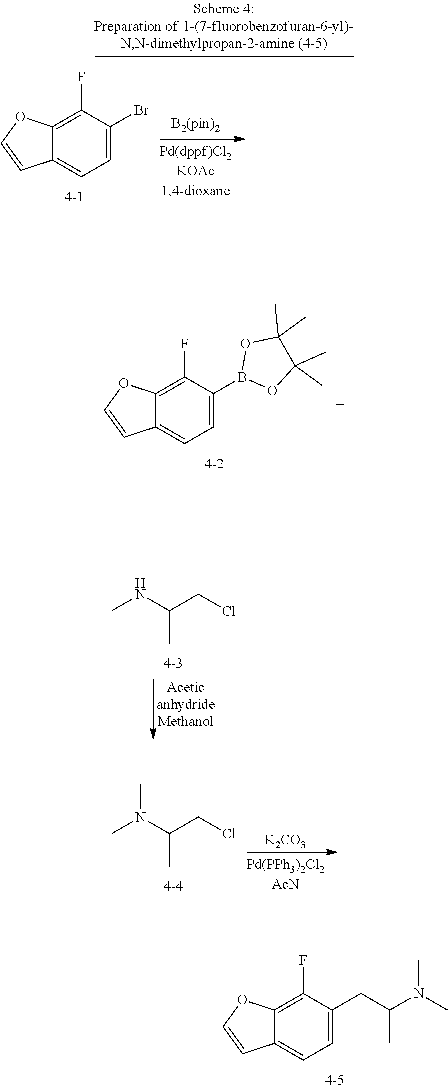

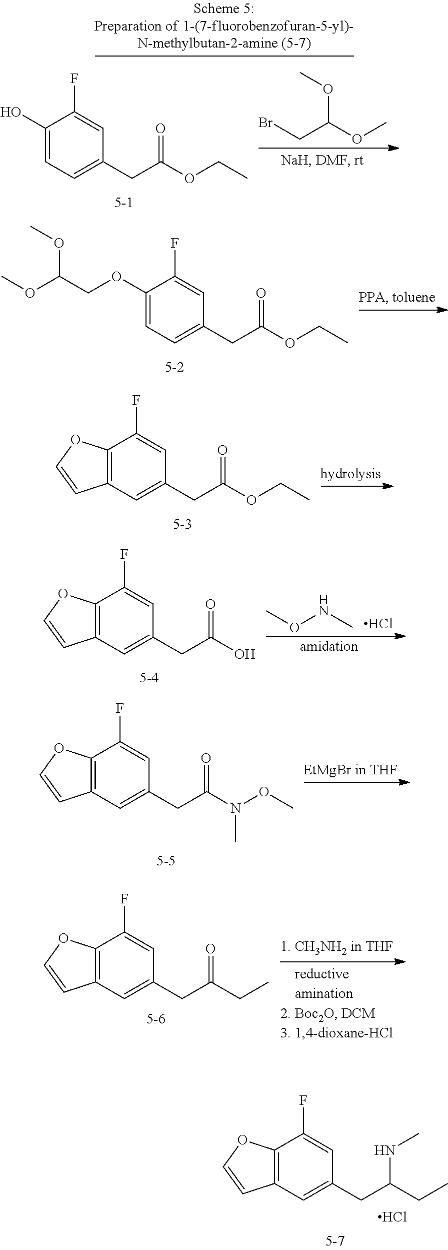

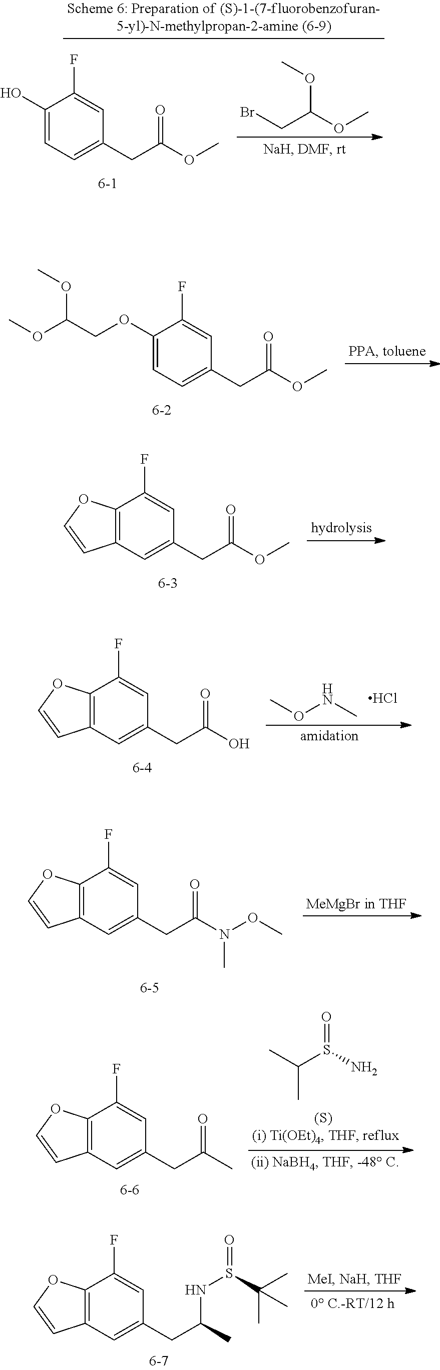

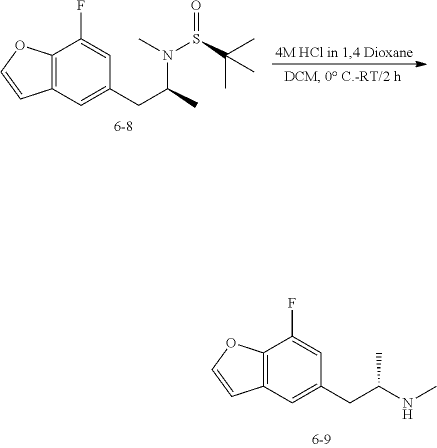 [SMILES: C[C@@H](Cc1cc(F)c2occc2c1)N(C)[S@@](=O)C(C)(C)C ; CN[C@@H](C)Cc1cc(F)c2occc2c1]

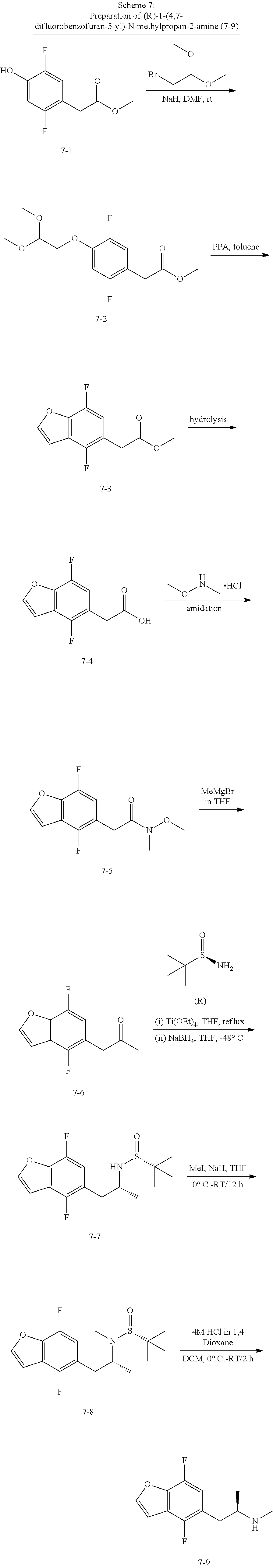

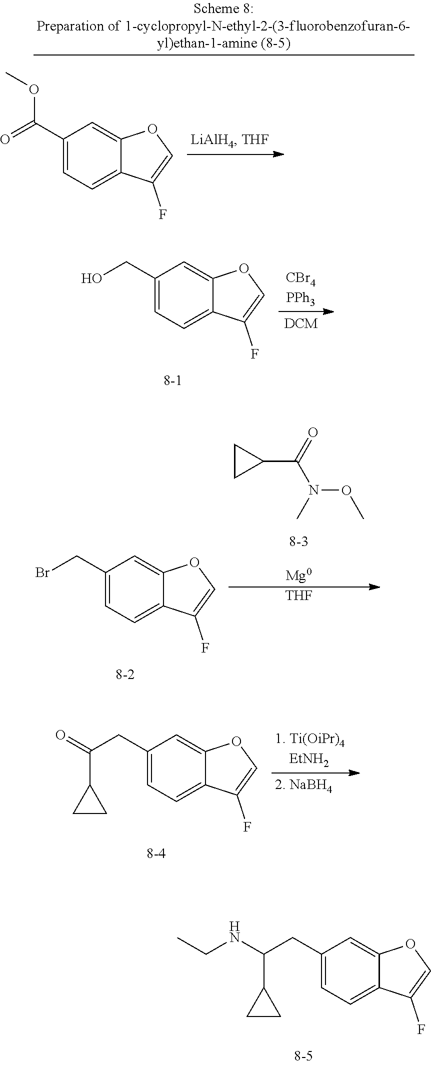

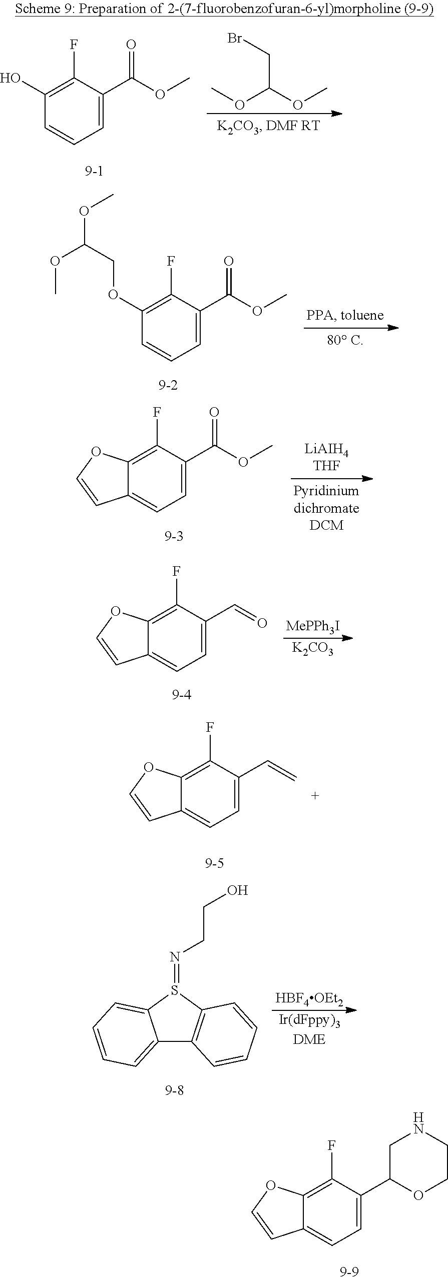

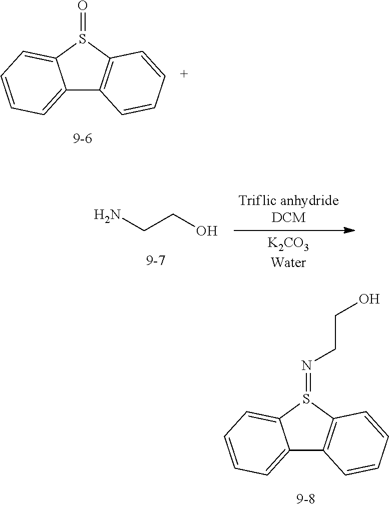

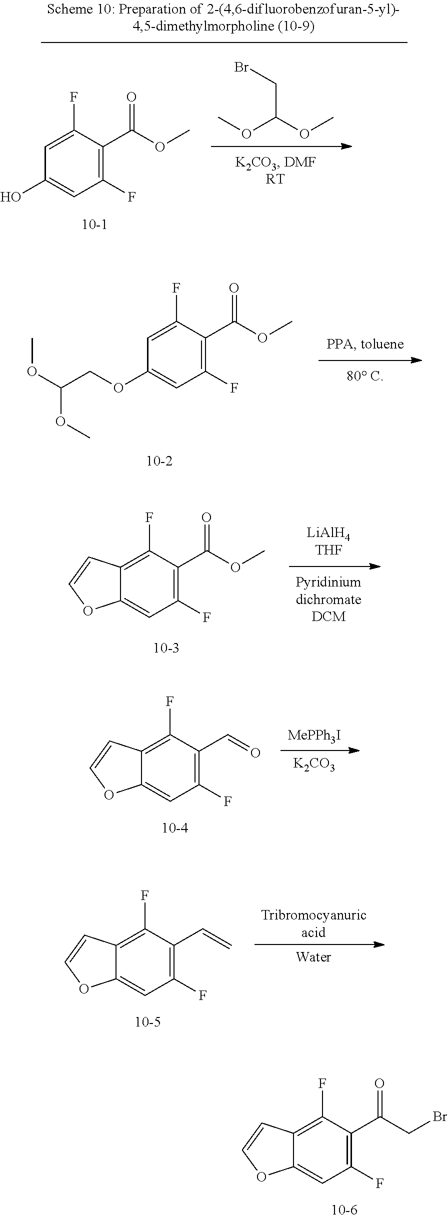

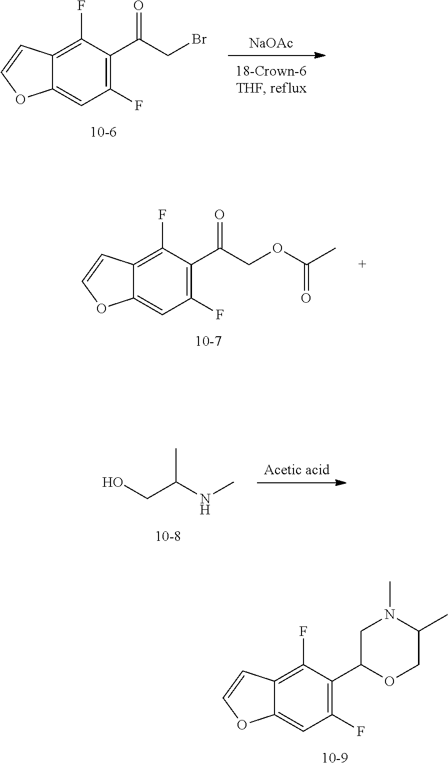

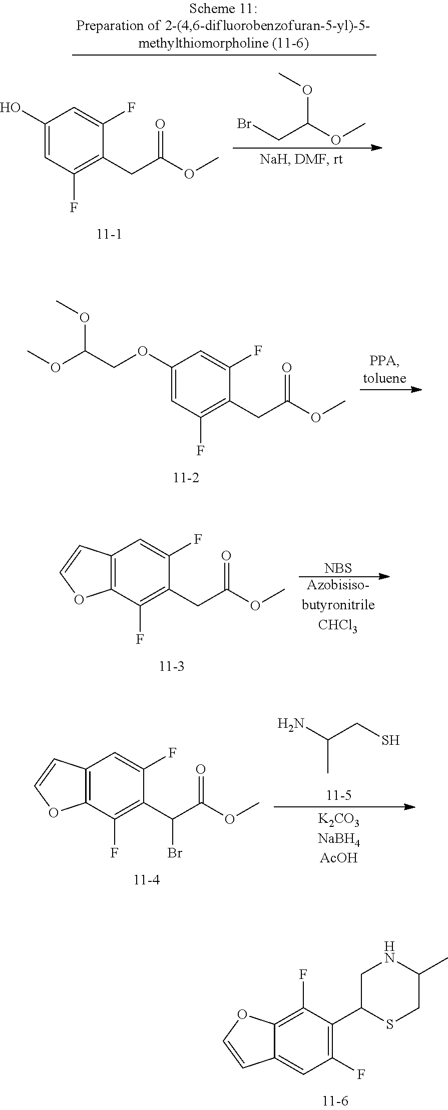

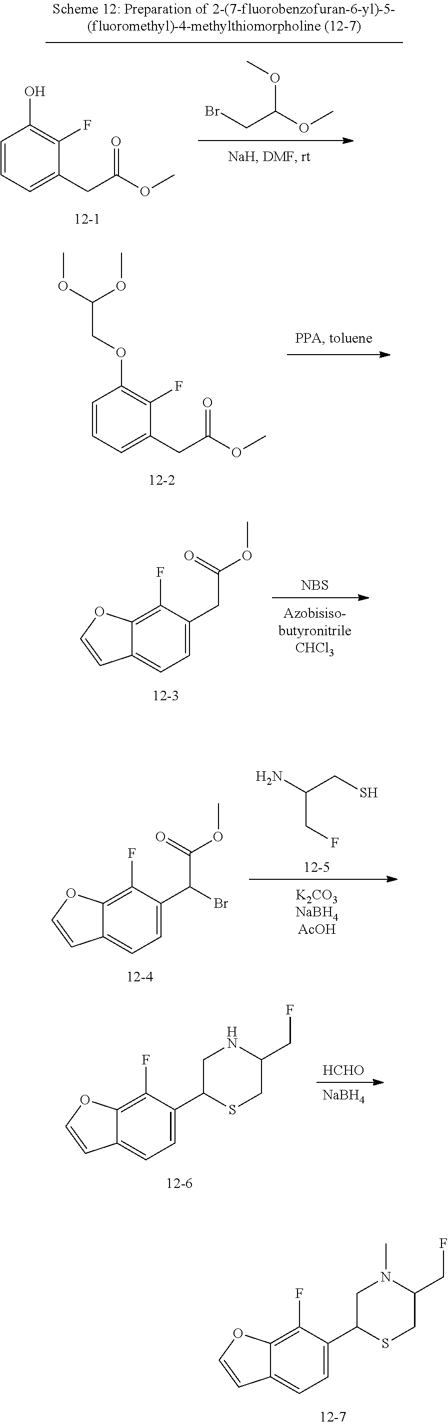

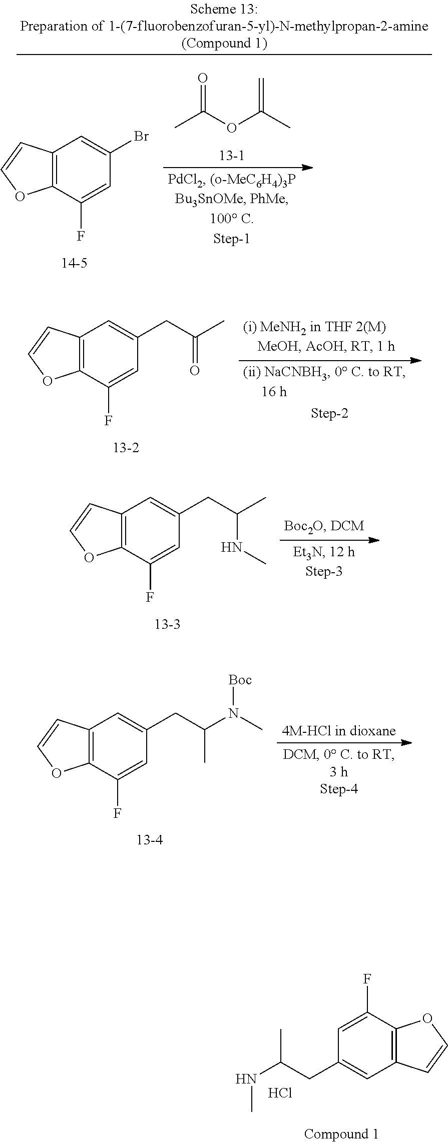

### Step-1:

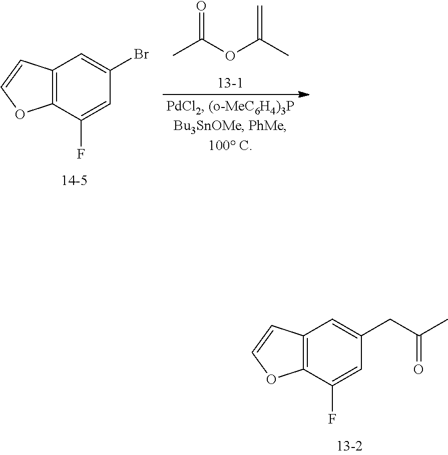

To a stirred solution of 5-bromo-7-fluorobenzofuran (14-5) (1 g, 4.67 mmol, 1 equiv.) in dry Toluene (20 ml) was added tri(o-tolyl)phosphine (142 mg, 0.467 mmol, 0.1 equiv.), tributyl tin methoxide (2.04 ml, 7 mmol, 1.5 equiv.) followed by isopropenyl acetate (0.81 ml, 7.47 mmol, 1.6 equiv.) and the resulting reaction mixture was degassed under argon for 10 minutes. Then palladium (II) chloride (58 mg, 0.32 mmol, 0.07 equiv.) was added to the reaction mixture and the resulting reaction mixture was heated to 100° C. for 16 hours. Upon completion, monitored by thin-layer chromatography (10% ethyl acetate in hexane), the reaction mixture was filtered through a celite bed, added water then extracted with ethyl acetate (2×100 ml). The organic layer was collected and stirred with saturated potassium fluoride solution for 1 hour. The organic layer was collected and washed with a brine solution. The combined organic layer was dried over anhydrous sodium sulphate. The Solvent was removed under vacuum and purified by silica gel column chromatography using ethyl acetate/hexane (5:95 v/v) as eluent to afford 1-(7-fluorobenzofuran-5-yl)propan-2-one (13-2) as a light yellow sticky gum (450 mg, 50%). ¹H NMR (400 MHz, CDCl₃) δ 7.64 (d, J=2 Hz, 1H), 7.19 (s, 1H), 6.89 (d, J=11.24 Hz, 1H), 6.76-6.74 (q, 1H), 3.75 (s, 2H), 2.17 (s, 3H). GCMS: Rt 9.382 min. MS (ES) C₁₁H₉FO₂ requires 192.06, found 192.2 [m/z].

### Step-2:

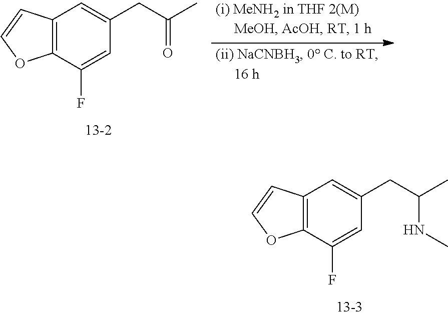

To a stirred solution of 1-(7-fluorobenzofuran-5-yl)propan-2-one (13-2) (1.2 g, 6.25 mmol, 1 equiv.) in dry methanol (20 ml) was added acetic acid (0.357 nil, 6.25 mmol, 1 equiv.) and methyl amine in tetrahydrofuran (2M) (6.25 ml, 12.5 mmol, 2 equiv.) (in a sealed Round bottom flask) and the resulting reaction mixture was allowed to stir at room temperature for 1 hour. Then NaCNBH₃ (0.78 g, 12.5 mmol, 2 equiv.) was added to the reaction mixture at 0° C. and the reaction mixture was allowed to stir at room temperature for 16 hours. Upon completion, monitored by thin-layer chromatography (20% ethyl acetate in hexane), the volatiles were removed under vacuum and the crude was extracted with ethyl acetate (2×100 ml), washed with water, followed by brine solution. The combined organic layer was dried over anhydrous sodium sulphate, solvent was removed under vacuum to afford crude 1-(7-fluorobenzofuran-5-yl)N-methylpropan-2-amine (13-3) as yellow sticky gum (1.2 g, 92%). Proceed to the next step with the crude without further purification. LCMS: Rt 1.34 min. MS (ES) C₁₂H₁₄FNO, requires 207.11, found 208.2 [M+H].

### Step-3:

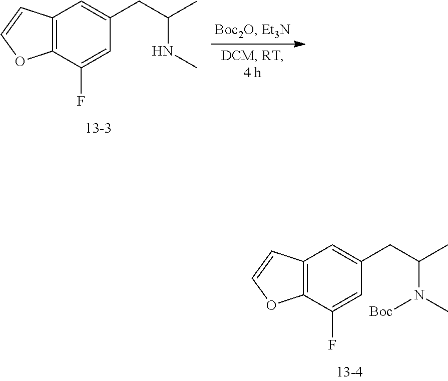 [SMILES: CNC(C)Cc1cc(F)c2occc2c1 ; CC(Cc1cc(F)c2occc2c1)N(C)C(=O)OC(C)(C)C]

To a stirred solution of crude 1-(7-fluorobenzofuran-5-yl)N-methylpropan-2-amine (13-3) (1.2 g, 4.97 mmol, 1 equiv.) in dry dichloromethane (20 ml) was added Triethylamine (1.4 ml, 9.95 mmol, 2 equiv.) and Boc anhydride (2.28 ml, 9.95 mmol, 2 equiv.) and the resulting reaction mixture was allowed to stir at room temperature for 4 hours. Upon completion, monitored by thin-layer chromatography (10% ethyl acetate in hexane), the reaction mixture was extracted with dichloromethane (2×50 ml), and washed with water, followed by brine solution. The organic layer was collected and dried over anhydrous sodium sulphate. Filtered and the solvent was evaporated under vacuum and purified by silica gel column chromatography using ethyl acetate/hexane (3:97 v/v) as eluent to afford tert-butyl (1-(7-fluorobenzofuran-5-yl)propan-2-yl)(methyl)carbamate (13-4) as a yellow sticky gum (900 mg, 58%). ¹H NMR (400 MHz, DMSO-d⁶) δ 8.03 (s, 1H), 7.24 (bs, 1H), 7.04-7.00 (d, J=16.4 Hz, 2H), 4.34-4.31 (m, 1H), 2.76-2.74 (m, 2H), 2.66 (s, 3H), 1.25 (d, J=13.6 Hz, 3H), 1.15-1.09 (m, 9H). LCMS: Rt 2.13 min. MS (ES) C₁₇H₂₂FNO₃, requires 307.16, found 308.3 [M+H]. Rotamer observed in NMR.

### Step-4:

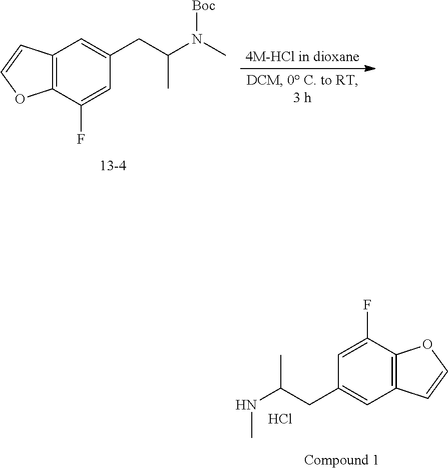 [SMILES: CC(Cc1cc(F)c2occc2c1)N(C)C(=O)OC(C)(C)C ; CNC(C)Cc1cc(F)c2occc2c1 ; Cl]

To a stirred solution of tert-butyl (1-(7-fluorobenzofuran-5-yl)propan-2-yl)(methyl)carbamate (13-4) (900 mg, 2.90 mmol, 1 equiv.) in dry dichloromethane (15 ml) was added (4M) HCl in 1,4 dioxane (8.17 ml, 32.7 mmol, 10 equiv.) at 0° C. and the resulting reaction mixture was allowed to stir at room temperature for 3 hours. Upon completion of reaction (monitored by thin-layer chromatography, 10% ethyl acetate in hexane), the solvent was evaporated, and the crude was washed twice with 1% methanol-diethyl ether (2×50 ml) and dried under vacuum to afford 1-(7-fluorobenzofuran-5-yl)N-methylpropan-2-amine hydrochloride (Compound 1) as an off white solid (690 mg, 86%). ¹HNMR (400 MHz, DMSO-d⁶) δ 8.92 (bs, 2H), 8.10 (s, 1H), 7.39 (s, 1H), 7.21 (d, J=12.08 Hz, 1H), 7.05 (s, 1H), 3.42 (bs, 1H), 3.25-3.21 (d, J=13.4 Hz, 1H), 2.80-2.74 (m, 1H), 2.56 (s, 3H), 1.13 (d, J=6.40 Hz, 3H). LCMS: Rt 1.96 min. MS (ES) C₁₂H₁₅ClFNO, requires 207.11, found 207.8 [M+H]. HPLC: Rt 6.19 min. Purity (λ 240 nm): 99.84%.

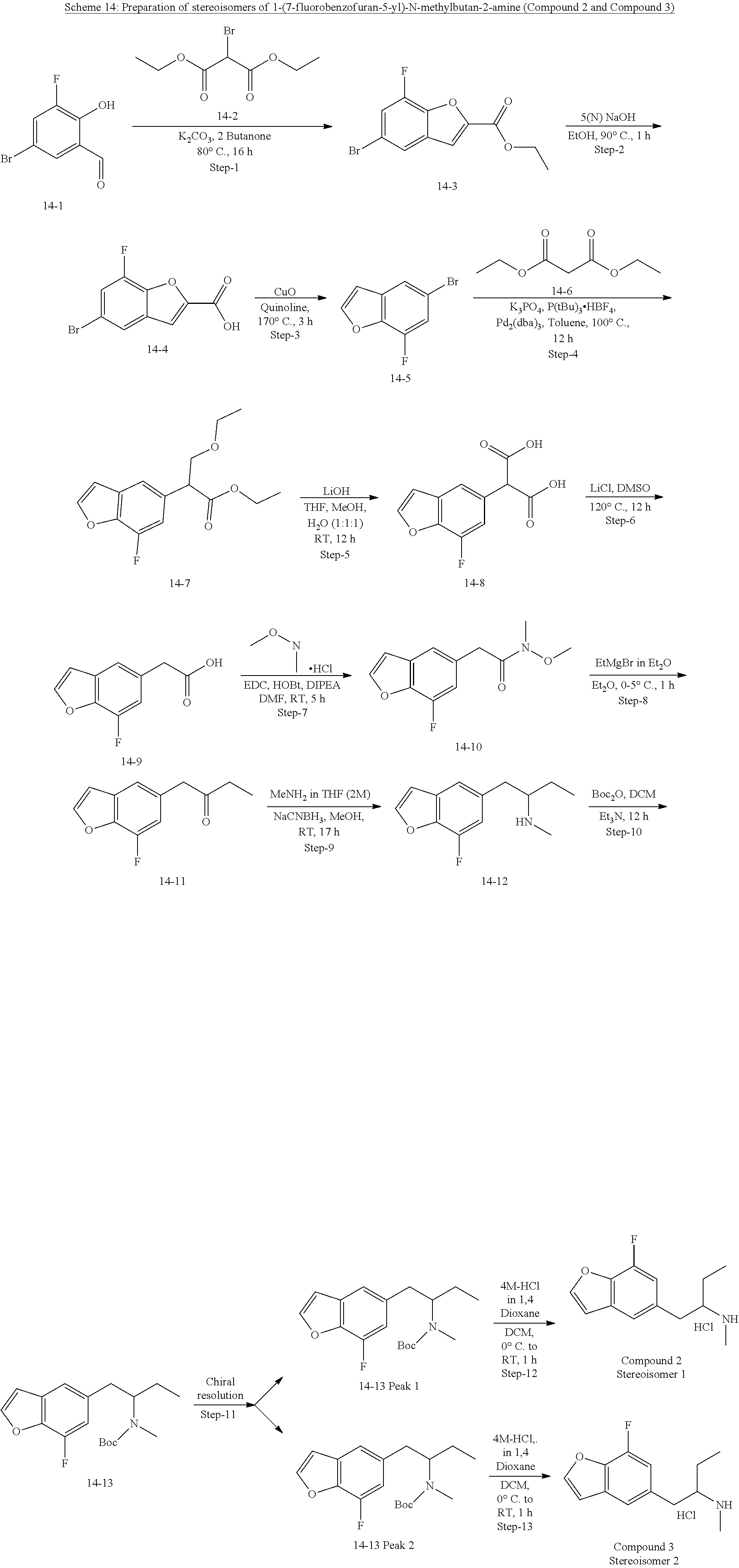

### Step-1:

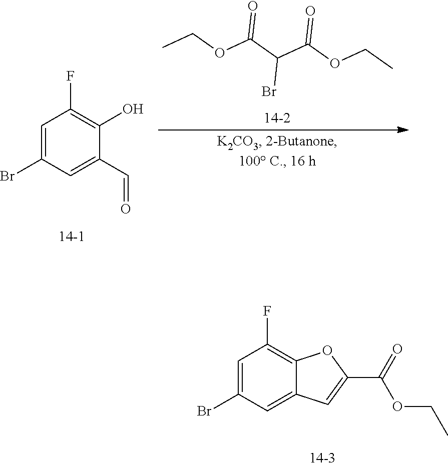 [SMILES: O=Cc1cc(Br)cc(F)c1O ; CCOC(=O)C(Br)C(=O)OCC ; CCOC(=O)c1cc2cc(Br)cc(F)c2o1]

To a stirred solution of 5-bromo-3-fluoro-2-hydroxybenzaldehyde (14-1) (15 g, 68.82 mmol, 1 equiv.) in 2-butanone (300 ml) were added K₂CO₃ (10.46 g, 75.70 nmol, 1.1 equiv.) and diethyl 2-bromonalonate (14-2) (14.1 ml, 82.59 mmol, 1.2 equiv.) then the resulting reaction mixture was heated to 100° C. for 16 hours. Upon completion, monitored by thin-layer chromatography (20% ethyl acetate in hexane), the reaction mixture was extracted with ethyl acetate (2×250 ml), and washed with water, followed by brine solution. The combined organic layer was dried over anhydrous sodium sulphate. Filtered and the solvent was removed under vacuum and purified by silica gel column chromatography using ethyl acetate/hexane (5:95 v/v) as eluent to afford ethyl-5-bromo-7-fluorobenzofuran-2-carboxylate (14-3) as off white solid (11.3 g, 57%). ¹H NMR (400 MHz, CDCl₃) δ 7.60 (s, 1H), 7.47 (s, 1H), 7.34 (d, J=9.6 Hz, 1H), 4.47-4.41 (m, 2H), 1.43-1.40 (t, J=7.04 Hz, 7 Hz, 3H). GCMS: Rt 10.29 min. MS (ES) C₁₁H₈BrFO₃ requires 285.96, found 286.1 [M+H].

### Step-2:

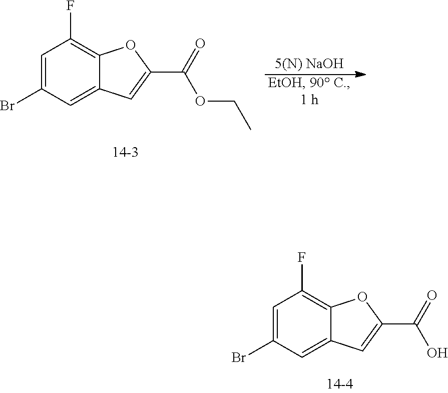

To a stirred solution of ethyl-5-bromo-7-fluorobenzofuran-2-carboxylate (14-3) (7.5 g, 34.96 mmol, 1 equiv.) in dry Ethanol (100 ml) was added (5N) NaOH (12 ml) and the resulting reaction mixture was allowed to stir at 90° C. for 1 h. Upon completion, monitored by thin-layer chromatography (20% ethyl acetate in hexane), the reaction mixture was acidified by (1N) HCl up to pH 3-4 and was extracted with ethyl acetate (2×150 ml). Then washed with water, followed by a brine solution. The combined organic layer was dried over anhydrous sodium sulphate. Filtered and the solvent was removed under vacuum to afford the crude 5-bromo-7-fluorobenzofuran-2-carboxylic acid (14-4) as an off-white solid (6 g, 88%). Proceed to the next step with the crude without further purification. ¹H NMR (400 MHz, DMSO-d⁶) δ 13.96 (bs, 1H), 7.87 (d, J=1.64 Hz, 1H), 7.76-7.73 (dd, J=1.68 Hz, 10.36 Hz, 1H), 7.71 (d, J=2.84 Hz, 1H). LCMS: Rt 1.34 min. MS (ES) C₉H₄BrFO₃, requires 257.93, found 256.80 [M−H].

### Step-3:

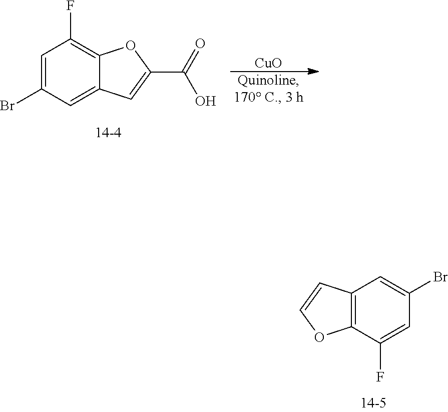 [SMILES: O=C(O)c1cc2cc(Br)cc(F)c2o1 ; "CuO" ; "Quinoline,170° C., 3 h" ; Fc1cc(Br)cc2ccoc12]

To a stirred solution of crude 5-bromo-7-fluorobenzofuran-2-carboxylic acid (14-4) (6 g, 23.16 mmol, 1 equiv.) in quinoline (60 ml) was added copper (11) oxide (1.84 g, 23.16 mmol, 1 equiv.) and the resulting reaction mixture was heated to 170° C. for 3 hours. Upon completion, monitored by thin layer chromatography (10% ethyl acetate in hexane), the reaction mixture was extracted with ethyl acetate (2×100 ml). Then washed with water, then (2N) HCl (twice), and finally with brine solution. The combined organic layer was dried over anhydrous sodium sulphate. Filtered and the solvent was removed under vacuum and purified by silica gel column chromatography using hexane as eluent to afford 5-bromo-7-fluorobenzofuran (14-5) as colorless sticky gum (3 g, 60%). ¹H NMR (400 MHz, CDCl₃) δ 7.65 (bs, 1H), 7.51 (bs, 1H), 7.20 (d, J=9.92 Hz, 1H), 6.75 (bs, 1H). GCMS: Rt 9.30 min. MS (ES) C₈H4BrFO requires 213.94, found 213.9 & 215.9 [M+H].

### Step-4:

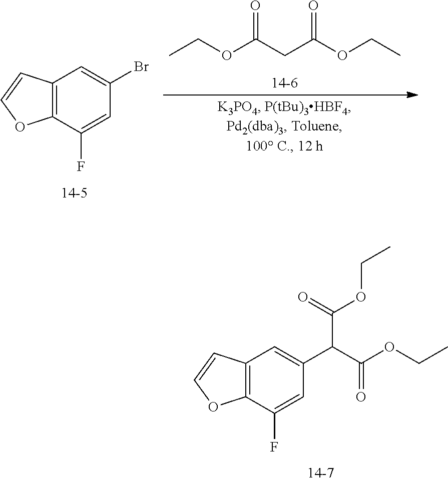

To a stirred solution of Diethyl malonate (14-6) (3.51 ml, 23.02 mmol, 1.1 equiv.) in dry toluene (100 ml) was added K₃PO₄ (13.32 g, 62.79 mmol, 3 equiv.) and the resulting reaction mixture was purged under nitrogen for 10 min. Then P(tBu)₃·HBF (0.607 g, 2.09 mmol, 0.1 equiv.) was added to the reaction mixture followed by the addition of 5-bromo-7-fluorobenzofuran (14-5) (4.5 g, 20.93 mmol, 1 equiv.) and Pd₂(dba)₃ (0.383 g, 0.41 mmol, 0.02 equiv.) at room temperature and the resulting reaction mixture was heated at 100° C. for 12 hours. Upon completion, monitored by thin-layer chromatography (20% ethyl acetate in hexane), the reaction mixture was extracted with ethyl acetate (2×150 ml), and washed with water, followed by brine solution. The combined organic layer was dried over anhydrous sodium sulphate. Filtered and the solvent was removed under vacuum and purified by silica gel column chromatography using ethyl acetate/hexane (10:90 v/v) as an eluent to afford diethyl 2-(7-fluorobenzofuran-5-yl)malonate (14-7) as yellowish sticky gum (5 g, 81%). ¹H NMR (400 MHz, DMSO-d⁶) δ 8.12 (d, J=2, 1H), 7.53 (s, 1H), 7.26 (d, J=12 Hz, 1H), 7.11-7.10 (m, 1H), 5.11 (s, 1H), 4.21-4.07 (m, 4H), 1.19-1.17 (m, 6H). LCMS: Rt 1.96 min. MS (ES) C₁₅H₁₅FO₅ requires 294, found 295.2 [M+H].

### Step-5:

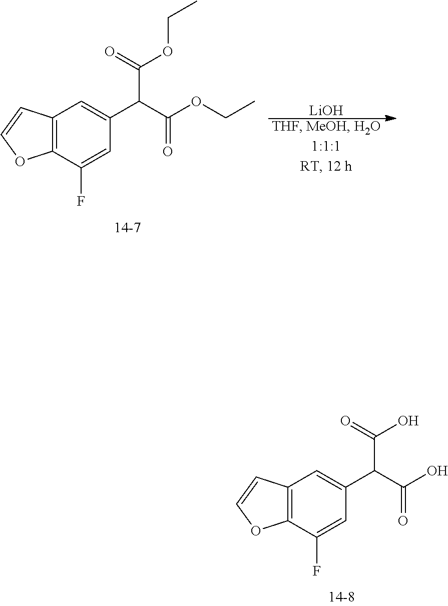 [SMILES: CCOC(=O)C(C(=O)OCC)c1cc(F)c2occc2c1 ; "LiOH" ; "THF, MeOH, H2O1:1:1RT, 12 h" ; O=C(O)C(C(=O)O)c1cc(F)c2occc2c1]

To a stirred solution of diethyl 2-(7-fluorobenzofuran-5-yl)malonate (14-7) (5 g, 17.00 mmol, 1 equiv.) in tetrahydrofuran:methanol (1:1) (50 ml each) was added LiOH·H₂O (3.57 g, 85.03 mmol, 5 equiv.) (dissolved in 50 ml water) at room temperature and the resulting reaction mixture was allowed to stir at same temperature for 12 hours. Upon completion, monitored by thin-layer chromatography (20% ethyl acetate in hexane), volatiles were evaporated to get the crude which was acidified with (2N) HCl up to pH 2 and extracted with 10% methanol-dichloromethane, washed with brine solution, dried over sodium sulphate. Filtered and the solvent was removed under vacuum to afford crude 2-(7-fluorobenzofuran-5-yl) malonic acid (14-8) as a yellowish solid (3.6 g, 88%). The next step was to proceed with this crude material without further purification. LCMS: Rt 1.10 min. MS (ES) C₁₁H₇FO₅ requires 238, found 237 [M−H].

### Step-6:

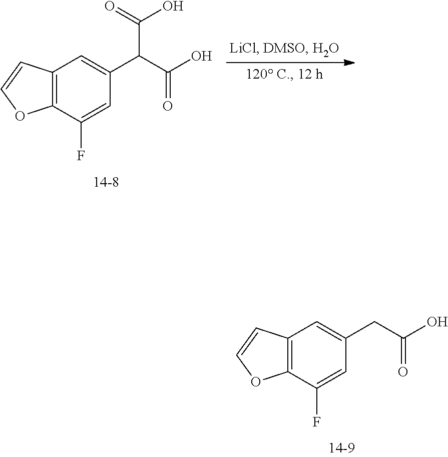 [SMILES: O=C(O)C(C(=O)O)c1cc(F)c2occc2c1 ; O=C(O)Cc1cc(F)c2occc2c1]

To a stirred solution of crude 2-(7-fluorobenzofuran-5-yl)malonic acid (14-8) (3.6 g, 15.12 mmol, 1 equiv.) in dry DMSO (30 ml) and H2 (3 ml) was added LiCl (2.56 g, 60.50 mmol, 4 equiv.) and the resulting reaction mixture was allowed to stir at 120° C. for 12 hours. Upon completion, monitored by thin-layer chromatography (5% methanol-dichloromethane) and crude LCMS, the reaction mixture was acidified up to pH-2 with (2N) HCl and extracted with ethyl acetate, washed with cold water (twice), followed by brine. The collected organic layer was dried over sodium sulphate, filtered and the solvent was removed under vacuum to afford crude 2-(7-fluorobenzofuran-5-yl) acetic acid (14-9) as a yellowish solid (2.3 g, 78%). The next step was to proceed with this crude without further purification ¹H NMR (400 MHz, DMSO-d⁶) δ 12.39 (s, 1H), 8.07 (s, 1H), 7.37 (s, 1H), 7.14 (d, J=12.12 Hz, 1H), 7.03 (s, 1H), 3.67 (s, 2H). LCMS: Rt 1.23 min. MS (ES) C₁₀H₇FO₃ requires 194, found 192.7 [M−H].

### Step-7:

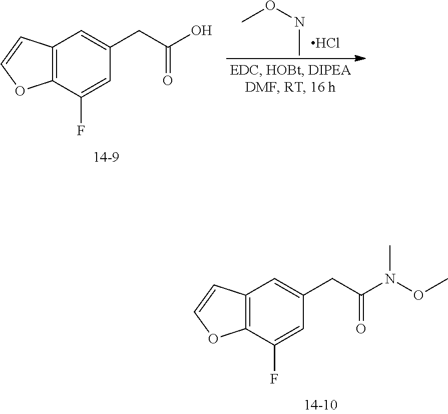

To a stirred solution of 2-(7-fluorobenzofuran-5-yl)acetic acid (14-9) (4.7 g, 24.22 mmol, 1 equiv.) in dry dichloromethane (100 ml) was added N,N-diisopropylethylamine (12.67 ml, 72.68 mmol, 3 equiv.), followed by N-(3-dimethylaminopropyl)-N′-ethylcarbodiimide hydrochloride (5.10 g, 26.64 mmol, 1.1 equiv.) and HOBT (4.90 g, 36.34 mmol, 1.5 equiv.) under N₂ atmosphere and the resulting reaction mixture was allowed to stir at the same temperature for 30 min. Then N, O-dimethylhydroxylamine hydrochloride (2.59 g, 26.64 mmol, 1.1 equiv.) was added to the resulting reaction mixture and was allowed to stir for 16 hours. Upon completion, monitored by thin-layer chromatography (50% ethyl acetate in hexane), the reaction mixture was extracted with dichloromethane twice (2×100 ml) and washed with water followed by brine solution. The combined organic layer was dried over anhydrous sodium sulphate. Filtered and the solvent was removed under vacuum and purified by silica gel column chromatography using ethyl acetate/hexane (30:70 v/v) as an eluent to afford 2-(7-fluorobenzofuran-5-yl)-N-methoxy-n-methylacetamide (14-10) as yellow sticky gum (3.5 g, 60%). ¹H NMR (400 MHz, DMSO-d⁶) δ 8.06 (s, 1H), 7.35 (s, 1H), 7.11 (d, J=12.16 Hz, 1H), 7.03 (s, 1H), 3.82 (s, 2H), 3.70 (s, 3H), 3.11 (s, 3H). LCMS: Rt 1.67 min. MS (ES) C₁₂12FNO₃ requires 237, found 238 [M+H].

### Step-8:

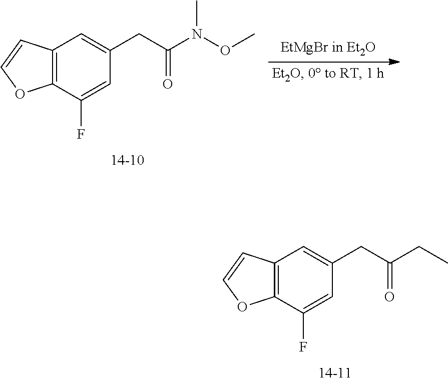 [SMILES: CON(C)C(=O)Cc1cc(F)c2occc2c1 ; CCC(=O)Cc1cc(F)c2occc2c1]

To a stirred solution of 2-(7-fluorobenzofuran-5-yl)-N-methoxy-n-methylacetamide (14-10) (3.5 g, 14.76 mmol, 1 equiv.) in dry diethyl ether (100 ml) was added 3 (M) solution of EtMgBr in diethylether (9.84 ml, 29.53 mmol, 2 equiv.) at 0° C. and the resulting reaction mixture was allowed to stir at room temperature for 1 hour. Upon completion, (monitored by thin-layer chromatography, 20% ethyl acetate in hexane) the reaction was quenched with saturated NH₄Cl solution and extracted with ethyl acetate, twice (2×150 ml), washed with water followed by brine solution. The combined organic layer was dried over anhydrous sodium sulphate. Filtered and the solvent was evaporated under a vacuum to get crude 1-(7-fluorobenzofuran-5-yl)butan-2-one (14-11) as pale yellow solid (3 g, 98%). The next step was to proceed with this crude without further purification. ¹H NMR (400 MHz, DMSO-d⁶) δ 8.07 (d, J=2.08, 1H), 7.29 (s, 1H), 7.07-7.02 (m, 2H), 3.85 (s, 2H), 2.58-2.50 (m, 2H), 0.94-0.92 (t, J=3.76 Hz, 3.52 Hz, 3H). GCMS: Rt 10.51 min. MS (ES) C₁₂H₁₁FO₂ requires 206, found 206.1 [m/z].

### Step-9:

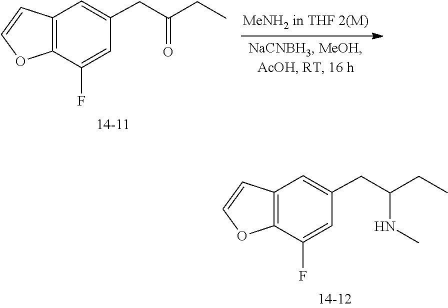 [SMILES: CCC(=O)Cc1cc(F)c2occc2c1 ; CCC(Cc1cc(F)c2occc2c1)NC]

To a stirred solution of crude 1-(7-fluorobenzofuran-5-yl)butan-2-one (14-11) (3 g, 14.56 mmol, 1 equiv.) in dry methanol (50 ml) was added acetic acid (0.83 ml, 14.56 mmol, 1 equiv.) and Methyl Amine in tetrahydrofuran (2M) (14.56 ml, 29.12 mmol, 2 equiv.) (in a sealed Round bottom flask) and the resulting reaction mixture was allowed to stir at room temperature for 1 hour. Then NaCNBH₃ (1.83 g, 29.12 mmol, 2 equiv.) was added to the reaction mixture at 0° C. and it was allowed to stir at room temperature for 16 hours. Upon completion, monitored by thin-layer chromatography (20% ethyl acetate in hexane), the volatiles were removed under vacuum and the crude was extracted with ethyl acetate (2×50 ml), washed with water, followed by the brine solution. The combined organic layer was dried over anhydrous sodium sulphate, filtered and the solvent was removed under vacuum to afford the crude 1-(7-fluorobenzofuran-5-yl)N-methylbutan-2-amine (14-12) as a yellow sticky gum (3 g, 93%). The next step was to proceed with this crude without further purification. LCMS: Rt 1.43 min. MS (ES) C₁₃H₁₆FNO, requires 221, found 222 [M+H]+.

### Step-10:

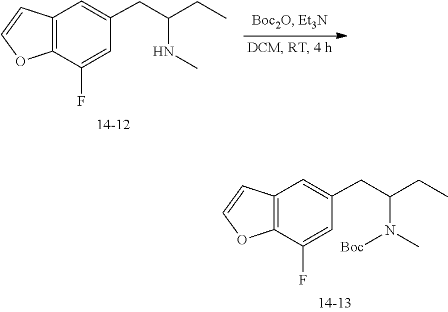 [SMILES: CCC(Cc1cc(F)c2occc2c1)NC ; CCC(Cc1cc(F)c2occc2c1)N(C)C(=O)OC(C)(C)C]

To a stirred solution of crude 1-(7-fluorobenzofuran-5-yl)N-methylbutan-2-amine (14-12) (3 g, 13.57 mmol, 1 equiv.) in dry dichloromethane (50 ml) was added Triethylamine (3.81 ml, 27.14 mmol, 2 equiv.) and Boc anhydride (6.23 ml, 27.14 mmol, 2 equiv.). The resulting reaction mixture was allowed to stir at room temperature for 4 hours. Upon completion, monitored by thin-layer chromatography (10% ethyl acetate in hexane), the reaction mixture was extracted with dichloromethane (2×100 ml), and washed with water, followed by brine solution. The combined organic solution was dried over anhydrous sodium sulphate and filtered. The solvent was evaporated under vacuum and purified by silica gel column chromatography using ethyl acetate/hexane (4:96 v/v) as eluent to afford tert-butyl (1-(7-fluorobenzofuran-5-yl) butan-2-yl)(methyl)carbamate (14-13) as a yellow sticky gum (2 g, 46%). LCMS: Rt 6.94 min. MS (ES) C₁₈H₂₄FNO₃ requires 321, found 322 [M+H].

### Step-11:

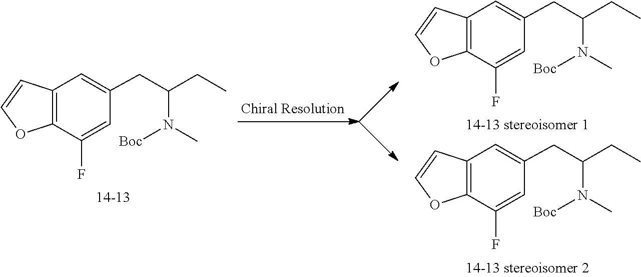

Isomeric separation of 14-13 was done by Chiral-HPLC. The method of chiral separation mentioned below was used.
Column Name—Chiralpak AY-H (4.6×250 mm), 5μ
Flow rate—1 mL/min.
Mobile phase—Hexane/EtOH/IP AMINE: 80/20/0.1
Solubility—Methanol
Wavelength −240 nm

1.5 g of 14-13 was submitted and after chiral resolution ˜650 mg of 14-13 stereoisomer 1 (peak 1) and ˜550 mg of 14-13 stereoisomer 2 (peak 2) were obtained. ˜200 mg of racemic mixture was recovered. Peak 1 was obtained at 3.77 min. Peak 2 was obtained at 4.63 min.

### Step-12:

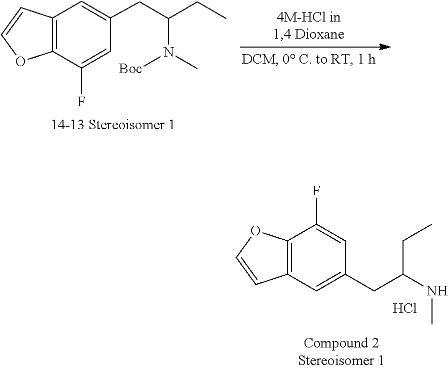 [SMILES: CCC(Cc1cc(F)c2occc2c1)N(C)C(=O)OC(C)(C)C ; CCC(Cc1cc(F)c2occc2c1)NC ; Cl]

To a stirred solution of 14-13 stereoisomer 1 (650 mg, 2.02 mmol, 1 equiv.) in dry dichloromethane (15 ml) was added (4M) HCl in 1,4 dioxane (10.1 ml, 40.4 mmol, 20 equiv.) at 0° C. and the resulting reaction mixture was allowed to stir at room temperature for 2 hours. Upon completion of reaction (monitored by thin-layer chromatography, 10% methanol-dichloromethane), the solvent was evaporated, and the crude was washed twice with 1% methanol-diethyl ether (2×30 ml) and dried under vacuum to afford Compound 2 as an off white solid (440 mg, 84%). ¹HNMR (400 MHz, DMSO-d⁶) δ 8.74 (bs, 2H), 8.10 (d, J=2.04, 1H), 7.42 (s, 1H), 7.24 (d, J=12.08, 1H), 7.06 (t, J=2.32 Hz, 2.76 Hz, 1H), 3.38-3.35 (m, 1H), 3.15-3.10 (m, 1H), 2.93-2.88 (m, 1H), 2.55 (s, 3H), 1.59-1.52 (m, 2H), 0.92-0.89 (t, J=7.44 Hz, 7.48 Hz, 3H). LCMS: Rt 2.00 min. MS (ES) C₁₃H₁₇ClFNO, requires 221, found 222 [M+H]. HPLC: Rt 6.01 min. Purity (λ 210 nm): 95.01%. Chiral purity: 97.05%; ee: 94.10%; optical rotation 0.044°; specific rotation (calculated) 17.564°.

### Step-13:

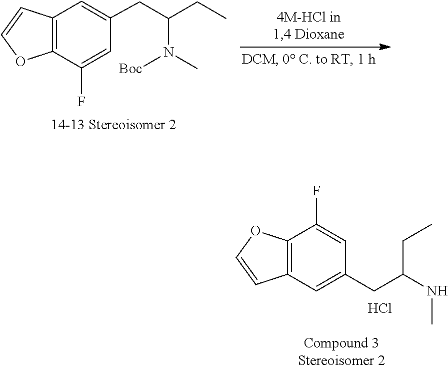 [SMILES: CCC(Cc1cc(F)c2occc2c1)N(C)C(=O)OC(C)(C)C ; CCC(Cc1cc(F)c2occc2c1)NC ; Cl]

To a stirred solution of 14-13 stereoisomer 2 (500 mg, 1.55 mmol, 1 equiv.) in dry dichloromethane (15 ml) was added (4M) HCl in 1,4 dioxane (7.75 ml, 31 mmol, 20 equiv.) at 0° C. and the resulting reaction mixture was allowed to stir at room temperature for 2 hours. Upon completion of reaction (monitored by thin-layer chromatography, 10% methanol-dichloromethane), the solvent was evaporated, and the crude was washed twice with 1% methanol-diethyl ether (2×30 ml) and dried under vacuum to afford (Compound 3) as an off white solid (330 mg, 82%). ¹HNMR (400 MHz, DMSO-d⁶) δ 8.77 (bs, 2H), 8.10 (d, J=1.56, 1H), 7.42 (s, 1H), 7.24 (d, J=12.04, 1H), 7.05 (s, 1H), 3.36 (m, 1H), 3.16-3.11 (m, 1H), 2.93-2.88 (m, 1H), 2.55 (s, 3H), 1.58-1.53 (m, 2H), 0.92-0.89 (t, J=7.4 Hz, 7.44 Hz, 3H). LCMS: Rt 1.44 min. MS (ES) C₁₃H₁₇ClFNO, requires 221, found 222 [M+H]. HPLC: Rt 6.22 min. Purity (λ 210 nm): 99.82%. Chiral purity: 99.86%; ee: 99.72%; optical rotation −0.038°; specific rotation (calculated) −16.065°.

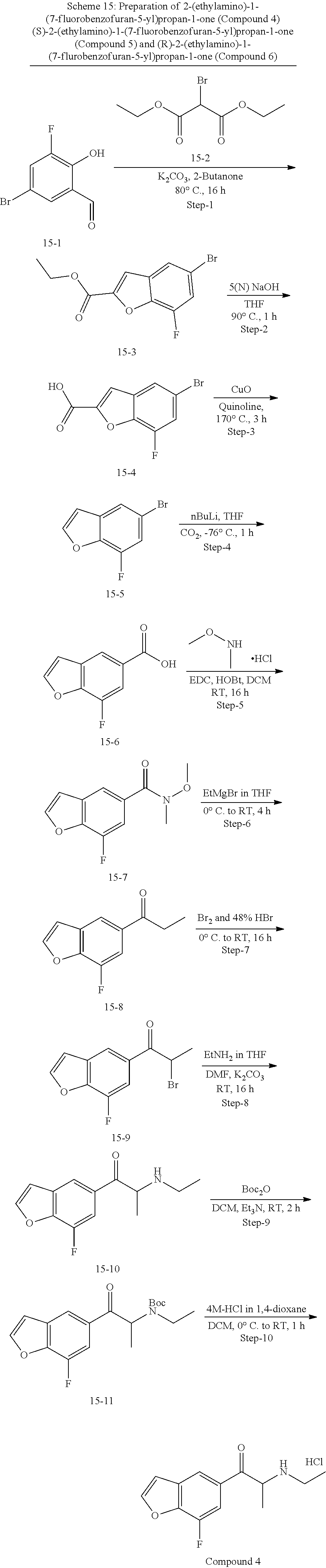

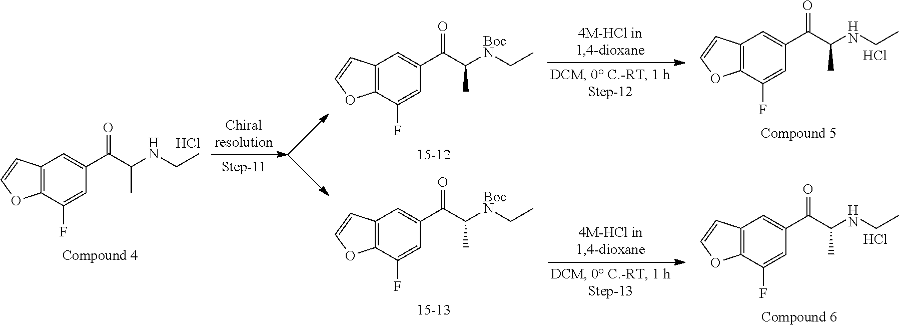

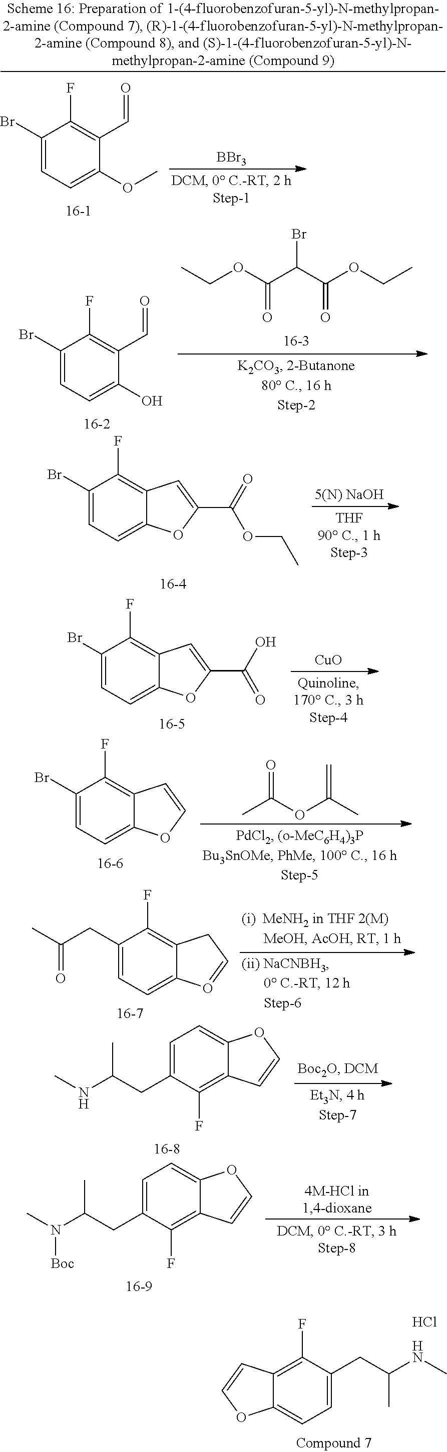

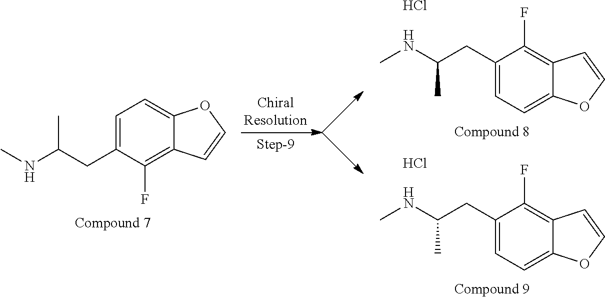

### Step-1:

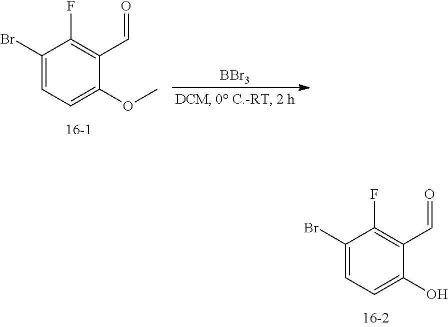

To a stirred solution of 3-bromo-2-fluoro-6-methoxybenzaldehyde (16-1) (10 g, 42.91 mmol, 1 equiv.) in dry dichloromethane (150 ml) was added BBr₃ (1.0 M) in dichloromethane (6436 ml, 64.36 mm ol, 1.5 equiv.) dropwise at 0° C. and the resulting reaction mixture was allowed to stir at room temperature for 2 hours. Upon completion, monitored by thin-layer chromatography (10% ethyl acetate in hexane), the reaction mixture was extracted with dichloromethane (2×200 ml), and washed with water, followed by brine solution. The combined organic layer was dried over anhydrous sodium sulphate. The solvent was removed under vacuum to afford crude 3-bromo-2-fluoro-6-hydroxybenzaldehyde (16-2) as off white solid (9 g, 95%). Proceed for the next with the crude without further purification. ¹H NMR (400 MHz, DMSO-d₆) δ 11.34 (s, 1H), 10.22 (s, 1H), 7.79-7.75 (t, J=8.48 Hz, 8.44 Hz, 1H), 6.83 (d, J=9 Hz, 1H). GCMS: Rt 8.297 min. MS (ES) C₇H₄BrFO₂ requires 217.94, found 218 [m/z].

### Step-2:

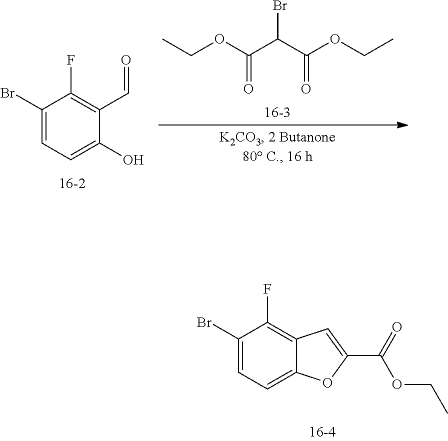

To a stirred solution of crude 3-bromo-2-fluoro-6-hydroxybenzaldehyde (16-2) (9 g, 41.09 mmol, 1 equiv.) in 2-butanone (150 ml) was added K₂CO₃ (14.19 g, 102.73 mmol, 2.5 equiv.) and diethyl 2-bromomalonate (16-3) (7.01 ml, 41.09 mmol, 1 equiv.) and the resulting reaction mixture was heated to 100° C. for 16 hours. Upon completion, monitored by thin-layer chromatography (10% ethyl acetate in hexane), the reaction mixture was extracted with ethyl acetate (2×200 ml), and washed with water, followed by brine solution. The combined organic layer was dried over anhydrous sodium sulphate. The solvent was removed under vacuum to afford the crude ethyl-5-bromo-4-fluorobenzofuran-2-carboxylate (16-4) as off white solid (10.5 g, 89%). Proceed for the next step with the crude without further purification. ¹H NMR (400 MHz, DMSO-d⁶) δ 7.89 (s, 1H), 7.82-7.78 (t, J=8.48 Hz, 7.12 Hz, 1H), 7.65 (d, J=8.84 Hz, 1H), 4.40-4.35 (q, 2H), 1.35-0.132 (t, J=6.96 Hz, 7.12 Hz, 3H). GCMS: Rt 7.55 min. MS (ES) C₁₁H₈BrFO3 requires 285.96, found 286.1 [m/z].

### Step-3:

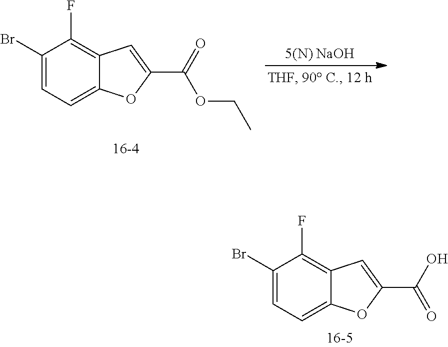

To a stirred solution of crude ethyl-5-bromo-4-fluorobenzofuran-2-carboxylate (16-4) (10.5 g, 36.57 mmol, 1 equiv.) in dry tetrahydrofuran (100 ml) was added 5(N) NaOH (50 ml) and the resulting reaction mixture was allowed to stir at 90° C. for 12 hours. Upon completion, monitored by thin-layer chromatography (10% ethyl acetate in hexane), the reaction mixture was acidified by (1N) HCl up to pH 3-4 and was extracted with ethyl acetate (2×200 ml), washed with water, followed by brine solution. The combined organic layer was dried over anhydrous sodium sulphate. The solvent was removed under vacuum to afford the crude 5-bromo-4-fluorobenzofuran-2-carboxylic acid (16-5) as off white solid (8.5 g, 89%). Proceed to the next step with the crude without further purification. ¹H NMR (400 MHz, DMSO-d₆) δ 13.93-13.90 (bs, 1H), 7.78-7.75 (m, 2H), 7.61 (d, J=8.84 Hz, 1H). LCMS: Rt 1.36 min. MS (ES) C₉H₄BrFO₃, requires 257.93, found 256.80 [M−H]⁻.

### Step-4:

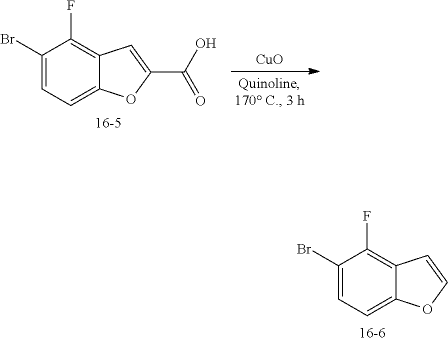 [SMILES: O=C(O)c1cc2c(F)c(Br)ccc2o1 ; "CuO" ; Fc1c(Br)ccc2occc12]

To a stirred solution of crude 5-bromo-4-fluorobenzofuran-2-carboxylic acid (16-5) (1.7 g, 6.56 mmol, 1 equiv.) in Quinoline (15 mL) was added copper (11) oxide (0.522 g, 6.56 mmol, 1 equiv.) and the resulting reaction mixture was heated to 170° C. for 3 hours. Upon completion, monitored by thin-layer chromatography (10% ethyl acetate in hexane), the reaction mixture was extracted with ethyl acetate (2×50 ml), washed with water, and then washed with (2N) HCl (twice), followed by brine solution. The combined organic layer was dried over anhydrous sodium sulphate. The solvent was removed under vacuum and purified by silica gel column chromatography using hexane as eluent to afford 5-bromo-4-fluorobenzofuran (16-6) as a light-yellow sticky gum (900 mg, 63%). ¹H NMR (400 MHz, DMSO-d⁶) δ 8.12 (s, 1H), 7.60-7.57 (t, J=8.04 Hz, 7.40 Hz, 1H), 7.51 (d, J=8.72 Hz, 1H), 7.14 (s, 1H). GCMS: Rt 8.38 min. MS (ES) C₈H₄BrFO requires 213.94, found 214.1 [m/z].

### Step-5:

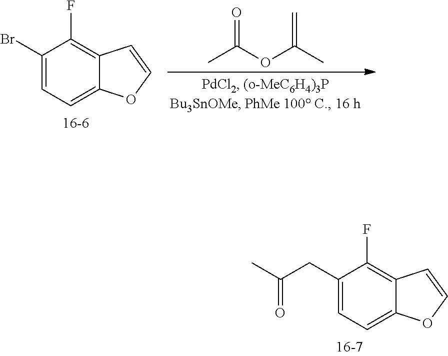

To a stirred solution of 5-bromo-4-fluorobenzofuran (16-6) (900 mg, 4.18 mmol, 1 equiv.) in dry Toluene (20 ml) was added tri(o-tolyl)phosphine (127.24 mg, 0.41 mmol, 0.1 equiv.), tributyl tin methoxide (1.83 ml, 6.27 mmol, 1.5 equiv.) followed by Isopropenyl acetate (0.698 ml, 6.27 mmol, 1.5 equiv.) and the resulting reaction mixture was degassed under argon for 10 minutes. Then palladium (II) chloride (74.22 mg, 0.41 mmol, 0.1 equiv.) was added to the reaction mixture and the resulting reaction mixture was heated to 100° C. for 16 hours. Upon completion, monitored by thin-layer chromatography (10% ethyl acetate in hexane), the reaction mixture was filtered through a celite bed, added water then extracted with ethyl acetate (2×100 ml). The organic layer was collected and stirred with saturated potassium fluoride solution for 1 hour. The organic layer was collected and washed with brine solution. The combined organic layer was dried over anhydrous sodium sulphate. The Solvent was removed under vacuum and purified by silica gel column chromatography using ethyl acetate/hexane (5:95 v/v) as eluent to afford 1-(4-fluorobenzofuran-5-yl)propan-2-one (16-7) as a light yellow sticky gum (400 mg, 49%). ¹H NMR (400 MHz, DMSO-d₆) δ 8.03 (s, 1H), 7.43 (d, J=8.16 Hz, 1H), 7.19-7.15 (t, J=7.2 Hz, 1H), 7.04 (s, 1H), 3.91 (s, 2H), 2.18 (d, J=1.28 Hz, 3H). GCMS: Rt 9.25 min. MS (ES) C₁₁H₉FO₂ requires 192.06, found 192.1 [m/z].

### Step-6:

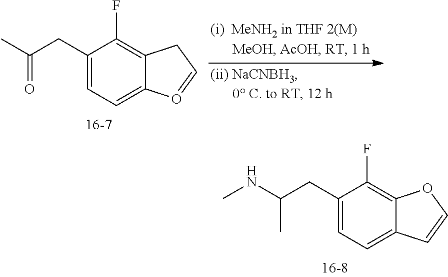

To a stirred solution of 1-(4-fluorobenzofuran-5-yl)propan-2-one (16-7) (500 mg, 2.6 mmol, 1 equiv.) in dry methanol (10 ml) was added acetic acid (0.15 ml, 2.6 mmol, 1 equiv.) and Methyl Amine in tetrahydrofuran (2M) (2.6 ml, 5.20 mmol, 2 equiv.) (in a sealed round bottom flask) and the resulting reaction mixture was allowed to stir at room temperature for 1 hour. Then NaCNBH₃ (326.97 mg, 5.20 mmol, 2 equiv.) was added to the reaction mixture at 0° C. and it was allowed to stir at room temperature for 12 hours. Upon completion, monitored by thin-layer chromatography (20% ethyl acetate in hexane), the volatiles were removed under vacuum and the crude was extracted with ethyl acetate (2×50 ml), washed with water, followed by brine solution. The combined organic layer was dried over anhydrous sodium sulphate, filtered and the solvent was removed under vacuum to afford the crude 1-(4-fluorobenzofuran-5-yl)N-methylpropan-2-amine (16-8) as yellow sticky gum (500 mg, 92%). Proceed for the next step with this crude without further purification. LCMS: Rt 1.39 min. MS (ES) C₁₂H₁₄FNO, requires 207.11, found 208.11 [M+H]+.

### Step-7:

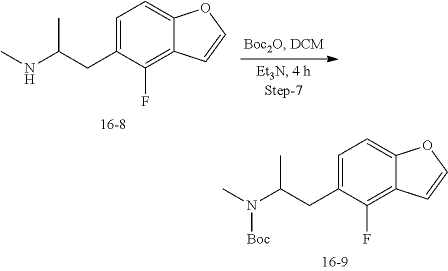 [SMILES: CNC(C)Cc1ccc2occc2c1F ; CC(Cc1ccc2occc2c1F)N(C)C(=O)OC(C)(C)C]

To a stirred solution of crude 1-(4-fluorobenzofuran-5-yl)N-methylpropan-2-amine (16-8) (500 mg, 2.41 mmol, 1 equiv.) in dry dichloromethane (10 ml) was added triethylamine (0.678 ml, 4.82 mmol, 2 equiv.) and Boc anhydride (1.10 ml, 4.82 mmol, 2 equiv.) and the resulting reaction mixture was allowed to stir at room temperature for 4 hours. Upon completion, monitored by thin-layer chromatography (10% ethyl acetate in hexane), the reaction mixture was extracted with dichloromethane (2×50 ml), and washed with water, followed by brine solution. Then dried over anhydrous sodium sulphate, the solvent was evaporated under vacuum and purified by silica gel column chromatography using ethyl acetate/hexane (3:97 v/v) as eluent to afford tert-butyl (1-(4-fluorobenzofuran-5-yl)propan-2-yl)(methyl)carbamate (16-9) as yellow sticky gum (440 mg, 59%). ¹H NMR (400 MHz, DMSO-d₆) δ 7.99 (bs, 1H), 7.38 (d, J=7.96 Hz, 1H), 7.15-7.13 (d, J=6.48 Hz, 1H), 7.02 (s, 1H), 4.37-4.35 (bs, 1H), 2.79 (d, J=6.72 Hz, 2H), 2.66 (s, 3H), 1.25-1.02 (m, 12H). LCMS: Rt 4.27 min. MS (ES) C₁₇H₂₂FNO₃, requires 307.16, found 308.2 [M+H]+. Rotamer was observed in NMR.

### Step-8:

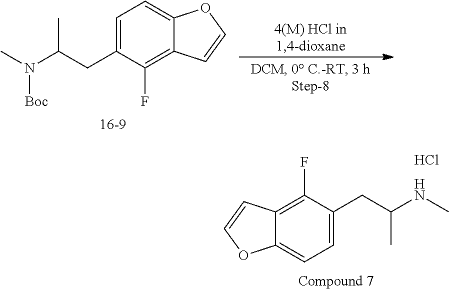 [SMILES: CC(Cc1ccc2occc2c1F)N(C)C(=O)OC(C)(C)C ; "4(M) HCl in1,4-dioxane" ; Cl ; CNC(C)Cc1ccc2occc2c1F]

To a stirred solution of tert-butyl (1-(4-fluorobenzofuran-5-yl)propan-2-yl)(methyl)carbamate (16-9) (440 mg, 1.43 mmol, 1 equiv.) in dry dichloromethane (10 ml) was added (4M) HCl in 1,4 dioxane (3.57 ml, 14.31 mmol, 10 equiv.) at 0° C. and the resulting reaction mixture was allowed to stir at room temperature for 3 hours. Upon completion of reaction (monitored by thin-layer chromatography, 10% ethyl acetate in hexane), the solvent was evaporated, and the crude was washed twice with 1% methanol-diethyl ether (2×30 ml) and dried under vacuum to afford 1-(4-fluorobenzofuran-5-yl)N-methylpropan-2-amine hydrochloride (Compound 7) as a white solid (300 mg, 86%). ¹HNMR (400 MHz, DMSO-d⁶) δ 8.87 (bs, 2H), 8.06 (d, J=1.8 Hz, 1H), 7.49 (d, J=8.36 Hz, 1H), 7.31-7.27 (t, J=7.8 Hz, 7.84 Hz, 1H), 7.07 (s, 1H), 3.39 (bs, 1H), 3.21-3.18 (m, 1H), 2.90-2.84 (m, 1H), 2.60 (s, 3H), 1.12 (d, J=6.36 Hz, 3H). LCMS: Rt 1.36 min. MS (ES) C₁₂H₁₅ClFNO, requires 207.11, found 208.08 [M+H]+. HPLC: Rt 5.47 min. Purity (λ 220 nm): 98.83%.

### Alternative Route for Preparation of 16-7

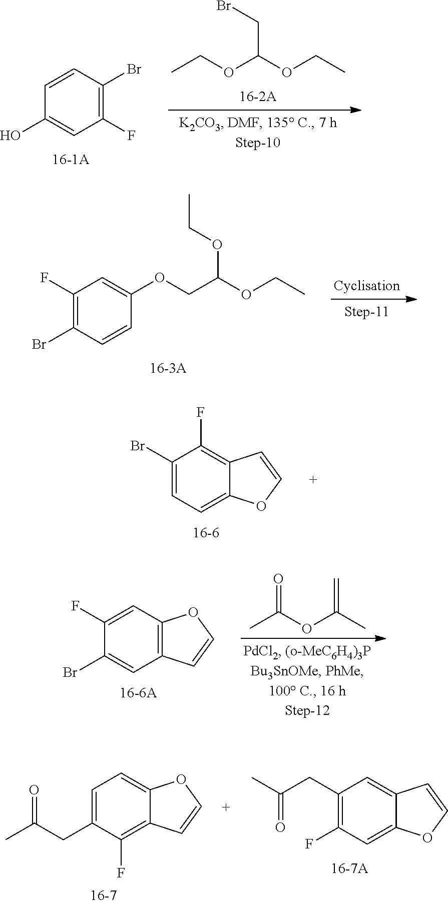

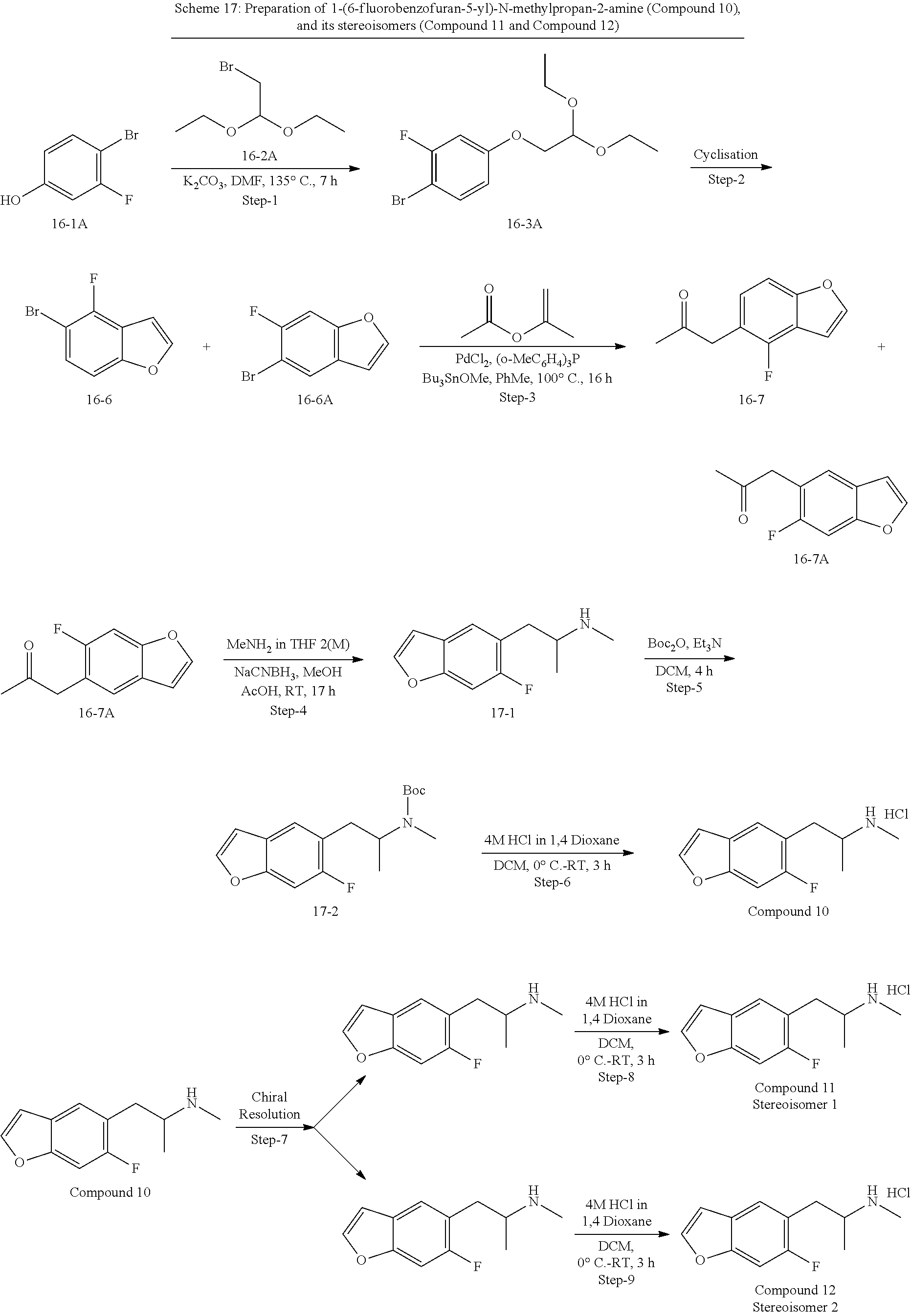

### Step-1:

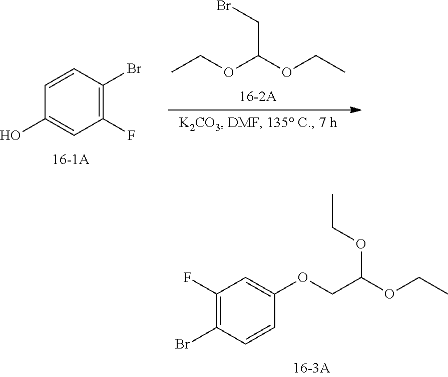

To a stirred solution of 4-bromo-3-fluorophenol (16-1A) (20 g, 104.71 mmol, 1 equiv.) in dry dimethylformamide (200 ml) was added K₂CO₃ (15.91 g, 115.18 mmol, 1.1 equiv.) and 2-bromo-1,1-diethoxyethane (16-2A) (14.43 ml, 107.71 mmol, 1 equiv.) then the resulting reaction mixture was heated to 135° C. for 7 hours. Upon completion, monitored by thin-layer chromatography (10% ethyl acetate in hexane), the reaction mixture was extracted with ethyl acetate (2×500 ml), and washed with water, followed by brine solution. The combined organic layer was dried over anhydrous sodium sulphate. The solvent was removed under vacuum and purified by silica gel column chromatography using ethyl acetate/hexane (5:95 v/v) as eluent to afford 1-bromo-4-(2,2-diethoxyethoxy)-2-fluorobenzene (16-3A) as a colorless sticky gum (25 g, 77%). ¹H NMR (400 MHz, DMSO-d⁶) δ 7.58-7.53 (t, J=8.64 Hz, 1H), 7.10-7.06 (dd, J=2.84 Hz, 11.04 Hz, 1H), 6.82-6.79 (dd, J=2.08 Hz, 8.88 Hz, 1H), 4.80-4.77 (t, J=5.16 Hz, 1H), 3.98 (d, J=5.16 Hz, 2H), 3.69-3.62 (m, 2H), 3.59-3.51 (m, 2H), 1.14-1.11 (m, 6H). GCMS: Rt 6.52 min. MS (ES) C₁₂H₁₆BrFO₃ requires 306, found 306.1 [m/z].

### Step-2:

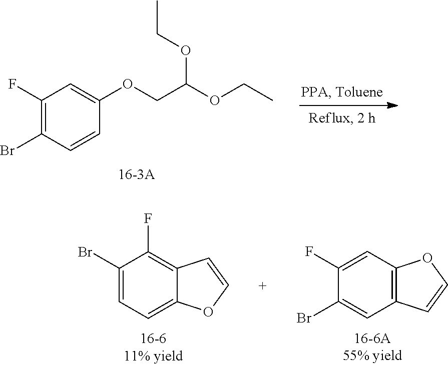 [SMILES: CCOC(COc1ccc(Br)c(F)c1)OCC ; Fc1c(Br)ccc2occc12 ; Fc1cc2occc2cc1Br]

Polyphosphoric Acid (9.57 g, 97.67 mmol, 3 equiv.) was taken in a dry two-neck round bottom flask and was added dry toluene (70 ml) followed by the addition of 1-bromo-4-(2,2-diethoxyethoxy)-2-fluorobenzene (16-3A) (10 g, 32.55 mmol, 1 equiv.) and the resulting reaction mixture was allowed to reflux for 2 hours. Reaction was monitored by thin-layer chromatography (5% ethyl acetate in hexane), and upon completion, the reaction mixture was extracted with ethyl acetate (2×200 ml) and washed with water. Then again washed with aqueous NaOH solution followed by brine solution. The combined organic layer was dried over anhydrous sodium sulphate. The solvent was removed under vacuum to afford crude 5-bromo-6-fluorobenzofuran (16-6A) as yellow sticky gum (3.85 g, 55%), and 5-bromo-4-fluorobenzofuran (16-6) (770 mg, 11%). GCMS (Desired compound): Rt 9.35 min. MS (ES) C₈H4BrFO requires 214, found 214 [m/z].

### Step-2:

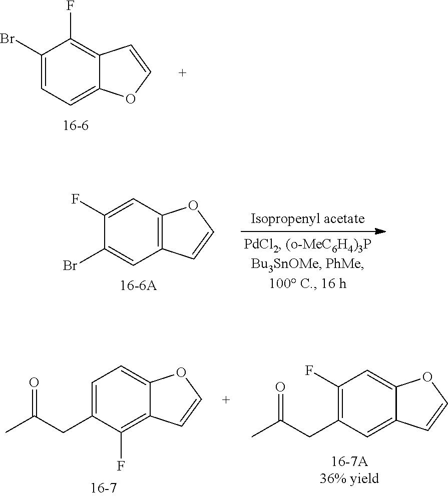

To a stirred solution of 5-bromo-6-fluorobenzofuran (16-6A) (contaminated with 16-6 in 5:1 ratio) (5 gm, 23.25 mmol, 1 equiv.) in dry Toluene (100 ml) was added tri(o-tolyl)phosphine (0.707 g, 2.32 mmol, 0.1 equiv.), tributyl tin methoxide (10.17 ml, 34.88 mmol, 1.5 equiv.) and isopropenyl acetate (3.88 ml, 34.88 mmol, 1.5 equiv.) and the resulting reaction mixture was degassed under argon for 10 minutes. Then palladium (II) chloride (0.41 g, 2.32 mmol, 0.1 equiv.) was added to the reaction mixture and the resulting reaction mixture was heated to 100° C. for 16 hours. The reaction was monitored by thin-layer chromatography (10% ethyl acetate in hexane), and upon completion, the reaction mixture was filtered through a celite bed, water was added to it then extracted with ethyl acetate (2×100 ml). The organic layer was collected and stirred with saturated potassium fluoride solution for 1 hour. The organic layer was collected and washed with brine solution. The combined organic layer was dried over anhydrous sodium sulphate. The solvent was removed under vacuum and purified by silica gel column chromatography using ethyl acetate/hexane (5:95 v/v) as eluent to afford 1-(6-fluorobenzofuran-5-yl) propan-2-one (16-7A) as a light-yellow sticky gum (1.6 g, 36%). ¹H NMR (400 MHz, MeOD) δ 7.72 (d, J=2.2 Hz, 1H), 7.43 (d, J=7.36 Hz, 1H), 7.28 (d, J=9.92 Hz, 1H), 6.79 (d, J=1.6 Hz, 1H), 3.86 (d, J=1.24 Hz, 2H), 2.19 (s, 3H). GCMS: Rt 10.08 min. MS (ES) C₁₁H₉FO₂ requires 192, found 192 [m/z].

### Step-4:

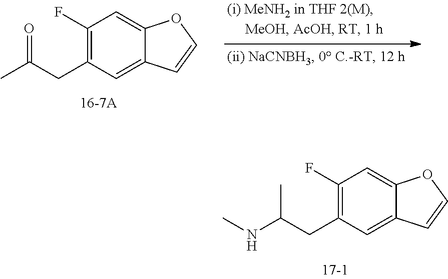

To a stirred solution of 1-(6-fluorobenzofuran-5-yl)propan-2-one (16-8) (2.5 g, 13.00 mmol, 1 equiv.) in dry methanol (50 ml) was added acetic acid (0.74 ml, 13.00 mmol, 1 equiv.) and methyl amine in tetrahydrofuran (2 M) (13 ml, 26.01 mmol, 2 equiv.) (in a sealed Round bottom flask) and the resulting reaction mixture was allowed to stir at room temperature for 1 hour. Then NaCNBH₃ (1.63 g, 26.01 mmol, 2 equiv.) was added to the reaction mixture at 0° C. and then it was allowed to stir at room temperature for 12 hours. Upon completion, monitored by thin-layer chromatography (20% ethyl acetate in hexane), the volatiles were removed under vacuum and the crude was extracted with ethyl acetate (2×50 ml), washed with water, followed by brine solution. The combined organic layer was dried over anhydrous sodium sulphate, solvent was removed under vacuum to afford the crude 1-(6-fluorobenzofuran-5-yl)N-methylpropan-2-amine (17-1) as a yellow sticky gum (2.5 g, 92%). Proceed to the next step with the crude without further purification. LCMS: Rt 2.30 min. MS (ES) C₁₂H₁₄FNO, requires 207, found 208 [M+H]+.

### Step-5:

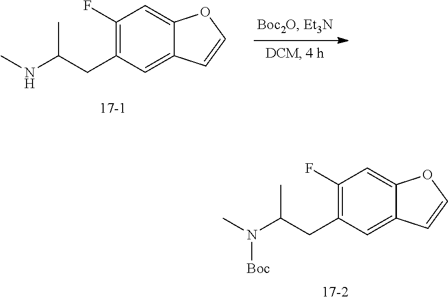 [SMILES: CNC(C)Cc1cc2ccoc2cc1F ; CC(Cc1cc2ccoc2cc1F)N(C)C(=O)OC(C)(C)C]

To a stirred solution of crude 1-(6-fluorobenzofuran-5-yl)-methylpropan-2-amine (17-1) (2.5 g, 12.06 mmol, 1 equiv.) in dry dichloromethane (25 ml) was added triethylamine (3.39 ml, 24.12 mmol, 2 equiv.) and Boc anhydride (5.54 ml, 24.12 mmol, 2 equiv.). The resulting reaction mixture was allowed to stir at room temperature for 4h. Upon completion, monitored by thin-layer chromatography (10% ethyl acetate in hexane), the reaction mixture was extracted with dichloromethane (2×100 ml), washed with water, followed by brine solution, and dried over anhydrous sodium sulphate. The solvent was collected and evaporated under vacuum and purified by silica gel column chromatography using ethyl acetate/hexane (5:95 v/v) as eluent to afford tert-butyl (1-(6-fluorobenzofuran-5-yl)propan-2-yl)(methyl)carbamate (17-2) as a yellow sticky gum (2.2 g, 59%). ¹H NMR (400 MHz, DMSO-d⁶) δ 7.94 (s, 1H), 7.50-7.42 (m, 2H), 6.90 (s, 1H), 4.42-4.34 (m, 1H), 2.77-2.75 (d, J=7.08 Hz, 2H), 2.66 (s, 3H), 1.16-104 (m, 12H). LCMS: Rt 3.74 min. MS (ES) C₁₇H₂₂FNO₃, requires 307, found 308 [M+H]+. Rotamer was observed in NMR.

### Step-6:

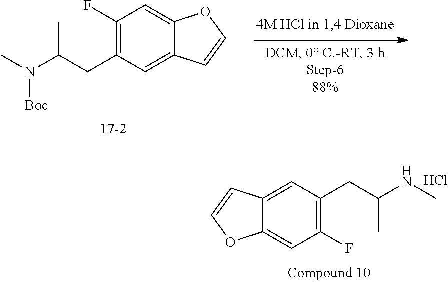 [SMILES: CC(Cc1cc2ccoc2cc1F)N(C)C(=O)OC(C)(C)C ; Cl ; CNC(C)Cc1cc2ccoc2cc1F]

To a stirred solution of tert-butyl (1-(6-fluorobenzofuran-5-yl)propan-2-yl)(methyl)carbamate (17-2) (1 g, 3.25 mmol, 1 equiv.) in dry dichloromethane (15 ml) was added 4(M) HCl in 1,4 dioxane (8.13 ml, 32.53 mmol, 10 equiv.) at 0° C. and the resulting reaction mixture was allowed to stir at room temperature for 3 hours. Upon completion of reaction (monitored by thin-layer chromatography, 10% ethyl acetate in hexane), the solvent was evaporated, and the crude was washed twice with 1% methanol-diethyl ether (2×30 ml) and dried under vacuum to afford 1-(6-fluorobenzofuran-5-yl)N-methylpropan-2-amine hydrochloride (Compound 10) as a white solid (700 mg, 88%). ¹HNMR (400 MHz, DMSO-d⁶) δ 9.01 (bs, 2H), 8.02 (d, J=2.16 Hz, 1H), 7.63-7.57 (m, 2H), 6.96 (q, 1H), 3.38-3.33 (m, 1H), 3.24-3.20 (dd, J=4.24 Hz, 4.16 Hz, 1H), 2.87-2.81 (q, 1H), 2.59-2.57 (t, J=5.2 Hz, 5.24 Hz, 3H), 1.13-1.11 (d, J=6.48 Hz, 3H). LCMS: Rt 1.38 min. MS (ES) C₁₂H₁₅ClFNO, requires 207, found 208 [M+H]+. HPLC: Rt 6.276 min. Purity (λ 210 nm): 98.69%.

### Step-7:

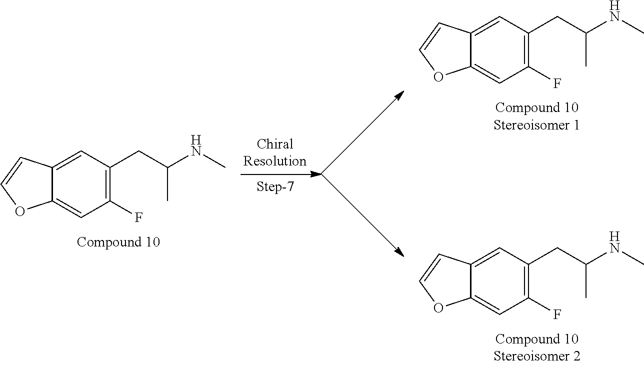

Chiral resolution of Compound 10 was done by Chiral-HPLC.

### Method:

Column Name—CHIRALPAK IG (250×21) mm, 5μ
Flow rate—21 mL/min.
Mobile phase—Hexane/EtOH/IP AMINE: 95/05/0.1
Solubility—Methanol+dichloromethane
Wavelength −240 nm

500 mg of Compound 10 was submitted and after chiral resolution ˜200 mg of stereoisomer 1 (peak 1) and ˜150 mg of stereoisomer 2 (peak 2) were obtained, and ˜70 mg of racemic mixture was recovered. Peak 1 was obtained at 7.970 min. Peak 2 was obtained at 10.556 min.

### Step-8:

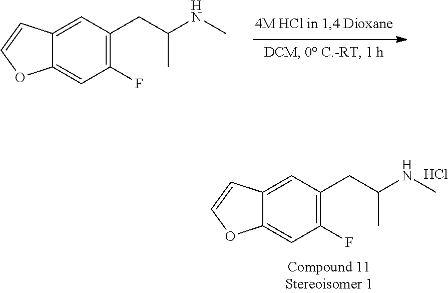

To a stirred solution of stereoisomer 1 (200 mg, 0.96 mmol, 1 equiv.) in dry dichloromethane (5 ml) was added 4(M) HCl in 1,4 dioxane (0.24 ml, 0.96 mmol, 1 equiv.) at 0° C. and the resulting reaction mixture was allowed to stir at room temperature for 1 hour. Upon completion of reaction (monitored by thin-layer chromatography, 10% methanol-dichloromethane), the solvent was evaporated, and the crude was washed twice with 1% methanol-diethyl ether (2×30 ml) and dried under vacuum to afford Compound 11 as a white solid (190 mg, 80%). ¹HNMR (400 MHz, DMSO-d⁶) δ 8.94 (bs, 2H), 8.02 (s, 1H), 7.63-7.57 (m, 2H), 6.96 (s, 1H), 3.37-3.36 (m, 1H), 3.24-3.20 (dd, J=3.8 Hz, 3.72 Hz, 1H), 2.87-2.81 (m, 1H), 2.59 (s, 3H), 1.13-1.11 (d, J=6.32 Hz, 3H). LCMS: Rt 1.38 min. MS (ES) C₁₂H₁₅ClFNO, requires 207, found 208 [M+H]+. HPLC: Rt 6.280 min. Purity (λ 210 nm): 99.87%. Chiral purity: 100%; ee: 100%.

### Step-9:

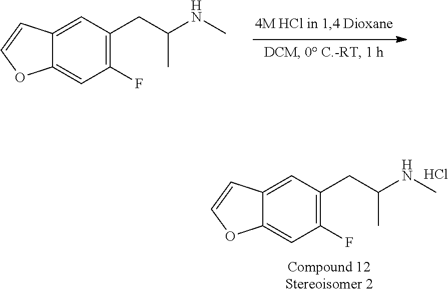

To a stirred solution of stereoisomer 2 (150 mg, 0.72 mmol, 1 equiv.) in dry dichloromethane (5 ml) was added (4 M) HCl in 1,4 dioxane (0.18 ml, 0.72 mmol, 1 equiv.) at 0° C. and the resulting reaction mixture was allowed to stir at room temperature for 1 hour. Upon completion of the reaction (monitored by thin-layer chromatography, 10% methanol-dichloromethane), the solvent was evaporated, and the crude was washed twice with 1% methanol—diethyl ether (2×30 ml) and dried under vacuum to afford Compound 12 as white solid (140 mg, 79%). ¹HNMR (400 MHz, DMSO-d⁶) δ 8.93 (bs, 2H), 8.02 (s, 1H), 7.63-7.58 (m, 2H), 6.96 (s, 1H), 3.36-3.32 (m, 1H), 3.23-3.20 (d, J=12.84 Hz, 1H), 2.87-2.81 (t, J=10.96 Hz, 11.48 Hz, 1H), 2.59 (s, 3H), 1.13-1.11 (d, J=5.64 Hz, 3H). LCMS: Rt 1.37 min. MS (ES) C₁₂H₁₅ClFNO, requires 207, found 208 [M+H]⁺. HPLC: Rt 6.287 min. Purity (λ 210 nm): 99.87%. Chiral purity: 99.93%; ee: 99.86%.

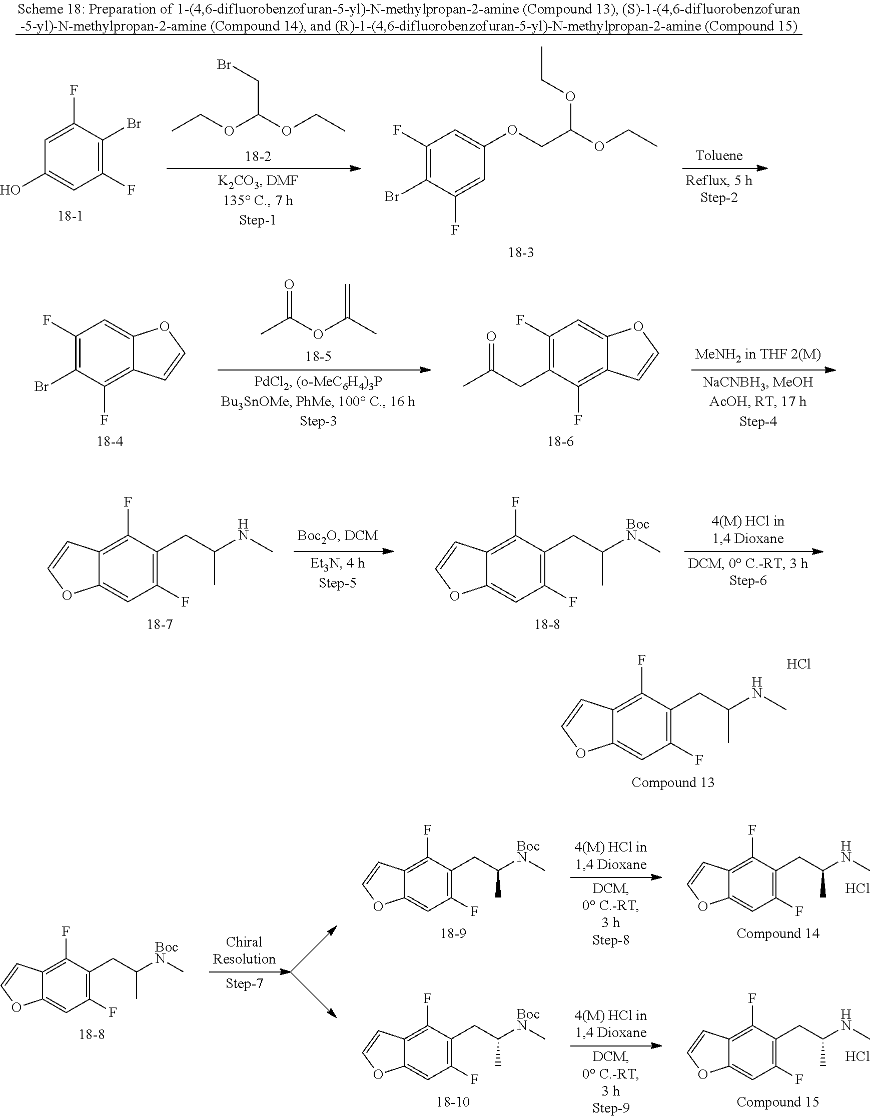

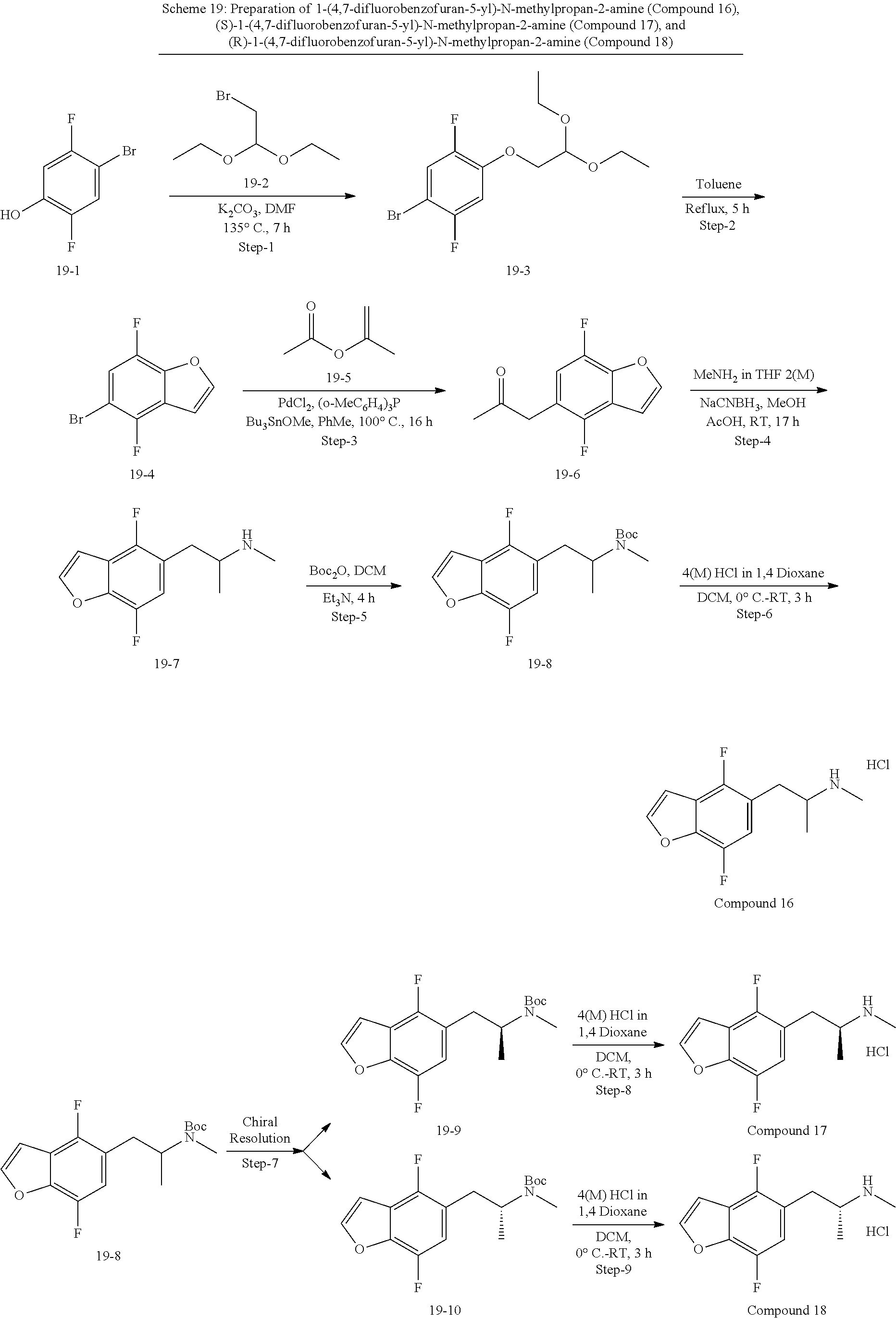

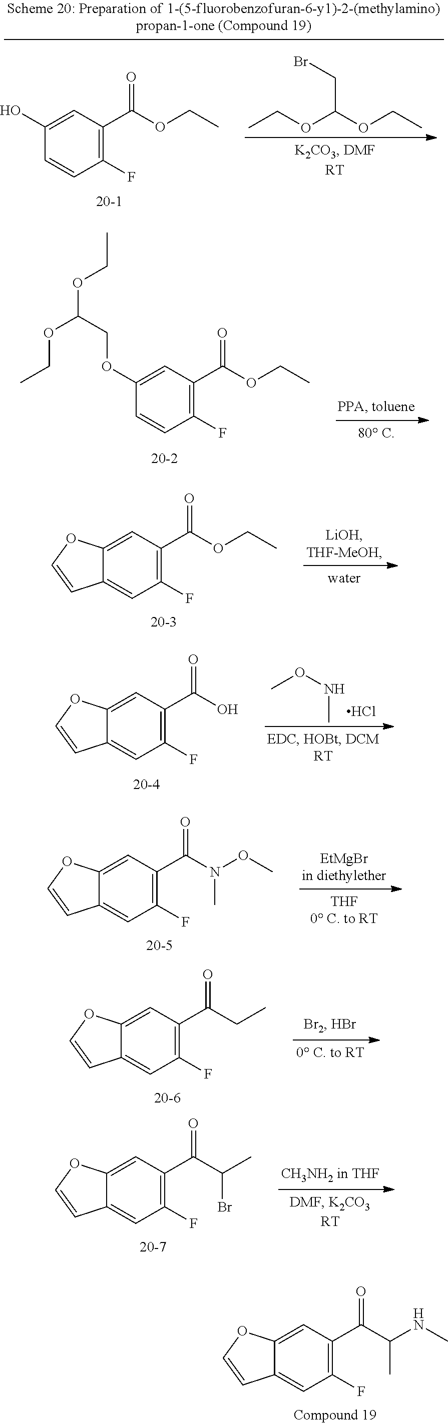

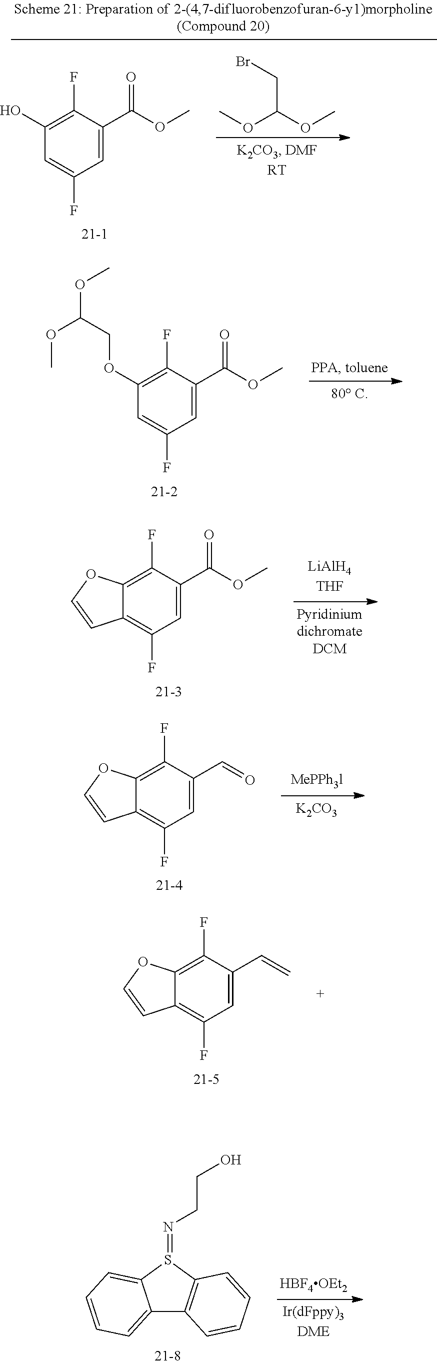

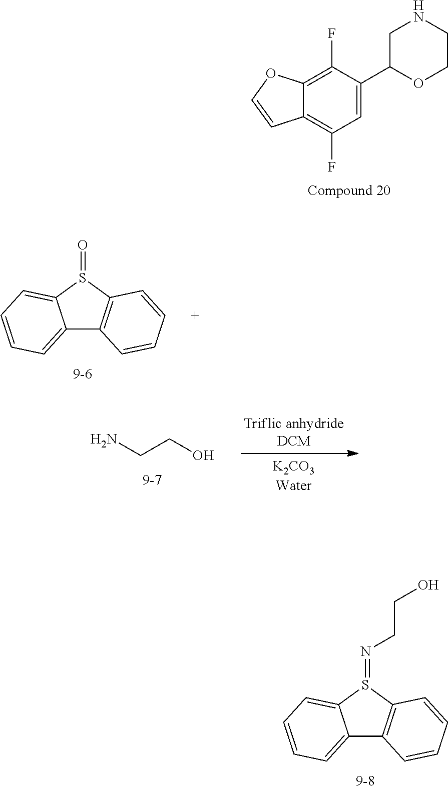

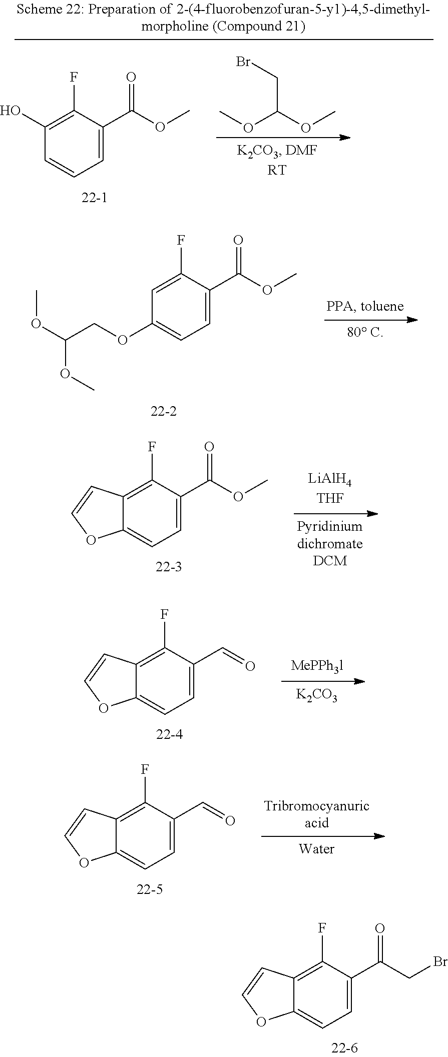

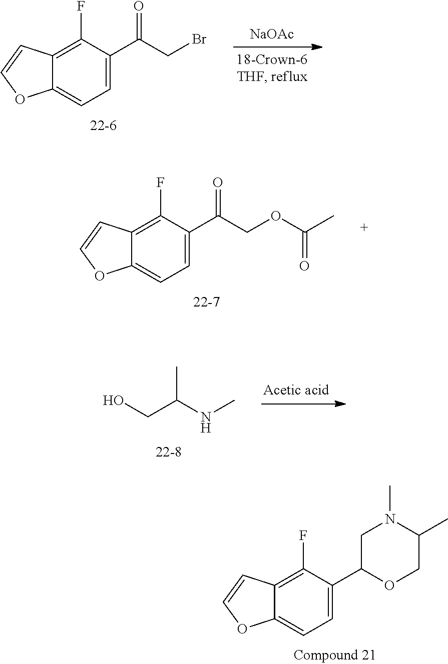

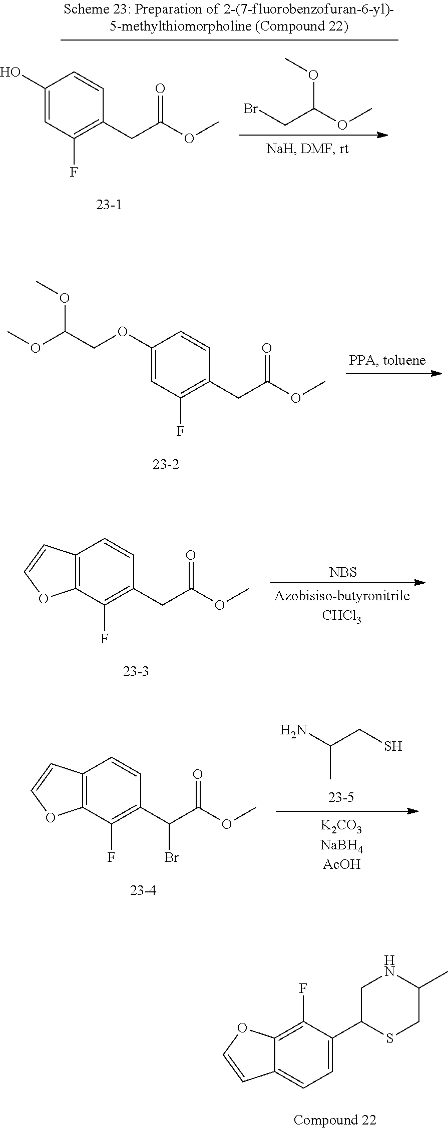

### Example 2: Evaluation of Therapeutic Properties

The clinical and therapeutic effects of compounds that increase extracellular monoamine neurotransmitters are thought to be correlated with their relative tendencies to increase serotonin and dopamine. Liechti and colleagues have proposed that new psychoactive drugs can be classified based on their DAT/SERT inhibition ratios, defined as 1/IC₅₀ at DAT divided by 1/IC₅₀ at SERT (e.g., Luethi and Liechti. 2020. Archives of toxicology, 94(4), pp. 1085-1133). These authors use IC₅₀ measuring uptake inhibition rather than EC₅₀ measuring neurotransmitter release, presumably because drugs that release neurotransmitter also have measurable effects in uptake inhibition assays, producing a metric that can accommodate both reuptake inhibitors and releasers.

In the classification system of Liechti and colleagues, DAT/SERT ratios >1 are thought to predict psychostimulant effects and compounds with this profile have potential value in treating attention deficit hyperactivity disorder (ADHD) and stimulant use disorders. Example compounds with this profile include dextroamphetamine and methylphenidate (Ritalin, Concerta).

In contrast, serotonin release and a DAT/SERT ratio of 0.01-0.1 is said to result in a psychoactive drug profile similar to that of MDMA, which includes feelings of emotional openness, authenticity, and decreased neuroticism. MDMA is an experimental adjunct to psychotherapy that shows great potential for treating PTSD and substance use disorders. It may also be able to generally accelerate progress in psychotherapy and aid emotional decision making. MDMA has a reported DAT/SERT ratio of 0.08 (Simmler and Liechti, New Psychoactive Substances, pp. 143-164).

Compounds with intermediate DAT/SERT ratios (between 0.1 and 1) appear to sometimes have antidepressant-like or nootropic (cognitive enhancement) qualities and have been proposed as antidepressants, cognitive enhancers, or treatments for substance use disorders. For example, 4-bromomethcathinone (4-BMC, Brephedrone; IUPAC: 1-(4-bromophenyl)-2-(methylamino)propan-1-one) does not have typical psychostimulant effects and has been proposed as a potential antidepressant (Foley and Cozzi. 2003. Drug development research, 60(4), pp. 252-260). The different therapeutic profiles of these intermediate compounds are believed to be at least partially the result of serotonin inhibiting and modifying the stimulating effects of dopamine (Kimmel et al. 2009. Pharmacology Biochemistry and Behavior, 94(2), pp. 278-284; Suyama et al. 2019. Psychopharmacology, 236(3), pp. 1057-1066; Wee et al. 2005. Journal of Pharmacology and Experimental Therapeutics, 313(2), pp. 848-854).

One caveat to Liechti's classification system is that compounds that release neurotransmitter may be misclassified if their relative abilities to release dopamine and serotonin are substantially different from their relative abilities to inhibit uptake of dopamine and serotonin. Compounds that appear misclassified in this manner include 3,4,-methylenedioxyethylamphetamine (MDEA; IUPAC [1-(2H-1,3-benzodioxol-5-yl)propan-2-yl](ethyl)amine), which has a reported DAT/SERT ratio of 3.2 (Simmler et al. 2013. British journal of pharmacology, 168(2), pp. 458-470) but is also reported to have MDMA-like effects in humans (e.g., Hermle et al. 1993. Neuropsychopharmacology, 8(2), pp. 171-176).

Such releasing compounds may be alternatively classified according to their DAT/SERT EC₅₀ ratios, where MDEA has been reported as 0.76 (Rothman et al. 2012. Journal of Pharmacology and Experimental Therapeutics, 341(1), pp. 251-262). In this release-based system, MDMA-like therapeutic effects appear present at ratios below 2, with compounds having DAT/SERT EC₅₀ ratios between 2 and 5 having diminished but often still noticeable MDMA-like effects. These intermediate compounds may prove useful for treating ADHD, substance use disorders, and other conditions in individuals who experience significant anxiety from approved psychostimulant pharmacotherapies such as d-amphetamine. Similar to the IC₅₀ system, compounds with higher DAT/SERT EC₅₀ ratios are potential treatments for ADHD and psychostimulant use disorders.

Although MDMA has significant therapeutic potential, it has a number of features that limit its clinical use and may make it contraindicated for some patients. This includes its moderate abuse liability (likely related to its ability to increase extracellular dopamine), acute hypertensive effects (likely related to its norepinephrine release), variable inter-individual metabolism that includes inhibition of the liver enzyme CYP2D6 (increasing risk of drug-drug interactions), potential to induce hyponatremia in women, oxidative stress (likely related to its extensive, though variable, metabolism and formation of reactive metabolites), ability to produce decreases in SERT density after high doses, diminishing therapeutic benefits with repeated use; and a hangover-like after-effects including poor mood and lowered energy. There is therefore a need for additional pharmacologic agents that have similar therapeutic properties while having different pharmacological profiles compared to MDMA.

Compounds that increase extracellular dopamine also often increase extracellular norepinephrine to a similar or greatest extent. For example, d-methamphetamine has a reported DAT/NET EC₅₀ ratio of 0.5, while d-amphetamine has a ratio of 0.9 (Rothman et al. 2001. Synapse, 39(1), pp. 32-41), indicating both are more potent at increasing norepinephrine than dopamine. Differences in the relative balance of dopamine and norepinephrine increases can yield compounds with valuable therapeutic profiles. Norepinephrine increases contribute to cognitive improvements in ADHD but, in excess, can also lead to cardiovascular changes. Dopamine similarly modulates impulsive action but, in excess, can produce compounds with high abuse liability. Nonetheless, individuals with histories of substance abuse who have desensitized dopamine receptors can benefit from compounds that adequately stimulate these receptors. Thus, there is a need for novel treatment compounds that differently balance therapeutic benefits against cardiovascular and abuse liability side effects.

As previously noted, increases in extracellular serotonin and direct stimulation of serotonin receptors present ways for compounds (or compound combinations) to decrease off-target effects and increase select therapeutic effects. For example, compounds that release dopamine and/or norepinephrine and also stimulate 5-HT_{1A} or 5-HT_{1B} receptors can provide fast acting therapeutic effects on mood and attention while decreasing social anxiety. Similarly, compounds that stimulate 5-HT_{2A} receptors while increasing extracellular neurotransmitter can provide the therapeutic benefits of 5-HT_{2A} agonists while having predictable positive effects on mood that decrease the need for clinical monitoring.

### Example 3: Serum Serotonin Concentrations to Index Drug Interactions with the Serotonin Transporter (SERT, SLC6A4)

Serum serotonin can be measured using High Performance Liquid Chromatography and Fluorescence Detection. Venipuncture collects at least 1 mL of sample, which is spun with serum frozen to below −20° C. within 2 hours of collection. For active compounds, assay results will show increases in serum serotonin, indicating that the compound is a releaser of serotonin.

### Example 4: Human Serotonin Transporter (SERT, SLC6A4) Functional Antagonist Uptake Assay

Human recombinant serotonin transporter expressed in HEK-293 cells are plated. Test compound and/or vehicle is preincubated with cells (1×10E5/ml) in modified Tris-HEPES buffer pH 7.1 for 20 minutes at 25° C. and 65 nM. [3H]Serotonin is then added for an additional 15 minute incubation period. Bound cells are filtered and counted to determine [3H]Serotonin uptake. Compounds are screened at concentrations from 10 to 0.001 μM or similar. Reduction of [3H]Serotonin uptake relative to 1 M fluoxetine indicates inhibitory activity.

### Example 5: Monoamine Transporter Uptake and Release Assays

An alternative, invasive method of measuring compound interactions with the serotonin, dopamine, or norepinephrine transporter can be conducted according to the methods of Solis et al (2017. Neuropsychopharmacology, 42(10), 1950-1961) and Rothman and Baumann (Partilla et al. 2016. In: Bonisch S, Sitte H H (eds) Neurotransmitter Transporters Springer; New York, pp 41-52).

Male Sprague-Dawley rats (Charles River, Kingston, NY, USA) are used for the synaptosome assays. Rats are group-housed with free access to food and water, under a 12 h light/dark cycle with lights on at 0700 h. Rats are euthanized by CO2 narcosis, and synaptosomes prepared from brains using standard procedures (Rothman, R. B., & Baumann, M. H. (2003). Monoamine transporters and psychostimulant drugs. European journal of pharmacology, 479(1-3), 23-40). Transporter uptake and release assays are performed as described previously (Solis et al. (2017). N-Alkylated analogs of 4-methylamphetamine (4-MA) differentially affect monoamine transporters and abuse liability. Neuropsychopharmacology, 42(10), 1950-1961). In brief, synaptosomes are prepared from caudate tissue for dopamine transporter (DAT) assays, and from whole brain minus caudate and cerebellum for norepinephrine transporter (NET) and serotonin (5-HT) transporter (SERT) assays.

For uptake inhibition assays, 5 nM [3H]dopamine, [3H]norepinephrine, or [3H]5-HT are used for DAT, NET, or SERT assays respectively. To optimize uptake for a single transporter, unlabeled blockers are included to prevent the uptake of [3H]transmitter by competing transporters. Uptake inhibition is initiated by incubating synaptosomes with various doses of test compound and [3H]transmitter in Krebs-phosphate buffer. Uptake assays were terminated by rapid vacuum filtration and retained radioactivity is quantified with liquid scintillation counting (Baumann et al. (2013). Powerful cocaine-like actions of 3, 4-methylenedioxypyrovalerone (MDPV), a principal constituent of psychoactive ‘bath salts’ products. Neuropsychopharmacology, 38(4), 552-562).

For release assays, 9 nM [3H]MPP+ is used as the radiolabeled substrate for DAT and NET, whereas 5 nM [3H]5-HT is used for SERT. Alternatively [3H]dopamine and [3H]norepinephrine may be used for DAT and NET assays, respectively. All buffers used in the release assay contain 1 M reserpine to block vesicular uptake of substrates. The selectivity of release assays is optimized for a single transporter by including unlabeled blockers to prevent the uptake of [3H]MPP+ or [3H]5-HT by competing transporters. Synaptosomes are preloaded with radiolabeled substrate in Krebs-phosphate buffer for 1 h to reach steady state. Release assays are initiated by incubating preloaded synaptosomes with various concentrations of the test drug. Release is terminated by vacuum filtration and retained radioactivity quantified by liquid scintillation counting.

Effects of test drugs on release are expressed as a percent of maximal release, with maximal release (i.e., 100% Emax) defined as the release produced by tyramine at doses that evoke the efflux of all ‘releasable’ tritium by synaptosomes (10 μM tyramine for DAT and NET assay conditions, and 100 μM tyramine for SERT assay conditions). Effects of test drugs on uptake inhibition and release are analyzed by nonlinear regression. Dose-response values for the uptake inhibition and release are fit to the equation, Y(x)=Ymin+(Ymax−Ymin)/(1+10 exp[(log P50−log x)]×n), where x is the concentration of the compound tested, Y(x) is the response measured, Ymax is the maximal response, P50 is either IC50 (the concentration that yields half-maximal uptake inhibition response) or EC50 (the concentration that yields half-maximal release), and n is the Hill slope parameter. EC50s for release of less than 10 uM, but often less than 1 uM, are usually considered indicative of substrate-type releasers.

### Example 6: Marble Burying Measure of Decreased Anxiety and Neuroticism

The marble burying test is a model of neophobia, anxiety, and obsessive-compulsive behavior. Moreover, it has been proposed to have predictive validity for the screening of novel antidepressants and anxiolytics. It is well established to be sensitive to the effects of SSRIs as well as serotonin releasers such as fenfluramine and MDMA (De Brouwer et al., Cognitive, Affective, and Behavioral Neuroscience, 2019, 19(1), 1-39).

The test involves the placement of a standardized number of marbles gently onto the surface of a layer of bedding material within a testing arena. Mice are then introduced into the arena for a standardized amount of time and allowed to explore the environment. The outcome measure of the test is the number of marbles covered, as scored by automatic scoring software or blinded observers. General locomotor activity, often operationalized as total distance traveled, is often used as a control measure. A compound that attenuates anxiety, neuroticism, or obsessive-compulsive behavior decreases marble burying. A halogenated benzofuran compound is given to mice and decreases in marble burying, indicates an acute decrease in anxiety and neuroticism.

### Example 7: Neuroplasticity Assay in Primary Cortical Neurons

Halogenated benzofuran compounds may be considered psychoplastogens, that is, small molecules that are able to induce rapid neuroplasticity (Olson, 2018, Journal of experimental neuroscience, 12, 1179069518800508). One exemplary method for measuring this, a neurite outgrowth assay conducted in murine primary cortical neurons, is provided below. Other methods are well known in the literature (e.g. Olson, 2018, Journal of experimental neuroscience, 12, 1179069518800508; Ly et al. Cell reports 23, no. 11 (2018): 3170-3182; and references therein).

Primary cortical neurons are prepared from timed pregnant wild-type C57BL/6JRccHsd mice at E18. Animals are sacrificed (see section 3.3.1) and embryos are dissected in Calcium and Magnesium free Hanks Balanced Salt Solution (CMF-HBSS) containing 15 mM HEPES and 10 mM NaHCO₃, pH 7.2. Embryos are decapitated, skin and skull gently removed and hemispheres are separated. After removing meninges and brain stem, the hippocampi are isolated, chopped with a sterile razor blade in Chop solution (Hibernate-E without Calcium containing 2% B-27) and digested in 2 mg/mL papain (Worthington) dissolved in Hibernate-E without Calcium for 30 minutes (±5 min) at 30° C. Hippocampi are triturated for 10-15 times with a fire-polished silanized Pasteur pipette in Hibernate-E without Calcium containing 2% B-27, 0.01% DNaseI, 1 mg/mL BSA, and 1 mg/mL Ovomucoid Inhibitor. Undispersed pieces are allowed to settle by gravity for 1 min and the supernatant is centrifuged for 3 min at 228 g. The pellet is resuspended in Hibernate-E containing 2% B-27, 0.01% DNaseI, 1 mg/ml BSA, 1 mg/mL Ovomucoid Inhibitor and diluted with Hibernate-E containing 2% B-27. After the second centrifugation step (3 min at 228 g), the pellet is resuspended in nutrition medium (Neurobasal, 2% B-27, 0.5 mM glutamine, 1% Penicillin-Streptomycin).

Cells are counted in a hemacytometer and seeded in nutrition medium on poly-D-lysine pre-coated 96-well plates at a density of 2.6×104 cells/well. Cells are cultured at 37° C.; 95% humidity and 5% CO2. All wells are handled the same way.

The experiment is performed in adequate technical replicates for all groups, for example five replicates.

On the day of preparation (DIV1), mouse cortical neurons are seeded on poly-D-lysine pre-coated 96-well plates at a density of 2.6×104 cells per well.

On DIV2, cells are treated with test compounds at concentrations selected based on their EC50 at SERT release or 5-HT receptor agonism for three different time points (4 h, 8 h and 24 h), followed by a complete medium change. Additionally, cells are treated with 40 ng/mL of a positive control (Fibroblast growth factor, FGF) or vehicle control (VC) for 48 h.

The experiment is carried out with several, for example five, technical replicates per condition, vehicle treated cells serve as control.

Treated primary neurons are fixed on DIV4 by addition of equal volume 4% paraformaldehyde (PFA) to the medium at room temperature (RT) for 30 minutes.

Cells are rinsed two times with PBS and are permeabilized with 0.1% Triton X-100 in PBS for 30 minutes at RT. Next, cells are blocked for 90 min at RT with 20% horse serum, 0.1% Triton X-100 in PBS.

Then, samples are incubated with the primary antibody against Beta Tubulin Isotype III at 4° C. overnight.

Next day, cells are further incubated for another 30 min at RT. After three washing steps with PBS, cells are incubated with a fluorescently labelled secondary antibody and DAPI (nucleus) for 1.5 hours at RT in the darkness. Cells are again rinsed four times with PBS and imaged with the Cytation 5 Multimode reader (BioTek). From each well, images are taken at 10× magnification.

Digital images from cortical neurons are analyzed for the following parameter using a software-supported automatic quantification method: Number of neurites, number of branches, total length of neurites and length of the longest neurite. Analysis is performed using HCA-Vision software or similar standard software.

Basic statistical analysis is performed. If appropriate, data are presented as mean±standard error of mean (SEM) and group differences are evaluated by e.g. one or two-way ANOVA or T-test. EC50 may be calculated as described elsewhere.

### Example 8: Evaluation of Entactogenic Effect of Decreased Neuroticism

The entactogenic effect of decreased neuroticism can be measured as a decrease in social anxiety using the Brief Fear of Negative Evaluation-revised (BFNE) (Carleton et al., 2006, Depression and Anxiety, 23(5), 297-303; Leary, 1983, Personality and Social Psychology bulletin, 9(3), 371-375). This 12-item Likert scale questionnaire measures apprehension and distress due to concerns about being judged disparagingly or with hostility by others. Ratings use a five-point Likert scale with the lowest, middle, and highest values labeled with “much less than normal,” “normal,” and “much more than normal.” The BFNE can be administered before and repeatedly during therapeutic drug effects. Participants are instructed to answer how they have been feeling for the past hour, or otherwise during the effect of the drug. Baseline-subtracted responses are typically used in statistical models.

### Example 9: Evaluation of Entactogenic Effect of Authenticity

The entactogenic effect of authenticity can be measured using the Authenticity Inventory (Kernis & Goldman. 2006. Advances in experimental social psychology, 38, 283-357) as modified by Baggott et al (Journal of Psychopharmacology 2016, 30.4: 378-87). Administration and scoring of the instrument is almost identical to that of the BFNE. The Authenticity Inventory consists of the following items, which are each rated on a 1-5 scale, with select items reverse scored as specified by Kernis & Goldman:
I am confused about my feelings.
I feel that I would pretend to enjoy something when in actuality I really didn't.
For better or worse, I am aware of who I truly am.
I understand why I believe the things I do about myself
I want the people with whom I am close to understand my strengths.
I actively understand which of my self-aspects fit together to form my core or true self.
I am very uncomfortable objectively considering my limitations and shortcomings.
I feel that I would use my silence or head-nodding to convey agreement with someone else's statement or position even though I really disagreed.
I have a very good understanding of why I do the things I do.
I am willing to change myself for others if the reward is desirable enough.
I would find it easy to pretend to be something other than my true self.
I want people with whom I am close to understand my weaknesses.
I find it difficult to critically assess myself (unchanged)
I am not in touch with my deepest thoughts and feelings.
I feel that I would make it a point to express to those I am close with how much I truly care for them.
I have difficulty accepting my personal faults, so I try to cast them in a more positive way.
I feel that I idealize the people close to me rather than objectively see them as they truly are.
If asked, people I am close to could accurately describe what kind of person I am.
I prefer to ignore my darkest thoughts and feelings.
I am aware of times when I am not being my true self.
I am able to distinguish the self-aspects that are important to my core or true self from those that are unimportant.
People close to me would be shocked or surprised if they discovered what I am keeping inside me.
It is important for me to understand the needs and desires of those with whom I am close.
I want people close to me to understand the real me, rather than just my public persona or “image”.
I could act in a manner that is consistent with my personally held values, even if others criticized me or rejected me for doing so.
If a close other and I were in disagreement, I would rather ignore the issue than constructively work it out.
I feel that I would do things that I don't want to do merely to avoid disappointing people.
My behavior expresses my values.
I actively attempt to understand myself as well as possible.
I feel that I'd rather feel good about myself than objectively assess my personal limitations and shortcomings.
My behavior expresses my personal needs and desires.
I have on a “false face” for others to see.
I feel that I would spend a lot of energy pursuing goals that are very important to other people even though they are unimportant to me.
I am not in touch with what is important to me.
I try to block out any unpleasant feelings I have about myself.
I question whether I really know what I want to accomplish in my lifetime.
I am overly critical about myself.
I am in touch with my motives and desires.
I feel that I would deny the validity of any compliments that I receive.
I place a good deal of importance on people close to me understanding who I truly am.
I find it difficult to embrace and feel good about the things I have accomplished.
If someone pointed out or focused on one of my shortcomings, I would quickly try to block it out of my mind and forget it.
The people close to me could count on me being who I am, regardless of what setting we were in.
My openness and honesty in close relationships are extremely important to me.
I am willing to endure negative consequences by expressing my true beliefs about things.

### Example 10: Evaluation of Side Effects of Entactogens

Adverse effects of an entactogen include formation of tolerance to entactogens, headache, difficulty concentrating, lack of appetite, lack of energy, and decreased mood. In addition to these mild toxicities, MDMA is associated with a number of more severe toxicities, including but not limited to acute and chronic cardiovascular changes, hepatotoxicity, hyperthermic syndromes, hyponatremia, and neurotoxicity (see the MDMA Investigator's Brochure, 13th Edition: Mar. 22, 2021, and references therein, available from the sponsor of MDMA clinical trials at MAPS.org).

Acute physiological changes can be measured in humans with standard clinical methods (blood pressure cuffs, 3-lead EKG, tympanic or oral temperature, serum sodium, etc), with measures usually collected before and at scheduled intervals after an entactogen. For example, measures may be collected before, 0.5, 0.75, 1, 1.5, 2, 2.5, 3, 4, 5, 6, and 8 hours after an entactogen. Maximum change from baseline and area-under-the-effects-versus-time-curve may be used as summary measures and statistically compared to a placebo control condition.

To measure adverse symptoms, patients can be asked to complete a self-report symptom questionnaire, such as the Subjective Drug Effects Questionnaire (SDEQ) or List of Complaints. The SDEQ is a 272-item self-report instrument measuring perceptual, mood, and somatic changes caused by drugs including hallucinogens like LSD (Katz et al. 1968. J Abnorm Psychology 73:1-14). It has also been used to measure the therapeutic and adverse effects of MDMA (Harris et al. 2002. Psychopharmacology, 162(4), 396-405). The List of Complaints is a 66-item questionnaire that measures physical and general discomfort and is sensitive to entactogen-related complaints (e.g., Vizeli & Liechti. 2017. Journal of Psychopharmacology, 31(5), 576-588).

Alternatively, individual items can be taken from the SDEQ or List of Complaints in order to create more focused questionnaires and reduce the burden of filling out time-consuming paperwork on participants. To measure tolerance formation, a global measure of the intensity of therapeutic effects can be used, such as the question “on a scale from 0 to 100 where 0 is no ‘good drug effect’ and 100 is the most ‘good drug effect’ you have ever felt, how would you rate this drug experience?”

In some embodiments, the questionnaire will be administered approximately 7 hours after a patient takes MDMA or another entactogen (with instructions to answer for the time since taking the entactogen) and then daily (with instructions to answer for the last 24 hours) for up to 96 hours after the entactogen was taken. Decreases in adverse effects of a compound compared to MDMA can be shown by comparing the intensity (for the tolerance question) or prevalence (for other symptom questions) of effects that occur. Prevalence of adverse effects including formation of tolerance to entactogens, headache, difficulty concentrating, lack of appetite, lack of energy, and decreased mood may be decreased by approximately 10%, 20%, 30%, 40%, 50%, 60%, 70%, 80%, 90%, 99%, or 100%.

As an alternative to measuring side effects of entactogens in clinical trials, preclinical studies in rodents may also be used. Appropriate tasks and behaviors that may be used to measure side effects include physiological measures (heart rate, blood pressure, body temperature), the modified Irwin procedure or functional observational battery (Irwin, Psychopharmacologia, 13, 222-257, 1968), and locomotor activity (such as distance traveled, rearing frequency, and rearing duration; Piper et la., J Pharmacol Exp Ther, 317, 838-849, 2006). In these studies, an entactogen is administered at different doses (including a vehicle only placebo) to different groups of animals and measures are made at scheduled times before and after administration. For example, 0, 1.5, 3, 15, and 30 mg/kg of a compound may be administered intraperitoneally and measures made before and 15, 30, 60, 120 and 180 minutes and 12, 24, 36, and 48 hours after administration of the test substance.

While the present invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as described in the appended claims.

I claim:

1. A compound of Formula: 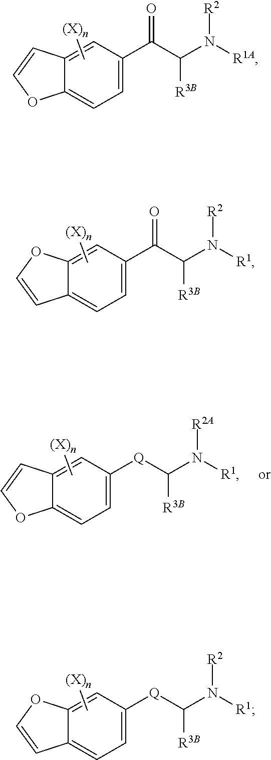
or a pharmaceutically acceptable salt or salt mixture thereof,
wherein:
R¹ and R² are independently selected from the group consisting of H, C₁-C₄ alkyl, C₁-C₄ haloalkyl, —CH₂OH, and —CH₂CH₂OH;
R^{1A} and R^{2A} are independently selected from the group consisting of C₁-C₄ alkyl, C₁-C₄ haloalkyl, —CH₂OH, and —CH₂CH₂OH;
R^{3B} is C₁-C₄ alkyl, C₁-C₄ haloalkyl, —CH₂OH, or —CH₂CH₂OH;
Q is 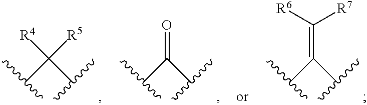
R⁴ and R⁵ are independently selected from the group consisting of H, C₁-C₄ alkyl, C₁-C₄ haloalkyl, —F, —Cl, and —Br;
R⁶ and R⁷ are independently selected from the group consisting of H, C₁-C₄ alkyl, C₁-C₄ haloalkyl, —F, —Cl, and —Br;
each X is independently selected from —F, —Cl, and —Br; and
n is 1 or 2.

2. The compound of claim 1, wherein the compound is of Formula: 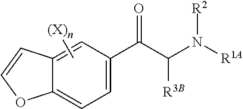
or a pharmaceutically acceptable salt or salt mixture thereof.

3. The compound of claim 1, wherein the compound is of Formula: 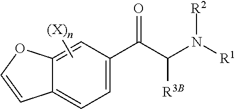
or a pharmaceutically acceptable salt or salt mixture thereof.

4. The compound of claim 1, wherein the compound is of Formula: 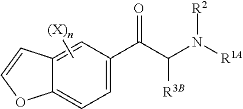
or a pharmaceutically acceptable salt or salt mixture thereof.

5. The compound of claim 1, wherein the compound is of Formula: 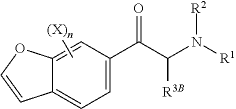
or a pharmaceutically acceptable salt or salt mixture thereof.

6. The compound of claim 1, wherein R¹ is hydrogen.

7. The compound of claim 1, wherein R¹ and R^{1A} are C₁-C₄ alkyl.

8. The compound of claim 1, wherein R¹ and R^{1A} are methyl or ethyl.

9. The compound of claim 1, wherein R² is hydrogen.

10. The compound of claim 1, wherein R² and R^{2A} is C₁-C₄ alkyl.

11. The compound of claim 1, wherein R² and R^{2A} is methyl or ethyl.

12. The compound of claim 1, wherein R^{3B} is C₁-C₄ alkyl.

13. The compound of claim 1, wherein R^{3B} is methyl or ethyl.

14. The compound of claim 1, wherein Q is

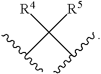

15. The compound of claim 1 selected from the group consisting of: 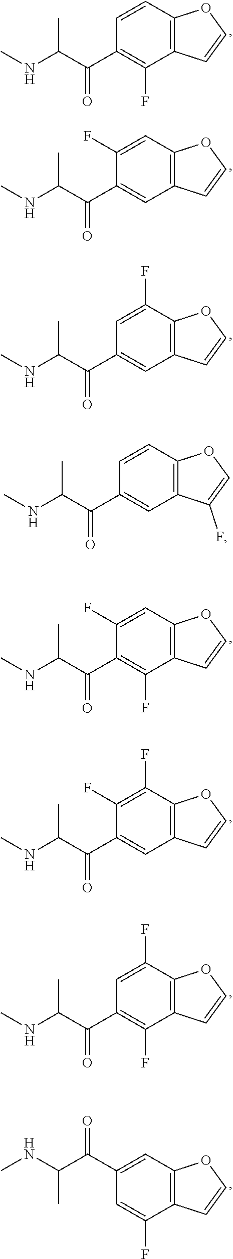 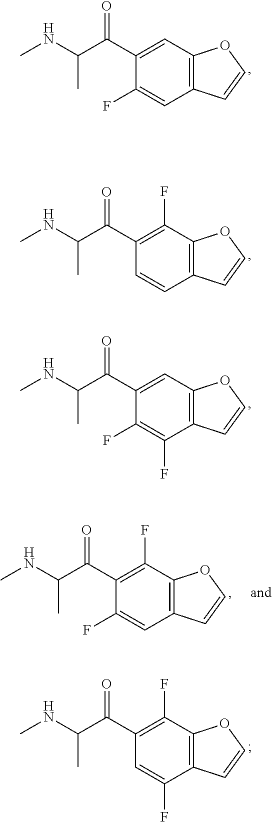 [SMILES: CNC(C)C(=O)c1cc2occc2cc1F ; CNC(C)C(=O)c1ccc2ccoc2c1F ; CNC(C)C(=O)c1cc2occc2c(F)c1F ; CNC(C)C(=O)c1c(F)cc2ccoc2c1F ; CNC(C)C(=O)c1cc(F)c2ccoc2c1F]
or a pharmaceutically acceptable salt or salt mixture thereof.

16. The compound of claim 1 selected from the group consisting of: 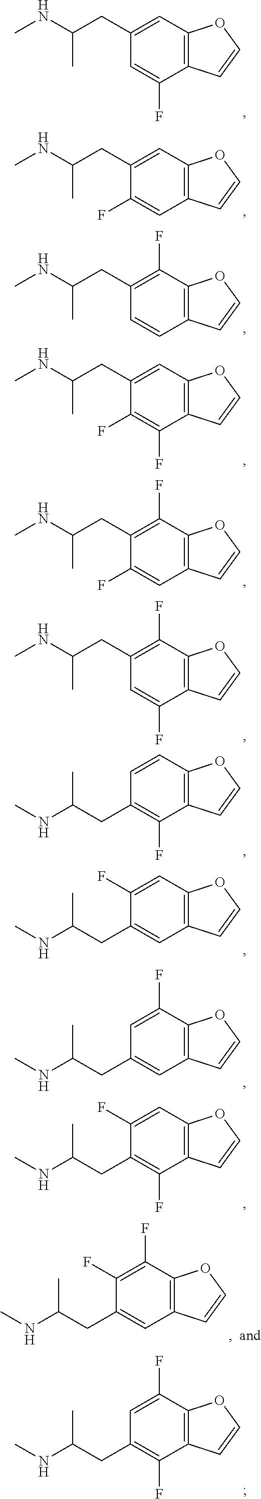
or a pharmaceutically acceptable salt or salt mixture thereof.

17. The compound of claim 1 selected from the group consisting of: 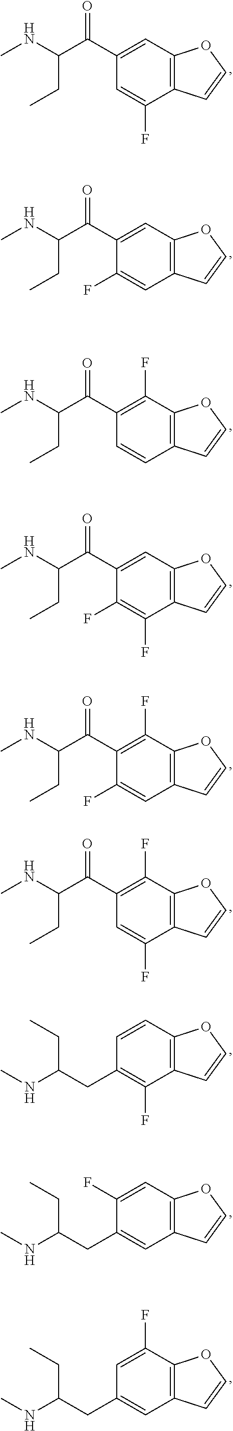 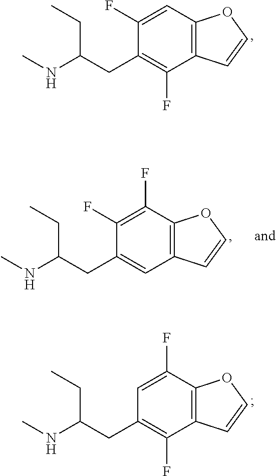 [SMILES: CCC(Cc1c(F)cc2occc2c1F)NC ; CCC(Cc1cc2ccoc2c(F)c1F)NC ; CCC(Cc1cc(F)c2occc2c1F)NC]
or a pharmaceutically acceptable salt or salt mixture thereof.

18. The compound of claim 1 selected from the group consisting of: 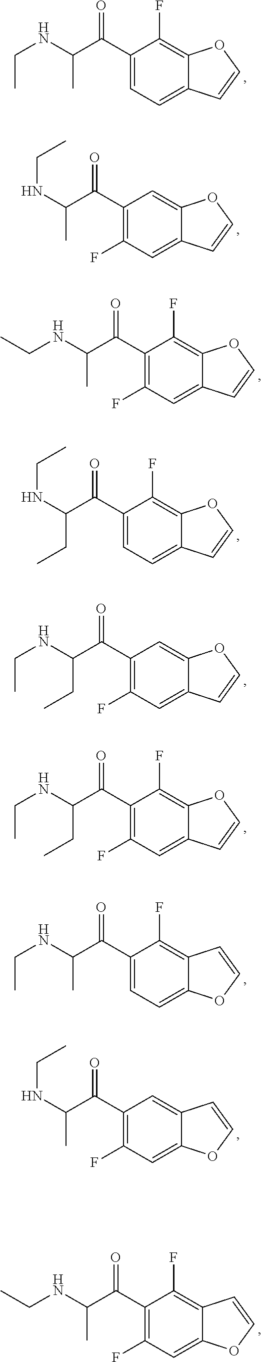 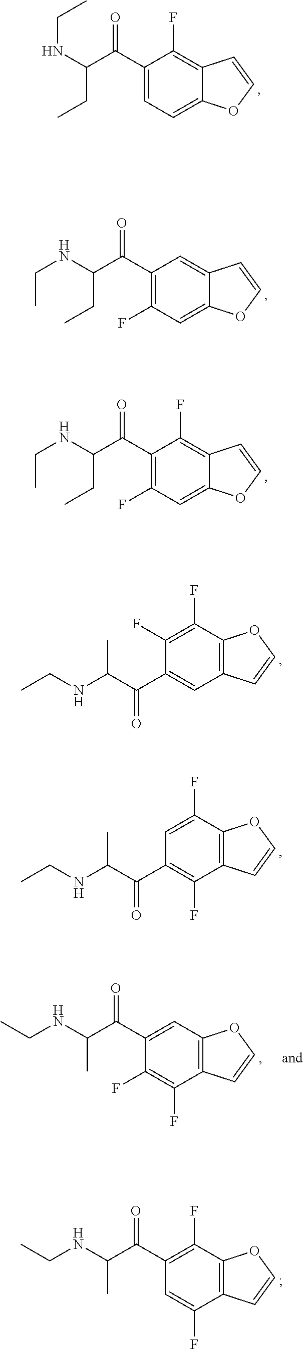
or a pharmaceutically acceptable salt or salt mixture thereof.

19. A pharmaceutical composition comprising a compound of claim 1 or a pharmaceutically acceptable salt or salt mixture thereof and a pharmaceutically acceptable carrier or excipient.

20. A method for treating a central nervous system disorder comprising administering an effective amount of a compound of claim 1 or a pharmaceutically acceptable salt or salt mixture thereof or a pharmaceutical composition thereof to a human patient in need thereof.
